(19) United States
(12) Patent Application Publication
(10) Pub. No.: US 2022/0347285 A1
(43) Pub. Date: Nov. 3, 2022

(54) ATTENUATED DENGUE VIRUSES

(71) Applicant: CODAGENIX INC., Farmingdale, NY (US)

(72) Inventors: Steffen Mueller, Great Neck, NY (US); John Robert Coleman, Blauvelt, NY (US); Charles Stauft, Columbia, MD (US); Ying Wang, South Setauket, NY (US)

(73) Assignee: CODAGENIX INC., Farmingdale, NY (US)

(21) Appl. No.: 17/621,125

(22) PCT Filed: Jun. 23, 2020
(86) PCT No.: PCT/US2020/039166

§ 371 Date: Dec. 20, 2021

Related U.S. Application Data
(60) Provisional application No. 62/866,477, filed on Jun. 25, 2019.

(51) Int. Cl. A61K 39/12; C12N 7/00; A61P 31/14

(52) U.S. Cl. CPC A61K 39/12; C12N 7/00; A61P 31/14; C12N 2770/24121; C12N 2770/24122; C12N 2770/24134; C12N 2770/24162; A61K 2039/5254

(57) ABSTRACT

The present invention provides for modified Flavivirus such as a modified dengue virus type 1, 2, 3, 4, a combination of these, or a tetravalent combination of these. The modification according to various aspects of the invention results in reduced viral protein expression compared to a parent virus, wherein the reduction in expression is the result of recoding one or more regions of the virus. For example, the prM, or envelope (E) region can be recoded. In various embodiments one or more regions are recoded by reducing the codon pair bias or codon usage bias of the protein-encoding sequence. These modified Flaviviruses are used as vaccine compositions to provide a protective immune response.

### CROSS-REFERENCE TO RELATED APPLICATIONS

This application includes a claim of priority under 35 U.S.C. § 119(e) to U.S. provisional patent application No. 62/866,477, filed Jun. 25, 2019, the entirety of which is hereby incorporated by reference.

### FIELD OF THE INVENTION

This invention provides highly attenuated flaviviruses and particularly, attenuated dengue viruses and vaccines. The attenuated viruses provide protective immunity from challenge by virus of the same lineage, as well as cross protection against heterologous viruses.

### BACKGROUND OF THE INVENTION

Dengue virus (DENV) is a single-stranded positive-sense RNA virus belonging to the Flaviviridae family, in the same genus (Flavivirus) that includes Zika virus (ZIKV), West Nile virus (WNV), and yellow fever virus (YFV). According to the World Health Organization (WHO) about 2.5 billion people, nearly half the world's population, are now at risk from DENV and estimates that there may be 50 million cases of DENV infection worldwide every year. DENV is closely related phylogenetically to ZIKV and shares a common mosquito vector, Aedes aegypti, with both ZIKV and Chikungunya virus (an alphavirus). There is special concern for tourists at risk for DENV infection, with an added concern of importation and spread ofthe epidemic to places like the southern U.S. The vector for DENV, Aedes aegypti, is found throughout the southern U.S. Aedes albopictus, the Asian tiger mosquito, is also competent for transmitting DENV. If DENV enters A. albopictus populations in the U.S., it will have a vastly expanded range extending into the American Midwest and Northeast.

Flavivirus vaccine development is compounded by the phenomenon of Antibody-Dependent Enhancement (ADE). ADE occurs when prior infection with one flavivirus predisposes an individual to an enhanced severity of disease upon re-infection with a different serotype. During ADE, antibodies against the first virus bind, but do not neutralize the second virus, instead increasing its infectivity. DENV is prevalent in many countries, thus any vaccine strategy should consider the impact on a population with established dengue immunity. It is worth noting that people with underlying DENV immunity could experience increased adverse events from a live DENV vaccine, since their DENV immunity could enhance infectivity of the DENV vaccine strain, leading to increased adverse events. These are plausible scenarios that needs to be considered when developing a DENV vaccine, whether live, inactivated or antigen/VLP/backbone carrier-based. Because of the high likelihood of enhancing flavivirus infection due to ADE, all vaccine development strategies need to consider how any given DENV vaccine might interact with existing antibodies to a flavivirus or a subsequent flavivirus infection.

Dengue virus comprised four serotypes (DENV1-4) of the mosquito-borne Flaviviruses in the family Flaviviridae. Dengue viruses are enveloped viruses with an icosahedral virion comprised of C (Core), M(Membrane), and E (Envelope) glycoproteins that is 40-65 nanometers in diameter. The Dengue virus genome is a single positive-strand RNA molecule of 10,000-11,000 bases in length encoding structural (C, prM, E) and nonstructural (NS1, NS2, NS3, NS4, and NS5) proteins. Dengue viruses transcribe and replicate their genome in the cell cytoplasm, with the genome translated into a single polypeptide that is cleaved and processed by both host and viral proteins. Dengue virus is an emerging agent of international concern in the tropics and the individual serotypes are genetically as well as antigenically distinct viruses.

Accordingly, there remains a need in the art to develop a vaccine to prevent or reduce infection.

### SUMMARY OF THE INVENTION

It is described herein that recoded dengue viruses made by modification of the E region by large numbers of synonymous nucleotide mutations are highly effective in providing protective immunity against lethal wild type challenge and cross protection against different lineages. Further, the viruses have exceptional safety profiles.

Accordingly, various embodiments of the invention provides an attenuated dengue virus in which expression of viral proteins is reduced through codon-pair deoptimization of the E coding regions. In certain embodiments, E is the only virus protein coding regions targeted. In certain embodiments, when another dengue virus protein encoding region other than E is deoptimized, the reduction is small compared to the reduction of E. According to the invention, reduction in expression of virus proteins of the invention is accomplished by changes in protein encoding sequence, for example by lowering the codon pair bias of the protein-encoding sequence, substituting rare codons, modifying G+C content, modifying CG and/or TA (or UA) dinucleotide content, or combinations. Reduced expression can also be accomplished by modifications to the regulatory sequences of the proteins.

In certain embodiments, reducing the codon-pair bias can comprise identifying a codon pair in the parent protein-encoding sequence having a codon-pair score that can be reduced, and reducing the codon-pair bias by substituting the codon pair with a codon pair that has a lower codon-pair score. In other embodiments, reducing the codon-pair bias comprises rearranging the codons of a parent protein-encoding sequence. In certain embodiments, the E protein-encoding sequence has a codon pair bias less than −0.1, or less than −0.2, or less than −0.3, or less than −0.4. Codon pair bias of a protein-encoding sequence (i.e., an open reading frame) is calculated as described in Coleman et al., 2000 and herein.

In an embodiment of the invention, expression of the E protein-encoding sequence is reduced by replacing one or more codons with synonymous codons that are less frequent in the host.

In an embodiment of the invention, the E protein-encoding sequence DENV serotype are present in a background from a different strain of the same DENV serotype or the homologous strain.

The invention also provides a dengue vaccine composition for inducing a protective immune response in a subject, wherein the vaccine composition comprises virus in which viral translation is reduced while maintaining at least 90% antigenic identity with wt virus. In some embodiments, the viral translation is reduced while maintaining at least 95, 96, 97, 98 or 99% antigenic identity with wt virus. In some embodiments, the viral translation is reduced while maintaining 100% antigenic identity with wt virus.

The invention also provides a method of eliciting a protective immune response in a subject comprising administering to the subject a prophylactically or therapeutically effective dose of a vaccine composition comprising an attenuated dengue virus, wherein expression of viral proteins is reduced by at least 10%. In various embodiments, the expression of viral proteins is reduced by at least 15%, at least 20% or at least 25%. In an embodiment of the invention, an immune response is elicited that is effective against dengue virus of the same lineage as the attenuated virus of the vaccine. In another embodiment, an immune response is elicited that is effective against a heterologous dengue virus.

The invention also provides a method of making an attenuated dengue virus genome comprising a) obtaining the genomic nucleotide b) recoding the envelope-encoding nucleotide sequence to reduce expression and recoding the nonstructural protein 3-encoding nucleotide sequence to reduce expression, and substituting the recoded nucleotide sequences into a dengue virus genome to make an attenuated dengue virus genome. In certain embodiments, only the E region is targeted. In some embodiments, expression of another virus protein encoding region is also reduced.

The invention also provides a method of constructing template dengue virus DNA sequences for transcription of infectious viral RNA genomes by T7 polymerase using overlapping PCR. All dengue genomes with homologous backbone were divided into three fragments starting from 5′ end (fragment 1: ntl-3596; fragment 2: nt3030-6959, and fragment 3: nt: nt6851-end) and chemically/biochemically synthesized. Instead of constructing an infectious cDNA clone, a long overlap extension PCR strategy was used to obtain full-length dengue genome (or: the syn-wt and min dengue genomes simultaneously). To construct the heterologous dengue genomes in DENV2 16681 backbone, the first 2.5 Kb fragments, containing the DENV2 16681 5′UTR, C domain and the prM/M, E (wildtype or deoptimized) domain from individual DENV serotype and a small fraction of the DENV2 16681 NS1 domain, were synthesized. The 8 Kb DENV2 16681 backbone containing all NS domains and the 3′UTR were obtained from infectious clones encoding wildtype or deoptimized DENV2 16681. The 2.5 Kb and 8 Kb fragments were fused together using an asymmetric-fusion PCR method.

Various embodiments of the present invention provide for a modified dengue virus, comprising a recoded prM protein, a recoded envelope (E) protein, or both, wherein the recoded prM protein has a reduced codon pair bias compared to its parent prM protein encoding sequence, or has at least 5 codons substituted with synonymous codons less frequently used, or has an increased number of CpG or UpA di-nucleotides compared its parent prM protein encoding sequence, and wherein the recoded E protein has a reduced codon pair bias compared to its parent E protein encoding sequence, or has at least 5 codons substituted with synonymous codons less frequently used, or has an increased number of CpG or UpA di-nucleotides compared its parent E protein encoding sequence.

In various embodiments, the expression of the prM protein or E protein or both can be reduced compared to its parent dengue virus.

In various embodiments, the recoded prM protein has a reduced codon pair bias compared to its parent prM protein encoding sequence. In various embodiments, the recoded prM protein can have at least 5 codons substituted with synonymous codons less frequently used. In various embodiments, the recoded prM protein can have an increased number of CpG or UpA di-nucleotides compared its parent prM protein encoding sequence. In various embodiments, the recoded E protein can have a reduced codon pair bias compared to its parent E protein encoding sequence. In various embodiments, the recoded E protein can have at least 5 codons substituted with synonymous codons less frequently used. In various embodiments, the recoded E protein can have an increased number of CpG or UpA di-nucleotides compared its parent E protein encoding sequence. In various embodiments, each of the recoded prM or E protein-encoding sequence can have a codon pair bias of less than −0.05. In various embodiments, the codon pair bias of each of the recoded prM or E protein-encoding sequence can be reduced by at least 0.05.

In various embodiments, the modified dengue virus can be selected from type 1, type 2, type 3, type 4 or a combination thereof. In various embodiments, the modified dengue virus is a modified tetravalent dengue virus.

Various embodiments of the invention provide for a dengue vaccine composition for inducing a protective immune response in a subject, comprising a modified dengue virus as described above and herein, and a pharmaceutically acceptable excipient or carrier. Various embodiments of the invention provide a method of eliciting an immune response in a subject, comprising: administering to the subject an effective dose of a composition comprising a modified dengue virus as described above and herein, and a pharmaceutically acceptable excipient or carrier.

In various embodiments, the immune response can be a protective immune response, and a prophylactically effective or therapeutically effective dose of a vaccine composition of claims can be administered. In various embodiments, the immune response can be cross-protective against a heterologous dengue virus.

In various embodiments, the method can further comprise administering to the subject at least one adjuvant.

Various embodiments of the present invention provide for a method of eliciting an immune response in a subject in need thereof, comprising: administering a prime dose of (i) an attenuated dengue virus produced by a method other than codon-pair deoptimization or codon deoptimization, or increasing of CpG or UpA di-nucleotides, or (ii) a modified dengue virus comprising a recoded prM protein, a recoded envelope (E) protein, or both, wherein the recoded prM protein has a reduced codon pair bias compared to its parent prM protein encoding sequence, or has at least 5 codons substituted with synonymous codons less frequently used, or has an increased number of CpG or UpA di-nucleotides compared its parent prM protein encoding sequence, and wherein the recoded E protein has a reduced codon pair bias compared to its parent E protein encoding sequence, or has at least 5 codons substituted with synonymous codons less frequently, or has an increased number of CpG or UpA di-nucleotides compared its parent E protein encoding sequence; and administering one or more boost dose of (i) the attenuated dengue virus produced by methods other than codon-pair deoptimization or codon deoptimization, or increasing of CpG or UpA di-nucleotides, or (ii) the modified dengue virus to the subject in need thereof, wherein at least the prime dose or the one or more boost dose is the modified dengue virus.

In various embodiments, a first of the one or more boost dose can be administered about 2 weeks after the prime dose.

In various embodiments, the expression of the prM protein or E protein or both can be reduced compared to its parent dengue virus.

In various embodiments, the recoded prM protein can have a reduced codon pair bias compared to its parent prM protein encoding sequence. In various embodiments, the recoded prM protein can have at least 5 codons substituted with synonymous codons less frequently used. In various embodiments, the recoded prM protein can have an increased number of CpG or UpA di-nucleotides compared its parent prM protein encoding sequence. In various embodiments, the recoded E protein can have a reduced codon pair bias compared to its parent E protein encoding sequence. In various embodiments, the recoded E protein can have at least 5 codons substituted with synonymous codons less frequently used. In various embodiments, the recoded E protein can have an increased number of CpG or UpA di-nucleotides compared its parent E protein encoding sequence. In various embodiments, each of the recoded prM or E protein-encoding sequence has a codon pair bias of less than −0.05. In various embodiments, the codon pair bias of each of the recoded prM or E protein-encoding sequence is reduced by at least 0.05.

In various embodiments, the modified dengue virus is selected from type 1, type 2, type 3, type 4 or a combination thereof. In various embodiments, the modified dengue virus is a modified tetravalent dengue virus.

Various embodiments of the present invention provide for a method of making a modified dengue virus genome comprising: obtaining a nucleotide sequence encoding the envelope protein of a dengue virus; recoding the envelope encoding nucleotide sequence to reduce protein expression, and substituting a nucleic acid having the recoded envelope-encoding nucleotide sequence into a parent dengue virus genome to make a modified dengue virus genome; whereby expression of the recoded envelope-encoding nucleotide sequence is reduced compared to the parent virus.

### BRIEF DESCRIPTION OF THE FIGURES

FIG. 1 depicts rapid construction of SAVE-deoptimized, live-attenuated dengue vaccine candidate with growth in Vero cells under animal component-free conditions. Codon pair bias of the dengue prM/E genes and their SAVE-deoptimized counterparts in relation to the human ORFeome. Codon-Pair Bias (CPB) is expressed as the average codon pair score of a given gene's open reading frame (ORF). Positive and negative CPB value signifies the predominance of statistically over- or under-represented codon-pairs, respectively in an ORF. Red circles indicate the CPB of each of the 14,795 human ORFs, representing the majority of the known, annotated human genes at the time of our analysis (ORFeome). The CPB of wild-type E gene fall within the normal range of human host cell's genes. Following codon pair ‘deoptimization’ via SAVE, the resulting deoptimized prM/E gene segments were now encoded predominately by under-represented human codon-pairs as evident by their extremely negative CPB, and are drastically different from any human gene. B. cDNA genomes of wild-type and synthetically ‘de-optimized’ chimeric dengue vaccine variants. The SAVE-deoptimized synthetic E were synthesized de novo and using overlapping PCR subcloned individually into the WT DENV genomes—yielding eight independent cDNA genomes each containing a synthetically ‘de-optimized’ fragment(s). We constructed infectious cDNA genomes for wt DENV1-4 and then recovered fully infectious, replicating virus for the deoptimized DENV vaccine candidates via transfection of RNA into Vero (all DENV WT, E-W/MIN, and E-MIN viruses) or BHK cells.

FIG. 2 depicts diagram of subcloning strategies for decreasing attenuation or increasing immunogenicity by reducing the length of deoptimized sequence in the E encoding region. The second generation of dengue vaccine candidates leverages the flexibility of the SAVE platform to reduce the deoptimized region from full-length (E-min) to approximately half of the length (W-E-Min) while keeping the amino acid sequence 100% identical.

FIG. 3 depicts growth of heterologous backbone dengue vaccine candidates in Vero cells under animal component-free conditions. DENV1-4 E-Min were used to infect Vero cells under animal-component free conditions at a MOI of 0.01 and supernatant titrated daily in a multiple-step growth curve over the course of 10 days post-infection. DENV2, DENV3, and DENV4 candidates reached titers of 1-2×10⁵ FFU/ml while DENV1 E-Min titer was reduced by ˜1 log₁₀ compared to the others.

FIG. 4A-4C depicts multiple step growth curves that were conducted in Vero cells for synthetic DENV1 WT virus in a full-length DENV1 backbone (FIG. 4A) and attenuated live vaccine candidates DENV1 E-W/MIN (FIG. 4B) and E-MIN (FIG. 4C) derived from DENV1 WT. Synthetic DENV1 WT grows well in Vero cells at 33° C. and 37° C. Typical of DENV, a transient reduction in virus yield was observed at 39° C., however, titers recovered to 37° C. levels by 7 dpi. DENV1 E-W/MIN, however, reached serviceable titers ˜10-fold lower than DENV1 WT at both 33° C. and 37° C. DENV1 E-MIN was highly attenuated in Vero cells and only reached detectable titers at 33° C. starting on days 10-14 post-infection.

FIG. 5 depicts multiple step growth curves that were conducted in Vero cells for synthetic DENV2 WT virus (in a full-length DENV2 backbone) and attenuated live vaccine candidates DENV2 E-W/MIN and E-MIN derived from DENV2 WT. DENV2 E-W/MIN and E-MIN were both attenuated in vitro with a 1-2 log₁₀ FFU/ml reduction for E-W/MIN and a more pronounced 2-3 log₁₀ FFU/ml reduction for DENV2 E-MIN (FIG. 3). While growth kinetics were delayed, with regular medium replacement every 1-2 days virus titers reached >10⁶ FFU/ml for DENV2 E-W/MIN. Importantly, attenuation was correlated with the extent of deoptimization in the E region. While temperature sensitivity (difference between titers at 37° C. and 39° C.) was similar for DENV2-WT and DENV2 E-W/MIN through day 4 post-infection, there were significant differences on days 5, 6, and 7. Temperature sensitivity was not observed for DENV2 E-MIN because there was no replication at any temperature through 6 DPI. However, starting at day 7-14 DENV2 E-MIN started producing detectable virus (but only at 33° C. and 37° C.).

FIG. 6 depicts multiple step growth curves that were conducted in Vero cells for synthetic DENV3 WT virus (in a full-length DENV3 backbone) and attenuated live vaccine candidates DENV3 E-W/MIN and E-MIN derived from DENV3 WT. It was apparent that DENV3 E-W/MIN was more temperature sensitive at 39° C. than the WT with a significantly greater difference observable on days 3, 4, 5, and 7 post-infection. Because DENV3 E-MIN had minimal levels of replication in Vero cells at 39° C., the difference in virus yield from 3TC to 39° C. were as high as 10⁷ FFU/ml. When grown at permissible temperatures, however, DENV3 E-MIN reached high titers which is promising for cGMP manufacture.

FIG. 7 depicts multiple step growth curves that were conducted in Vero cells for synthetic DENV4 WT virus (in a full-length DENV4 backbone) and attenuated live vaccine candidates DENV4 E-W/MIN and E-MIN derived from DENV4 WT. Both DENV4 WT and E-W/MIN grew to high titers in Vero cells, with the peak DENV4 E-W/MIN titers approximately 10-fold lower but still high (˜10⁷ FFU/ml) at 33° C. and 37° C. Both viruses were partially restricted at 39° C., but not to the extent seen with DENV1-3. On day 5 a small but significant (˜5-fold) change in titer from 37° C. to 39° C. compared to WT was observed. DENV4 E-MIN was attenuated in Vero cells with peak titers 100-fold lower than WT. DENV4 E-MIN replication at 33° C. and 37° C. was similar, however, DENV4 E-MIN was not viable at 39° C. with no detectable virus on days 1-14 post-infection.

FIG. 8A-8B depicts evaluation of the attenuation, immunogenicity, and efficacy of DENV2 vaccine candidates in AG129 mice. DENV-2 E-min based on a DENV2 16681 backbone was tested in an immunogenicity/dose escalation study in AG129 mice lacking interferon alpha or gamma receptors. Animals were immunized with DENV-2 E-min or DENV-2 16681 at two different concentrations. Resultant neutralizing antibody titers were determined, and the protective efficacy afforded evaluated against a mouse-adapted lethal strain of DENV2, D2S10.

FIG. 9 depicts constructed D2-E-min and D2-1/2-E-min with E sequence derived from DENV-2/NI/BID-V533/2005 in the heterologous DENV2 16681 backbone to improve the clinical relevance of our DENV2 candidate. We tested D2-1/2-E-min, with the E region consisting of half wt DENV-2/NI/BID-V533/2005 sequence and half CPD, to improve the immunogenicity of our virus. AG129 and BALB/c mice (n=5) were vaccinated with 10⁶ or 10⁴ FFU of D2-E-min, D2-1/2-E-min, or D2-WTE (DENV2 16681 backbone with WT DENV-2/NI/BID-V533/2005 E region). No mortality or morbidity was observed in the DENV2 D2-E-min, D2-1/2-E-min group indicating at least a 100-fold attenuation. The immunogenicity of our candidate was improved by reducing the extent of CPD with PRNT50 values of serum from D2-1/2-E-min being higher compared to D2-E-Min vaccinated AG129 mice. Additionally, high neutralizing antibody titers were maintained with D2-1/2-E-min vaccination as no significant difference was observed between the 10⁶ (GM=3447) and 10⁴ (GM=2867) FFU vaccinated groups. In immune-competent BALB/c mice, both D2-E-min and D2-1/2-E-min engendered levels of neutralizing antibody comparable to wild-type.

FIG. 10A-10B depicts AG129 mice used to test the immunogenicity and protective efficacy of DENV-3 vaccine candidate DENV D2/D3-E-min, which comprises the DENV-2 strain 16681 backbone with a codon-deoptimized prME cassette from DENV-3/VE/BID-V2268/2008. This study was designed to evaluate the immunogenicity and efficacy of this candidate vaccine against challenge with virulent DENV3 CO360/94. The first immunization was well tolerated by all of the animals with no major associated weight loss and none of the animals developed clinical signs. After lethal challenge with 1.2×10⁷ PFU DENV-3 CO360/94, animals in the media control group developed a rapid progressive infection that was lethal in 8/9 animals with the mean day of death (MDD) among the animals that died of 4.0±0.8 days. Immunization with the higher inoculum of DENV D2/D3-E-min provided significant protection with no lethality or morbidity (as measured by >10% weight loss) among the animals in the group.

FIG. 11A-11B depicts AG129 mice used to test the immunogenicity and protective efficacy of DENV-4 vaccine candidates DENV D2/D4-E-min and D2/D4-1/2-E-min, which comprise the DENV-2 strain 16681 backbone with a codon-deoptimized prME cassette from DENV4. AG 129 Mice were vaccinated with 10⁶ D2/D4-E-min (N=12), 10⁶ D2/D4-1/2-E-min (N=12), 10⁶ D2/D4-WTE, or media as a control (N=9). This study was designed to evaluate the immunogenicity and efficacy of this candidate vaccine against challenge with virulent DENV4 703/4. The first immunization with all strains was well tolerated by all groups of animals with no sustained weight loss over the 5 day observation period, however, in the D2/D4-WTE group one mouse had to be euthanized prior to the second immunization and additional animals died after the second immunization. No animals in any of the other vaccine groups experienced progressive weight loss. Sera were collected on day 35 to test for neutralizing antibodies prior to challenge and on day 30 post-challenge from all surviving animals to test final titers by FRNT50. Using a 96-well plate method with Vero cells, each serum sample was tested in duplicate using serum dilutions from 1:64-1:32784 incubated with 50 FFU of DENV4 WT virus. Each vaccine candidate was immunogenic at 35 DPI with vaccination with D2/D4-E-min (GMT 332.9) and D2/D4-1/2-E-min (GMT 738.4) lower than vaccination with D2/D4-WTE (GMT 1640). At 2 days post-challenge (DPC), viremia was detected in 4/12 mice inoculated with D2/D4-WTE. For all of the mice immunized with the other two viruses, viremia levels were below the limit of detection ofthe assay (500 genome equivalents/ml). DENV-4 703/4 challenge produced rapid progressive infection with universal lethality in the media control group. In addition, three animals immunized with D2/D4-E-min experienced lethal infection with the other animals in this group remaining healthy for the duration of the study. Importantly, no lethality or evidence of morbidity as determined by weight loss was seen in any of the D2/D4-1/2-E-min immunized animals after DENV-4 703/4 challenge.

FIG. 12 depicts tested DENV1-4 WT as well as vaccine candidates for DENV2-4 for immunogenicity in IFNα receptor knockout mice. All WT viruses were highly immunogenic in these mice, with neutralizing antibody titers >1024. DENV2-E-W/MIN was equally immunogenic to DENV2 WT virus at both a 10⁶ and 10⁴ FFU dose. Immunogenicity of DENV3 viruses waned with decreasing dose and increased deoptimization, however, DENV3 E-W/MIN was still highly immunogenic at a 10⁴ FFU dose (˜1024 FRNT50). We noticed a pronounced improvement in the immunogenicity of DENV4 E-W/MIN and WT as compared to the heterologous DENV4 WT in the DENV2 16681 backbone, which was poorly immunogenic in mice.

FIG. 13 depicts immunogenicity and efficacy of tetravalent vaccination in AG129 mice against lethal challenge with DENV3 CO360/94. AG129 mice were vaccinated on days 0 and 21 with 10⁶ IFU monovalent D2/D3-E-min vaccine (N=16; 10 male and 6 female), tetravalent vaccine containing 10⁶ IFU of each of the four monovalent DENV E-min viruses (N=16; 10 male and 6 female), or mock vaccinated with media (N=13; 8 male and 5 female). Serum was collected prior to challenge for measurement of neutralizing antibodies by FRNT50. All of the mice immunized with the monovalent vaccine developed detectable neutralizing antibody titers (range 20-160). In mice immunized with the tetravalent vaccine formulation, titers were generally lower (range <20-80). On day 35, all remaining mice were challenged with 10⁷ IFU DENV3 CO360/94 delivered by the intraperitoneal route. As anticipated, animals in the media control group developed a rapid progressive infection that was lethal in 8/9 (89%) animals with the mean day of death (MDD) among the animals that died of 4.1±0.4 days. Immunization with monovalent DENV D2/D3-E-min or the tetravalent vaccine provided significant protection with no lethality or morbidity (as measured by >10% weight loss) among the animals in either group.

FIG. 14 depicts attenuation of homologous backbone viruses—focus size of vaccine strains vs wt-vaccine viruses spread less in vitro as compared to wt. Individual foci areas (mm²) were calculated for Vero cells infected with DENV1-4 WT, E-W/MIN, or E-MIN and incubated for 3 days at temperatures of 33° C., 37° C., or 39° C. We completed analysis of focus-forming unit (FFU) size of each virus using ImageJ software (NIH, v1.52a). Each image of the FFA plates was converted into a binary 8-bit image using ImageJ (an example is presented in FIG. 2), and the scale for measuring FFU area was set to the diameter of the wells of 24-well plates (15.5 mm). Using the “analyze particles” function, the area of each FFU was calculated by ImageJ and the results compared by Sidak's multiple comparisons test (GraphPad Prism 8.1.2).

FIG. 15 depicts in vivo immunogenicity data for tetravalent homologous backbone vaccine Tetravalent and monovalent homologous backbone dengue vaccine candidates were immunogenic in IFNα receptor knockout mice after vaccination with 10⁶ or 10⁴ FFU delivered by the subcutaneous route on day 0 and 21. Neutralizing antibodies were tested using a focus-reduction neutralization 50% test at days 0, 21, and 35 post-vaccination against each strain of DENV1-4 wt.

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

Codon-Pair Bias (CPB) is expressed as the average codon pair score of a given gene's open reading frame (ORF).

A “subject” means any animal or artificially modified animal. Animals include, but are not limited to, humans, non-human primates, cows, horses, sheep, pigs, dogs, cats, rabbits, ferrets, rodents such as mice, rats and guinea pigs, and birds. Artificially modified animals include, but are not limited to, SCID mice with human immune systems. In a preferred embodiment, the subject is a human. Embodiments of birds are domesticated poultry species, including, but not limited to, chickens, turkeys, ducks, and geese.

A “viral host” means any animal or artificially modified animal, or insect that the virus can infect. Animals include, but are not limited to, humans, non-human primates, cows, horses, sheep, pigs, dogs, cats, rabbits, ferrets, rodents such as mice, rats and guinea pigs, and birds. Artificially modified animals include, but are not limited to, SCID mice with human immune systems. In a specific embodiment, the viral host is a human. Embodiments of birds are domesticated poultry species, including, but not limited to, chickens, turkeys, ducks, and geese. Insects include, but are not limited to mosquitos.

A “prophylactically effective dose” is any amount of a vaccine that, when administered to a subject prone to viral infection or prone to affliction with a virus-associated disorder, induces in the subject an immune response that protects the subject from becoming infected by the virus or afflicted with the disorder. “Protecting” the subject means either reducing the likelihood of the subject's becoming infected with the virus, or lessening the likelihood ofthe disorder's onset in the subject, by at least two-fold, preferably at least ten-fold, 25-fold, 50-fold, or 100-fold. For example, if a subject has a 1% chance of becoming infected with a virus, a two-fold reduction in the likelihood of the subject becoming infected with the virus would result in the subject having a 0.5% chance of becoming infected with the virus.

As used herein, a “therapeutically effective dose” is any amount of a vaccine that, when administered to a subject afflicted with a disorder against which the vaccine is effective, induces in the subject an immune response that causes the subject to experience a reduction, remission or regression of the disorder and/or its symptoms. In preferred embodiments, recurrence of the disorder and/or its symptoms is prevented. In other preferred embodiments, the subject is cured of the disorder and/or its symptoms.

The present invention relates to attenuated dengue viruses and the production of attenuated dengue viruses that can be used to protect against viral infection and disease. A basic premise in vaccination is adequate delivery of protective antigens to vaccine recipients assuming that a very high dose (“Peptide or Virus-Like Particle”) or a dose corresponding to live viral infection (“ChimeriVax”) of these traditionally dominant antigenic polypeptides alone are sufficient for adequate vaccine efficacy. Those expectations aside, the present invention benefits from a contrary approach. The invention provides attenuated dengue viruses in which expression of viral proteins is reduced, which have excellent growth properties useful to vaccine production, yet possess an extraordinary safety profile and enhanced protective characteristics. The attenuated viruses proliferate nearly as well as wild type virus, have highly attenuated phenotypes, as revealed by LD₅₀ values, are unusually effective in providing protective immunity against challenge by dengue virus of the same strain, and also provide protective immunity against challenge by dengue virus of other strains.

In certain embodiments of the invention, the attenuated dengue viruses of the invention comprise a recoded pre-membrane (prM)/Envelope (E) encoding region. In embodiments wherein the C, NS1, NS2, NS3, NS4, or NS5 protein encoding regions are not recoded does not exclude mutations and other variations in those sequences, but only means that any mutations or variations made in those sequences have little or no effect on attenuation. Little or no effect on attenuation includes one or both of the following: 1) The mutations or variations in the C, NS1, NS2, NS3, NS4, or NS5 encoding regions do not reduce viral replication or viral infectivity more than 20% when the variant C, NS1, NS2, NS3, NS4, or NS5 encoding region is the only variant in a test dengue virus; 2) Mutations or variations in any ofthe C, NS1, NS2, NS3, NS4, or NS5 encoding regions represent fewer than 10% of the nucleotides in that coding sequence. If specifically provided for in the claims, little or no effect on attenuation includes one or both of the following: 1) The mutations or variations in the C, NS1, NS2, NS3, NS4, or NS5 encoding regions do not reduce viral replication or viral infectivity more than 10% when the variant C, NS1, NS2, NS3, NS4, or NS5 encoding region is the only variant in a test dengue virus; 2) Mutations or variations in any ofthe C, NS1, NS2, NS3, NS4, or NS5 encoding regions represent fewer than 5% of the nucleotides in that coding sequence.

In various embodiments, viruses of the invention are attenuated. In embodiments of the invention, compared to wild type, the viruses are at least 10 fold attenuated, at least 50 fold attenuated, or at least 100 fold attenuated, or at least 200 fold attenuated, or at least 500 fold attenuated, or at least 1000 fold attenuated, of at least 2000 fold attenuated in the AG129 mouse model compared to a wild type virus having proteins ofthe same amino acid sequence but encoded by a different nucleotide sequence.

The attenuated viruses are also highly protective against wild type virus of the same strain. In embodiments of the invention, the protective dose (PD₅₀) of the viruses is, when measured by a mouse model, such as exemplified herein.

The attenuated viruses of the invention also exhibit a large margin of safety (i.e., the difference between LD₅₀ and PD₅₀), thus have high safety factors, defined herein as the ratio of LD₅₀/PD₅₀. In certain embodiments of the invention, the safety factor is at least 10², or at least 103, or at least 10⁴, or at least 10⁵, or at least 2×10⁵, or at least 3×10⁵, or at least 4×10⁵ or at least 5×10⁵, or at least 10⁶, or at least 2×10⁶, or at least 3×10⁶, or at least 4×10⁶, or at least 5×10⁶. In certain embodiments, the safety factor is from 10² to 10³, or from 10³ to 10⁴, or from 10⁴ to 10⁵, or from 10⁵ to 10⁶.

The attenuated viruses of the invention are also highly protective against heterologous strains of the dengue virus within the same serotype. In certain embodiments of the invention, the protective dose (PD₅₀) of an attenuated virus of the invention is less than 1000 PFU, or less than 750 PFU, or less than 500 PFU, or less than 200 PFU, or less than 100 PFU, or less than 50 PFU when measured by a mouse model, such as exemplified herein.

The recoding of E protein encoding sequences of the attenuated viruses of the invention have been made or can be made by one of skill in the art in light of disclosure discussed herein. According to the invention, nucleotide substitutions are engineered in multiple locations in the E coding sequence, wherein the substitutions introduce a plurality of synonymous codons into the genome. In certain embodiments, the synonymous codon substitutions alter codon bias, codon pair bias, the density of infrequent codons or infrequently occurring codon pairs, RNA secondary structure, CG and/or TA (or UA) dinucleotide content, C+G content, translation frameshift sites, translation pause sites, the presence or absence of microRNA recognition sequences or any combination thereof, in the genome. The codon substitutions may be engineered in multiple locations distributed throughout the E coding sequence, or in the multiple locations restricted to a portion of the E coding sequence. Because of the large number of defects (i.e., nucleotide substitutions) involved, the invention provides a means of producing stably attenuated viruses and live vaccines.

As discussed further below, in some embodiments, a virus coding sequence is recoded by substituting one or more codon with synonymous codons used less frequently in the dengue host (e.g., mammals, humans, mosquitoes). In some embodiments, a virus coding sequence is recoded by substituting one or more codons with synonymous codons used less frequently in the dengue virus. In certain embodiments, the number of codons substituted with synonymous codons is at least 5. In some embodiments, at least 10, or at least 20 codons are substituted with synonymous codons. In some embodiments, the number of codons substituted with synonymous codons is at least 30, or at least 40, or at least 50, or at least 75, or at least 100, or at least 125, or at least 150, or at least 175.

In some embodiments, virus codon pairs are recoded to reduce (i.e., lower the value of) codon-pair bias. In certain embodiments, codon-pair bias is reduced by identifying a codon pair in an E coding sequence having a codon-pair score that can be reduced and reducing the codon-pair bias by substituting the codon pair with a codon pair that has a lower codon-pair score. In some embodiments, this substitution of codon pairs takes the form of rearranging existing codons of a sequence. In some such embodiments, a subset of codon pairs is substituted by rearranging a subset of synonymous codons. In other embodiments, codon pairs are substituted by maximizing the number of rearranged synonymous codons. It is noted that while rearrangement of codons leads to codon-pair bias that is reduced (made more negative) for the virus coding sequence overall, and the rearrangement results in a decreased CPS at many locations, there may be accompanying CPS increases at other locations, but on average, the codon pair scores, and thus the CPB of the modified sequence, is reduced. In some embodiments, recoding of codons or codon-pairs can take into account altering the G+C content of the E coding sequence. In some embodiments, recoding of codons or codon-pairs can take into account altering the frequency of CG and/or TA dinucleotides in the E coding sequence.

In certain embodiments, the recoded E protein-encoding sequence has a codon pair bias less than −0.05, or less than −0.06, or less than −0.07, or less than −0.08, or less than −0.09, or less than −0.1, or less than −0.11, or less than −0.12, or less than −0.13, or less than −0.14, or less than −0.15, or less than −0.16, or less than −0.17, or less than −0.18, or less than −0.19, or less than −0.2, or less than −0.25, or less than −0.3, or less than −0.35, or less than −0.4, or less than −0.45, or less than −0.5. In certain embodiments, the codon pair bias of the recoded E protein encoding sequence is reduced by at least 0.05, or at least 0.06, or at least 0.07, or at least 0.08, or at least 0.09, or at least 0.1, or at least 0.11, or at least 0.12, or at least 0.13, or at least 0.14, or at least 0.15, or at least 0.16, or at least 0.17, or at least 0.18, or at least 0.19, or at least 0.2, or at least 0.25, or at least 0.3, or at least 0.35, or at least 0.4, or at least 0.45, or at least 0.5, compared to the parent E protein encoding sequence from which it is derived. In certain embodiments, it is in comparison to an E protein encoding sequence from which the calculation is to be made; for example, the E protein encoding sequence of a wild type virus.

In certain embodiments, rearrangement of synonymous codons ofthe E protein-encoding sequence provides a codon-pair bias reduction of at least 0.05, or at least 0.06, or at least 0.07, or at least 0.08, or at least 0.09, or at least 0.1, or at least 0.11, or at least 0.12, or at least 0.13, or at least 0.14, or at least 0.15, or at least 0.16, or at least 0.17, or at least 0.18, or at least 0.19, or at least 0.2, or at least 0.25, or at least 0.3, or at least 0.35, or at least 0.4, or at least 0.45, or at least 0.5, compared to the parent E protein encoding sequence from which it is derived. In certain embodiments, it is in comparison to an E protein encoding sequence from which the calculation is to be made; for example, the E protein encoding sequence of a wild type virus.

Usually, these substitutions and alterations are made and reduce expression of the encoded virus proteins without altering the amino acid sequence of the encoded protein. In certain embodiments, the invention also includes alterations in the E coding sequence that result in substitution of non-synonymous codons and amino acid substitutions in the encoded protein, which may or may not be conservative. In various embodiments, there are up to 1, 2, 3, 4, 5, 6, 7, 8, 9, 10, 11, 12, 13, 14, or 15 nucleotide substitutions.

Most amino acids are encoded by more than one codon. See the genetic code in Table 1. For instance, alanine is encoded by GCU, GCC, GCA, and GCG. Three amino acids (Leu, Ser, and Arg) are encoded by six different codons, while only Trp and Met have unique codons. “Synonymous” codons are codons that encode the same amino acid. Thus, for example, CUU, CUC, CUA, CUG, UUA, and UUG are synonymous codons that code for Leu. Synonymous codons are not used with equal frequency. In general, the most frequently used codons in a particular organism are those for which the cognate tRNA is abundant, and the use of these codons enhances the rate and/or accuracy of protein translation. Conversely, tRNAs for the rarely used codons are found at relatively low levels, and the use of rare codons is thought to reduce translation rate and/or accuracy.

TABLE 1
Genetic Code

| U | C | A | G | | |
|---|---|---|---|---|---|
| U | Phe | Ser | Tyr | Cys | U |
| | Phe | Ser | Tyr | Cys | C |
| | Leu | Ser | STOP | STOP | A |
| | Leu | Ser | STOP | Trp | G |
| C | Leu | Pro | His | Arg | U |
| | Leu | Pro | His | Arg | C |
| | Leu | Pro | Gln | Arg | A |
| | Leu | Pro | Gln | Arg | G |
| A | Ile | Thr | Asn | Ser | U |
| | Ile | Thr | Asn | Ser | C |
| | Ile | Thr | Lys | Arg | A |
| | Met | Thr | Lys | Arg | G |
| G | Val | Ala | Asp | Gly | U |
| | Val | Ala | Asp | Gly | C |
| | Val | Ala | Glu | Gly | A |
| | Val | Ala | Glu | Gly | G |

^{a} The first nucleotide in each codon encoding a particular amino acid is shown in the left-most column; the second nucleotide is shown in the top row; and the third nucleotide is shown in the right-most column.

### Codon Bias

As used herein, a “rare” codon is one of at least two synonymous codons encoding a particular amino acid that is present in an mRNA at a significantly lower frequency than the most frequently used codon for that amino acid. Thus, the rare codon may be present at about a 2-fold lower frequency than the most frequently used codon. Preferably, the rare codon is present at least a 3-fold, more preferably at least a 5-fold, lower frequency than the most frequently used codon for the amino acid. Conversely, a “frequent” codon is one of at least two synonymous codons encoding a particular amino acid that is present in an mRNA at a significantly higher frequency than the least frequently used codon for that amino acid. The frequent codon may be present at about a 2-fold, preferably at least a 3-fold, more preferably at least a 5-fold, higher frequency than the least frequently used codon for the amino acid. For example, human genes use the leucine codon CTG 40% of the time, but use the synonymous CTA only 7% of the time (see Table 2). Thus, CTG is a frequent codon, whereas CTA is a rare codon. Roughly consistent with these frequencies of usage, there are 6 copies in the genome for the gene for the tRNA recognizing CTG, whereas there are only 2 copies of the gene for the tRNA recognizing CTA. Similarly, human genes use the frequent codons TCT and TCC for serine 18% and 22% of the time, respectively, but the rare codon TCG only 5% of the time. TCT and TCC are read, via wobble, by the same tRNA, which has 10 copies of its gene in the genome, while TCG is read by a tRNA with only 4 copies. It is well known that those mRNAs that are very actively translated are strongly biased to use only the most frequent codons. This includes genes for ribosomal proteins and glycolytic enzymes. On the other hand, mRNAs for relatively non-abundant proteins may use the rare codons.

TABLE 2
Codon usage in Homo sapiens
(source: www.kazusa.or.jp/codon/)

| Amino Acid | Codon | Number | /1000 | Fraction |
|---|---|---|---|---|
| Gly | GGG | 636457.00 | 16.45 | 0.25 |
| Gly | GGA | 637120.00 | 16.47 | 0.25 |
| Gly | GGT | 416131.00 | 10.76 | 0.16 |
| Gly | GGC | 862557.00 | 22.29 | 0.34 |
| Glu | GAG | 1532589.00 | 39.61 | 0.58 |
| Glu | GAA | 1116000.00 | 28.84 | 0.42 |
| Asp | GAT | 842504.00 | 21.78 | 0.46 |
| Asp | GAC | 973377.00 | 25.16 | 0.54 |
| Val | GTG | 1091853.00 | 28.22 | 0.46 |
| Val | GTA | 273515.00 | 7.07 | 0.12 |
| Val | GTT | 426252.00 | 11.02 | 0.18 |
| Val | GTC | 562086.00 | 14.53 | 0.24 |
| Ala | GCG | 286975.00 | 7.42 | 0.11 |
| Ala | GCA | 614754.00 | 15.89 | 0.23 |
| Ala | GCT | 715079.00 | 18.48 | 0.27 |
| Ala | GCC | 1079491.00 | 27.90 | 0.40 |
| Arg | AGG | 461676.00 | 11.93 | 0.21 |
| Arg | AGA | 466435.00 | 12.06 | 0.21 |
| Ser | AGT | 469641.00 | 12.14 | 0.15 |
| Ser | AGC | 753597.00 | 19.48 | 0.24 |
| Lys | AAG | 1236148.00 | 31.95 | 0.57 |
| Lys | AAA | 940312.00 | 24.30 | 0.43 |
| Asn | AAT | 653566.00 | 16.89 | 0.47 |
| Asn | AAC | 739007.00 | 19.10 | 0.53 |
| Met | ATG | 853648.00 | 22.06 | 1.00 |
| Ile | ATA | 288118.00 | 7.45 | 0.17 |
| Ile | ATT | 615699.00 | 15.91 | 0.36 |
| Ile | ATC | 808306.00 | 20.89 | 0.47 |
| Thr | ACG | 234532.00 | 6.06 | 0.11 |
| Thr | ACA | 580580.00 | 15.01 | 0.28 |
| Thr | ACT | 506277.00 | 13.09 | 0.25 |
| Thr | ACC | 732313.00 | 18.93 | 0.36 |
| Trp | TGG | 510256.00 | 13.19 | 1.00 |
| End | TGA | 59528.00 | 1.54 | 0.47 |
| Cys | TGT | 407020.00 | 10.52 | 0.45 |
| Cys | TGC | 487907.00 | 12.61 | 0.55 |
| End | TAG | 30104.00 | 0.78 | 0.24 |
| End | TAA | 38222.00 | 0.99 | 0.30 |
| Tyr | TAT | 470083.00 | 12.15 | 0.44 |
| Tyr | TAC | 592163.00 | 15.30 | 0.56 |
| Leu | TTG | 498920.00 | 12.89 | 0.13 |
| Leu | TTA | 294684.00 | 7.62 | 0.08 |
| Phe | TTT | 676381.00 | 17.48 | 0.46 |
| Phe | TTC | 789374.00 | 20.40 | 0.54 |
| Ser | TCG | 171428.00 | 4.43 | 0.05 |
| Ser | TCA | 471469.00 | 12.19 | 0.15 |
| Ser | TCT | 585967.00 | 15.14 | 0.19 |
| Ser | TCC | 684663.00 | 17.70 | 0.22 |
| Arg | CGG | 443753.00 | 11.47 | 0.20 |
| Arg | CGA | 239573.00 | 6.19 | 0.11 |
| Arg | CGT | 176691.00 | 4.57 | 0.08 |
| Arg | CGC | 405748.00 | 10.49 | 0.18 |
| Gln | CAG | 1323614.00 | 34.21 | 0.74 |
| Gln | CAA | 473648.00 | 12.24 | 0.26 |
| His | CAT | 419726.00 | 10.85 | 0.42 |
| His | CAC | 583620.00 | 15.08 | 0.58 |
| Leu | CTG | 1539118.00 | 39.78 | 0.40 |
| Leu | CTA | 276799.00 | 7.15 | 0.07 |
| Leu | CTT | 508151.00 | 13.13 | 0.13 |
| Leu | CTC | 759527.00 | 19.63 | 0.20 |
| Pro | CCG | 268884.00 | 6.95 | 0.11 |
| Pro | CCA | 653281.00 | 16.88 | 0.28 |
| Pro | CCT | 676401.00 | 17.48 | 0.29 |
| Pro | CCC | 767793.00 | 19.84 | 0.32 |

The propensity for highly expressed genes to use frequent codons is called “codon bias.” A gene for a ribosomal protein might use only the 20 to 25 most frequent of the 61 codons, and have a high codon bias (a codon bias close to 1), while a poorly expressed gene might use all 61 codons, and have little or no codon bias (a codon bias close to 0). It is thought that the frequently used codons are codons where larger amounts of the cognate tRNA are expressed, and that use of these codons allows translation to proceed more rapidly, or more accurately, or both. The PV capsid protein, for example, is very actively translated, and has a high codon bias.

### Codon Pair Bias

In addition, a given organism has a preference for the nearest codon neighbor of a given codon A, referred to a bias in codon pair utilization. A change of codon pair bias, without changing the existing codons, can influence the rate of protein synthesis and production of a protein.

Codon pair bias may be illustrated by considering the amino acid pair Ala-Glu, which can be encoded by 8 different codon pairs. If no factors other than the frequency of each individual codon (as shown in Table 2) are responsible for the frequency ofthe codon pair, the expected frequency of each ofthe 8 encodings can be calculated by multiplying the frequencies of the two relevant codons. For example, by this calculation the codon pair GCA-GAA would be expected to occur at a frequency of 0.097 out of all Ala-Glu coding pairs (0.23×0.42; based on the frequencies in Table 2). In order to relate the expected (hypothetical) frequency of each codon pair to the actually observed frequency in the human genome the Consensus CDS (CCDS) database of consistently annotated human coding regions, containing a total of 14,795 human genes, was used. This set of genes is the most comprehensive representation of human coding sequences. Using this set of genes, the frequencies of codon usage were re-calculated by dividing the number of occurrences of a codon by the number of all synonymous codons coding for the same amino acid. As expected the frequencies correlated closely with previously published ones such as the ones given in Table 2. Slight frequency variations are possibly due to an oversampling effect in the data provided by the codon usage database at Kazusa DNA Research Institute (www.kazusa.or.jp/codon/codon.html) where 84949 human coding sequences were included in the calculation (far more than the actual number of human genes). The codon frequencies thus calculated were then used to calculate the expected codon-pair frequencies by first multiplying the frequencies of the two relevant codons with each other (see Table 3 expected frequency), and then multiplying this result with the observed frequency (in the entire CCDS data set) with which the amino acid pair encoded by the codon pair in question occurs. In the example of codon pair GCA-GAA, this second calculation gives an expected frequency of 0.098 (compared to 0.097 in the first calculation using the Kazusa dataset). Finally, the actual codon pair frequencies as observed in a set of 14,795 human genes was determined by counting the total number of occurrences of each codon pair in the set and dividing it by the number of all synonymous coding pairs in the set coding for the same amino acid pair (Table 3; observed frequency). Frequency and observed/expected values for the complete set of 3721 (61²) codon pairs, based on the set of 14,795 human genes, are provided herewith as Table 3.

TABLE 3
Codon Pair Scores Exemplified
by the Amino Pair Ala-Glu

| amino | | | | |
|---|---|---|---|---|
| acid | codon | expected | observed | obs/exp |
| pair | pair | frequency | frequency | ratio |
| AE | GCAGAA | 0.098 | 0.163 | 1.65 |
| AE | GCAGAG | 0.132 | 0.198 | 1.51 |
| AE | GCCGAA | 0.171 | 0.031 | 0.18 |
| AE | GCCGAG | 0.229 | 0.142 | 0.62 |
| AE | GCGGAA | 0.046 | 0.027 | 0.57 |
| AE | GCGGAG | 0.062 | 0.089 | 1.44 |
| AE | GCTGAA | 0.112 | 0.145 | 1.29 |
| AE | GCTGAG | 0.150 | 0.206 | 1.37 |
| Total | | 1.000 | 1.000 | |

If the ratio of observed frequency/expected frequency of the codon pair is greater than one the codon pair is said to be overrepresented. If the ratio is smaller than one, it is said to be underrepresented. In the example, the codon pair GCA-GAA is overrepresented 1.65 fold while the coding pair GCC-GAA is more than 5-fold underrepresented.

Many other codon pairs show very strong bias; some pairs are under-represented, while other pairs are over-represented. For instance, the codon pairs GCCGAA (AlaGlu) and GATCTG (AspLeu) are three- to six-fold under-represented (the preferred pairs being GCAGAG and GACCTG, respectively), while the codon pairs GCCAAG (AlaLys) and AATGAA (AsnGlu) are about two-fold over-represented. It is noteworthy that codon pair bias has nothing to do with the frequency of pairs of amino acids, nor with the frequency of individual codons. For instance, the under-represented pair GATCTG (AspLeu) happens to use the most frequent Leu codon, (CTG).

As discussed more fully below, codon pair bias takes into account the score for each codon pair in a coding sequence averaged over the entire length of the coding sequence. According to the invention, codon pair bias is determined by

$$CPB = {\sum }_{i = 1}^{k} \frac{C P S i}{K - 1}$$

Accordingly, similar codon pair bias for a coding sequence can be obtained, for example, by minimized codon pair scores over a subsequence or moderately diminished codon pair scores over the full length ofthe coding sequence.

### Calculation of Codon Pair Bias

Every individual codon pair of the possible 3721 non-“STOP” containing codon pairs (e.g., GTT-GCT) carries an assigned “codon pair score,” or “CPS” that is specific for a given “training set” of genes. The CPS of a given codon pair is defined as the log ratio of the observed number of occurrences over the number that would have been expected in this set of genes (in this example the human genome). Determining the actual number of occurrences of a particular codon pair (or in other words the likelihood of a particular amino acid pair being encoded by a particular codon pair) is simply a matter of counting the actual number of occurrences of a codon pair in a particular set of coding sequences. Determining the expected number, however, requires additional calculations. The expected number is calculated so as to be independent of both amino acid frequency and codon bias similarly to Gutman and Hatfield. That is, the expected frequency is calculated based on the relative proportion of the number of times an amino acid is encoded by a specific codon. A positive CPS value signifies that the given codon pair is statistically over-represented, and a negative CPS indicates the pair is statistically under-represented in the human genome.

To perform these calculations within the human context, the most recent Consensus CDS (CCDS) database of consistently annotated human coding regions, containing a total of 14,795 genes, was used. This data set provided codon and codon pair, and thus amino acid and amino-acid pair frequencies on a genomic scale.

The paradigm of Federov et al. (2002), was used to further enhanced the approach of Gutman and Hatfield (1989). This allowed calculation of the expected frequency of a given codon pair independent of codon frequency and non-random associations of neighboring codons encoding a particular amino acid pair. The detailed equations used to calculate CPB are disclosed in WO 2008/121992 and WO 2011/044561, which are incorporated by reference.

$$S ( {P}_{i j} ) = ln ( \frac{{N}_{O} ( {P}_{i j} )}{{N}_{E} ( {P}_{i j} )} ) = ln ( \frac{{N}_{O} ( {P}_{i j} )}{F ( {C}_{i} ) F ( {C}_{j} ) {N}_{O} ( {X}_{i j} )} )$$

In the calculation, Pᵢⱼ is a codon pair occurring with a frequency of N_{O}(Pᵢⱼ) in its synonymous group. Cᵢ and Cⱼ are the two codons comprising Pᵢⱼ, occurring with frequencies F(Cᵢ) and F(Cⱼ) in their synonymous groups respectively. More explicitly, F(Cᵢ) is the frequency that corresponding amino acid Xᵢᵢ s coded by codon Cᵢ throughout all coding regions and F(Cᵢ)=N_{O}(Cⱼ)/N_{O}(Xᵢ), where N_{O}(Cᵢ) and N_{O}(Xᵢ) are the observed number of occurrences of codon Cᵢ and amino acid Xᵢ respectively. F(Cⱼ) is calculated accordingly. Further, N_{O}(Xᵢⱼ) is the number of occurrences of amino acid pair Xᵢⱼ throughout all coding regions. The codon pair bias score S(Pᵢⱼ) of Pᵢⱼ was calculated as the log-odds ratio of the observed frequency N_{O}(Pᵢⱼ) over the expected number of occurrences of Nₒ(Pᵢⱼ).

Using the formula above, it was then determined whether individual codon pairs in individual coding sequences are over- or under-represented when compared to the corresponding genomic Nₑ(Pᵢⱼ) values that were calculated by using the entire human CCDS data set. This calculation resulted in positive S(Pᵢⱼ) score values for over-represented and negative values for under-represented codon pairs in the human coding regions.

The “combined” codon pair bias of an individual coding sequence was calculated by averaging all codon pair scores according to the following formula:

$$S ( {P}_{i j} ) = {\sum }_{l = 1}^{k} \frac{S ( {P}_{i j} ) l}{k - 1}$$

The codon pair bias of an entire coding region is thus calculated by adding all of the individual codon pair scores comprising the region and dividing this sum by the length of the coding sequence.

### Calculation of Codon Pair Bias, Implementation of Algorithm to Alter Codon-Pair Bias.

An algorithm was developed to quantify codon pair bias. Every possible individual codon pair was given a “codon pair score”, or “CPS”. CPS is defined as the natural log of the ratio of the observed over the expected number of occurrences of each codon pair over all human coding regions, where humans represent the host species of the instant vaccine virus to be recoded.

$$C P S = ln ( \frac{{F ( A B )}_{O}}{\frac{F ( A ) \times F ( B )}{F ( X ) \times F ( Y )} \times F ( XY )} )$$

Although the calculation of the observed occurrences of a particular codon pair is straightforward (the actual count within the gene set), the expected number of occurrences of a codon pair requires additional calculation. We calculate this expected number to be independent both of amino acid frequency and of codon bias, similar to Gutman and Hatfield. That is, the expected frequency is calculated based on the relative proportion ofthe number of times an amino acid is encoded by a specific codon. A positive CPS value signifies that the given codon pair is statistically over-represented, and a negative CPS indicates the pair is statistically under-represented in the human genome.

Using these calculated CPSs, any coding region can then be rated as using over- or under-represented codon pairs by taking the average of the codon pair scores, thus giving a Codon Pair Bias (CPB) for the entire gene.

$$C P B = {\sum }_{i = 1}^{k} \frac{C P S i}{k - 1}$$

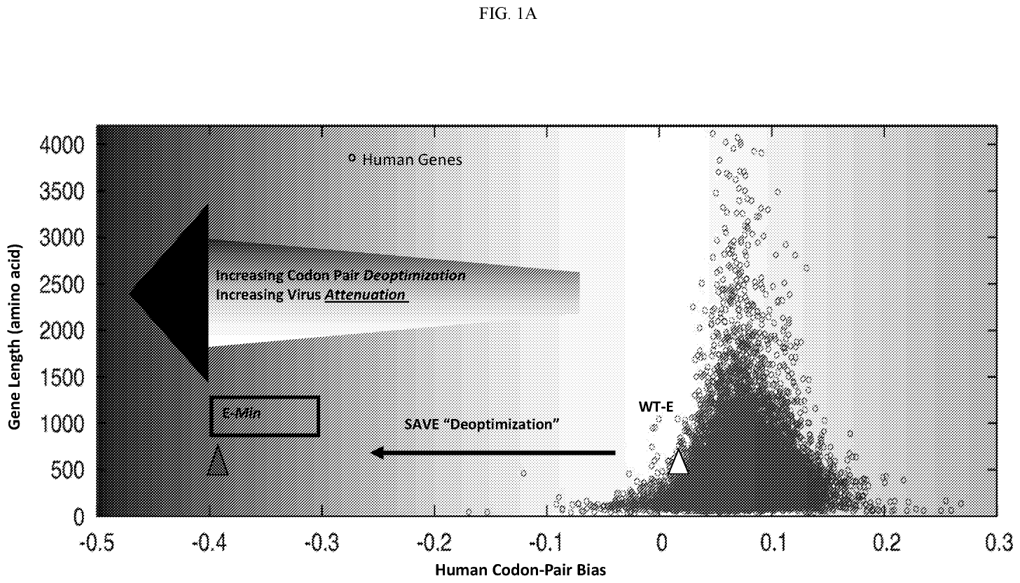

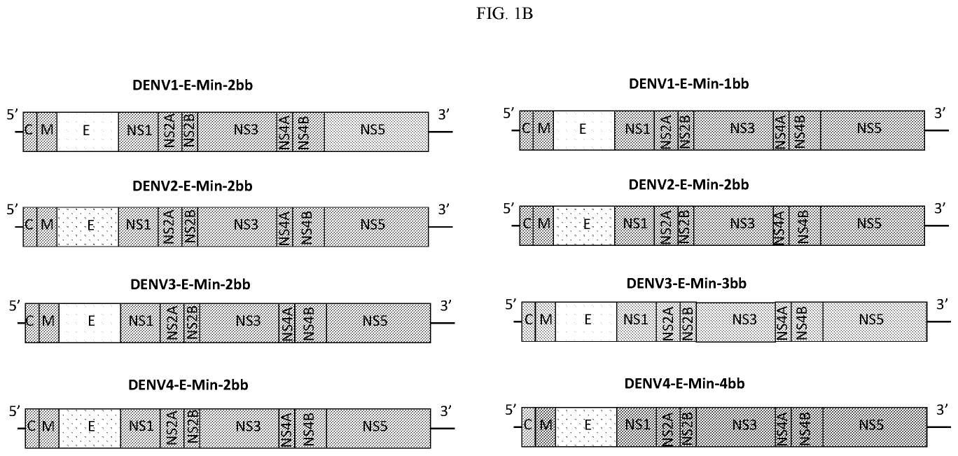

The CPB has been calculated for all annotated human genes using the equations shown and plotted (FIG. 1). Each point in the graph corresponds to the CPB of a single human gene. The peak of the distribution has a positive codon pair bias of 0.07, which is the mean score for all annotated human genes. Also, there are very few genes with a negative codon pair bias. Equations established to define and calculate CPB were then used to manipulate this bias.

Algorithm for Reducing Codon-Pair Bias to Attenuate.

Recoding of protein-encoding sequences may be performed with or without the aid of a computer, using, for example, a gradient descent, or simulated annealing, or other minimization routine. An example of the procedure that rearranges codons present in a starting sequence can be represented by the following steps:
(1) Obtain wildtype viral genome sequence.
(2) Select protein coding sequences to target for attenuated design.
(3) Lock down known or conjectured DNA segments with non-coding functions.
(4) Select desired codon distribution for remaining amino acids in redesigned proteins.
(5) Perform random shuffle of at least two synonymous unlocked codon positions and calculate codon-pair score.
(6) Further reduce (or increase) codon-pair score optionally employing a simulated annealing procedure.
(7) Inspect resulting design for excessive secondary structure and unwanted restriction site:
   if yes->go to step (5) or correct the design by replacing problematic regions with wildtype sequences and go to step (8).
(8) Synthesize DNA sequence corresponding to virus design.
(9) Create viral construct and assess viral phenotype:
   if too attenuated, prepare subclone construct and go to 9;
   if insufficiently attenuated, go to 2.

Attenuation of viruses by reducing codon pair bias is disclosed in WO 2008/121992 and WO 2011/044561, which are incorporated by reference.

Methods of obtaining full-length Flavivirus or dengue genome sequence or codon pair deoptimized sequences embedded in a wild-type Flavivirus or dengue genome sequence can include for example, constructing an infectious cDNA clone, using an overlap extension PCR strategy, or long PCR-based fusion strategy.

### Modified Flavivirus

Various embodiments of the invention provide for a modified Flavivirus virus in which expression of viral proteins is reduced compared to a parent virus. The reduction in expression is the result of recoding the prM, or envelope (E) region or both. In some embodiments the parent virus is a wild type Flavivirus, and thus, comparisons are made to the wild-type virus or sequences in the wild-type virus.

In various embodiments, the E protein-encoding sequence is recoded by reducing the codon pair bias or codon usage bias of the protein-encoding sequence. In various embodiments, reducing the codon-pair bias comprises identifying a codon pair in the parent protein-encoding sequence having a codon-pair score that can be reduced, and reducing the codon-pair bias by substituting the codon pair with a codon pair that has a lower codon-pair score. In other embodiments, reducing the codon-pair bias comprises rearranging the codons of a parent protein-encoding sequence.

In various embodiments, each of the recoded prM/E protein-encoding sequence have a codon pair bias less than, −0.05, −0.1, or less than −0.2, or less than −0.3, or less than −0.4.

In certain embodiments, the recoded prM protein-encoding sequence has a codon pair bias less than −0.05, or less than −0.06, or less than −0.07, or less than −0.08, or less than −0.09, or less than −0.1, or less than −0.11, or less than −0.12, or less than −0.13, or less than −0.14, or less than −0.15, or less than −0.16, or less than −0.17, or less than −0.18, or less than −0.19, or less than −0.2, or less than −0.25, or less than −0.3, or less than −0.35, or less than −0.4, or less than −0.45, or less than −0.5.

In certain embodiments, the recoded E protein-encoding sequence has a codon pair bias less than −0.05, or less than −0.06, or less than −0.07, or less than −0.08, or less than −0.09, or less than −0.1, or less than −0.11, or less than −0.12, or less than −0.13, or less than −0.14, or less than −0.15, or less than −0.16, or less than −0.17, or less than −0.18, or less than −0.19, or less than −0.2, or less than −0.25, or less than −0.3, or less than −0.35, or less than −0.4, or less than −0.45, or less than −0.5.

In certain embodiments, the codon pair bias of the recoded prM protein encoding sequence is reduced by at least 0.05, or at least 0.06, or at least 0.07, or at least 0.08, or at least 0.09, or at least 0.1, or at least 0.11, or at least 0.12, or at least 0.13, or at least 0.14, or at least 0.15, or at least 0.16, or at least 0.17, or at least 0.18, or at least 0.19, or at least 0.2, or at least 0.25, or at least 0.3, or at least 0.35, or at least 0.4, or at least 0.45, or at least 0.5, compared to the parent prM protein encoding sequence from which it is derived.

In certain embodiments, the codon pair bias of the recoded E protein encoding sequence is reduced by at least 0.05, or at least 0.06, or at least 0.07, or at least 0.08, or at least 0.09, or at least 0.1, or at least 0.11, or at least 0.12, or at least 0.13, or at least 0.14, or at least 0.15, or at least 0.16, or at least 0.17, or at least 0.18, or at least 0.19, or at least 0.2, or at least 0.25, or at least 0.3, or at least 0.35, or at least 0.4, or at least 0.45, or at least 0.5, compared to the parent E protein encoding sequence from which it is derived. In certain embodiments, it is in comparison to an E protein encoding sequence from which the calculation is to be made; for example, the E protein encoding sequence of a wild type virus.

In various embodiments, the E protein-encoding sequence is recoded by increasing the number of CpG or UpA di nucleotides compared to its parent virus. In various embodiments, the E protein-encoding sequence is recoded by modifying G+C content compared to its parent virus.

In various embodiments, the E protein-encoding sequence is recoded by replacing one or more codons with synonymous codons that are less frequent in the viral host; for example, human.

In various embodiments, the E protein-encoding sequence is recoded by replacing one or more codons with synonymous codons that are less frequent in the virus itself.

In some embodiments, the number of codons substituted in the prM protein encoding sequence with synonymous codons is at least 5, or at least 10, or at least 30, or at least 30, or at least 40, or at least 50, or at least 75, or at least 100, or at least 150.

In some embodiments, the number of codons substituted in the E protein encoding sequence with synonymous codons is at least 5, or at least 10, or at least 30, or at least 30, or at least 40, or at least 50, or at least 75, or at least 100, or at least 125 or at least 150, or at least 175.

In various embodiments, the parent virus is a Flavivirus selected from the group consisting of dengue fever virus, West Nile virus, yellow fever virus, Japanese encephalitis virus, Spondweni virus, Zika virus, Saint Louis encephalitis virus, and Powassan virus. In various embodiments, the parent virus is a natural isolate. In various embodiments, the parent virus is a mutant of a natural isolate.

### Modifed Dengue Viruses

Various embodiments of the present invention provide for modified dengue viruses as the modified Flavivirus.

In various embodiments, a modified dengue virus is provided in which expression of viral proteins is reduced compared to a parent virus, wherein the reduction in expression is the result of recoding the prM, or envelope (E) region, or both. In some embodiments, a parent dengue virus is a wild-type dengue virus. As such, comparisons can be made in reference to a wild-type dengue virus.

In various embodiments, one or both of the E protein-encoding sequence is recoded by reducing the codon pair bias or codon usage bias of the protein-encoding sequence. In various embodiments, reducing the codon-pair bias comprises identifying a codon pair in the parent protein-encoding sequence having a codon-pair score that can be reduced, and reducing the codon-pair bias by substituting the codon pair with a codon pair that has a lower codon-pair score. In various embodiments, reducing the codon-pair bias comprises rearranging the codons of a parent protein-encoding sequence.

Various embodiments of the present invention provide for a modified dengue virus, comprising a recoded prM protein, a recoded envelope (E) protein, or both, wherein the recoded prM protein has a reduced codon pair bias compared to its parent prM protein encoding sequence, or has at least 5 codons substituted with synonymous codons less frequently used, or has an increased number of CpG or UpA di-nucleotides compared its parent prM protein encoding sequence, and wherein the recoded E protein has a reduced codon pair bias compared to its parent E protein encoding sequence, or has at least 5 codons substituted with synonymous codons less frequently used, or has an increased number of CpG or UpA di-nucleotides compared its parent E protein encoding sequence. In various embodiments, “its parent protein encoding sequence” is “a wild-type dengue protein encoding sequence”, for example, “a wild-type dengue E protein encoding sequence”, “a wild-type dengue prM protein encoding sequence”.

In various embodiments, the expression of the prM protein or E protein or both are reduced compared to its parent dengue virus.

In various embodiments, the recoded prM protein has a reduced codon pair bias compared to its parent prM protein encoding sequence. In various embodiments, the codon pair bias of the recoded prM encoding sequence is reduced by at least 0.05. In certain embodiments, the codon pair bias of the recoded prM protein encoding sequence of the dengue virus is reduced by at least 0.06, or at least 0.07, or at least 0.08, or at least 0.09, or at least 0.1, or at least 0.11, or at least 0.12, or at least 0.13, or at least 0.14, or at least 0.15, or at least 0.16, or at least 0.17, or at least 0.18, or at least 0.19, or at least 0.2, or at least 0.25, or at least 0.3, or at least 0.35, or at least 0.4, or at least 0.45, or at least 0.5, compared to the parent dengue virus' prM protein encoding sequence from which it is derived. In certain embodiments, it is in comparison to a prM protein encoding sequence from which the calculation is to be made; for example, a prM protein encoding sequence of a wild-type dengue virus.

In various embodiments, the recoded prM protein has at least 5 codons substituted with synonymous codons less frequently used. In various embodiments, the recoded prM protein has at least 10, 20, 25, 30, 35, 40, 45, 50 or 55 codons substituted with synonymous codons less frequently used. In some embodiments, the substitution with synonymous codons less frequently used are one that are less frequently used in the viral host; for example, human, mosquitos. In some embodiments, the substitution with synonymous codons less frequently used are one that are less frequently used in the virus itself.

In various embodiments, the recoded prM protein has an increased number of CpG or UpA di-nucleotides compared its parent prM protein encoding sequence. In various embodiments, the recoded prM protein has an increase of 15-55 CpG or UpA di-nucleotides compared its parent prM protein encoding sequence. In various embodiments, the recoded prM protein has an increase of about 15, 20, 25, 30, 35, 40, 45 or 55 CpG or UpA di-nucleotides compared its parent prM protein encoding sequence.

In various embodiments, the recoded E protein has a reduced codon pair bias compared to its parent E protein encoding sequence. In various embodiments, the codon pair bias of E protein-encoding sequence is reduced by at least 0.05 compared to its parent E protein encoding sequence. In certain embodiments, the codon pair bias of the recoded E protein encoding sequence of the dengue virus is reduced by at least 0.06, or at least 0.07, or at least 0.08, or at least 0.09, or at least 0.1, or at least 0.11, or at least 0.12, or at least 0.13, or at least 0.14, or at least 0.15, or at least 0.16, or at least 0.17, or at least 0.18, or at least 0.19, or at least 0.2, or at least 0.25, or at least 0.3, or at least 0.35, or at least 0.4, or at least 0.45, or at least 0.5, compared to its parent dengue virus' E protein encoding sequence from which it is derived. In certain embodiments, it is in comparison to an E protein encoding sequence from which the calculation is to be made; for example, the E protein encoding sequence of a wild-type dengue virus.

In various embodiments, the recoded E protein has at least 5 codons substituted with synonymous codons less frequently used. In various embodiments, the recoded E protein has at least 10, 20, 25, 30, 35, 40, 45, 50, 55, 60, 65, 70, 75, 80, 85, 90, 95, 100, 125, 150, or 175 codons substituted with synonymous codons less frequently used. In some embodiments, the substitution with synonymous codons less frequently used are one that are less frequently used in the viral host; for example, human, mosquitos. In some embodiments, the substitution with synonymous codons less frequently used are one that are less frequently used in the virus itself.

In various embodiments, the recoded E protein has an increased number of CpG or UpA di-nucleotides compared its parent E protein encoding sequence. In various embodiments, the recoded prM protein has an increase of 5-12 CpG or UpA di-nucleotides compared its parent E protein encoding sequence. In various embodiments, the recoded prM protein has an increase of about 5, 6, 7, 8, 9, 10 11 or 12 CpG or UpA di-nucleotides compared its parent E protein encoding sequence.

In various embodiments, each of the recoded prM or E protein-encoding sequence has a codon pair bias of less than −0.05. In certain embodiments, the recoded prM protein encoding sequence has a codon pair bias of less than −0.05, or less than −0.06, or less than −0.07, or less than −0.08, or less than −0.09, or less than −0.1, or less than −0.11, or less than −0.12, or less than −0.13, or less than −0.14, or less than −0.15, or less than −0.16, or less than −0.17, or less than −0.18, or less than −0.19, or less than −0.2, or less than −0.25, or less than −0.3, or less than −0.35, or less than −0.4, or less than −0.45, or less than −0.5. In certain embodiments, the recoded E protein encoding sequence has a codon pair bias of less than −0.05, or less than −0.06, or less than −0.07, or less than −0.08, or less than −0.09, or less than −0.1, or less than −0.11, or less than −0.12, or less than −0.13, or less than −0.14, or less than −0.15, or less than −0.16, or less than −0.17, or less than −0.18, or less than −0.19, or less than −0.2, or less than −0.25, or less than −0.3, or less than −0.35, or less than −0.4, or less than −0.45, or less than −0.5.

In various embodiments, the modified dengue virus is selected from type 1, type 2, type 3, type 4 or a combination thereof. For example, type 1 and type 2, type 1 and type 3, type 1 and type 4, type 2 and type 3, type 2 and type 4, or type 3 and type 4. Additional examples are type 1, type 2 and type 3; type 1, type 3 and type 4; or type 2, type 3 and type 4. In various embodiments, the modified dengue virus is a modified tetravalent dengue virus (i.e., type 1, type 2, type 3 and type 4).

### Methods

Various embodiments provide for a method of eliciting an immune response in a subject, comprising: administering to the subject an effective dose of a composition comprising a modified dengue virus of the present invention as described above and herein.

In various embodiments, the immune response is a protective immune response, and a prophylactically effective or therapeutically effective dose from 10³ to 10⁷ of a vaccine composition of claims is administered. In various embodiments, the immune response is a protective immune response, and a prophylactically effective or therapeutically effective dose of 10³, 10⁴, 10⁵, 10⁶, or 10⁷ of a vaccine composition of claims is administered. In various embodiments, the method further comprises administering to the subject at least one adjuvant. In various embodiments, the immune response is cross-protective against a heterologous dengue virus.

Various embodiments provide for a method of eliciting an immune response in a subject in need thereof, comprising: administering a prime dose of (i) an attenuated dengue virus produced by a method other than codon-pair deoptimization or codon deoptimization, or increasing of CpG or UpA di-nucleotides, or (ii) a modified dengue virus comprising a recoded prM protein, a recoded envelope (E) protein, or both, wherein the recoded prM protein has a reduced codon pair bias compared to its parent prM protein encoding sequence, or has at least 5 codons substituted with synonymous codons less frequently used, or has an increased number of CpG or UpA di-nucleotides compared its parent prM protein encoding sequence, and wherein the recoded E protein has a reduced codon pair bias compared to its parent E protein encoding sequence, or has at least 5 codons substituted with synonymous codons less frequently, or has an increased number of CpG or UpA di-nucleotides compared its parent E protein encoding sequence; and administering one or more boost dose of (i) the attenuated dengue virus produced by methods other than codon-pair deoptimization, or codon deoptimization, or increasing of CpG or UpA di-nucleotides or (ii) the modified dengue virus to the subject in need thereof, wherein at least the prime dose or the one or more boost dose is the modified dengue virus.

In various embodiments, a first of the one or more boost dose is administered about 2 weeks after the prime dose.

In various embodiments, the expression of the prM protein or E protein or both are reduced compared to its parent dengue virus. In various embodiments the parent dengue virus is a wild-type dengue virus.

In various embodiments, the recoded prM protein has a reduced codon pair bias compared to its parent prM protein encoding sequence. In various embodiments, the codon pair bias of the recoded prM encoding sequence is reduced by at least 0.05. In certain embodiments, the codon pair bias of the recoded prM protein encoding sequence of the dengue virus is reduced by at least 0.06, or at least 0.07, or at least 0.08, or at least 0.09, or at least 0.1, or at least 0.11, or at least 0.12, or at least 0.13, or at least 0.14, or at least 0.15, or at least 0.16, or at least 0.17, or at least 0.18, or at least 0.19, or at least 0.2, or at least 0.25, or at least 0.3, or at least 0.35, or at least 0.4, or at least 0.45, or at least 0.5, compared to the parent dengue virus' prM protein encoding sequence from which it is derived. In certain embodiments, it is in comparison to a prM protein encoding sequence from which the calculation is to be made; for example, a prM protein encoding sequence of a wild-type dengue virus.

In various embodiments, the recoded prM protein has at least 5 codons substituted with synonymous codons less frequently used. In various embodiments, the recoded prM protein has at least 10, 20, 25, 30, 35, 40, 45, 50 or 55 codons substituted with synonymous codons less frequently used. In some embodiments, the substitution with synonymous codons less frequently used are one that are less frequently used in the viral host; for example, human, mosquitos. In some embodiments, the substitution with synonymous codons less frequently used are one that are less frequently used in the virus itself.

In various embodiments, the recoded prM protein has an increased number of CpG or UpA di-nucleotides compared its parent prM protein encoding sequence. In various embodiments, the recoded prM protein has an increase of 15-55 CpG or UpA di-nucleotides compared its parent prM protein encoding sequence. In various embodiments, the recoded prM protein has an increase of about 15, 20, 25, 30, 35, 40, 45 or 55 CpG or UpA di-nucleotides compared its parent prM protein encoding sequence. In certain embodiments, it is in comparison to a prM protein encoding sequence from which the calculation is to be made; for example, a prM protein encoding sequence of a wild-type dengue virus.

In various embodiments, the recoded E protein has a reduced codon pair bias compared to its parent E protein encoding sequence. In various embodiments, the codon pair bias of E protein-encoding sequence is reduced by at least 0.05 compared to its parent E protein encoding sequence. In certain embodiments, the codon pair bias of the recoded E protein encoding sequence of the dengue virus is reduced by at least 0.06, or at least 0.07, or at least 0.08, or at least 0.09, or at least 0.1, or at least 0.11, or at least 0.12, or at least 0.13, or at least 0.14, or at least 0.15, or at least 0.16, or at least 0.17, or at least 0.18, or at least 0.19, or at least 0.2, or at least 0.25, or at least 0.3, or at least 0.35, or at least 0.4, or at least 0.45, or at least 0.5, compared to its parent dengue virus' E protein encoding sequence from which it is derived. In certain embodiments, it is in comparison to an E protein encoding sequence from which the calculation is to be made; for example, the E protein encoding sequence of a wild-type dengue virus.

In various embodiments, the recoded E protein has at least 5 codons substituted with synonymous codons less frequently used. In various embodiments, the recoded E protein has at least 10, 20, 25, 30, 35, 40, 45, 50, 55, 60, 65, 70, 75, 80, 85, 90, 95, 100, 125, 150, or 175 codons substituted with synonymous codons less frequently used. In some embodiments, the substitution with synonymous codons less frequently used are one that are less frequently used in the viral host; for example, human, mosquitos. In some embodiments, the substitution with synonymous codons less frequently used are one that are less frequently used in the virus itself.

In various embodiments, the recoded E protein has an increased number of CpG or UpA di-nucleotides compared its parent E protein encoding sequence. In various embodiments, the recoded prM protein has an increase of 5-12 CpG or UpA di-nucleotides compared its parent E protein encoding sequence. In various embodiments, the recoded prM protein has an increase of about 5, 6, 7, 8, 9, 10 11 or 12 CpG or UpA di-nucleotides compared its parent E protein encoding sequence. In certain embodiments, it is in comparison to an E protein encoding sequence from which the calculation is to be made; for example, the E protein encoding sequence of a wild-type dengue virus.

In various embodiments, each of the recoded prM or E protein-encoding sequence has a codon pair bias of less than −0.05. In certain embodiments, the recoded prM protein encoding sequence has a codon pair bias of less than −0.05, or less than −0.06, or less than −0.07, or less than −0.08, or less than −0.09, or less than −0.1, or less than −0.11, or less than −0.12, or less than −0.13, or less than −0.14, or less than −0.15, or less than −0.16, or less than −0.17, or less than −0.18, or less than −0.19, or less than −0.2, or less than −0.25, or less than −0.3, or less than −0.35, or less than −0.4, or less than −0.45, or less than −0.5. In certain embodiments, the recoded E protein encoding sequence has a codon pair bias of less than −0.05, or less than −0.06, or less than −0.07, or less than −0.08, or less than −0.09, or less than −0.1, or less than −0.11, or less than −0.12, or less than −0.13, or less than −0.14, or less than −0.15, or less than −0.16, or less than −0.17, or less than −0.18, or less than −0.19, or less than −0.2, or less than −0.25, or less than −0.3, or less than −0.35, or less than −0.4, or less than −0.45, or less than −0.5.

In various embodiments, the modified dengue virus is selected from type 1, type 2, type 3, type 4 or a combination thereof. For example, type 1 and type 2, type 1 and type 3, type 1 and type 4, type 2 and type 3, type 2 and type 4, or type 3 and type 4. Additional examples are type 1, type 2 and type 3; type 1, type 3 and type 4; or type 2, type 3 and type 4. In various embodiments, the modified dengue virus is a modified tetravalent dengue virus (i.e., type 1, type 2, type 3 and type 4).

Methods of Making Modified Flavivirus virus genome

Various embodiments of the present invention provide for a method of making a modified Flavivirus virus genome. The method comprises obtaining the nucleotide sequence encoding the envelope protein of a Flavivirus virus and the nucleotide sequence encoding the nonstructural 3 proteins of a Flavivirus virus; recoding the envelope encoding nucleotide sequence to reduce protein expression and recoding the nonstructural protein 3-encoding nucleotide sequence to reduce protein expression, and substituting a nucleic acid having the recoded envelope-encoding nucleotide sequence and a nucleic acid having the recoded nonstructural protein 3-encoding nucleotide sequence into a parent Flavivirus virus genome to make a modified Flavivirus virus genome; whereby expression of the recoded envelope-encoding nucleotide sequence and expression of the recoded nonstructural protein 3-encoding nucleotide sequence is reduced compared to the parent virus.

Various embodiments of the present invention provide for a method of making a modified dengue virus genome comprising: obtaining the nucleotide sequence encoding the envelope protein of a dengue virus and the nucleotide sequence encoding the nonstructural 3 proteins of a dengue virus; recoding the envelope encoding nucleotide sequence to reduce protein expression and recoding the nonstructural protein 3-encoding nucleotide sequence to reduce protein expression, and substituting a nucleic acid having the recoded envelope-encoding nucleotide sequence and a nucleic acid having the recoded nonstructural protein 3-encoding nucleotide sequence into a parent dengue virus genome to make a modified dengue virus genome; whereby expression of the recoded envelope-encoding nucleotide sequence and expression of the recoded nonstructural protein 3-encoding nucleotide sequence is reduced compared to the parent virus.

Various embodiments of the present invention provide for a method of making a modified dengue virus genome comprising: obtaining a nucleotide sequence encoding the envelope protein of a dengue virus; recoding the envelope encoding nucleotide sequence to reduce protein expression, and substituting a nucleic acid having the recoded envelope-encoding nucleotide sequence into a parent dengue virus genome to make a modified dengue virus genome; whereby expression of the recoded envelope-encoding nucleotide sequence is reduced compared to the parent virus.

According to various embodiments the invention, viral attenuation is accomplished by reducing expression viral proteins through codon pair deoptimization of E coding sequence. One way to reduce expression of the coding sequences is by a reduction in codon pair bias, but other methods can also be used, alone or in combination. While codon bias may be changed, adjusting codon pair bias is particularly advantageous. For example, attenuating a virus through codon bias generally requires elimination of common codons, and so the complexity of the nucleotide sequence is reduced. In contrast, codon pair bias reduction or minimization can be accomplished while maintaining far greater sequence diversity, and consequently greater control over nucleic acid secondary structure, annealing temperature, and other physical and biochemical properties.

Codon pair bias of a protein-encoding sequence (i.e., an open reading frame) is calculated as set forth above and described in Coleman et al., 2008.

Viral attenuation and induction or protective immune responses can be confirmed in ways that are well known to one of ordinary skill in the art, including but not limited to, the methods and assays disclosed herein. Non-limiting examples include plaque assays, growth measurements, reduced lethality in test animals, and protection against subsequent infection with a wild type virus.

In various embodiments, the invention provides viruses that are highly attenuated, and induce immunity against a plurality of dengue types and/or subtypes. Such dengue virus varieties include viruses in serogroups 1, 2, 3, and 4. Examples of attenuated dengue protein coding sequences are provided below.

TABLE 4
Reduced-Expression Dengue Virus Genes

| | WT Coding Sequence | Recoded Coding Sequence |
|---|---|---|
| Gene | SEQ ID NO: | SEQ ID NO: |
| DENV1-WT | 1 | |
| DENV1-E-Min | | 2 |
| DENV2-WT | 3 | |
| DENV2-E-Min | | 4 |
| DENV3-WT | 5 | |
| DENV3-E-Min | | 6 |
| DENV4-WT | 7 | |
| DENV4-E-Min | | 8 |
| DENV1-E-W/Min | | 9 |
| DENV2-E-W/Min | | 10 |
| DENV3-E-W/Min | | 11 |
| DENV4-E-W/Min | | 12 |

### Compositions

Various embodiments provide for a Flavivirus composition for inducing an immune response in a subject, which comprises the modified Flavivirus of the present invention as described herein.

Various embodiments provide for a Flavivirus vaccine composition for inducing a protective immune response in a subject, which comprises the modified Flavivirus of the present invention as described herein.

Various embodiments provide for a modified dengue virus composition for inducing an immune response in a subject, comprising a modified dengue virus of the present invention as described above and herein, and a pharmaceutically acceptable excipient or carrier.

Various embodiments provide for a dengue vaccine composition for inducing a protective immune response in a subject, comprising a modified dengue virus of the present invention as described above and herein, and a pharmaceutically acceptable excipient or carrier.

In various embodiments, the modified dengue virus is selected from type 1, type 2, type 3, type 4 or a combination thereof.

In various embodiments, the modified dengue virus is a type 1 and type 2 modified dengue virus; a type 1 and type 3 modified dengue virus; a type 1 and type 4 modified dengue virus; a type 2 and type 3 modified dengue virus; a type 2 and type 4 modified dengue virus; a type 3 and type 4 modified dengue virus.

In various embodiments, the modified dengue virus is a type 1, type 2 and type 3 modified dengue virus; a type 1, type 2 and type 4 modified dengue virus; a type 1, type 3 and type 4 modified dengue virus; or a type 2, type 3 and type 4 modified dengue virus.

In various embodiments, the modified dengue virus modified tetravalent dengue virus; (type 1, type 2, type 3 and type 4).

Non-limiting examples of wild-type and modified dengue viruses are herein:

DENV-1-VN-BID-V1774-2007(WT)
SEQ ID NO: 1
AGTTGTTAGTCTACGTGGACCGACAAGAACAGTTTCGAATCGGAA
GCTTGCTTAACGTAGTTCTAACAGTTTTTTATTAGAGAGCAGATC
TCTGATGAACAACCAACGGAAAAAGACGGCTCGACCGTCTTTCAA
TATGCTGAAACGCGCGAGAAACCGCGTGTCAACTGTTTCACAGTT
GGCGAAGAGATTCTCAAAAGGATTGCTCTCAGGCCAAGGACCCAT
GAAATTGGTGATGGCTTTCATAGCATTCCTAAGATTTCTAGCCAT
ACCCCCAACAGCAGGAATTTTGGCTAGATGGGGTTCATTCAAGAA
GAGTGGAGCGATCAAAGTGCTACGGGGTTTCAAGAAAGAAATCTC
AAACATGTTGAATATAATGAATAGAAGGAAAAGATCTGTGACCAT
GCTCCTTATGCTGATGCCTACAGCCTTGGCGTTCCATTTGACTAC
ACGAGGGGGAGAGCCGCACATGATAGTCAGCAAGCAGGAAAGAGG
AAAGTCACTCTTGTTTAAGACCTCAGCAGGTGTCAACATGTGCAC
CCTTATAGCGATGGATTTGGGAGAGTTATGTGAGGACACAATGAC
TTACAAATGCCCTCGAATCACTGAAACTGAACCAGATGACGTTGA
TTGTTGGTGTAATGCCACAGACACATGGGTGACCTATGGAACATG
TTCCCAAACTGGCGAGCACCGACGAGACAAACGTTCCGTCGCACT
GGCCCCACACGTGGGACTTGGTTTGGAAACAAGAACCGAAACGTG
GATGTCCTCTGAAGGCGCTTGGAAACAGATACAAAGAGTGGAGAC
TTGGGCCCTGAGACACCCAGGATTCACGGTGATAGCCCTTTTTCT
AGCACATGCCATAGGAACATCCATCACCCAGAAAGGGATTATTTT
CATTTTGTTAATGCTGGTAACACCATCCATGGCCATGCGATGCGT
GGGAATAGGCAGCAGGGACTTCGTGGAAGGACTGTCAGGAGCAAC
TTGGGTAGATGTGGTACTGGAACATGGAAGTTGCGTCACCACCAT
GGCAAAAGACAAACCAACATTGGACATTGAACTCTTGAAGACGGA
AGTCACAAACCCTGCCGTCCTGCGCAAACTGTGCATTGAAGCTAA
AATATCAAACACCACCACCGACTCAAGATGTCCAACACAAGGAGA
AGCCACACTAGTGGAAGAACAAGACGCGAACTTTGTGTGTCGACG
AACGTTTGTGGACAGAGGCTGGGGCAATGGCTGTGGCCTCTTCGG
AAAAGGAAGCCTAATAACGTGTGCAAAGTTCAAGTGTGTGACAAA
ACTGGAAGGAAAGATAGTTCAATATGAGAACTTGAAATATTCAGT
AATAGTCACCGTTCACACCGGAGACCAGCATCAAGTGGGAAATGA
AAGCACAGAACATGGGACAACTGCAACTATAACACCTCAAGCTCC
TACGACGGAAATACAGCTGACCGACTACGGAGCTCTTACATTGGA
TTGTTCACCTAGAACAGGACTAGACTTTAATGAAATGGTGTTGTT
GACAATGAAAGAAAAATCATGGCTAGTCCACAAACAATGGTTTTT
AGACCTACCACTGCCTTGGACCTCGGGAGCTTCAACATCACAA
GAGACTTGGAACAGACAAGATTTGCTGGTGACATTTAAGACAGCT
CATGCAAAGAAGCAGGAAGTAGTCGTACTAGGATCACAAGAAGGA
GCAATGCACACTGCGTTGACCGGAGCGACAGAAATCCAAACGTCT
GGAACGACAACAATTTTTGCAGGACACTTGAAATGCAGACTAAAG
ATGGACAAACTGACTCTAAAAGGGATGTCATATGTGATGTGCACA
GGCTCATTCAAGCTAGAGAAAGAAGTGGCTGAGACCCAGCATGGA
ACCGTTCTAGTGCAGATTAAATACGAAGGAACAGATGCACCATGC
AAGATCCCTTTTTCGACCCAAGATGAAAAAGGAGTAACCCAGAAT
GGGAGATTGATAACAGCTAACCCCATAGTTACTGACAAAGAAAAA
CCAGTCAACATTGAGGCAGAACCGCCCTTTGGTGAGAGTTACATC
GTAATAGGAGCAGGTGAAAAAGCTTTGAAACTAAGCTGGTTCAAG
AAAGGAAGCAGCATAGGGAAAATGTTTGAGGCAACTGCCAGAGGA
GCACGAAGGATGGCCATACTGGGAGACACCGCATGGGACTTTGGT
TCTATAGGAGGAGTGTTCACGTCTGTTGGAAAATTAGTACACCAG
ATTTTTGGAACTGCATATGGAGTTTTGTTCAGCGGTGTTTCCTGG
ACCATGAAAATAGGAATAGGGGTTCTGCTGACATGGCTGGGATTA
AACTCAAGGAGCACGTCCCTTTCGATGACGTGCATTGCAGTTGGC
CTAGTAACACTATACCTAGGAGTCATGGTTCAGGCGGATTCAGGA
TGTGTAATTAATTGGAAAGGTAGAGAACTCAAGTGTGGAAGTGGC
ATTTTTGTCACCAATGAAGTTCACACTTGGACAGAGCAATACAAA
TTTCAAGCTGACTCCCCTAAGAGACTATCAGCAGCCATCGGGAAG
GCATGGGAGGAGGGTGTGTGTGGAATTCGATCAGCAACTCGTCTC
GAGAACATCATGTGGAAGCAAATATCAAATGAACTGAATCACATC
TTACTTGAAAATGATATGAAATTCACAGTGGTTGTAGGAGATGTT
GCTGGGATCTTGGCTCAAGGAAAGAAAATGATTAGGCCACAACCC
ATGGAATACAAATACTCGTGGAAAAGCTGGGGAAAGGCTAAAATC
ATAGGGGCAGATGTACAGAACACCACCTTCATCATCGATGGCCCA
AACACCCCAGAATGCCCTGATGACCAAAGAGCATGGAACATTTGG
GAAGTTGAGGACTATGGATTTGGAATTTTCACGACAAACATATGG
CTGAAATTGCGTGATTCCTACACCCAAGTGTGTGACCACCGGCTA
ATGTCAGCTGCCATCAAGGACAGCAAGGCAGTTCACGCTGACATG
GGGTACTGGATAGAAAGTGAAAAGAACGAGACCTGGAAGCTGGCA
AGAGCCTCATTCATAGAAGTTAAAACATGTATCTGGCCAAAATCC
CACACTCTATGGAGCAATGGAGTTCTGGAAAGTGAAATGATAATT
CCAAAGATCTATGGAGGACCAATATCTCAGCACAACTACAGACCA
GGATATTTTACACAAGCAGCAGGGCCGTGGCACCTAGGCAAGTTG
GAACTGGATTTTGATTTGTGTGAGGGTACCACAGTTGTTGTGGAT
GAACATTGTGGAAATCGAGGACCATCTCTTAGGACCACAACAGTC
ACAGGAAAGATAATTCATGAATGGTGTTGCAGATCTTGCACGCTA
CCACCCTTACGTTTCAGAGGAGAAGATGGGTGCTGGTACGGTATG
GAAATCAGACCAGTCAAGGAAAAGGAAGAAAATCTAGTCAAATCA
ATGGTCTCTGCAGGGTCAGGGGAAGTGGACAGCTTTTCACTAGGA
CTGCTATGCATATCAATAATGATCGAGGAGGTGATGAGATCCAGA
TGGAGTAGAAGAATGCTGATGACTGGAACACTGGCTGTGTTCTTC
CTTCTCATAATGGGACAATTGACATGGAACGATCTGATCAGATTA
TGCATCATGGTTGGAGCCAACGCTTCCGACAGGATGGGGATGGGA
ACGACGTACCTAGCCCTGATGGCCACTTTTAAAATGAGACCGATG
TTCGCTGTAGGGCTATTATTTCGCAGACTAACATCCAGAGAAGTT
CTTCTTCTAACAATTGGATTGAGTCTAGTGGCATCTGTGGAGTTA
CCAAATTCCTTGGAGGAGCTGGGGGATGGACTTGCAATGGGCATT
ATGATTTTAAAATTATTGACTGACTTTCAATCACATCAGCTGTGG
GCTACCTTGCTGTCCTTGACATITATCAAAACAACGTTTTCCTTG
CACTACGCATGGAAGACAATGGCTATGGTACTGTCAATTGTATCT
CTCTTCCCTTTATGCCTGTCCACGACCTCCCAAAAAACAACATGG
CTTCCGGTGCTATTGGGATCCCTTGGATGCAAACCACTAACCATG
TTTCTTATAGCAGAAAACAAAATCTGGGGAAGGAGAAGTTGGCCC
CTCAATGAAGGAATCATGGCTGTTGGAATAGTCAGCATCCTACTA
AGTTCACTCCTCAAAAATGATGTACCGCTAGCTGGGCCACTAATA
GCTGGAGGCATGCTAATAGCATGTTATGTTATATCTGGAAGCTCA
GCCGACCTATCATTAGAGAAAGCGGCTGAGGTCTCCTGGGAAGAA
GAAGCAGAACACTCTGGTGCCTCACACAACATATTAGTGGAAGTC
CAAGATGATGGAACCATGAAGATAAAGGATGAAGAGAGAGATGAC
ACGCTAACCATTCTCCTTAAAGCAACTCTGTTAGCAGTTTCAGGG
GTGTACCCATTATCAATACCAGCGACCCTTTTCGTGTGGTACTTT
TGGCAGAAAAAGAAACAGAGATCTGGAGTGTTATGGGACACACCC
AGCCCTCCAGAAGTGGAAAGAGCAGTTCTTGATGATGGTATCTAT
AGAATTATGCAGAGAGGACTGTTGGGCAGGTCCCAAGTAGGGGTA
GGAGTTTTCCAAGAAAACGTGTTCCACACAATGTGGCATGTCACC
AGGGGAGCTGTACTCATGTATCAAGGGAAGAGATTGGAACCGAGC
TGGGCCAGTGTCAAAAAAGACCTGATCTCATATGGAGGAGGTTGG
AGGCTTCAAGGATCCTGGAACACAGGAGAAGAAGTGCAGGTGATT
GCTGTTGAACCAGGGAAAAACCCCAAAAATGTACAAACAGCGCCG
GGCACCTTTAAGACCCCTGAAGGTGAAGTTGGAGCCATTGCCCTA
GACTTTAAACCTGGCACATCTGGATCTCCCATCGTGAACAGAGAA
GGAAAAATAGTAGGTCTTTATGGAAATGGAGTAGTGACAACAAGT
GGAACCTACGTCAGTGCCATAGCTCAAGCCAAAGCATCACAAGAA
GGGCCCCTACCAGAGATTGAAGACGAGGTGTTTAGGAAAAGAAAC
TTAACAATAATGGACCTACATCCAGGATCGGGGAAAACAAGAAGA
TATCTTCCAGCCATAGTCCGTGAGGCCATAAAAAGGAAGCTGCGC
ACACTAATCCTGGCTCCCACAAGGGTTGTCGCTTCCGAAATGGCA
GAGGCGCTCAAGGGAATGCCAATAAGGTACCAAACAACAGCAGTG
AAGAGTGAACATACAGGAAAAGAGATAGTTGACCTCATGTGTCAC
GCCACTTTCACCATGCGCCTCCTGTCTCCCGTAAGAGTTCCCAAT
TACAACATGATCATCATGGATGAAGCACATTTCACCGATCCATCC
AGTATAGCGGCCAGAGGGTACATCTCAACCCGAGTGGGCATGGGT
GAAGCAGCTGCAATCTTCATGACAGCCACTCCCCCAGGATCAGTG
GAGGCCTTTCCACAGAGCAACGCAGTTATCCAAGATGAGGAAAGA
GACATTCCTGAGAGATCATGGAACTCAGGCTATGAGTGGATCACT
GACTTCCCAGGTAAAACAGTTTGGTTTGTTCCAAGCATTAAATCA
GGAAATGACATTGCCAACTGCTTAAGA
AAGAATGGGAAACGGGTGATTCAATTGAGCAGGAAAACCTTTGAT
ACAGAGTACCAAAAAACAAAAAACAACGACTGGGACTACGTCGTC
ACAACAGACATCTCCGAAATGGGAGCAAATTTCCGAGCCGACAGG
GTGATAGACCCAAGACGGTGTCTGAAACCGGTAATACTAAAAGAT
GGTCCAGAGCGTGTCATTTTAGCAGGACCAATGCCAGTGACTGTG
GCCAGTGCCGCTCAGAGGAGAGGAAGAATTGGAAGGAACCACAAT
AAGGAAGGTGATCAGTACATCTACATGGGACAGCCTTTAAACAAC
GATGAAGATCACGCTCACTGGACAGAAGCAAAAATGCTCCTTGAT
AATATAAACACACCAGAAGGGATTATCCCAGCCCTCTTTGAGCCA
GAGAGAGAAAAGAGTGCAGCAATAGACGGGGAATACAGACTGCGG
GGTGAAGCAAGGAAAACGTTTGTGGAGCTCATGAGAAGAGGAGAT
CTACCTGTCTGGCTATCCTACAAAGTTGCCTCAGAAGGCTTCCAG
TACTCTGACAGAAGATGGTGCTTTGACGGGGAAAGGAACAACCAG
GTGTTGGAGGAGAACATGGACGTGGAGATCTGGACAAAAGAAGGA
GAAAGAAAGAAACTACGACCCCGCTGGCTGGATGCCAGAACATAC
TCAGACCCACTAGCCCTGCGCGAGTTTAAAGAGTTTGCAGCAGGG
AGAAGAAGCGTCTCAGGTGATTTAATATTAGAAATAGGGAAGCTT
CCACAACACTTGACGCAAAGGGCCCAGAATGCCCTGGACAACCTG
GTTATGTTGCACAACTCCGAACAAGGAGGCAGAGCCTATAGACAT
GCAATGGAAGAACTGCCAGACACCATAGAAACGTTGATGCTCCTA
GCTTTGATAGCTGTGTTAACTGGTGGAGTGACACTGTTCTTCCTA
TCAGGAAGGGGCCTAGGGAAAACATCTATCGGCCTACTCTGCGTA
ATGGCTTCAAGCGTACTGCTATGGATGGCCAGTGTGGAGCCCCAT
TGGATAGCGGCCTCCATCATACTGGAGTTCTTCCTGATGGTGCTG
CTTATTCCAGAGCCAGACAGACAACGCACTCCGCAGGACAACCAG
CTGGCATATGTGGTGATAGGTTTGTTATTCATGATACTGACAGTA
GCAGCCAATGAGATGGGACTGCTGGAAACCACAAAGAAAGACTTA
GGGATTGGCCATGTGGCTGTTGAAAATCACCACCATGCCGCAATG
CTGGACGTAGACTTACATCCAGCTTCAGCCTGGACCCTCTATGCA
GTGGCCACAACAATTATCACTCCCATGATGAGGCACACAATCGAA
AACACAACGGCAAACATTTCCCTGACAGCCATTGCAAACCAGGCA
GCTATATTGATGGGACTTGACAAAGGATGGCCAATATCGAAGATG
GACATAGGAGTTCCACTTCTCGCCTTGGGGTGCTATTCCCAGGTG
AATCCACTGACGCTGACAGCGGCGGTATTGATGCTAGTGGCTCAT
TACGCCATAATTGGACCTGGACTGCAAGCAAAAGCTACTAGAGAA
GCTCAAAAAAGGACAGCGGCCGGAATAATGAAAAATCCAACCGTT
GATGGAATTGTTGCAATAGATTTGGACCCTGTGGTTTATGATGCA
AAATTTGAAAAACAACTAGGCCAAATAATGTTGTTGATACTATGC
ACATCACAGATCCTCTTGATGCGGACTACATGGGCCTTGTGCGAG
TCCATCACACTGGCCACTGGACCTCTGACCACGCTTTGGGAGGGA
TCTCCAGGAAAATTTTGGAACACCACGATAGCGGTTTCCATGGCA
AACATTTTCAGAGGAAGTTATCTAGCAGGAGCAGGTCTGGCCTTC
TCATTAATGAAATCTCTAGGAGGAGGTAGGAGAGGCACGGGAGCC
CAAGGGGAAACACTGGGAGAGAAATGGAAAAGACAGCTGAACCAA
CTGAGCAAGTCAGAATTTAACACCTATAAAAGGAGTGGGATTATG
GAAGTGGACAGATCCGAAGCCAAAGAGGGATTGAAAAGAGGAGAA
ACAACCAAACATGCAGTGTCGAGAGGAACCGCTAAACTGAGGTGG
TTCGTGGAGAGGAACCTTGTGAAACCAGAAGGGAAAGTCATAGAC
CTCGGTTGTGGAAGAGGTGGCTGGTCATACTATTGCGCTGGGCTG
AAGAAAGTCACAGAAGTGAAGGGGTATACAAAAGGAGGACCTGGA
CATGAGGAACCAATCCCAATGGCGACCTATGGATGGAACCTAGTA
AAGCTGCACTCTGGGAAAGACGTATTTTTTATACCACCTGAGAAA
TGTGACACCCTTTTGTGTGATATTGGTGAGTCCTCTCCAAACCCA
ACTATAGAGGAAGGAAGAACGCTACGCGTCCTAAAGATGGTGGAA
CCATGGCTCAGAGGAAACCAATTTTGCATAAAAATTCTGAATCCC
TACATGCCAAGTGTGGTGGAAACTCTGGAGCAAATGCAAAGAAAA
CATGGAGGAATGCTAGTGCGAAATCCACTTTCAAGAAATTCTACT
CATGAAATGTATTGGGTTTCATGTGGAACAGGAAACATTGTGTCA
GCAGTAAACATGACATCTAGAATGTTGCTAAATCGATTCACAATG
GCTCACAGGAAACCAACATATGAAAGAGACGTGGACCTAGGCGCC
GGAACAAGACATGTGGCAGTGGAACCAGAGGTAGCCAACCTAGAT
ATCATTGGCCAGAGGATAGAGAACATAAAACATGAACATAAGTCA
ACATGGCATTATGATGAGGACAATCCATATAAAACATGGGCCTAT
CATGGATCATATGAGGTCAAGCCATCAGGATCAGCCTCATCCATG
GTCAATGGCGTGGTGAAACTGCTCACCAAACCATGGGATGTCATC
CCTATGGTCACACAAATAGCCATGACTGACACTACACCCTTTGGA
CAACAGAGGGTGTTTAAAGAGAAAGTTGACACACGCACACCAAAA
GCAAAACGGGGCACAGCACAAATCATGGAGGTGACAGCCAAGTGG
TTATGGGGTTTTCTTTCTAGAAACAAGAAACCAAGAATTTGCACA
AGAGAGGAGTTCACAAGAAAAGTTAGGTCAAACGCAGCCATTGGA
GCAGTGTTCGTTGATGAAAATCAATGGAACTCAGCAAAAGAAGCA
GTGGAAGATGAGCGGTTCTGGGACCTTGTGCATAGAGAGAGGGAG
CTTCACAAACAGGGAAAATGTGCTACGTGTGTTTACAACATGATG
GGGAAGAGAGAGAAAAAGCTAGGAGAGTTCGGAAAGGCAAAAGGA
AGTCGTGCAATATGGTACATGTGGTTGGGAGCACGCTTTCTAGAG
TTCGAAGCTCTTGGTTTCATGAACGAAGATCACTGGTTCAGCAGA
GAGAATTCACTCAGCGGAGTGGAAGGAGAAGGACTCCACAAACTT
GGATATATACTCAGAGACATATCAAAGATTCCAGGGGGAAACATG
TATGCAGATGACACAGCCGGATGGGATACAAGGATAACAGAGGAT
GACCTTCAGAATGAGGCCAGAATTACTGACATCATGGAACCCGAA
CATGCCCTCCTGGCTAAGTCAATCTTCAAGTTAACCTACCAAAAT
AAGGTGGTAAGGGTACAGAGACCAGCAAAAAATGGAACCGTGATG
GATGTCATATCCAGACGTGACCAGAGAGGAAGTGGTCAGGTCGGA
ACTTATGGCTTAAACACTTTCACCAACATGGAAGCCCAGCTGATA
AGACAAATGGAGTCTGAGGGAATCTTTTCACCCAGCGAATTAGAG
ACCCCAAATTTAGCCGAGAGAGTTCTCGACTGGCTGGAAAAATAT
GGCGTCGAAAGGCTGAAAAGAATGGCAATCAGCGGAGATGACTGC
GTAGTGAAACCAATTGATGATAGGTTTGCAACAGCCTTGACAGCT
CTGAATGATATGGGAAAAGTAAGAAAAGATATACCACAATGGGAA
CCTTCAAAAGGATGGAATGATTGGCAACAAGTGCCTTTTTGTTCA
CACCACTTCCACCAGCTGATTATGAAGGATGGGAGGGAAATAGTG
GTGCCATGCCGCAACCAAGATGAACTTGTGGGTAGGGCTAGAGTA
TCACAAGGTGCTGGATGGAGCCTGAGAGAAACTGCATGCCTAGGC
AAGTCATATGCACAAATGTGGCAGCTGATGTACTTCCACAGGAGA
GACCTGAGACTAGCCGCTAATGCTATCTGTTCAGCCGTTCCAGTT
GATTGGATCCCAACCAGCCGTACCACCTGGTCGATCCATGCCCAT
CACCAATGGATGACAACAGAAGACATGCTGTCAGTGTGGAATAGG
GTTTGGATAGAGGAAAACCCATGGATGGAGGACAAAACCCACATA
TCCAGTTGGGGAGATGTTCCATATTTAGGAAAAAGGGAAGACCAA
TGGTGTGGATCCCTGATAGGCTTAACAGCAAGGGCCACCTGGGCC
ACCAACATACAAGTGGCCATAAACCAAGTGAGAAGACTAATTGGG
AATGAGAATTATCTAGATTACATGACATCAATGAAGAGATTCAAG
AACGAGAGTGATCCCGAAGGGGCACTCTGGTGAGTCAACACATTT
ACAAAATAAAGGAAAATAAGAAATCAAACAAGGCAAGAAGTCAGG
CCGGATTAAGCCATAGTACGGTAAGAGCTATGCTGCCTGTGAGCC
CCGTCTAAGGACGTAAAATGAAGTCAGGCCGAAAGCCACGGCTTG
AGCAAACCGTGCTGCCTGTAGCTCCATCGTGGGGATGTAAAAACC
TGGGAGGCTGCAACCCATGGAAGCTGTACGCATGGGGTAGCAGAC
TAGTGGTTAGAGGAGACCCCTCCCGAAACATAACGCAGCAGCGGG
GCCCAACACCAGGGGAAGCTGTACCCTGGTGGTAAGGACTAGAGG
TTAGAGGAGACCCCCCGCATAACAATAAACAGCATATTGACGCTG
GGAGAGACCAGAGATCCTGCTGTCTCTACAGCATCATTCCAGGCA
CAGAACGCCAGAAAATGGAATGGTGCTGTTGAATCAACAGGTTCT
DENV-1-VN-BID-V1774-2007-Emin
SEQ ID NO: 2
AGTTGTTAGTCTACGTGGACCGACAAGAACAGTTTCGAATCGGAA
GCTTGCTTAACGTAGTTCTAACAGTTTTTTATTAGAGAGCAGATC
TCTGATGAACAACCAACGGAAAAAGACGGCTCGACCGTCTTTCAA
TATGCTGAAACGCGCGAGAAACCGCGTGTCAACTGTTTCACAGTT
GGCGAAGAGATTCTCAAAAGGATTGCTCTCAGGCCAAGGACCCAT
GAAATTGGTGATGGCTTTCATAGCATTCCTAAGATTTCTAGCCAT
ACCCCCAACAGCAGGAATTTTGGCTAGATGGGGTTCATTCAAGAA
GAGTGGAGCGATCAAAGTGCTACGGGGTTTCAAGAAAGAAATCTC
AAACATGTTGAATATAATGAATAGAAGGAAAAGATCTGTGACCAT
GCTCCTTATGCTGATGCCTACAGCCTTGGCGTTCCATTTGACTAC
ACGAGGGGGAGAGCCGCACATGATAGTCAGCAAGCAGGAAAGAGG
AAAGTCACTCTTGTTTAAGACCTCAGCAGGTGTCAACATGTGCAC
CCTTATAGCGATGGATTTGGGAGAGTTATGTGAGGACACAATGAC
TTACAAATGCCCTCGAATCACTGAAACTGAACCAGATGACGTTGA
TTGTTGGTGTAATGCCACAGACACATGGGTGACCTATGGAACATG
TTCCCAAACTGGCGAGCACCGACGAGACAAACGTTCCGTCGCACT
GGCCCCACACGTGGGACTTGGTTTGGAAACAAGAACCGAAACGTG
GATGTCCTCTGAAGGCGCTTGGAAACAGATACAAAGAGTGGAGAC
TTGGGCCCTGAGACACCCAGGATTCACGGTGATAGCCCTTTTTCT
AGCACATGCCATAGGAACATCCATCACCCAGAAAGGGATTATTTT
CATTTTGTTAATGCTGGTAACACCATCCATGGCCATGCGATGCGT
AGGGATAGGGTCACGCGATTTCGTCGAAGGACTATCCGGAGCGAC
ATGGGTCGACGTCGTACTCGAACACGGATCATGCGTTACGACTAT
GGCTAAAGACAAACCTACACTAGACATAGAGTTACTGAAAACCGA
AGTTACGAATCCCGCCGTACTGCGAAAATTGTGTATCGAAGCGAA
AATTAGCAATACGACTACAGACTCTAGGTGTCCTACACAAGGCGA
AGCGACATTGGTCGAAGAACAGGACGCTAACTTCGTATGTAGACG
AACATTCGTCGATAGGGGGTGGGGAAACGGATGCGGATTGTTCGG
TAAAGGATCACTGATTACATGCGCAAAGTTCAAATGCGTTACGAA
ACTGGAAGGTAAAATAGTGCAATACGAAAACCTAAAGTATAGCGT
AATCGTTACAGTGCATACCGGAGACCAACATCAGGTCGGAAACGA
ATCAACCGAACACGGAACAACCGCAACAATTACACCACAGGCACC
TACAACCGAAATTCAACTAACCGATTACGGAGCTCTTACACTAGA
TTGCTCACCTAGAACCGGATTGGACTTTAACGAAATGGTACTGTT
GACTATGAAGGAGAAATCATGGTTAGTGCATAAACAATGGTTTCT
AGACCTACCACTACCATGGACTAGCGGAGCGAGTACGTCACAGGA
AACATGGAATAGACAGGACCTATTGGTGACATTTAAGACCGCACA
CGCTAAGAAGCAGGAAGTCGTAGTGTTAGGGTCACAAGAGGGAGC
AATGCATACCGCACTAACCGGAGCAACCGAAATACAGACTAGCGG
AACTACAACGATTTTTGCCGGTCACCTGAAATGTAGACTGAAGAT
GGACAAACTGACACTTAAAGGAATGTCATACGTTATGTGTACGGG
ATCCTTTAAGCTCGAAAAAGAGGTTGCCGAAACGCAACACGGAAC
AGTGCTAGTGCAAATTAAATATGAGGGAACCGACGCACCATGTAA
GATACCGTTTAGTACGCAAGACGAAAAGGGCGTTACGCAAAACGG
AAGACTGATTACCGCTAACCCTATCGTTACAGACAAAGAGAAACC
CGTTAACATAGAGGCCGAACCACCTTTTGGCGAATCATATATAGT
GATAGGCGCAGGCGAAAAAGCACTAAAGCTGTCATGGTTCAAAAA
AGGATCTAGCATAGGGAAGATGTTCGAAGCAACCGCTAGGGGAGC
TAGACGAATGGCAATACTGGGAGATACCGCATGGGACTTCGGATC
GATAGGGGGAGTGTTTACTAGCGTAGGTAAGTTGGTGCATCAGAT
ATTCGGAACCGCATACGGAGTGTTGTTTAGCGGAGTGTCATGGAC
TATGAAGATAGGGATAGGAGTGCTATTGACATGGCTCGGACTGAA
TTCGAGATCGACTAGCCTATCTATGACATGCATAGCCGTCGGACT
GGTTACACTGTATCTAGGCGTAATGGTGCAAGCAGATTCAGGATG
TGTAATTAATTGGAAAGGTAGAGAACTCAAGTGTGGAAGTGGCAT
TTTTGTCACCAATGAAGTTCACACTTGGACAGAGCAATACAAATT
TCAAGCTGACTCCCCTAAGAGACTATCAGCAGCCATCGGGAAGGC
ATGGGAGGAGGGTGTGTGTGGAATTCGATCAGCAACTCGTCTCGA
GAACATCATGTGGAAGCAAATATCAAATGAACTGAATCACATCTT
ACTTGAAAATGATATGAAATTCACAGTGGTTGTAGGAGATGTTGC
TGGGATCTTGGCTCAAGGAAAGAAAATGATTAGGCCACAACCCAT
GGAATACAAATACTCGTGGAAAAGCTGGGGAAAGGCTAAAATCAT
AGGGGCAGATGTACAGAACACCACCTTCATCATCGATGGCCCAAA
CACCCCAGAATGCCCTGATGACCAAAGAGCATGGAACATTTGGGA
AGTTGAGGACTATGGATTTGGAATTTTCACGACAAACATATGGCT
GAAATTGCGTGATTCCTACACCCAAGTGTGTGACCACCGGCTAAT
GTCAGCTGCCATCAAGGACAGCAAGGCAGTTCACGCTGACATGGG
GTACTGGATAGAAAGTGAAAAGAACGAGACCTGGAAGCTGGCAAG
AGCCTCATTCATAGAAGTTAAAACATGTATCTGGCCAAAATCCCA
CACTCTATGGAGCAATGGAGTTCTGGAAAGTGAAATGATAATTCC
AAAGATCTATGGAGGACCAATATCTCAGCACAACTACAGACCAGG
ATATTTTACACAAGCAGCAGGGCCGTGGCACCTAGGCAAGTTGGA
ACTGGATTTTGATTTGTGTGAGGGTACCACAGTTGTTGTGGATGA
ACATTGTGGAAATCGAGGACCATCTCTTAGGACCACAACAGTCAC
AGGAAAGATAATTCATGAATGGTGTTGCAGATCTTGCACGCTACC
ACCCTTACGTTTCAGAGGAGAAGATGGGTGCTGGTACGGTATGGA
AATCAGACCAGTCAAGGAAAAGGAAGAAAATCTAGTCAAATCAAT
GGTCTCTGCAGGGTCAGGGGAAGTGGACAGCTTTTCACTAGGACT
GCTATGCATATCAATAATGATCGAGGAGGTGATGAGATCCAGATG
GAGTAGAAGAATGCTGATGACTGGAACACTGGCTGTGTTCTTCCT
TCTCATAATGGGACAATTGACATGGAACGATCTGATCAGATTATG
CATCATGGTTGGAGCCAACGCTTCCGACAGGATGGGGATGGGAAC
GACGTACCTAGCCCTGATGGCCACTTTTAAAATGAGACCGATGTT
CGCTGTAGGGCTATTATTTCGCAGACTAACATCCAGAGAAGTTCT
TCTTCTAACAATTGGATTGAGTCTAGTGGCATCTGTGGAGTTACC
AAATTCCTTGGAGGAGCTGGGGGATGGACTTGCAATGGGCATTAT
GATTTTAAAATTATTGACTGACTTTCAATCACATCAGCTGTGGGC
TACCTTGCTGTCCTTGACATTTATCAAAACAACGTTTTCCTTGCA
CTACGCATGGAAGACAATGGCTATGGTACTGTCAATTGTATCTCT
CTTCCCTTTATGCCTGTCCACGACCTCCCAAAAAACAACATGGCT
TCCGGTGCTATTGGGATCCCTTGGATGCAAACCACTAACCATGTT
TCTTATAGCAGAAAACAAAATCTGGGGAAGGAGAAGTTGGCCCCT
CAATGAAGGAATCATGGCTGTTGGAATAGTCAGCATCCTACTAAG
TTCACTCCTCAAAAATGATGTACCGCTAGCTGGGCCACTAATAGC
TGGAGGCATGCTAATAGCATGTTATGTTATATCTGGAAGCTCAGC
CGACCTATCATTAGAGAAAGCGGCTGAGGTCTCCTGGGAAGAAGA
AGCAGAACACTCTGGTGCCTCACACAACATATTAGTGGAAGTCCA
AGATGATGGAACCATGAAGATAAAGGATGAAGAGAGAGATGACAC
GCTAACCATTCTCCTTAAAGCAACTCTGTTAGCAGTTTCAGGGGT
GTACCCATTATCAATACCAGCGACCCTTTTCGTGTGGTACTTTTG
GCAGAAAAAGAAACAGAGATCTGGAGTGTTATGGGACACACCCAG
CCCTCCAGAAGTGGAAAGAGCAGTTCTTGATGATGGTATCTATAG
AATTATGCAGAGAGGACTGTTGGGCAGGTCCCAAGTAGGGGTAGG
AGTTTTCCAAGAAAACGTGTTCCACACAATGTGGCATGTCACCAG
GGGAGCTGTACTCATGTATCAAGGGAAGAGATTGGAACCGAGCTG
GGCCAGTGTCAAAAAAGACCTGATCTCATATGGAGGAGGTTGGAG
GCTTCAAGGATCCTGGAACACAGGAGAAGAAGTGCAGGTGATTGC
TGTTGAACCAGGGAAAAACCCCAAAAATGTACAAACAGCGCCGGG
CACCTTTAAGACCCCTGAAGGTGAAGTTGGAGCCATTGCCCTAGA
CTTTAAACCTGGCACATCTGGATCTCCCATCGTGAACAGAGAAGG
AAAAATAGTAGGTCTTTATGGAAATGGAGTAGTGACAACAAGTGG
AACCTACGTCAGTGCCATAGCTCAAGCCAAAGCATCACAAGAAGG
GCCCCTACCAGAGATTGAAGACGAGGTGTTTAGGAAAAGAAACTT
AACAATAATGGACCTACATCCAGGATCGGGGAAAACAAGAAGATA
TCTTCCAGCCATAGTCCGTGAGGCCATAAAAAGGAAGCTGCGCAC
ACTAATCCTGGCTCCCACAAGGGTTGTCGCTTCCGAAATGGCAGA
GGCGCTCAAGGGAATGCCAATAAGGTACCAAACAACAGCAGTGAA
GAGTGAACATACAGGAAAAGAGATAGTTGACCTCATGTGTCACGC
CACTTTCACCATGCGCCTCCTGTCTCCCGTAAGAGTTCCCAATTA
CAACATGATCATCATGGATGAAGCACATTTCACCGATCCATCCAG
TATAGCGGCCAGAGGGTACATCTCAACCCGAGTGGGCATGGGTGA
AGCAGCTGCAATCTTCATGACAGCCACTCCCCCAGGATCAGTGGA
GGCCTTTCCACAGAGCAACGCAGTTATCCAAGATGAGGAAAGAGA
CATTCCTGAGAGATCATGGAACTCAGGCTATGAGTGGATCACTGA
CTTCCCAGGTAAAACAGTTTGGTTTGTTCCAAGCATTAAATCAGG
AAATGACATTGCCAACTGCTTAAGAAAGAATGGGAAACGGGTGAT
TCAATTGAGCAGGAAAACCTTTGATACAGAGTACCAAAAAACAAA
AAACAACGACTGGGACTACGTCGTCACAACAGACATCTCCGAAAT
GGGAGCAAATTTCCGAGCCGACAGGGTGATAGACCCAAGACGGTG
TCTGAAACCGGTAATACTAAAAGATGGTCCAGAGCGTGTCATTTT
AGCAGGACCAATGCCAGTGACTGTGGCCAGTGCCGCTCAGAGGAG
AGGAAGAATTGGAAGGAACCACAATAAGGAAGGTGATCAGTACAT
CTACATGGGACAGCCTTTAAACAACGATGAAGATCACGCTCACTG
GACAGAAGCAAAAATGCTCCTTGATAATATAAACACACCAGAAGG
GATTATCCCAGCCCTCTTTGAGCCAGAGAGAGAAAAGAGTGCAGC
AATAGACGGGGAATACAGACTGCGGGGTGAAGCAAGGAAAACGTT
TGTGGAGCTCATGAGAAGAGGAGATCTACCTGTCTGGCTATCCTA
CAAAGTTGCCTCAGAAGGCTTCCAGTACTCTGACAGAAGATGGTG
CTTTGACGGGGAAAGGAACAACCAGGTGTTGGAGGAGAACATGGA
CGTGGAGATCTGGACAAAAGAAGGAGAAAGAAAGAAACTACGACC
CCGCTGGCTGGATGCCAGAACATACTCAGACCCACTAGCCCTGCG
CGAGTTTAAAGAGTTTGCAGCAGGGAGAAGAAGCGTCTCAGGTGA
TTTAATATTAGAAATAGGGAAGCTTCCACAACACTTGACGCAAAG
GGCCCAGAATGCCCTGGACAACCTGGTTATGTTGCACAACTCCGA
ACAAGGAGGCAGAGCCTATAGACATGCAATGGAAGAACTGCCAGA
CACCATAGAAACGTTGATGCTCCTAGCTTTGATAGCTGTGTTAAC
TGGTGGAGTGACACTGTTCTTCCTATCAGGAAGGGGCCTAGGGAA
AACATCTATCGGCCTACTCTGCGTAATGGCTTCAAGCGTACTGCT
ATGGATGGCCAGTGTGGAGCCCCATTGGATAGCGGCCTCCATCAT
ACTGGAGTTCTTCCTGATGGTGCTGCTTATTCCAGAGCCAGACAG
ACAACGCACTCCGCAGGACAACCAGCTGGCATATGTGGTGATAGG
TTTGTTATTCATGATACTGACAGTAGCAGCCAATGAGATGGGACT
GCTGGAAACCACAAAGAAAGACTTAGGGATTGGCCATGTGGCTGT
TGAAAATCACCACCATGCCGCAATGCTGGACGTAGACTTACATCC
AGCTTCAGCCTGGACCCTCTATGCAGTGGCCACAACAATTATCAC
TCCCATGATGAGGCACACAATCGAAAACACAACGGCAAACATTTC
CCTGACAGCCATTGCAAACCAGGCAGCTATATTGATGGGACTTGA
CAAAGGATGGCCAATATCGAAGATGGACATAGGAGTTCCACTTCT
CGCCTTGGGGTGCTATTCCCAGGTGAATCCACTGACGCTGACAGC
GGCGGTATTGATGCTAGTGGCTCATTACGCCATAATTGGACCTGG
ACTGCAAGCAAAAGCTACTAGAGAAGCTCAAAAAAGGACAGCGGC
CGGAATAATGAAAAATCCAACCGTTGATGGAATTGTTGCAATAGA
TTTGGACCCTGTGGTTTATGATGCAAAATTTGAAAAACAACTAGG
CCAAATAATGTTGTTGATACTATGCACATCACAGATCCTCTTGAT
GCGGACTACATGGGCCTTGTGCGAGTCCATCACACTGGCCACTGG
ACCTCTGACCACGCTTTGGGAGGGATCTCCAGGAAAATTTTGGAA
CACCACGATAGCGGTTTCCATGGCAAACATTTTCAGAGGAAGTTA
TCTAGCAGGAGCAGGTCTGGCCTTCTCATTAATGAAATCTCTAGG
AGGAGGTAGGAGAGGCACGGGAGCCCAAGGGGAAACACTGGGAGA
GAAATGGAAAAGACAGCTGAACCAACTGAGCAAGTCAGAATTTAA
CACCTATAAAAGGAGTGGGATTATGGAAGTGGACAGATCCGAAGC
CAAAGAGGGATTGAAAAGAGGAGAAACAACCAAACATGCAGTGTC
GAGAGGAACCGCTAAACTGAGGTGGTTCGTGGAGAGGAACCTTGT
GAAACCAGAAGGGAAAGTCATAGACCTCGGTTGTGGAAGAGGTGG
CTGGTCATACTATTGCGCTGGGCTGAAGAAAGTCACAGAAGTGAA
GGGGTATACAAAAGGAGGACCTGGACATGAGGAACCAATCCCAAT
GGCGACCTATGGATGGAACCTAGTAAAGCTGCACTCTGGGAAAGA
CGTATTTTTTATACCACCTGAGAAATGTGACACCCTTTTGTGTGA
TATTGGTGAGTCCTCTCCAAACCCAACTATAGAGGAAGGAAGAAC
GCTACGCGTCCTAAAGATGGTGGAACCATGGCTCAGAGGAAACCA
ATTTTGCATAAAAATTCTGAATCCCTACATGCCAAGTGTGGTGGA
AACTCTGGAGCAAATGCAAAGAAAACATGGAGGAATGCTAGTGCG
AAATCCACTTTCAAGAAATTCTACTCATGAAATGTATTGGGTTTC
ATGTGGAACAGGAAACATTGTGTCAGCAGTAAACATGACATCTAG
AATGTTGCTAAATCGATTCACAATGGCTCACAGGAAACCAACATA
TGAAAGAGACGTGGACCTAGGCGCCGGAACAAGACATGTGGCAGT
GGAACCAGAGGTAGCCAACCTAGATATCATTGGCCAGAGGATAGA
GAACATAAAACATGAACATAAGTCAACATGGCATTATGATGAGGA
CAATCCATATAAAACATGGGCCTATCATGGATCATATGAGGTCAA
GCCATCAGGATCAGCCTCATCCATGGTCAATGGCGTGGTGAAACT
GCTCACCAAACCATGGGATGTCATCCCTATGGTCACACAAATAGC
CATGACTGACACTACACCCTTTGGACAACAGAGGGTGTTTAAAGA
GAAAGTTGACACACGCACACCAAAAGCAAAACGGGGCACAGCACA
AATCATGGAGGTGACAGCCAAGTGGTTATGGGGTTTTCTTTCTAG
AAACAAGAAACCAAGAATTTGCACAAGAGAGGAGTTCACAAGAAA
AGTTAGGTCAAACGCAGCCATTGGAGCAGTGTTCGTTGATGAAAA
TCAATGGAACTCAGCAAAAGAAGCAGTGGAAGATGAGCGGTTCTG
GGACCTTGTGCATAGAGAGAGGGAGCTTCACAAACAGGGAAAATG
TGCTACGTGTGTTTACAACATGATGGGGAAGAGAGAGAAAAAGCT
AGGAGAGTTCGGAAAGGCAAAAGGAAGTCGTGCAATATGGTACAT
GTGGTTGGGAGCACGCTTTCTAGAGTTCGAAGCTCTTGGTTTCAT
GAACGAAGATCACTGGTTCAGCAGAGAGAATTCACTCAGCGGAGT
GGAAGGAGAAGGACTCCACAAACTTGGATATATACTCAGAGACAT
ATCAAAGATTCCAGGGGGAAACATGTATGCAGATGACACAGCCGG
ATGGGATACAAGGATAACAGAGGATGACCTTCAGAATGAGGCCAG
AATTACTGACATCATGGAACCCGAACATGCCCTCCTGGCTAAGTC
AATCTTCAAGTTAACCTACCAAAATAAGGTGGTAAGGGTACAGAG
ACCAGCAAAAAATGGAACCGTGATGGATGTCATATCCAGACGTGA
CCAGAGAGGAAGTGGTCAGGTCGGAACTTATGGCTTAAACACTTT
CACCAACATGGAAGCCCAGCTGATAAGACAAATGGAGTCTGAGGG
AATCTTTTCACCCAGCGAATTAGAGACCCCAAATTTAGCCGAGAG
AGTTCTCGACTGGCTGGAAAAATATGGCGTCGAAAGGCTGAAAAG
AATGGCAATCAGCGGAGATGACTGCGTAGTGAAACCAATTGATGA
TAGGTTTGCAACAGCCTTGACAGCTCTGAATGATATGGGAAAAGT
AAGAAAAGATATACCACAATGGGAACCTTCAAAAGGATGGAATGA
TTGGCAACAAGTGCCTTTTTGTTCACACCACTTCCACCAGCTGAT
TATGAAGGATGGGAGGGAAATAGTGGTGCCATGCCGCAACCAAGA
TGAACTTGTGGGTAGGGCTAGAGTATCACAAGGTGCTGGATGGAG
CCTGAGAGAAACTGCATGCCTAGGCAAGTCATATGCACAAATGTG
GCAGCTGATGTACTTCCACAGGAGAGACCTGAGACTAGCCGCTAA
TGCTATCTGTTCAGCCGTTCCAGTTGATTGGATCCCAACCAGCCG
TACCACCTGGTCGATCCATGCCCATCACCAATGGATGACAACAGA
AGACATGCTGTCAGTGTGGAATAGGGTTTGGATAGAGGAAAACCC
ATGGATGGAGGACAAAACCCACATATCCAGTTGGGGAGATGTTCC
ATATTTAGGAAAAAGGGAAGACCAATGGTGTGGATCCCTGATAGG
CTTAACAGCAAGGGCCACCTGGGCCACCAACATACAAGTGGCCAT
AAACCAAGTGAGAAGACTAATTGGGAATGAGAATTATCTAGATTA
CATGACATCAATGAAGAGATTCAAGAACGAGAGTGATCCCGAAGG
GGCACTCTGGTGAGTCAACACATTTACAAAATAAAGGAAAATAAG
AAATCAAACAAGGCAAGAAGTCAGGCCGGATTAAGCCATAGTACG
GTAAGAGCTATGCTGCCTGTGAGCCCCGTCTAAGGACGTAAAATG
AAGTCAGGCCGAAAGCCACGGCTTGAGCAAACCGTGCTGCCTGTA
GCTCCATCGTGGGGATGTAAAAACCTGGGAGGCTGCAACCCATGG
AAGCTGTACGCATGGGGTAGCAGACTAGTGGTTAGAGGAGACCCC
TCCCGAAACATAACGCAGCAGCGGGGCCCAACACCAGGGGAAGCT
GTACCCTGGTGGTAAGGACTAGAGGTTAGAGGAGACCCCCCGCAT
AACAATAAACAGCATATTGACGCTGGGAGAGACCAGAGATCCTGC
TGTCTCTACAGCATCATTCCAGGCACAGAACGCCAGAAAATGGAA
TGGTGCTGTTGAATCAACAGGTTCT
DENV-2-NI-BID-V533-2005
SEQ ID NO: 3
AGTTGTTAGTCTACGTGGACCGACAAAGACAGATTCTTTGAGGGA
GCTAAGCTCAACGTAGTTACGCCTTTCAATATGCTGAAACGCGAG
AGAAACCGCGTGTCAACTGTGCAACAGCTGACAAAGAGATTCTCA
CTTGGAATGCTGCAAGGACGCGGACCATTAAAACTGTTCATGGCC
CTTGTGGCGTTCCTTCGTTTCCTAACAATCCCACCAACAGCAGGG
ATACTAAAAAGATGGGGAACGATCAAAAAATCAAAAGCTATCAAT
GTTTTGAGAGGGTTCAGGAAAGAGATTGGAAGGATGCTGAACATC
TTGAACAAGAGACGCAGGACAGCAGGCGTGATTGTTATGTTGATT
CCAACAGCGATGGCGTTCCATTTAACCACACGCAATGGAGAACCA
CACATGATCGTTGGTAGGCAGGAGAAAGGGAAAAGTCTTCTGTTC
AAAACAGAGGATGGTGTTAACATGTGTACTCTCATGGCCATAGAC
CTTGGTGAATTGTGTGAAGATACAATCACGTACAAGTGTCCTCTC
CTCAGACAAAATGAACCAGAAGACATAGATTGTTGGTGCAACTCT
ACGTCCACATGGGTAACTTATGGGACATGTACCACCACAGGAGAA
CACAGAAGAGAAAAAAGATCAGTGGCGCTCGTTCCACATGTAGGT
ATGGGACTGGAGACACGAACTGAAACATGGATGTCATCAGAAGGG
GCCTGGAAACATGTTCAGAGAATTGAAACCTGGATCTTGAGACAT
CCAGGTTTTACCATAATGGCAGCAATCCTGGCATACACCATAGGA
ACGACACATTTCCAAAGGGCCTTGATTTTCATTTTACTGACAGCT
GTCGCTCCTTCAATGACAATGCGCTGCATAGGAATATCAAATAGA
GACTTCGTAGAAGGGGTTTCAGGAGGAAGCTGGGTTGACATAGTC
TTAGAACATGGAAGTTGTGTGACGACGATGGCAAAAAACAAACCA
ACATTGGATTTTGAACTGATAAAAACAGAAGCCAAACAACCTGCC
ACTCTAAGGAAGTACTGTATAGAAGCAAAGCTGACCAACACAACA
ACAGAATCGCGTTGCCCAACACAAGGGGAACCCAGTCTAAATGAG
GAGCAGGACAAAAGGTTCATCTGCAAACACTCCATGGTAGACAGA
GGATGGGGAAATGGATGTGGATTATTTGGAAAGGGAGGCATTGTG
ACCTGTGCTATGTTTACATGCAAAAAGAACATGGAAGGAAAAGTC
GTGCAGCCAGAAAATTTGGAATACACCATCGTGATAACACCTCAC
TCAGGAGAGGAGCACGCTGTAGGTAATGACACAGGAAAGCATGGC
AAGGAAATCAAAATAACACCACAGAGCTCCATCACAGAAGCAGAA
CTGACAGGCTATGGCACTGTCACGATGGAGTGCTCTCCGAGAACG
GGCCTCGACTTCAATGAGATGGTACTGCTGCAGATGGAAGACAAA
GCTTGGCTGGTGCACAGGCAATGGTTCCTAGACCTGCCGTTACCA
TGGCTACCCGGAGCGGACACACAAGGATCAAATTGGATACAGAAA
GAGACATTGGTCACTTTCAAAAATCCCCACGCGAAGAAACAGGAT
GTCGTTGTCTTAGGGTCTCAAGAAGGGGCCATGCACACGGCACTC
ACAGGGGCCACAGAAATCCAGATGTCATCAGGAAACTTACTGTTC
ACAGGACATCTCAAGTGCAGGCTGAGAATGGACAAACTACAGCTC
AAAGGAATGTCATACTCTATGTGTACAGGAAAGTTTAAAATTGTG
AAGGAAATAGCAGAAACACAACATGGAACAATAGTTATCAGAGTA
CAATATGAAGGGGACGGTTCTCCATGCAAGATCCCTTTTGAGATA
ACAGATTTGGAAAAAAGACACGTCTTAGGTCGCTTGATTACAGTT
AACCCAATCGTAACAGAAAAAGATAGCCCAGTCAACATAGAAGCA
GAACCTCCATTCGGAGACAGCTACATCATTATAGGAGTAGAGCCG
GGACAATTGAAACTCAATTGGTTTAAGAAGGGAAGTTCCATCGGC
CAAATGTTTGAGACAACAATGAGAGGAGCAAAGAGAATGGCCATT
TTAGGTGACACAGCCTGGGACTTTGGATCCCTGGGAGGAGTGTTT
ACATCTATAGGAAAGGCTCTCCACCAAGTTTTCGGAGCAATCTAT
GGGGCTGCTTTTAGTGGGGTCTCATGGACTATGAAAATCCTCATA
GGAGTCATCATCACATGGATAGGAATGAATTCACGTAGCACCTCA
CTGTCTGTGTCGCTAGTATTGGTGGGCGTTGTGACACTGTACCTG
GGAGCTATGGTGCAAGCTGATAGTGGTTGCGTTGTGAGCTGGAAA
AATAAAGAACTGAAATGTGGCAGCGGGATCTTCATCACAGATAAC
GTACACACATGGACAGAACAATATAAGTTCCAACCAGAATCCCCT
TCAAAACTAGCTTCAGCTATCCAAAAAGCTCATGAAGAGGGCATT
TGTGGAATCCGCTCAGTAACAAGATTGGAGAATCTGATGTGGAAA
CAAATAACACCAGAATTGAATCATATTCTATCAGAAAATGAGGTA
AAGTTGACCATTATGACAGGAGACATTAGAGGAATCATGCAGGCA
GGAAAACGATCCTTGCGGCCCCAGCCCACTGAGCTGAAGTACTCA
TGGAAAACATGGGGAAAGGCGAAAATGCTCTCCACAGAGTCTCAC
AATCAGACCTTTCTTATTGATGGCCCTGAAACAGCAGAATGCCCC
AACACAAACAGAGCCTGGAACTCGCTGGAAGTTGAAGACTATGGT
TTTGGAGTTTTCACCACCAATATATGGCTGAAATTGAGAGAAAAA
CAGGATGTATTTTGTGACTCAAAACTCATGTCAGCGGCCATTAAA
GACAACAGAGCCGTTCATGCTGATATGGGTTATTGGATAGAAAGT
GCACTCAATGACACATGGAAGATGGAGAAAGCCTCCTTCATTGAA
GTTAAAAGCTGCCACTGGCCAAAGTCACACACCCTTTGGAGCAAT
GGAGTATTAGAAAGTGAGATGATAATCCCAAAAAATTTTGCCGGG
CCAGTGTCACAACACAACTACAGACCAGGCTACCATACACAAACA
GCAGGACCTTGGCATCTAGGTAAGCTTGAGATGGACTTTGATCTC
TGCGAAGGAACTACAGTGGTGGTGACTGAGGACTGTGGAAATAGA
GGACCCTCTTTAAGAACGACCACTGCCTCTGGAAAACTCATAACA
GAATGGTGCTGCCGATCCTGCACACTACCACCTCTAAGATACAGA
GGTGAGGATGGATGCTGGTACGGGATGGAAATCAGACCATTGAAA
GAGAAAGAAGAGAATTTGGTCAACTCCTTGGTCACAGCCGGACAT
GGGCAGATTGACAACTTTTCACTAGGAGTCTTGGGAATGGCACTG
TTCCTGGAAGAAATGCTCAGGACCCGAATAGGAACGAAACATGCA
ATACTGCTAGTTGCAGTATCTTTTGTGACATTGATTACTGGGAAC
ATGTCTTTTAGAGACCTGGGAAGAGTGATGGTTATGGTGGGCGCT
ACCATGACGGATGACATAGGTATGGGAGTGACTTATCTTGCCCTA
CTAGCAGCTTTTAAGGTTAGACCAACTTTTGCAGCTGGACTACTC
TTAAGAAAACTGACCTCCAAGGAATTGATGATGGCCACCATAGGA
ATCGCACTCCTTTCCCAAAGCACCATACCAGAGACCATTCTTGAA
CTGACTGATGCATTAGCCCTGGGCATGATGGTCCTCAAAATAGTG
AGAAATATGGAAAAATACCAATTGGCAGTGACTATCATGGCTATT
TCATGTGTCCCAAATGCAGTGATACTGCAAAACGCATGGAAGGTG
AGTTGCACAATATTGGCAGCGGTGTCCGTTTCACCACTGCTCTTA
ACATCCTCACAGCAGAAAGCGGATTGGATACCACTGGCATTGACG
ATAAAAGGTCTCAATCCAACAGCCATTTTTTTAACAACTCTCTCG
AGGACCAGCAAGAAAAGGAGCTGGCCGCTAAATGAAGCTATCATG
GCAGTTGGGATGGTGAGCATTTTAGCCAGTTCTCTCCTAAAGAAT
GATATTCCTATGACAGGTCCATTAGTGGCTGGAGGACTCCTCACC
GTATGTTACGTGCTCACTGGACGATCGGCCGATTTGGAACTGGAG
AGAGCTGCCGATGTAAAATGGGAAGATCAGGCAGAAATATCAGGA
AGCAGCCCAATCCTGTCAATAACAATATCAGAAGATGGCAGCATG
TCGATAAAAAATGAAGAGGAAGAACAAACACTGACCATACTCATC
AGAACGGGATTGTTGGTGATCTCAGGAGTCTTTCCAGTATCGATA
CCAATTACGGCAGCAGCATGGTACCTGTGGGAAGTAAAGAAACAA
CGGGCTGGAGTACTGTGGGACGTCCCTTCACCCCCACCAGTGGAA
AAAGCCGAACTGGAGGATGGAGCCTACAGAATCAAGCAAAGAGGG
ATCCTTGGATATTCTCAGATTGGAGCCGGAGTTTACAAAGAAGGA
ACATTCCATACAATGTGGCACGTCACACGTGGTGCTGTTCTGATG
CATAGAGGGAAGAGGATTGAACCATCATGGGCAGATGTCAAGAAA
GACCTAATATCATATGGAGGAGGCTGGAAGCTAGAAGGAGAATGG
AAGGAAGGAGAGGAAGTCCAAGTCCTGGCATTGGAACCTGGAAAA
AATCCAAGAGCCGTCCAAACGAAACCTGGAATATTCAAAACCAAC
ACCGGAACCATAGGCGCCGTATCTCTGGACTTTTCCCCTGGAACG
TCAGGATCTCCAATCGTCGACAGAAAAGGAAAAGTTGTGGGTCTT
TACGGTAATGGTGTTGTCACAAGGAGTGGAGCATATGTAAGTGCT
ATAGCCCAGACCGAAAAAAGCATTGAAGACAATCCAGAGATCGAA
GATGACATTTTCCGAAAGAAAAGATTGACCATCATGGACCTCCAT
CCAGGAGCAGGAAAGACAAAAAGATACCTTCCAGCCATAGTTAGA
GAAGCCATAAAACGTGGCTTGAGAACATTGATCCTGGCTCCCACT
AGAGTAGTGGCAGCTGAAATGGAGGAAGCTCTTAGAGGACTTCCA
ATAAGATACCAAACTACAGCCATCAAAACCGAGCATACCGGGCGG
GAGATCGTGGACCTAATGTGTCATGCCACATTTACTATGAGGCTG
TTATCACCAGTCAGAGTGCCAAATTACAACCTGATCATCATGGAC
GAAGCCCACTTCACAGACCCAGCAAGTATAGCAGCTAGAGGATAC
ATTTCAACTCGAGTAGAGATGGGTGAAGCAGCCGGGATTTTCATG
ACAGCCACTCCTCCGGGAAGTAGAGACCCATTTCCTCAGAGCAAT
GCACCAATTATGGATGAGGAAAGAGAAATCCCTGAGCGTTCATGG
AATTCAGGACACGAATGGGTCACGGATTTTAAGGGGAAGACTGTT
TGGTTTGTTCCAAGTATAAAAGCAGGAAATGATATAGCAGCTTGT
CTTAGGAAAAATGGAAAGAAAGTGATACAACTCAGTAGGAAGACT
TTTGACTCTGAGTATGTTAAGACTAGAGCCAATGATTGGGACTTT
GTGGTCACAACTGACATTTCAGAAATGGGTGCCAACTTCAAGGCT
GAGAGGGTTATAGACCCCAGACGTTGCATGAAACCAGTTATACTA
ACAGATGGCGAGGAGCGGGTGATCTTGGCTGGACCTATGCCAGTG
ACCCACTCTAGTGCAGCGCAAAGAAGAGGGAGAATAGGAAGAAAT
CCAAAAAATGAAAATGACCAGTACATATACATGGGGGAACCTCTT
GAAAATGATGAAGACTGTGCACATTGGAAAGAAGCTAAAATGCTC
CTAGATAACATCAACACACCTGAAGGAATCATTCCTAGTATGTTC
GAACCAGAGCGTGAAAAAGTGGATGCCATTGATGGTGAATACCGT
TTGAGAGGAGAAGCAAGGAAAACCTTTGTGGACCTAATGAGAAGA
GGGGACTTACCAGTCTGGTTGGCCTACAAAGTGGCAGCTGAAGGC
ATCAACTACGCAGACAGAAAGTGGTGTTTTGATGGAATTAAGAAC
AACCAAATACTGGAAGAAAATATGGAAGTGGAAATCTGGACAAAA
GAAGGGGAAAGGAAAAAATTAAAACCCAGATGGTTGGATGCTAGG
ATCTATTCTGACCCACTAGCACTAAAAGAATTCAAGGAATTTGCA
GCTGGAAGAAAATCTTTGACCCTGAACCTAATCACAGAAATGGGT
AGGCTTCCAACTTTCATGACTCAGAAAGCAAGAAACGCACTGGAC
AACCTGGCTGTGCTGCATACGGCTGAGGCAGGTGGAAGGGCGTAC
AATCATGCTCTCAGTGAACTGCCGGAGACCCTGGAGACACTGCTC
CTACTGACACTTCTGGCAACAGTCACAGGAGGAATCTTCTTATTC
TTAATGAGCGGAAAAGGTATAGGGAAGATGACCCTGGGAATGTGT
TGCATAATCACGGCTAGTATCCTCCTATGGTATGCACAGATACAA
CCACACTGGATAGCAGCTTCAATAATACTGGATTTTTTCTCATAG
TTTTGCTCATTCCAGAACCAGAAAAACAGAGAACACCCCAAGACA
ACCAATTGACCTACGTTGTCATAGCCATCCTCACAGTGGTGGCCG
CAACCATGGCAAACGAGATGGGTTTCCTGGAAAAAACCAAGAAAG
ACCTCGGATTGGGAAGCATTACAACCCAGGAATCTGAGAGCAATA
TCCTGGACATAGATCTACGCCCTGCATCAGCATGGACGCTGTATG
CCGTAGCTACAACATTTGTCACACCAATGTTGAGACATAGCATTG
AAAATTCCTCAGTGAATGTCTCCCTAACAGCCATTGCTAACCAAG
CTACAGTGCTAATGGGTCTTGGGAAAGGATGGCCATTGTCAAAGA
TGGACATTGGAGTTCCCCTCCTTGCCATTGGATGCTATTCACAAG
TCAACCCTATAACTCTCACAGCAGCTCTCCTTTTATTGGTAGCAC
ATTATGCCATTATAGGGCCAGGACTTCAAGCAAAAGCAACCAGAG
AAGCCCAGAAAAGAGCAGCAGCAGGCATCATGAAAAACCCAACAG
TCGATGGAATAACAGTGATTGACCTAGAACCAATACCCTATGATC
CAAAATTTGAAAAGCAGTTAGGACAAGTAATGCTCCTAATCCTCT
GCGTGACTCAAGTATTAATGATGAGGACTACATGGGCTTTATGTG
AGGCTCTAACCCTAGCGACCGGGCCCATCTCCACACTGTGGGAAG
GAAATCCAGGGAGGTTTTGGAACACTACCATTGCAGTGTCAATGG
CTAACATCTTTAGGGGGAGCTACTTGGCCGGAGCTGGACTTCTCT
TTTCCATCATGAAAAACACAACAAACACAAGAAGAGGAACTGGCA
ACATAGGAGAGACACTTGGAGAAAAATGGAAAAGTCGATTAAACG
CACTGGGGAAAAGTGAATTTCAAATCTACAAGAAAAGTGGAATCC
AGGAAGTGGATAGAACCCTAGCAAAAGAAGGTATCAAAAGAGGAG
AAACGGACCACCATGCTGTGTCGCGAGGTTCAGCAAAACTGAGAT
GGTTCGTCGAGAGAAATATGGTCACACCGGAAGGGAAGGTGGTGG
ATCTCGGTTGCGGCAGAGGGGGCTGGTCATACTATTGCGGGGGAC
TAAAGAATGTAAGAGAAGTCAAAGGCCTAACAAAAGGAGGACCAG
GACATGAAGAACCCATCCCCATGTCAACATATGGGTGGAATCTAG
TGCGTCTGCAAAGTGGAGTTGACGTTTTCTTCACCCCGCCAGAAA
AGTGTGATACATTGTTGTGTGACATAGGGGAGTCGTCACCAAATC
CCACGATAGAAGCAGGACGAACACTCAGAGTCCTCAACTTAGTGG
AAAATTGGTTGAACAATAACACCCAATTTTGCATAAAGGTCCTCA
ACCCATATATGCCTTCAGTCATAGAAAAAATGGAAGCATTACAAA
GGAAATATGGAGGAGCCTTAGTGAGGAATCCACTCTCACGAAACT
CCACGCATGAAATGTACTGGGTATCTAATGCCACCGGGAACATAG
TGTCATCAGTGAACATGATTTCAAGGATGTTGATTAACAGATTCA
CAATGAAACACAAGAAAGCCACTTACGAGCCAGATGTTGACCTAG
GAAGTGGAACCCGCAACATTGGAATTGAAAGTGAGATACCAAATC
TAGACATAATAGGAAAGAGAATAGAGAAAATAAAACAAGAGCATG
AAACATCATGGCACTATGATCAAGACCACCCATACAAAACGTGGG
CTTACCATGGCAGCTATGAAACAAAACAAACTGGATCAGCATCAT
CTATGGTGAACGGAGTGGTCAGATTGCTGACAAAACCTTGGGACG
TCGTCCCTATGGTGACACAGATGGCAATGACAGACACGACTCCTT
TTGGACAACAGCGCGTTTTCAAAGAGAAAGTAGACACGAGAACCC
AAGAACCGAAGGAAGGCACAAAGAAACTGATGAAAATTACGGCAG
AGTGGCTTTGGAAAGAACTAGGAAAGAAAAAGACACCTAGGATGT
GTACCAGAGAAGAATTCACAAGAAAAGTGAGAAGCAATGCAGCCT
TGGGGGCCATATTCACTGATGAGAACAAATGGAAATCGGCACGTG
AGGCTGTTGAAGATGGTAGGTTCTGGGAGCTGGTTGACAGGGAAA
GAAATCTCCATCTTGAAGGAAAGTGTGAAACATGTGTGTACAACA
TGATGGGAAAAAGAGAGAAGAAACTAGGGGAGTTCGGCAAGGCAA
AAGGTAGCAGAGCCATATGGTACATGTGGCTTGGAGCACGCTTCT
TAGAGTTTGAAGCCCTAGGATTCCTGAATGAAGATCACTGGTTCT
CCAGAGGGAACTCCCTGAGTGGAGTGGAAGGAGAAGGGCTGCACA
GGCTAGGCTACATTTTAAGAGACGTGGGCAAGAAGGAAGGGGGGG
CAATGTACGCCGATGATACAGCAGGATGGGACACAAGAATCACAC
TAGAAGACTTAAAAAATGAAGAAATGGTAACGAACCACATGAAAG
GAGAACACAAGAAACTAGCCGAGGCCATATTCAAACTAACGTACC
AAAACAAGGTGGTGCGTGTGCAAAGACCAACACCAAGAGGTACAG
TAATGGATATCATATCGAGAAGAGACCAAAGAGGCAGTGGGCAAG
TCGGCACCTATGGCCTTAATACCTTCACCAATATGGAAGCCCAAT
TAATTAGACAGATGGAGGGAGAAGGAATCTTCAAAAGCATTCAGC
ACCTGACAGCCACAGAAGAAATCGCTGTACAGAACTGGCTAGCAA
GAGTGGGGCGTGAAAGGCTATCAAGAATGGCAATCAGTGGAGATG
ATTGTGTTGTAAAACCTATAGATGACAGATTTGCAAGTGCTTTAA
CAGCTCTAAATGACATGGGAAAAGTTAGAAAAGATATACAACAAT
GGGAACCTTCAAGAGGATGGAACGATTGGACACAAGTGCCTTTCT
GTTCACACCACTTTCATGAGTTAGTCATGAAAGATGGTCGCGTGC
TCGTAGTCCCATGCAGAAACCAAGATGAACTGATTGGTAGAGCCC
GAATTTCCCAGGGAGCTGGGTGGTCTTTGAAAGAGACGGCCTGTT
TGGGAAAGTCTTACGCCCAAATGTGGACTCTGATGTACTTCCACA
GACGTGACCTCAGACTGGCGGCAAATGCCATCTGCTCGGCAGTCC
CGTCACATTGGGTTCCAACAAGTCGAACAACCTGGTCCATACACG
CTAAGCATGAATGGATGACGACGGAAGACATGCTGGCAGTCTGGA
ACAGGGTGTGGATCCAAGAAAACCCGTGGATGGAAGATAAAACTC
CAGTGGAATCATGGGAAGAAGTCCCATACTTGGGAAAAAGAGAAG
ACCAATGGTGCGGCTCATTGATTGGGCTAACAAGCAGGGCTACCT
GGGCAAAGAACATCCAAACAGCAATAAATCAAGTCAGATCCCTTA
TAGGCAATGAGGAATACACAGACTACATGCCATCCATGAAGAGAT
TTAGAAGGGAAGAGGAAGAGGCAGGTGTCCTGTGGTAGAAGGCAA
AACTAACATGAAACAAGGCTAAAAGTCAGGTCGGATTAAGCCATA
GTACGGAAAAAACTATGCTACCTGTGAGCCCCGTCCAAGGACGTT
AAAAGAAGTCAGGCCATCACAAAATGCCACAGCTTGAGTAAACTG
TGCAGCCTGTAGCTCCACCTGAGGAGGTGTAAAAAACCTGGGAGG
CCACAAACCATGGAAGCTGTACGCATGGCGTAGTGGACTAGCGGT
TAGAGGAGACCCCTCCCTTACAAATCGCAGCAAACAACGGGGGCC
CAAGGTGAGATGAAGCTGTAATCTCACTGGAAGGACTAGAGGTTA
GAGGAGACCCCCCCAAAACAAAAAACAGCATATTGACGCTGGGAA
AGACCAGAGATCCTGCTGTCTCCTCAGCATCATTCCAGGCACAGA
ACGCCAGAAAATGGAATGGTGCTGTTGAATCAACAGGTTCT
DENV-2-NI-BID-V533-2005-E-Min
SEQ ID NO: 4
AGTTGTTAGTCTACGTGGACCGACAAAGACAGATTCTTTGAGGGA
GCTAAGCTCAACGTAGTTCTAACAGTTTTTTAATTAGAGAGCAGA
TCTCTGATGAATAACCAACGAAAAAAGGCGAGAAGTACGCCTTTC
AATATGCTGAAACGCGAGAGAAACCGCGTGTCAACTGTGCAACAG
CTGACAAAGAGATTCTCACTTGGAATGCTGCAAGGACGCGGACCA
TTAAAACTGTTCATGGCCCTTGTGGCGTTCCTTCGTTTCCTAACA
ATCCCACCAACAGCAGGGATACTAAAAAGATGGGGAACGATCAAA
AAATCAAAAGCTATCAATGTTTTGAGAGGGTTCAGGAAAGAGATT
GGAAGGATGCTGAACATCTTGAACAAGAGACGCAGGACAGCAGGC
GTGATTGTTATGTTGATTCCAACAGCGATGGCGTTCCATTTAACC
ACACGCAATGGAGAACCACACATGATCGTTGGTAGGCAGGAGAAA
GGGAAAAGTCTTCTGTTCAAAACAGAGGATGGTGTTAACATGTGT
ACTCTCATGGCCATAGACCTTGGTGAATTGTGTGAAGATACAATC
ACGTACAAGTGTCCTCTCCTCAGACAAAATGAACCAGAAGACATA
GATTGTTGGTGCAACTCTACGTCCACATGGGTAACTTATGGGACA
TGTACCACCACAGGAGAACACAGAAGAGAAAAAAGATCAGTGGCG
CTCGTTCCACATGTAGGTATGGGACTGGAGACACGAACTGAAACA
TGGATGTCATCAGAAGGGGCCTGGAAACATGTTCAGAGAATTGAA
ACCTGGATCTTGAGACATCCAGGTTTTACCATAATGGCAGCAATC
CTGGCATACACCATAGGAACGACACATTTCCAAAGGGCCTTGATT
TTCATTTTACTGACAGCTGTCGCTCCTTCAATGACAATGAGATGC
ATAGGGATATCGAATCGCGATTTCGTCGAAGGCGTATCCGGAGGG
TCATGGGTCGACATCGTACTCGAACACGGATCATGCGTTACGACT
ATGGCTAAGAATAAGCCTACACTAGACTTCGAACTGATTAAGACA
GAGGCTAAGCAACCGGCAACATTGCGTAAGTACTGTATCGAAGCG
AAACTGACTAACACTACAACCGAATCTAGATGCCCTACACAGGGC
GAACCTAGTCTGAACGAAGAGCAAGACAAAAGGTTTATATGCAAA
CACTCTATGGTCGATCGCGGATGGGGAAACGGATGCGGATTGTTC
GGTAAGGGGGGAATCGTTACATGCGCTATGTTTACATGTAAAAAA
AATATGGAGGGAAAGGTCGTGCAACCAGAGAATCTCGAATATACA
ATCGTAATCACACCGCATAGCGGAGAGGAACACGCAGTCGGAAAC
GATACCGGAAAACACGGAAAAGAGATTAAGATTACACCACAGAGC
TCCATAACCGAAGCCGAACTGACAGGGTACGGAACAGTGACAATG
GAATGCTCACCTAGAACAGGCCTAGACTTTAACGAAATGGTGTTA
CTGCAAATGGAAGACAAAGCATGGTTAGTGCATAGGCAATGGTTT
TTAGACCTACCACTACCATGGTTGCCCGGAGCCGATACACAGGGA
TCGAACTGGATACAGAAAGAGACACTCGTTACGTTTAAAAACCCA
CACGCTAAAAAACAGGACGTAGTCGTACTCGGATCACAGGAAGGC
GCAATGCATACCGCATTGACAGGCGCTACAGAGATACAGATGTCT
AGCGGAAATCTGTTGTTTACAGGGCATCTGAAATGTAGACTGAGA
ATGGACAAACTGCAATTGAAGGGAATGTCATACTCTATGTGTACG
GGTAAGTTTAAGATAGTCAAAGAGATAGCCGAAACACAACACGGA
ACAATCGTAATTAGGGTGCAATACGAAGGCGACGGGTCACCATGT
AAGATACCATTCGAAATTACAGACCTCGAAAAAAGACACGTACTC
GGAAGACTGATAACAGTGAATCCGATCGTTACGGAAAAAGACTCA
CCCGTTAATATCGAAGCCGAACCACCATTCGGAGACTCATACATA
ATAATCGGAGTCGAACCCGGACAATTGAAACTGAATTGGTTTAAA
AAAGGGTCATCAATCGGACAAATGTTCGAAACAACTATGAGAGGC
GCTAAGCGTATGGCTATACTCGGAGACACAGCATGGGACTTCGGA
TCCTTAGGGGGAGTGTTTACGTCAATCGGTAAGGCACTACACCAG
GTATTCGGAGCGATATACGGAGCCGCATTTAGCGGAGTGTCGTGG
ACAATGAAGATACTGATCGGAGTGATAATCACATGGATCGGAATG
AATAGTAGGTCTACAAGTCTATCCGTTAGCTTAGTGTTAGTCGGA
GTCGTTACACTGTATCTAGGCGCTATGGTGCAAGCCGATAGTGGT
TGCGTTGTGAGCTGGAAAAATAAAGAACTGAAATGTGGCAGCGGG
ATCTTCATCACAGATAACGTACACACATGGACAGAACAATATAAG
TTCCAACCAGAATCCCCTTCAAAACTAGCTTCAGCTATCCAAAAA
GCTCATGAAGAGGGCATTTGTGGAATCCGCTCAGTAACAAGATTG
GAGAATCTGATGTGGAAACAAATAACACCAGAATTGAATCATATT
CTATCAGAAAATGAGGTAAAGTTGACCATTATGACAGGAGACATT
AGAGGAATCATGCAGGCAGGAAAACGATCCTTGCGGCCCCAGCCC
ACTGAGCTGAAGTACTCATGGAAAACATGGGGAAAGGCGAAAATG
CTCTCCACAGAGTCTCACAATCAGACCTTTCTTATTGATGGCCCT
GAAACAGCAGAATGCCCCAACACAAACAGAGCCTGGAACTCGCTG
GAAGTTGAAGACTATGGTTTTGGAGTTTTCACCACCAATATATGG
CTGAAATTGAGAGAAAAACAGGATGTATTTTGTGACTCAAAACTC
ATGTCAGCGGCCATTAAAGACAACAGAGCCGTTCATGCTGATATG
GGTTATTGGATAGAAAGTGCACTCAATGACACATGGAAGATGGAG
AAAGCCTCCTTCATTGAAGTTAAAAGCTGCCACTGGCCAAAGTCA
CACACCCTTTGGAGCAATGGAGTATTAGAAAGTGAGATGATAATC
CCAAAAAATTTTGCCGGGCCAGTGTCACAACACAACTACAGACCA
GGCTACCATACACAAACAGCAGGACCTTGGCATCTAGGTAAGCTT
GAGATGGACTTTGATCTCTGCGAAGGAACTACAGTGGTGGTGACT
GAGGACTGTGGAAATAGAGGACCCTCTTTAAGAACGACCACTGCC
TCTGGAAAACTCATAACAGAATGGTGCTGCCGATCCTGCACACTA
CCACCTCTAAGATACAGAGGTGAGGATGGATGCTGGTACGGGATG
GAAATCAGACCATTGAAAGAGAAAGAAGAGAATTTGGTCAACTCC
TTGGTCACAGCCGGACATGGGCAGATTGACAACTTTTCACTAGGA
GTCTTGGGAATGGCACTGTTCCTGGAAGAAATGCTCAGGACCCGA
ATAGGAACGAAACATGCAATACTGCTAGTTGCAGTATCTTTTGTG
ACATTGATTACTGGGAACATGTCTTTTAGAGACCTGGGAAGAGTG
ATGGTTATGGTGGGCGCTACCATGACGGATGACATAGGTATGGGA
GTGACTTATCTTGCCCTACTAGCAGCTTTTAAGGTTAGACCAACTT
TTGCAGCTGGACTACTCTTAAGAAAACTGACCTCCAAGGAATTGAT
GATGGCCACCATAGGAATCGCACTCCTTTCCCAAAGCACCATACC
AGAGACCATTCTTGAACTGACTGATGCATTAGCCCTGGGCATGAT
GGTCCTCAAAATAGTGAGAAATATGGAAAAATACCAATTGGCAGT
GACTATCATGGCTATTTCATGTGTCCCAAATGCAGTGATACTGCA
AAACGCATGGAAGGTGAGTTGCACAATATTGGCAGCGGTGTCCGT
TTCACCACTGCTCTTAACATCCTCACAGCAGAAAGCGGATTGGAT
ACCACTGGCATTGACGATAAAAGGTCTCAATCCAACAGCCATTTT
TTTAACAACTCTCTCGAGGACCAGCAAGAAAAGGAGCTGGCCGCT
AAATGAAGCTATCATGGCAGTTGGGATGGTGAGCATTTTAGCCAG
TTCTCTCCTAAAGAATGATATTCCTATGACAGGTCCATTAGTGGC
TGGAGGACTCCTCACCGTATGTTACGTGCTCACTGGACGATCGGC
CGATTTGGAACTGGAGAGAGCTGCCGATGTAAAATGGGAAGATCA
GGCAGAAATATCAGGAAGCAGCCCAATCCTGTCAATAACAATATC
AGAAGATGGCAGCATGTCGATAAAAAATGAAGAGGAAGAACAAAC
ACTGACCATACTCATCAGAACGGGATTGTTGGTGATCTCAGGAGT
CTTTCCAGTATCGATACCAATTACGGCAGCAGCATGGTACCTGTG
GGAAGTAAAGAAACAACGGGCTGGAGTACTGTGGGACGTCCCTTC
ACCCCCACCAGTGGAAAAAGCCGAACTGGAGGATGGAGCCTACAG
AATCAAGCAAAGAGGGATCCTTGGATATTCTCAGATTGGAGCCGG
AGTTTACAAAGAAGGAACATTCCATACAATGTGGCACGTCACACG
TGGTGCTGTTCTGATGCATAGAGGGAAGAGGATTGAACCATCATG
GGCAGATGTCAAGAAAGACCTAATATCATATGGAGGAGGCTGGAA
GCTAGAAGGAGAATGGAAGGAAGGAGAGGAAGTCCAAGTCCTGGC
ATTGGAACCTGGAAAAAATCCAAGAGCCGTCCAAACGAAACCTGG
AATATTCAAAACCAACACCGGAACCATAGGCGCCGTATCTCTGGA
CTTTTCCCCTGGAACGTCAGGATCTCCAATCGTCGACAGAAAAGG
AAAAGTTGTGGGTCTTTACGGTAATGGTGTTGTCACAAGGAGTGG
AGCATATGTAAGTGCTATAGCCCAGACCGAAAAAAGCATTGAAGA
CAATCCAGAGATCGAAGATGACATTTTCCGAAAGAAAAGATTGAC
CATCATGGACCTCCATCCAGGAGCAGGAAAGACAAAAAGATACCT
TCCAGCCATAGTTAGAGAAGCCATAAAACGTGGCTTGAGAACATT
GATCCTGGCTCCCACTAGAGTAGTGGCAGCTGAAATGGAGGAAGC
TCTTAGAGGACTTCCAATAAGATACCAAACTACAGCCATCAAAAC
CGAGCATACCGGGCGGGAGATCGTGGACCTAATGTGTCATGCCAC
ATTTACTATGAGGCTGTTATCACCAGTCAGAGTGCCAAATTACAA
CCTGATCATCATGGACGAAGCCCACTTCACAGACCCAGCAAGTAT
AGCAGCTAGAGGATACATTTCAACTCGAGTAGAGATGGGTGAAGC
AGCCGGGATTTTCATGACAGCCACTCCTCCGGGAAGTAGAGACCC
ATTTCCTCAGAGCAATGCACCAATTATGGATGAGGAAAGAGAAAT
CCCTGAGCGTTCATGGAATTCAGGACACGAATGGGTCACGGATTT
TAAGGGGAAGACTGTTTGGTTTGTTCCAAGTATAAAAGCAGGAAA
TGATATAGCAGCTTGTCTTAGGAAAAATGGAAAGAAAGTGATACA
ACTCAGTAGGAAGACTTTTGACTCTGAGTATGTTAAGACTAGAGC
CAATGATTGGGACTTTGTGGTCACAACTGACATTTCAGAAATGGG
TGCCAACTTCAAGGCTGAGAGGGTTATAGACCCCAGACGTTGCAT
GAAACCAGTTATACTAACAGATGGCGAGGAGCGGGTGATCTTGGC
TGGACCTATGCCAGTGACCCACTCTAGTGCAGCGCAAAGAAGAGG
GAGAATAGGAAGAAATCCAAAAAATGAAAATGACCAGTACATATA
CATGGGGGAACCTCTTGAAAATGATGAAGACTGTGCACATTGGAA
AGAAGCTAAAATGCTCCTAGATAACATCAACACACCTGAAGGAAT
CATTCCTAGTATGTTCGAACCAGAGCGTGAAAAAGTGGATGCCAT
TGATGGTGAATACCGTTTGAGAGGAGAAGCAAGGAAAACCTTTGT
GGACCTAATGAGAAGAGGGGACTTACCAGTCTGGTTGGCCTACAA
AGTGGCAGCTGAAGGCATCAACTACGCAGACAGAAAGTGGTGTTT
TGATGGAATTAAGAACAACCAAATACTGGAAGAAAATATGGAAGT
GGAAATCTGGACAAAAGAAGGGGAAAGGAAAAAATTAAAACCCAG
ATGGTTGGATGCTAGGATCTATTCTGACCCACTAGCACTAAAAGA
ATTCAAGGAATTTGCAGCTGGAAGAAAATCTTTGACCCTGAACCT
AATCACAGAAATGGGTAGGCTTCCAACTTTCATGACTCAGAAAGC
AAGAAACGCACTGGACAACCTGGCTGTGCTGCATACGGCTGAGGC
AGGTGGAAGGGCGTACAATCATGCTCTCAGTGAACTGCCGGAGAC
CCTGGAGACACTGCTCCTACTGACACTTCTGGCAACAGTCACAGG
AGGAATCTTCTTATTCTTAATGAGCGGAAAAGGTATAGGGAAGAT
GACCCTGGGAATGTGTTGCATAATCACGGCTAGTATCCTCCTATG
GTATGCATTCCAGAACCAGAAAAACAGAGAACACCCCAAGACAAC
CAATTGACCTACGTTGTCATAGCCATCCTCACAGTGGTGGCCGCA
ACCATGGCAAACGAGATGGGTTTCCTGGAAAAAACCAAGAAAGAC
CTCGGATTGGGAAGCATTACAACCCAGGAATCTGAGAGCAATATC
CTGGACATAGATCTACGCCCTGCATCAGCATGGACGCTGTATGCC
GTAGCTACAACATTTGTCACACCAATGTTGAGACATAGCATTGAA
AATTCCTCAGTGAATGTCTCCCTAACAGCCATTGCTAACCAAGCT
ACAGTGCTAATGGGTCTTGGGAAAGGATGGCCATTGTCAAAGATG
GACATTGGAGTTCCCCTCCTTGCCATTGGATGCTATTCACAAGTC
AACCCTATAACTCTCACAGCAGCTCTCCTTTTATTGGTAGCACAT
TATGCCATTATAGGGCCAGGACTTCAAGCAAAAGCAACCAGAGAA
GCCCAGAAAAGAGCAGCAGCAGGCATCATGAAAAACCCAACAGTC
GATGGAATAACAGTGATTGACCTAGAACCAATACCCTATGATCCA
AAATTTGAAAAGCAGTTAGGACAAGTAATGCTCCTAATCCTCTGC
GTGACTCAAGTATTAATGATGAGGACTACATGGGCTTTATGTGAG
GCTCTAACCCTAGCGACCGGGCCCATCTCCACACTGTGGGAAGGA
AATCCAGGGAGGTTTTGGAACACTACCATTGCAGTGTCAATGGCT
AACATCTTTAGGGGGAGCTACTTGGCCGGAGCTGGACTTCTCTTT
TCCATCATGAAAAACACAACAAACACAAGAAGAGGAACTGGCAAC
ATAGGAGAGACACTTGGAGAAAAATGGAAAAGTCGATTAAACGCA
CTGGGGAAAAGTGAATTTCAAATCTACAAGAAAAGTGGAATCCAG
GAAGTGGATAGAACCCTAGCAAAAGAAGGTATCAAAAGAGGAGAA
ACGGACCACCATGCTGTGTCGCGAGGTTCAGCAAAACTGAGATGG
TTCGTCGAGAGAAATATGGTCACACCGGAAGGGAAGGTGGTGGAT
CTCGGTTGCGGCAGAGGGGGCTGGTCATACTATTGCGGGGGACTA
AAGAATGTAAGAGAAGTCAAAGGCCTAACAAAAGGAGGACCAGGA
CATGAAGAACCCATCCCCATGTCAACATATGGGTGGAATCTAGTG
CGTCTGCAAAGTGGAGTTGACGTTTTCTTCACCCCGCCAGAAAAG
TGTGATACATTGTTGTGTGACATAGGGGAGTCGTCACCAAATCCC
ACGATAGAAGCAGGACGAACACTCAGAGTCCTCAACTTAGTGGAA
AATTGGTTGAACAATAACACCCAATTTTGCATAAAGGTCCTCAAC
CCATATATGCCTTCAGTCATAGAAAAAATGGAAGCATTACAAAGG
AAATATGGAGGAGCCTTAGTGAGGAATCCACTCTCACGAAACTCC
ACGCATGAAATGTACTGGGTATCTAATGCCACCGGGAACATAGTG
TCATCAGTGAACATGATTTCAAGGATGTTGATTAACAGATTCACA
ATGAAACACAAGAAAGCCACTTACGAGCCAGATGTTGACCTAGGA
AGTGGAACCCGCAACATTGGAATTGAAAGTGAGATACCAAATCTA
GACATAATAGGAAAGAGAATAGAGAAAATAAAACAAGAGCATGAA
ACATCATGGCACTATGATCAAGACCACCCATACAAAACGTGGGCT
TACCATGGCAGCTATGAAACAAAACAAACTGGATCAGCATCATCT
ATGGTGAACGGAGTGGTCAGATTGCTGACAAAACCTTGGGACGTC
GTCCCTATGGTGACACAGATGGCAATGACAGACACGACTCCTTTT
GGACAACAGCGCGTTTTCAAAGAGAAAGTAGACACGAGAACCCAA
GAACCGAAGGAAGGCACAAAGAAACTGATGAAAATTACGGCAGAG
TGGCTTTGGAAAGAACTAGGAAAGAAAAAGACACCTAGGATGTGT
ACCAGAGAAGAATTCACAAGAAAAGTGAGAAGCAATGCAGCCTTG
GGGGCCATATTCACTGATGAGAACAAATGGAAATCGGCACGTGAG
GCTGTTGAAGATGGTAGGTTCTGGGAGCTGGTTGACAGGGAAAGA
AATCTCCATCTTGAAGGAAAGTGTGAAACATGTGTGTACAACATG
ATGGGAAAAAGAGAGAAGAAACTAGGGGAGTTCGGCAAGGCAAAA
GGTAGCAGAGCCATATGGTACATGTGGCTTGGAGCACGCTTCTTA
GAGTTTGAAGCCCTAGGATTCCTGAATGAAGATCACTGGTTCTCC
AGAGGGAACTCCCTGAGTGGAGTGGAAGGAGAAGGGCTGCACAGG
CTAGGCTACATTTTAAGAGACGTGGGCAAGAAGGAAGGGGGGGCA
ATGTACGCCGATGATACAGCAGGATGGGACACAAGAATCACACTA
GAAGACTTAAAAAATGAAGAAATGGTAACGAACCACATGAAAGGA
GAACACAAGAAACTAGCCGAGGCCATATTCAAACTAACGTACCAA
AACAAGGTGGTGCGTGTGCAAAGACCAACACCAAGAGGTACAGTA
ATGGATATCATATCGAGAAGAGACCAAAGAGGCAGTGGGCAAGTC
GGCACCTATGGCCTTAATACCTTCACCAATATGGAAGCCCAATTA
ATTAGACAGATGGAGGGAGAAGGAATCTTCAAAAGCATTCAGCAC
CTGACAGCCACAGAAGAAATCGCTGTACAGAACTGGCTAGCAAGA
GTGGGGCGTGAAAGGCTATCAAGAATGGCAATCAGTGGAGATGAT
TGTGTTGTAAAACCTATAGATGACAGATTTGCAAGTGCTTTAACA
GCTCTAAATGACATGGGAAAAGTTAGAAAAGATATACAACAATGG
GAACCTTCAAGAGGATGGAACGATTGGACACAAGTGCCTTTCTGT
TCACACCACTTTCATGAGTTAGTCATGAAAGATGGTCGCGTGCTC
GTAGTCCCATGCAGAAACCAAGATGAACTGATTGGTAGAGCCCGA
ATTTCCCAGGGAGCTGGGTGGTCTTTGAAAGAGACGGCCTGTTTG
GGAAAGTCTTACGCCCAAATGTGGACTCTGATGTACTTCCACAGA
CGTGACCTCAGACTGGCGGCAAATGCCATCTGCTCGGCAGTCCCG
TCACATTGGGTTCCAACAAGTCGAACAACCTGGTCCATACACGCT
AAGCATGAATGGATGACGACGGAAGACATGCTGGCAGTCTGGAAC
AGGGTGTGGATCCAAGAAAACCCGTGGATGGAAGATAAAACTCCA
GTGGAATCATGGGAAGAAGTCCCATACTTGGGAAAAAGAGAAGAC
CAATGGTGCGGCTCATTGATTGGGCTAACAAGCAGGGCTACCTGG
GCAAAGAACATCCAAACAGCAATAAATCAAGTCAGATCCCTTATA
GGCAATGAGGAATACACAGACTACATGCCATCCATGAAGAGATTT
AGAAGGGAAGAGGAAGAGGCAGGTGTCCTGTGGTAGAAGGCAAAA
CTAACATGAAACAAGGCTAAAAGTCAGGTCGGATTAAGCCATAGT
ACGGAAAAAACTATGCTACCTGTGAGCCCCGTCCAAGGACGTTAA
AAGAAGTCAGGCCATCACAAAATGCCACAGCTTGAGTAAACTGTG
CAGCCTGTAGCTCCACCTGAGGAGGTGTAAAAAACCTGGGAGGCC
ACAAACCATGGAAGCTGTACGCATGGCGTAGTGGACTAGCGGTTA
GAGGAGACCCCTCCCTTACAAATCGCAGCAAACAACGGGGGCCCA
AGGTGAGATGAAGCTGTAATCTCACTGGAAGGACTAGAGGTTAGA
GGAGACCCCCCCAAAACAAAAAACAGCATATTGACGCTGGGAAAG
ACCAGAGATCCTGCTGTCTCCTCAGCATCATTCCAGGCACAGAAC
GCCAGAAAATGGAATGGTGCTGTTGAATCAACAGGTTCT
DENV-3-VE-BID-V2268-2008
SEQ ID NO: 5
AGTTGTTAGTCTACGTGGACCGACAAGAACAGTTTCGACTCGGAA
GCTTGCTTAACGTAGTGCTGTCTATCAATATGCTGAAACGCGTGA
GAAACCGTGTGTCAACTGGATCACAGTTGGCGAAGAGATTCTCAA
AAGGACTGCTGAACGGCCAGGGACCAATGAAATTGGTTATGGCGT
TCATAGCTTTCCTCAGATTTCTAGCCATTCCACCAACAGCAGGAG
TCTTGGCTAGATGGGGAACCTTCAAGAAGTCGGGGGCCATTAAGG
TCCTGAAAGGCTTTAAGAAGGAGATCTCAAACATGCTGAGCATAA
TCAACAAACGGAAAAAGACATCGCTCTGTCTCATGATGATATTGC
CAGCAGCACTTGCTTTCCACTTGACTTCACGAGATGGAGAGCCGC
GCATGATTGTGGGGAAGAATGAAAGAGGAAAATCCCTACTTTTTA
AGACAGCCTCTGGAATTAACATGTGCACACTCATAGCCATGGACT
TGGGAGAGATGTGTGATGACACGGTCACTTACAAATGCCCCCATA
TTACCGAAGTGGAACCTGAAGACATTGACTGCTGGTGCAACCTCA
CATCAACATGGGTGACTTATGGAACGTGCAATCAAGCCGGAGAGC
ATAGACGCGATAAGAGATCAGTGGCGTTAGCTCCCCATGTCGGCA
TGGGACTGGATACACGCACCCAAACTTGGATGTCGGCTGAAGGAG
CTTGGAGGCAAGTCGAGAAGGTAGAGACATGGGCCCTTAGGCACC
CAGGGTTCACCATACTAGCCCTATTTCTTGCCCATTACATAGGCA
CTTCCTTGACCCAGAAGGTGGTTATTTTTATACTGCTAATGCTGG
TCACCCCATCCATGACAATGAGATGTGTGGGAGTAGGAAACAGAG
ATTTTGTGGAAGGACTATCAGGAGCTACGTGGGTTGACGTGGTGC
TCGAGCACGGGGGGTGTGTGACCACCATGGCTAAGAACAAGCCCA
CGTTGGATATAGAGCTTCAGAAGACCGAGGCCACCCAACTGGCGA
CCCTAAGGAAGCTATGCATTGAGGGGAAAATCACCAACATAACAA
CTGACTCAAGATGTCCTACCCAAGGGGAAGCGGTTTTGCCTGAGG
AGCAGGACCAAAACTACGTGTGTAAGCATACATACGTAGACAGAG
GCTGGGGGAACGGATGTGGTTTGTTTGGTAAGGGAAGCTTGGTAA
CATGTGCGAAATTTCAATGCCTGGAACCAATAGAGGGAAAAGTGG
TGCAATATGAGAACCTCAAATACACCGTTATCATCACAGTGCACA
CAGGAGACCAACACCAGGTGGGAAATGAAACGCAGGGAGTCACGG
CTGAGATAACACCTCAGGCATCAACCACTGAAGCCATCTTGCCTG
AATATGGAACCCTTGGGCTAGAATGCTCACCACGGACAGGTTTGG
ACTTCAATGAAATGATCTTGCTAACAATGAAGAACAAAGCATGGA
TGGTACATAGACAATGGTTTTTTGACCTACCTCTACCATGGACAT
CAGGAGCTACAACGGAAACACCAACCTGGAACAGGAAGGAGCTTC
TTGTGACATTTAAAAACGCACATGCGAAGAAACAAGAAGTAGTTG
TTCTTGGGTCGCAAGAGGGAGCAATGCATACCGCATTGACAGGAG
CCACAGAAATCCAAAACTCAGGAGGCACAAGCATTTTTGCGGGGC
ACTTAAAATGTAGACTTAAGATGGACAAATTAGAACTCAAGGGGA
TGAGCTATGCAATGTGCACGAATACCTTTGTGTTGAAGAAAGAAG
TCTCAGAAACGCAGCATGGGACAATACTTATCAAGGTCGAGTACA
AAGGGGAAGATGTACCTTGCAAGATTCCTTTCTCCACAGAGGATG
GACAAGGGAAAGCTCACAATGGCAGACTGATTACAGCCAACCCAG
TGGTGACTAAGAAGGAGGAGCCTGTCAATATTGAGGCTGAACCTC
CTTTTGGGGAAAGCAATATAGTAATTGGAATTGGAGACAACGCCT
TGAAAATCAACTGGTACAAGAAGGGAAGCTCTATTGGGAAGATGT
TCGAGGCCACTGCTAGAGGTGCAAGGCGCATGGCCATCTTGGGAG
ACACAGCTTGGGACTTTGGATCAGTGGGTGGTGTTCTGAACTCAT
TAGGCAAAATGGTGCACCAAATATTCGGAAGTGCTTACACAGCCC
TATTCAGTGGAGTCTCTTGGGTAATGAAAATTGGAATAGGAGTTC
TCTTGACTTGGATAGGGTTGAATTCAAAAAACACATCCATGTCAT
TTTCATGCATTGCGATAGGAATCATCACACTCTATCTGGGAGCTG
TGGTGCAAGCTGACATGGGGTGTGTTATAAACTGGAAAGGCAAAG
AACTCAAGTGTGGAAGTGGAATCTTCGTCACCAACGAGGTCCATA
CCTGGACAGAGCAATACAAATTCCAAGCAGACTCCCCAAAAAGAT
TGGCGACAGCCATTGCAGGCGCTTGGGAGAATGGAGTGTGCGGAA
TTAGGTCAACAACCAGAATGGAGAATCTCCTGTGGAAGCAAATAG
CCAATGAACTGAACTACATATTGTGGGAAAACAATATCAAATTAA
CGGTTGTTGTGGGCGATATAATTGGGGTCTTAGAGCAAGGGAAAA
GAACACTAACACCACAACCCATGGAGCTAAAATACTCATGGAAAA
CGTGGGGAAAGGCAAAAATAGTGACAGCTGAAACACAAAATTCTT
CTTTCATAATAGATGGACCAAACACACCGGAGTGTCCAAGTGCCT
CAAGAGCATGGAATGTGTGGGAGGTGGAAGATTACGGGTTCGGAG
TCTTCACAACCAACATATGGCTGAAACTCCGAGAGGTGTATACCC
AACTATGTGACCATAGGTTAATGTCGGCAGCCGTCAAGGATGAAA
GGGCCGTACATGCCGACATGGGCTATTGGATAGAAAGTCAAAAGA
ATGGAAGTTGGAAGCTAGAAAAAGCATCCCTCATAGAGGTGAAAA
CCTGCACATGGCCAAAATCACATACCCTTTGGAGTAATGGTGTGT
TAGAGAGTGACATGATCATTCCAAAAAGTCTAGCTGGTCCTATCT
CGCAACACAACTACAGGCCCGGGTACCACACCCAGACGGCGGGAC
CTTGGCATTTAGGAAAATTAGAGCTGGACTTCAACTATTGTGAAG
GAACAACAGTTGTCATCACAGAAAACTGTGGGACAAGAGGCCCAT
CATTGAGAACAACAACAGTGTCAGGGAAGTTAATACACGAATGGT
GCTGCCGCTCGTGCACACTTCCTCCCCTGCGATACATGGGAGAAG
ACGGTTGCTGGTATGGCATGGAAATCAGACCCATCAGTGAGAAAG
AAGAAAACATGGTAAAGTCTTTAGTCTCAGCGGGAAGTGGAGAGG
TGGACAACTTCACAATGGGTGTCTTGTGTTTGGCAATCCTCTTTG
AAGAGGTGATGAGAGGAAAATTTGGGAAGAAACACATGATTGCGG
GGGTTTTCTTCACGTTTGTACTCCTTCTCTCAGGGCAAATAACAT
GGAGAGATATGGCGCACACACTAATAATGATTGGGTCCAATGCAT
CTGACAGGATGGGAATGGGCGTCACCTACCTAGCTTTAATTGCAA
CATTTAAAATCCAGCCATTTTTGGCTTTGGGATTTTTCCTAAGAA
AACTGACATCCAGAGAAAATTTATTGTTAGGAGTTGGGCTGGCTA
TGGCAACAACGTTACAACTGCCAGAGGACATTGAACAAATGGCAA
ATGGAATCGCTCTGGGGCTCATGGCTCTCAAACTGATAACACAAT
TTGAAACATACCAACTATGGACAGCATTAATCTCCTTAACGTGTT
CAAATACAATTTTTACGTTGACTGTTGCCTGGAGAACAGCCACCC
TGATTTTGGCTGGAGTTTCACTTTTACCAGTGTGCCAGTCTTCGA
GTATGAGGAAAACAGACTGGCTTCCAATGACAGTGGCAGCTATGG
GAGTTCCACCTCTACCACTTTTTATTTTTAGCTTAAAAGACACAC
TCAAAAGGAGAAGCTGGCCACTGAATGAAGGGGTGATGGCTGTTG
GGCTTGTGAGCATTCTGGCCAGTTCTCTCCTTAGAAATGATGTAC
CCATGGCTGGACCATTAGTGGCCGGGGGCTTGCTGATAGCGTGCT
ACGTCATAACTGGCACGTCAGCAGACCTCACCGTAGAAAAAGCAG
CAGATGTAACATGGGAGGAAGAGGCTGAGCAAACAGGAGTGTCCC
ACAACTTAATGATCACAGTTGATGATGATGGAACAATGAGAATAA
AAGATGATGAGACTGAGAATATCTTAACAGTGCTTTTGAAAACAG
CATTACTAATAGTGTCAGGAATCTTTCCATACTCCATACCCGCAA
CATTGTTGGTCTGGCATACTTGGCAAAAGCAAACCCAAAGATCCG
GCGTTCTATGGGATGTACCCAGCCCTCCAGAGACACAGAAAGCAG
AACTGGAAGAAGGGGTCTATAGGATCAAACAGCAAGGAATTTTTG
GGAAAACCCAAGTAGGGGTTGGAGTACAGAAAGAAGGAGTCTTTC
ACACCATGTGGCACGTTACAAGAGGGGCAGTGTTGACATATAATG
GGAAAAGACTGGAACCAAATTGGGCTAGCGTGAAAAAAGATCTGA
TTTCATACGGAGGAGGATGGAGATTGAGCGCACAATGGCAAAAGG
GGGAGGAGGTGCAGGTTATTGCCGTAGAGCCTGGGAAGAACCCAA
AGAACTTTCAAACCATGCCAGGCACTTTTCAGACTACAACAGGGG
AAATAGGAGCAATTGCACTGGATTTCAAGCCCGGAACTTCAGGAT
CTCCTATCATAAACAGAGAGGGAAAGGTAGTGGGACTGTATGGCA
ATGGAGTGGTTACAAAGAATGGTGGCTACGTCAGCGGAATAGCGC
AAACGAATGCAGAACCAGATGGACCGACACCAGAATTGGAAGAAG
AGATGTTCAAAAAGCGAAATCTAACCATAATGGATCTTCATCCTG
GGTCAGGAAAGACACGGAAATACCTTCCAGCTATTGTTAGAGAGG
CAATCAAGAGACGTTTGAGAACTCTAATTCTGGCACCAACAAGGG
TGGTTGCAGCTGAGATGGAAGAAGCATTGAAAGGGCTCCCAATAA
GGTACCAAACAACAGCAACAAAATCTGAACACACAGGAAGAGAGA
TTGTTGATCTGATGTGCCACGCAACGTTCACAATGCGTCTGCTGT
CACCAGTTAGGGTTCCAAACTATAACTTGATAATAATGGATGAAG
CCCATTTCACAGACCCAGCCAGTATAGCTGCTAGAGGGTACATAT
CAACTCGTGTTGGAATGGGAGAAGCAGCCGCAATATTCATGACAG
CAACGCCCCCTGGAACAGCTGATGCCTTTCCCCAGAGCAACGCTC
CAATTCAAGATGAAGAAAGGGACATACCAGAACGCTCATGGAATT
CAGGCAATGAATGGATAACCGACTTCGCTGGGAAAACGGTGTGGT
TTGTCCCCAGCATTAAAGCCGGAAATGACATAGCAAATTGCCTGC
GGAAAAACGGGAAAAAGGTCATTCAACTTAGTAGGAAGACTTTTG
ACACAGAATATCAGAAAACCAAACTGAATGATTGGGACTTTGTGG
TGACGACTGACATTTCAGAAATGGGGGCCAATTTCAAAGCAGATA
GAGTGATCGACCCAAGAAGATGTCTCAAACCAGTGATCCTGACAG
ATGGACCAGAGCGGGTGATCCTGGCTGGACCAATGCCAGTCACCG
CAGCGAGTGCTGCGCAAAGGAGAGGGAGAGTTGGCAGGAACCCAC
AAAAAGAAAATGACCAGTACATATTCACGGGCCAGCCTCTCAACA
ATGATGAAGACCATGCTCACTGGACAGAAGCAAAAATGCTGCTGG
ACAACATTAACACACCAGAAGGGATCATACCAGCTCTCTTTGAGC
CAGAAAGGGAGAAGTCAGCCGCCATAGACGGTGAGTATCGCCTGA
AAGGTGAGTCCAGGAAGACTTTCGTGGAACTCATGAGGAGGGGTG
ACCTTCCAGTCTGGTTAGCCCATAAAGTAGCATCAGAAGGGATCA
AATATACAGATAGAAAATGGTGCTTTGATGGACAACGTAATAATC
AAATTTTAGAAGAGAACATGGATGTGGAAATCTGGACAAAGGAAG
GAGAAAAGAAAAAATTGAGACCTAGGTGGCTTGATGCTCGCACCT
ATTCAGATCCCTTAGCACTCAAGGAATTCAAGGACTTTGCGGCTG
GCAGGAAGTCAATAGCCCTTGATCTTGTGACAGAAATAGGAAGAG
TGCCTTCACACCTAGCTCATAGAACGAGAAACGCTCTGGACAATC
TGGTGATGCTGCATACGTCAGAACATGGCGGTAAGGCCTACAGGC
ATGCGGTGGAGGAACTACCAGAGACAATGGAAACACTCCTACTCT
TGGGACTCATGATCTTGTTGACAGGTGGAGCAATGCTTTTCTTAA
TATCAGGTAAAGGGATTGGAAAGACTTCAATAGGACTCATTTGTG
TAATTGCTTCCAGCGGCATGTTGTGGATGGCCGAAATCCCACTCC
AATGGATCGCGTCGGCCATAGTCCTGGAGTTTTTTATGATGGTGT
TGCTTATACCAGAACCAGAGAAGCAGAGAACCCCCCAAGACAACC
AACTCGCATATGTCGTGATAGGCATACTTACACTGGCAGCAATAA
TAGCAGCCAATGAAATGGGATTGTTGGAAACTACAAAGAGAGATT
TAGGAATGTCTAAGGAGCCAGGTGTTGTTTCTCCAACCAGCTATT
TAGATGTGGACTTGCACCCAGCATCAGCCTGGACATTGTACGCTG
TGGCCACTACAGTAATAACACCAATGTTAAGACATACCATAGAGA
ATTCCACAGCAAATGTGTCCTTGGCAGCTATAGCCAACCAGGCAG
TGGTCCTGATGGGTTTGGACAAAGGATGGCCAATATCAAAAATGG
ACTTAGGAGTACCCCTGCTGGCATTGGGTTGCTATTCACAAGTGA
ACCCACTGACTCTAACAGCGGCAGTACTCTTGCTGATCACACATT
ATGCCATTATAGGTCCAGGATTGCAGGCAAAAGCCACCCGTGAAG
CTCAGAAAAGGACAGCTGCTGGAATAATGAAGAATCCAACAGTGG
ATGGGATAATGACAATAGACCTAGATCCTGTAATATATGATTCAA
AATTTGAAAAGCAACTGGGACAGGTTATGCTCCTGGTTTTGTGTG
CAGTTCAACTTTTGTTAATGAGAACATCATGGGCCTTGTGTGAAG
CTTTAACCCTAGCTACAGGACCAATAACAACACTCTGGGAAGGAT
CACCTGGGAAGTTTTGGAACACCACGATAGCTGTTTCCATGGCGA
ACATTTTTAGAGGGAGCTATTTAGCAGGAGCTGGGCTTGCTTTCT
CTATTATGAAATCAGTTGGAACAGGGAAAAGAGGAACAGGCTCAC
AGGGTGAAACTTTGGGAGAAAAATGGAAAAAGAAGTTAAATCAAT
TATCCCGGAAAGAGTTTGACCTTTACAAGAAATCTGGAATCACTG
AGGTGGATAGAACAGAAGCCAAAGAAGGGTTGAAAAGAGGAGAAA
TAACACATCATGCCGTGTCCAGAGGTAGTGCAAAACTTCAATGGT
TTGTGGAGAGAAACATGGTCATTCCCGAAGGAAGAGTTATAGACT
TGGGCTGTGGAAGAGGAGGCTGGTCATATTACTGTGCAGGACTGA
AAAAAGTCACAGAAGTGCGAGGATACACAAAAGGCGGTCCAGGAC
ACGAAGAACCAGTACCTATGTCCACATATGGATGGAACATAGTTA
AGTTAATGAGTGGAAAGGATGTGTTTTATCTTCCACCTGAAAAGT
GTGACACCCTGTTGTGCGACATTGGAGAATCTTCACCAAGCCCAA
CAGTGGAAGAAAGCAGAACTATAAGAGTTTTGAAGATGGTTGAAC
CATGGCTAAAGAACAACCAATTTTGTATTAAAGTATTGAACCCTT
ACATGCCAACTGTGATTGAGCACCTAGAAAGACTACAAAGGAAAC
ATGGAGGAATGCTTGTGAGAAATCCACTTTCACGAAACTCCACGC
ACGAAATGTACTGGATATCCAATGGCACAGGTAACATTGTCGCTT
CAGTCAACATGGTATCTAGACTGCTACTGAACAGGTTCACGATGA
CACACAGAAGACCCACCATTGAGAAAGATGTGGATTTAGGAGCAG
GAACTCGACATGTTAATGCGGAACCAGAAACACCCAACATGGATG
TCATTGGGGAAAGAATAAAAAGGATCAAGGAGGAGCATAATTCAA
CATGGCACTACGATGACGAAAACCCCTACAAAACGTGGGCTTACC
ATGGATCTTATGAAGTCAAAGCCACAGGCTCAGCCTCCTCCATGA
TAAATGGAGTCGTGAAACTCCTCACTAAACCATGGGATGTGGTGC
CCATGGTGACACAGATGGCAATGACAGATACAACTCCATTTGGCC
AGCAGAGAGTCTTTAAAGAGAAAGTGGACACCAGGACACCCAGGT
CCATGCCAGGAACAAGAAGGGTTATGGGGATCACAGCGGAGTGGC
TCTGGAGAACCCTGGGAAGGAATAAAAAACCCAGGTTATGCACAA
GGGAAGAGTTTACAAAAAAGGTCAGAACTAACGCAGCCATGGGAG
CTGTTTTCACAGAGGAGAACCAATGGGACAGCGCGAAAGCTGCTG
TTGAGGATGAGGATTTTTGGAAACTTGTGGACAGAGAACGTGAAC
TCCACAAACTGGGCAAGTGTGGAAGCTGTGTTTACAACATGATGG
GTAAGAGAGAGAAGAAACTTGGAGAGTTTGGCAAAGCAAAAGGCA
GTAGAGCTATATGGTACATGTGGTTGGGAGCCAGGTACCTTGAGT
TCGAAGCCCTTGGATTCTTAAATGAAGACCACTGGTTCTCGCGTG
AGAACTCTTACAGTGGAGTGGAAGGAGAAGGACTGCACAAGCTAG
GCTATATATTAAGGGACATTTCCAAGATACCCGGAGGAGCTATGT
ATGCTGATGACACAGCTGGTTGGGACACAAGAATAACAGAAGATG
ACCTGCACAATGAGGAAAAGATCACACAGCAAATGGACCCTGAAC
ACAGGCAGTTAGCGAACGCTATATTTAAGCTCACATACCAAAACA
AAGTGGTCAAAGTTCAACGACCGACTCCAACAGGCACGGTAATGG
ATATCATATCTAGGAAAGACCAAAGAGGCAGTGGACAGGTAGGAA
CTTATGGTCTGAATACATTCACCAACATGGAAGCCCAGTTAATCA
GACAAATGGAAGGAGAAGGTGTGCTGTCAAAGGCAGACCTCGAGA
ACCCTCATCTGCCAGAGAAGAAAATTACACAATGGTTGGAAACCA
AAGGAGTGGAGAGGTTAAAAAGAATGGCCATAAGTGGGGATGACT
GCGTGGTGAAACCAATCGATGACAGGTTCGCTAATGCCCTGCTCG
CTCTGAACGACATGGGAAAGGTTCGGAAAGACATACCTCAATGGC
AGCCATCAAAGGGATGGCATGATTGGCAACAGGTTCCTTTCTGCT
CCCACCACTTTCATGAATTGATCATGAAAGATGGAAGAAAGTTGG
TGGTTCCCTGCAGACCCCAGGACGAACTAATAGGAAGGGCAAGAA
TCTCTCAAGGAGCGGGATGGAGCCTTAGAGAAACCGCATGTCTGG
GGAAAGCCTACGCTCAAATGTGGAGTCTCATGTATTTTCACAGAA
GAGACCTCAGACTAGCATCCAACGCCATATGTTCAGCAGTACCAG
TCCACTGGGTCCCCACAAGTAGAACGACATGGTCTATTCACGCTC
ACCATCAGTGGATGACCACAGAAGACATGCTTACTGTCTGGAACA
GAGTGTGGATCGAGGACAATCCATGGATGGAAGACAAAACTCCAG
TCACAACCTGGGAAAATGTTCCATATCTAGGGAAGAGAGAAGACC
AATGGTGCGGATCACTTATTGGTCTCACTTCCAGAGCAACCTGGG
CCCAGAACATACCCACAGCAATTCAACAGGTTAGAAGCCTTATAG
GCAATGAAGAGTTTCTGGACTACATGCCTTCAATGAAGAGATTTA
GGAAGGAGGAGGAGTCGGAGGGAGCCATTTGGTAAAACGTAGGAA
GTGAAAAAGAGGTTAACTGTCAGGCCATATTAAGCCACAGTACGG
AAGAAGCTGTGCTGCCTGTGAGCCCCGTCCAAGGACGTTAAAAGA
AGAAGTCAGGCCCCAAAGCCACGGTTTGAGCAAACCGTGCTGCCT
GTAGCTCCGTCGTGGGGACGTAAAACCTGGGAGGCTGCAAACTGT
GGAAGCTGTACGCACGGTGTAGCAGACTAGCGGTTAGAGGAGACC
CCTCCCATGACACAACGCAGCAGCGGGGCCCGAGCACTGAGGGAA
GCTGTACCTCCTTGCAAAGGACTAGAGGTTAGAGGAGACCCCCCG
CAAATAAAAACAGCATATTGACGCTGGGAGAGACCAGAGATCCTG
CTGTCTCCTCAGCATCATTCCAGGCACAGAACGCCAGAAAATGGA
ATGGTGCTGTTGAATCAACAGGTTCT
DENV-3-VE-BID-V2268-2008-E-Min
SEQ ID NO: 6
AGTTGTTAGTCTACGTGGACCGACAAGAACAGTTTCGACTCGGAA
GCTTGCTTAACGTAGTGCTAACAGTTTTTTATTAGAGAGCAGATC
TCTGATGAACAACCAACGGAAGAAGACGGGAAAACCGTCTATCAA
TATGCTGAAACGCGTGAGAAACCGTGTGTCAACTGGATCACAGTT
GGCGAAGAGATTCTCAAAAGGACTGCTGAACGGCCAGGGACCAAT
GAAATTGGTTATGGCGTTCATAGCTTTCCTCAGATTTCTAGCCAT
TCCACCAACAGCAGGAGTCTTGGCTAGATGGGGAACCTTCAAGAA
GTCGGGGGCCATTAAGGTCCTGAAAGGCTTTAAGAAGGAGATCTC
AAACATGCTGAGCATAATCAACAAACGGAAAAAGACATCGCTCTG
TCTCATGATGATATTGCCAGCAGCACTTGCTTTCCACTTGACTTC
ACGAGATGGAGAGCCGCGCATGATTGTGGGGAAGAATGAAAGAGG
AAAATCCCTACTTTTTAAGACAGCCTCTGGAATTAACATGTGCAC
ACTCATAGCCATGGACTTGGGAGAGATGTGTGATGACACGGTCAC
TTACAAATGCCCCCATATTACCGAAGTGGAACCTGAAGACATTGA
CTGCTGGTGCAACCTCACATCAACATGGGTGACTTATGGAACGTG
CAATCAAGCCGGAGAGCATAGACGCGATAAGAGATCAGTGGCGTT
AGCTCCCCATGTCGGCATGGGACTGGATACACGCACCCAAACTTG
GATGTCGGCTGAAGGAGCTTGGAGGCAAGTCGAGAAGGTAGAGAC
ATGGGCCCTTAGGCACCCAGGGTTCACCATACTAGCCCTATTTCT
TGCCCATTACATAGGCACTTCCTTGACCCAGAAGGTGGTTATTTT
TTACTGCTAATGCTGGTCACCCCATCCATGACAATGAGATGCGTG
GGCGTAGGGAATAGAGACTTTGTAGAGGGATTGAGCGGAGCGACA
TGGGTCGACGTCGTACTCGAACACGGGGGGTGCGTGACGACTATG
GCTAAGAATAAGCCTACACTCGATATCGAACTGCAAAAAACCGAA
GCGACACAATTGGCGACATTGCGCAAACTATGTATCGAAGGTAAG
ATTACGAACATTACAACCGATAGTAGATGCCCTACACAAGGCGAA
GCCGTTCTACCTGAGGAGCAAGACCAAAACTACGTATGTAAGCAT
ACATACGTTGATAGGGGGTGGGGGAACGGATGCGGATTGTTCGGT
AAGGGGTCATTGGTCACATGCGCTAAGTTCCAATGCTTAGAGCCT
ATCGAGGGTAAGGTGGTACAATACGAAAACCTTAAGTATACCGTG
ATAATTACAGTTCATACCGGAGACCAACACCAAGTGGGAAACGAG
ACACAGGGAGTGACAGCCGAAATTACGCCTCAGGCTAGTACAACC
GAAGCGATACTACCCGAATACGGAACATTGGGGTTGGAGTGTTCA
CCTAGAACCGGATTGGACTTTAACGAAATGATACTGTTGACTATG
AAAAACAAGGCATGGATGGTGCATAGGCAATGGTTTTTTGACCTA
CCATTGCCTTGGACTAGCGGAGCGACAACCGAAACACCTACATGG
AATAGAAAAGAGCTCCTAGTGACATTTAAAAACGCTCACGCTAAG
AAGCAGGAAGTGGTCGTTTTAGGGTCACAGGAGGGAGCTATGCAT
ACCGCACTAACCGGAGCGACTGAGATACAGAATAGCGGAGGGACA
TCAATCTTCGCCGGACACCTTAAGTGTAGATTGAAAATGGACAAA
CTTGAGCTTAAGGGGATGTCATACGCTATGTGTACGAACACATTC
GTGCTGAAAAAAGAGGTAAGCGAAACGCAACACGGAACGATACTG
ATTAAGGTTGAGTATAAGGGCGAAGACGTCCCATGTAAGATACCT
TTTTCGACAGAGGACGGACAGGGTAAGGCCCATAACGGAAGACTG
ATTACCGCTAACCCAGTGGTGACCAAAAAAGAGGAACCAGTCAAT
ATCGAAGCCGAACCTCCATTCGGAGAGTCAAACATAGTGATAGGG
ATAGGCGATAACGCACTTAAGATTAACTGGTACAAAAAAGGGTCA
TCAATCGGTAAGATGTTTGAGGCAACCGCTAGGGGGGCTAGACGG
ATGGCCATACTCGGAGACACCGCATGGGACTTCGGATCCGTGGGG
GGGGTACTTAACTCACTCGGTAAGATGGTGCACCAAATTTTCGGA
TCCGCATATACCGCTCTATTTAGCGGAGTGTCATGGGTGATGAAA
ATCGGAATCGGAGTTCTATTGACATGGATCGGATTGAACTCTAAG
AATACATCTATGTCATTTTCATGTATCGCAATCGGAATTATTACG
CTATATCTCGGAGCCGTAGTGCAAGCCGACATGGGGTGTGTTATA
AACTGGAAAGGCAAAGAACTCAAGTGTGGAAGTGGAATCTTCGTC
ACCAACGAGGTCCATACCTGGACAGAGCAATACAAATTCCAAGCA
GACTCCCCAAAAAGATTGGCGACAGCCATTGCAGGCGCTTGGGAG
AATGGAGTGTGCGGAATTAGGTCAACAACCAGAATGGAGAATCTC
CTGTGGAAGCAAATAGCCAATGAACTGAACTACATATTGTGGGAA
AACAATATCAAATTAACGGTTGTTGTGGGCGATATAATTGGGGTC
TTAGAGCAAGGGAAAAGAACACTAACACCACAACCCATGGAGCTA
AAATACTCATGGAAAACGTGGGGAAAGGCAAAAATAGTGACAGCT
GAAACACAAAATTCTTCTTTCATAATAGATGGACCAAACACACCG
GAGTGTCCAAGTGCCTCAAGAGCATGGAATGTGTGGGAGGTGGAA
GATTACGGGTTCGGAGTCTTCACAACCAACATATGGCTGAAACTC
CGAGAGGTGTATACCCAACTATGTGACCATAGGTTAATGTCGGCA
GCCGTCAAGGATGAAAGGGCCGTACATGCCGACATGGGCTATTGG
ATAGAAAGTCAAAAGAATGGAAGTTGGAAGCTAGAAAAAGCATCC
CTCATAGAGGTGAAAACCTGCACATGGCCAAAATCACATACCCTT
TGGAGTAATGGTGTGTTAGAGAGTGACATGATCATTCCAAAAAGT
CTAGCTGGTCCTATCTCGCAACACAACTACAGGCCCGGGTACCAC
ACCCAGACGGCGGGACCTTGGCATTTAGGAAAATTAGAGCTGGAC
TTCAACTATTGTGAAGGAACAACAGTTGTCATCACAGAAAACTGT
GGGACAAGAGGCCCATCATTGAGAACAACAACAGTGTCAGGGAAG
TTAATACACGAATGGTGCTGCCGCTCGTGCACACTTCCTCCCCTG
CGATACATGGGAGAAGACGGTTGCTGGTATGGCATGGAAATCAGA
CCCATCAGTGAGAAAGAAGAAAACATGGTAAAGTCTTTAGTCTCA
GCGGGAAGTGGAGAGGTGGACAACTTCACAATGGGTGTCTTGTGT
TTGGCAATCCTCTTTGAAGAGGTGATGAGAGGAAAATTTGGGAAG
AAACACATGATTGCGGGGGTTTTCTTCACGTTTGTACTCCTTCTC
TCAGGGCAAATAACATGGAGAGATATGGCGCACACACTAATAATG
ATTGGGTCCAATGCATCTGACAGGATGGGAATGGGCGTCACCTAC
CTAGCTTTAATTGCAACATTTAAAATCCAGCCATTTTTGGCTTTG
GGATTTTTCCTAAGAAAACTGACATCCAGAGAAAATTTATTGTTA
GGAGTTGGGCTGGCTATGGCAACAACGTTACAACTGCCAGAGGAC
ATTGAACAAATGGCAAATGGAATCGCTCTGGGGCTCATGGCTCTC
AAACTGATAACACAATTTGAAACATACCAACTATGGACAGCATTA
ATCTCCTTAACGTGTTCAAATACAATTTTTACGTTGACTGTTGCC
TGGAGAACAGCCACCCTGATTTTGGCTGGAGTTTCACTTTTACCA
GTGTGCCAGTCTTCGAGTATGAGGAAAACAGACTGGCTTCCAATG
ACAGTGGCAGCTATGGGAGTTCCACCTCTACCACTTTTTATTTTT
AGCTTAAAAGACACACTCAAAAGGAGAAGCTGGCCACTGAATGAA
GGGGTGATGGCTGTTGGGCTTGTGAGCATTCTGGCCAGTTCTCTC
CTTAGAAATGATGTACCCATGGCTGGACCATTAGTGGCCGGGGGC
TTGCTGATAGCGTGCTACGTCATAACTGGCACGTCAGCAGACCTCA
CCG
TAGAAAAAGCAGCAGATGTAACATGGGAGGAAGAGGCTGAGCAAA
CAGGAGTGTCCCACAACTTAATGATCACAGTTGATGATGATGGAA
CAATGAGAATAAAAGATGATGAGACTGAGAATATCTTAACAGTGC
TTTTGAAAACAGCATTACTAATAGTGTCAGGAATCTTTCCATACT
CCATACCCGCAACATTGTTGGTCTGGCATACTTGGCAAAAGCAAA
CCCAAAGATCCGGCGTTCTATGGGATGTACCCAGCCCTCCAGAGA
CACAGAAAGCAGAACTGGAAGAAGGGGTCTATAGGATCAAACAGC
AAGGAATTTTTGGGAAAACCCAAGTAGGGGTTGGAGTACAGAAAG
AAGGAGTCTTTCACACCATGTGGCACGTTACAAGAGGGGCAGTGTT
GACATATAA
TGGGAAAAGACTGGAACCAAATTGGGCTAGCGTGAAAAAAGATCT
GATTTCATACGGAGGAGGATGGAGATTGAGCGCACAATGGCAAAA
GGGGGAGGAGGTGCAGGTTATTGCCGTAGAGCCTGGGAAGAACCC
AAAGAACTTTCAAACCATGCCAGGCACTTTTCAGACTACAACAGG
GGAAATAGGAGCAATTGCACTGGATTTCAAGCCCGGAACTTCAGG
ATCTCCTATCATAAACAGAGAGGGAAAGGTAGTGGGACTGTATGG
CAATGGAGTGGTTACAAAGAATGGTGGCTACGTCAGCGGAATAGC
GCAAACGAATGCAGAACCAGATGGACCGACACCAGAATTGGAAGA
AGAGATGTTCAAAAAGCGAAATCTAACCATAATGGATCTTCATCC
TGGGTCAGGAAAGACACGGAAATACCTTCCAGCTATTGTTAGAGA
GGCAATCAAGAGACGTTTGAGAACTCTAATTCTGGCACCAACAAG
GGTGGTTGCAGCTGAGATGGAAGAAGCATTGAAAGGGCTCCCAAT
AAGGTACCAAACAACAGCAACAAAATCTGAACACACAGGAAGAGA
GATTGTTGATCTGATGTGCCACGCAACGTTCACAATGCGTCTGCT
GTCACCAGTTAGGGTTCCAAACTATAACTTGATAATAATGGATGA
AGCCCATTTCACAGACCCAGCCAGTATAGCTGCTAGAGGGTACAT
ATCAACTCGTGTTGGAATGGGAGAAGCAGCCGCAATATTCATGAC
AGCAACGCCCCCTGGAACAGCTGATGCCTTTCCCCAGAGCAACGC
TCCAATTCAAGATGAAGAAAGGGACATACCAGAACGCTCATGGAA
TTCAGGCAATGAATGGATAACCGACTTCGCTGGGAAAACGGTGTG
GTTTGTCCCCAGCATTAAAGCCGGAAATGACATAGCAAATTGCCT
GCGGAAAAACGGGAAAAAGGTCATTCAACTTAGTAGGAAGACTTT
TGACACAGAATATCAGAAAACCAAACTGAATGATTGGGACTTTGT
GGTGACGACTGACATTTCAGAAATGGGGGCCAATTTCAAAGCAGA
TAGAGTGATCGACCCAAGAAGATGTCTCAAACCAGTGATCCTGAC
AGATGGACCAGAGCGGGTGATCCTGGCTGGACCAATGCCAGTCAC
CGCAGCGAGTGCTGCGCAAAGGAGAGGGAGAGTTGGCAGGAACCC
ACAAAAAGAAAATGACCAGTACATATTCACGGGCCAGCCTCTCAA
CAATGATGAAGACCATGCTCACTGGACAGAAGCAAAAATGCTGCT
GGACAACATTAACACACCAGAAGGGATCATACCAGCTCTCTTTGA
GCCAGAAAGGGAGAAGTCAGCCGCCATAGACGGTGAGTATCGCCT
GAAAGGTGAGTCCAGGAAGACTTTCGTGGAACTCATGAGGAGGGG
TGACCTTCCAGTCTGGTTAGCCCATAAAGTAGCATCAGAAGGGAT
CAAATATACAGATAGAAAATGGTGCTTTGATGGACAACGTAATAA
TCAAATTTTAGAAGAGAACATGGATGTGGAAATCTGGACAAAGGA
AGGAGAAAAGAAAAAATTGAGACCTAGGTGGCTTGATGCTCGCAC
CTATTCAGATCCCTTAGCACTCAAGGAATTCAAGGACTTTGCGGC
TGGCAGGAAGTCAATAGCCCTTGATCTTGTGACAGAAATAGGAAG
AGTGCCTTCACACCTAGCTCATAGAACGAGAAACGCTCTGGACAA
TCTGGTGATGCTGCATACGTCAGAACATGGCGGTAAGGCCTACAG
GCATGCGGTGGAGGAACTACCAGAGACAATGGAAACACTCCTACT
CTTGGGACTCATGATCTTGTTGACAGGTGGAGCAATGCTTTTCTT
AATATCAGGTAAAGGGATTGGAAAGACTTCAATAGGACTCATTTG
TGTAATTGCTTCCAGCGGCATGTTGTGGATGGCCGAAATCCCACT
CCAATGGATCGCGTCGGCCATAGTCCTGGAGTTTTTTATGATGGT
GTTGCTTATACCAGAACCAGAGAAGCAGAGAACCCCCCAAGACAA
CCAACTCGCATATGTCGTGATAGGCATACTTACACTGGCAGCAAT
AATAGCAGCCAATGAAATGGGATTGTTGGAAACTACAAAGAGAGA
TTTAGGAATGTCTAAGGAGCCAGGTGTTGTTTCTCCAACCAGCTA
TTTAGATGTGGACTTGCACCCAGCATCAGCCTGGACATTGTACGC
TGTGGCCACTACAGTAATAACACCAATGTTAAGACATACCATAGA
GAATTCCACAGCAAATGTGTCCTTGGCAGCTATAGCCAACCAGGC
AGTGGTCCTGATGGGTTTGGACAAAGGATGGCCAATATCAAAAAT
GGACTTAGGAGTACCCCTGCTGGCATTGGGTTGCTATTCACAAGT
GAACCCACTGACTCTAACAGCGGCAGTACTCTTGCTGATCACACA
TTATGCCATTATAGGTCCAGGATTGCAGGCAAAAGCCACCCGTGA
AGCTCAGAAAAGGACAGCTGCTGGAATAATGAAGAATCCAACAGT
GGATGGGATAATGACAATAGACCTAGATCCTGTAATATATGATTC
AAAATTTGAAAAGCAACTGGGACAGGTTATGCTCCTGGTTTTGTG
TGCAGTTCAACTTTTGTTAATGAGAACATCATGGGCCTTGTGTGA
AGCTTTAACCCTAGCTACAGGACCAATAACAACACTCTGGGAAGG
ATCACCTGGGAAGTTTTGGAACACCACGATAGCTGTTTCCATGGC
GAACATTTTTAGAGGGAGCTATTTAGCAGGAGCTGGGCTTGCTTT
CTCTATTATGAAATCAGTTGGAACAGGGAAAAGAGGAACAGGCTC
ACAGGGTGAAACTTTGGGAGAAAAATGGAAAAAGAAGTTAAATCA
ATTATCCCGGAAAGAGTTTGACCTTTACAAGAAATCTGGAATCAC
TGAGGTGGATAGAACAGAAGCCAAAGAAGGGTTGAAAAGAGGAGA
AATAACACATCATGCCGTGTCCAGAGGTAGTGCAAAACTTCAATG
GTTTGTGGAGAGAAACATGGTCATTCCCGAAGGAAGAGTTATAGA
CTTGGGCTGTGGAAGAGGAGGCTGGTCATATTACTGTGCAGGACT
GAAAAAAGTCACAGAAGTGCGAGGATACACAAAAGGCGGTCCAGG
ACACGAAGAACCAGTACCTATGTCCACATATGGATGGAACATAGT
TAAGTTAATGAGTGGAAAGGATGTGTTTTATCTTCCACCTGAAAA
GTGTGACACCCTGTTGTGCGACATTGGAGAATCTTCACCAAGCCC
AACAGTGGAAGAAAGCAGAACTATAAGAGTTTTGAAGATGGTTGA
ACCATGGCTAAAGAACAACCAATTTTGTATTAAAGTATTGAACCC
TTACATGCCAACTGTGATTGAGCACCTAGAAAGACTACAAAGGAA
ACATGGAGGAATGCTTGTGAGAAATCCACTTTCACGAAACTCCAC
GCACGAAATGTACTGGATATCCAATGGCACAGGTAACATTGTCGC
TTCAGTCAACATGGTATCTAGACTGCTACTGAACAGGTTCACGAT
GACACACAGAAGACCCACCATTGAGAAAGATGTGGATTTAGGAGC
AGGAACTCGACATGTTAATGCGGAACCAGAAACACCCAACATGGA
TGTCATTGGGGAAAGAATAAAAAGGATCAAGGAGGAGCATAATTC
AACATGGCACTACGATGACGAAAACCCCTACAAAACGTGGGCTTA
CCATGGATCTTATGAAGTCAAAGCCACAGGCTCAGCCTCCTCCAT
GATAAATGGAGTCGTGAAACTCCTCACTAAACCATGGGATGTGGT
GCCCATGGTGACACAGATGGCAATGACAGATACAACTCCATTTGG
CCAGCAGAGAGTCTTTAAAGAGAAAGTGGACACCAGGACACCCAG
GTCCATGCCAGGAACAAGAAGGGTTATGGGGATCACAGCGGAGTG
GCTCTGGAGAACCCTGGGAAGGAATAAAAAACCCAGGTTATGCAC
AAGGGAAGAGTTTACAAAAAAGGTCAGAACTAACGCAGCCATGGG
AGCTGTTTTCACAGAGGAGAACCAATGGGACAGCGCGAAAGCTGC
TGTTGAGGATGAGGATTTTTGGAAACTTGTGGACAGAGAACGTGA
ACTCCACAAACTGGGCAAGTGTGGAAGCTGTGTTTACAACATGAT
GGGTAAGAGAGAGAAGAAACTTGGAGAGTTTGGCAAAGCAAAAGG
CAGTAGAGCTATATGGTACATGTGGTTGGGAGCCAGGTACCTTGA
GTTCGAAGCCCTTGGATTCTTAAATGAAGACCACTGGTTCTCGCG
TGAGAACTCTTACAGTGGAGTGGAAGGAGAAGGACTGCACAAGCT
AGGCTATATATTAAGGGACATTTCCAAGATACCCGGAGGAGCTAT
GTATGCTGATGACACAGCTGGTTGGGACACAAGAATAACAGAAGA
TGACCTGCACAATGAGGAAAAGATCACACAGCAAATGGACCCTGA
ACACAGGCAGTTAGCGAACGCTATATTTAAGCTCACATACCAAAA
CAAAGTGGTCAAAGTTCAACGACCGACTCCAACAGGCACGGTAAT
GGATATCATATCTAGGAAAGACCAAAGAGGCAGTGGACAGGTAGG
AACTTATGGTCTGAATACATTCACCAACATGGAAGCCCAGTTAAT
CAGACAAATGGAAGGAGAAGGTGTGCTGTCAAAGGCAGACCTCGA
GAACCCTCATCTGCCAGAGAAGAAAATTACACAATGGTTGGAAAC
CAAAGGAGTGGAGAGGTTAAAAAGAATGGCCATAAGTGGGGATGA
CTGCGTGGTGAAACCAATCGATGACAGGTTCGCTAATGCCCTGCT
CGCTCTGAACGACATGGGAAAGGTTCGGAAAGACATACCTCAATG
GCAGCCATCAAAGGGATGGCATGATTGGCAACAGGTTCCTTTCTG
CTCCCACCACTTTCATGAATTGATCATGAAAGATGGAAGAAAGTT
GGTGGTTCCCTGCAGACCCCAGGACGAACTAATAGGAAGGGCAAG
AATCTCTCAAGGAGCGGGATGGAGCCTTAGAGAAACCGCATGTCT
GGGGAAAGCCTACGCTCAAATGTGGAGTCTCATGTATTTTCACAG
AAGAGACCTCAGACTAGCATCCAACGCCATATGTTCAGCAGTACC
AGTCCACTGGGTCCCCACAAGTAGAACGACATGGTCTATTCACGC
TCACCATCAGTGGATGACCACAGAAGACATGCTTACTGTCTGGAA
CAGAGTGTGGATCGAGGACAATCCATGGATGGAAGACAAAACTCC
AGTCACAACCTGGGAAAATGTTCCATATCTAGGGAAGAGAGAAGA
CCAATGGTGCGGATCACTTATTGGTCTCACTTCCAGAGCAACCTG
GGCCCAGAACATACCCACAGCAATTCAACAGGTTAGAAGCCTTAT
AGGCAATGAAGAGTTTCTGGACTACATGCCTTCAATGAAGAGATT
TAGGAAGGAGGAGGAGTCGGAGGGAGCCATTTGGTAAAACGTAGG
AAGTGAAAAAGAGGTTAACTGTCAGGCCATATTAAGCCACAGTAC
GGAAGAAGCTGTGCTGCCTGTGAGCCCCGTCCAAGGACGTTAAAA
GAAGAAGTCAGGCCCCAAAGCCACGGTTTGAGCAAACCGTGCTGC
CTGTAGCTCCGTCGTGGGGACGTAAAACCTGGGAGGCTGCAAACT
GTGGAAGCTGTACGCACGGTGTAGCAGACTAGCGGTTAGAGGAGA
CCCCTCCCATGACACAACGCAGCAGCGGGGCCCGAGCACTGAGGG
AAGCTGTACCTCCTTGCAAAGGACTAGAGGTTAGAGGAGACCCCC
CGCAAATAAAAACAGCATATTGACGCTGGGAGAGACCAGAGATCC
TGCTGTCTCCTCAGCATCATTCCAGGCACAGAACGCCAGAAAATG
GAATGGTGCTGTTGAATCAACAGGTTCT
DENV4_Svn_WT
SEQ ID NO: 7
AGTTGTTAGTCTGTGTGGACCGACAAGGACAGTTCCAAATCGGAA
GCTTGCTTAACACAGTTCTAACAGTTTGTTTAAATAGAGAGCAGA
TCTCTGGAAAAATGAACCAACGAAAAAAGGTGGTTAGACCACCTT
TCAATATGCTGAAACGCGAGAGAAACCGCGTATCAACCCCTCAAG
GGTTGGTGAAGAGATTCTCAACCGGACTTTTTTCTGGGAAAGGAC
CCTTACGGATGGTGCTAGCATTCATCACGTTT
TTGCGAGTCCTTTCCATCCCACCAACAGCAGGGATTCTGAAGAGA
TGGGGACAGTTGAAGAAAAATAAGGCCATCAAGATACTGATTGGA
TTCAGGAAGGAGATAGGCCGCATGCTGAACATCTTGAACGGGAGA
AAAAGGTCAACGATAACATTGTTGTGCTTGATTCCCACCGTAATG
GCGTTTCACTTGTCAACAAGAGATGGCGAACCCCTCATGATAGTG
GCAAAACATGAAAGGGGGAGACCTCTCTTGTTTAAGACAACAGAG
GGGATCAACAAATGCACTCTCATTGCCATGGACTTGGGTGAAATG
TGTGAGGACACTGTCACGTATAAATGCCCCCTACTGGTCAATACC
GAACCTGAAGACATTGATTGCTGGTGCAACCTCACGTCTACCTGG
GTCATGTATGGGACATGCACCCAGAGCGGAGAACGGAGACGAGAG
AAGCGCTCAGTAGCTTTGACACCACATTCAGGAATGGGATTGGAA
ACAAGAGCTGAGACATGGATGTCATCGGAAGGGGCTTGGAAGCAT
GCTCAGAGAGTAGAGAGCTGGATACTCAGAAACCCAGGATTTGCG
CTCTTGGCAGGATTTATGGCTTATATGATTGGGCAAACAGGAATC
CAGCGAACTGTCTTCTTTGTCCTAATGATGCTGGTCGCCCCATCC
TACGGAATGCGGTGCGTAGGAGTAGGAAACAGAGACTTTGTGGAA
GGAGTCTCAGGTGGAGCATGGGTCGACCTGGTGCTAGAACATGGA
GGATGCGTCACAACCATGGCCCAGGGAAAACCAACCTTGGATTTT
GAACTGACTAAGACAACAGCTAAGGAAGTGGCTCTGTTAAGAACC
TATTGCATTGAAGCCTCAATATCAAACATAACTACGGCAACAAGA
TGTCCAACGCAAGGAGAGCCTTATCTGAAAGAGGAACAGGACCAA
CAGTACATCTGCCGGAGAGATGTGGTAGACAGAGGATGGGGCAAT
GGCTGTGGCTTGTTTGGAAAAGGAGGAGTTGTGACATGTGCGAAG
TTTTCATGTTCGGGGAAGATAACAGGCAATCTGGTCCAAATTGAG
AACCTTGAATACACAGTGGTTGTAACAGTCCACAATGGAGACACC
CATGCAGTAGGAAATGACACATCCAATCATGGAGTTACAGCCACG
ATAACTCCCAGGTCACCATCGGTAGAAGTCAAATTGCCGGACTAT
GGAGAACTAACACTCGATTGTGAACCCAGGTCTGGAATTGACTTT
AATGAGATGATCCTAATGAAAATGAAAAAGAAAACATGGCTCGTG
CATAAGCAATGGTTTTTGGATCTGCCTCTTCCATGGACAGCAGGA
GCAGACACATCAGAGGTTCACTGGAATTACAAAGAGAGAATGGTG
ACGTTCAAGGTTCCTCATGCCAAGAGACAGGATGTGACAGTGCTG
GGATCTCAGGAAGGAGCCATGCATTCTGCCCTCGCTGGAGCCACA
GAAGTGGACTCCGGTGATGGAAATCACATGTTTGCAGGACATCTC
AAGTGCAAAGTCCGTATGGAGAAATTGAGAATCAAGGGAATGTCA
TACACGATGTGTTCAGGAAAGTTTTCAATTGACAAAGAGATGGCA
GAAACACAGCATGGGACAACAGTGGTGAAAGTCAAGTATGAAGGT
GCTGGAGCTCCGTGTAAAGTCCCCATAGAGATAAGAGATGTAAAC
AAGGAAAAAGTGGTTGGGCGCGTTATCTCATCCACCCCTTTGGCT
GAGAATACCAACAGTGTAACCAACATAGAATTAGAACCCCCCTTT
GGGGACAGCTACATTGTGATAGGTGTTGGAAACAGCGCATTAACA
CTCCATTGGTTCAGGAAAGGGAGTTCCATTGGCAAGATGTTTGAG
TCCACATACAGAGGTGCAAAACGAATGGCCATTCTAGGTGAAACA
GCTTGGGATTTTGGTTCCGTTGGTGGACTGTTCACATCATTGGGA
AAGGCTGTACACCAGGTTTTTGGAAGCGTGTATACAACCATGTTT
GGAGGAGTCTCATGGATGATTAGAATCCTAATTGGGTTCTTAGTG
TTGTGGATTGGCACGAATTCAAGGAACACTTCAATGGCCATGACG
TGCATAGCTGTTGGAGGAATTACTCTGTTTTTGGGCTTCACAGTT
CAAGCGGACATGGGTTGTGTGGTGTCATGGAGTGGGAGAGAATTG
AAGTGTGGAAGCGGAATTTTTGTGGTTGACAACGTGCACACTTGG
ACAGAACAGTACAAATTCCAACCAGAGTCCCCAGCGAGACTAGCG
TCTGCAATATTAAATGCCCACAAAGATGGGGTCTGTGGAATTAGA
TCAACCACGAGGCTGGAAAATGTTATGTGGAAGCAAATAACCAAT
GAGCTAAACTATGTTCTCTGGGAAGGAGGACATGATCTCACTGTA
GTGGCTGGGGATGTGAAAGGGGTGTTGACCAAGGGCAAGAGAGCA
CTCACACCCCCAGCGAGTGATCTGAAATATTCATGGAAGACATGG
GGGAAAGCAAAAATCTTCACCCCTGAAGCAAGAAACAGCACATTT
TTAATAGACGGACCAGACACCTCTGAATGCCCCAATGAACGAAGG
GCATGGAATTCTTTTGAGGTGGAAGACTATGGATTTGGCATGTTC
ACGACCAACATATGGATGAAATTCCGAGAAGGAAGTTCAGAAGTG
TGTGACCACAGGTTAATGTCAGCTGCAATTAAAGACCAGAAAGCT
GTGCATGCTGACATGGGTTATTGGATAGAGAGCTCAAAAAACCAG
ACCTGGCAGATAGAGAGAGCATCTCTTATTGAAGTGAAAACATGT
CTGTGGCCCAAGACCCATACACTGTGGAGCAATGGAGTGCTGGAA
AGCCAGATGCTTATTCCAAAATCATATGCAGGCCCTTTTTCACAG
CACAATTACCGCCAGGGCTATGCTACGCAAACCGTGGGTCCATGG
CACTTAGGCAAACTAGAGATAGACTTTGGAGAATGCCCCGGAACA
ACAGTCACAATTCAGGAGAATTGTGACCATAGAGGCCCATCTTTG
AGGACCACCACTGCATCTGGAAAACTAGTCACGCAATGGTGTTGC
CGCTCCTGCACGATGCCCCCCTTAAGGTTCTTAGGAGAAGATGGG
TGCTGGTATGGGATGGAGATTAGGCCCTTGAGTGAAAAAGAAGAG
AACATGGTCAAATCACAGGTGACGGCCGGACAGGGCACATCGGAA
ACTTTTTCAATGGGTCTGTTGTGCCTGACCTTGTTTGTGGAAGAA
TGCTTGAGGAGAAGAGTCACCAGGAAACACATGATATTAGCTGTG
GTAATCACTCTTTGTGCTATCATCCTGGGGGGCCTCACATGGATG
GACTTGCTACGAGCCCTCATCATGTTGGGGGACACTATGTCTGGT
AGAATAGGAGGACAGACCCACCTAGCCATCATGGCAGTGTTCAAG
ATGTCACCGGGATACGTGCTGGGTGTGTTTTTAAGGAAACTCACT
TCAAGAGAGACAGCACTAATGGTAATAGGAATGGCCATGACAACA
ACACTTTCAATTCCACATGACCTCATGGAACTCATTGATGGAATA
TCACTAGGACTAATTTTGCTAAAAATAGTAACACAGTTTGACAAC
ACCCAAGTGGGAACCTTAGCTCTTTCCTTGACTTTCATAAGATCA
ACAATGTCATTGGTCATGGCTTGGAGGACCATTATGGCTGTGTTG
TTTGTGGTCACACTCATTCCTTTGTGCAGGACAAGCTGTCTTCAA
AAACAGTCTCATTGGGTAGAAATAACAGCACTCATCCTAGGAGCC
CAAGCTCTGCCAGTGTACCTAATGACTCTTATGAAAGGAGCCTCA
AGAAGATCTTGGCCTCTTAACGAAGGCATAATGGCTGTGGGTTTG
GTTAGTCTCTTAGGAAGCGCTCTTTTAAAGAATGATGTCCCTTTA
GCTGGCCCAATGGTGGCAGGAGGCTTACTTCTGGCGGCTTACGTA
ATGAGTGGCAGCTCAGCAGATCTGTCACTAGAGAAGGCCGCTAAT
GTGCAGTGGGATGAAATGGCAGACATAACAGGCTCAAGTCCAATC
ATAGAAGTGAAGCAAGATGAGGATGGCTCTTTCTCCATACGGGAC
GTCGAGGAAACCAATATGATAACCCTTTTGGTGAAACTGGCACTG
ATAACGGTGTCAGGTCTCTACCCCTTGGCAATTCCAATCACAATG
ACCTTATGGTACATGTGGCAAGTGAAAACACAAAGATCAGGAGCC
CTGTGGGACGTCCCTTCACCCGCCGCCACTCAAAAAGCCGCACTG
TCTGAAGGAGTGTACAGGATCATGCAAAGAGGGTTATTCGGGAAA
ACTCAGGTTGGAGTAGGGATACACATGGAAGGTGTATTTCACACA
ATGTGGCATGTTACAAGAGGATCGGTGATCTGCCACGAGACTGGG
AGATTGGAGCCATCTTGGGCTGATGTCAGGAATGACATGATATCA
TACGGTGGGGGATGGAGGCTTGGAGATAAATGGGACAAAGAAGAA
GACGTTCAGGTCCTCGCTATAGAACCAGGGAAAAATCCCAAACAT
GTCCAAACGAAACCTGGCCTTTTCAAGACCCTAACTGGAGAAATT
GGAGCAGTAACATTAGATTTCAAACCCGGAACGTCTGGTTCTCCC
ATTATCAACAGGAAAGGAAAAGTCATCGGACTCTATGGAAATGGA
GTGGTCACCAAATCAGGTGATTACGTCAGTGCCATAACACAAGCC
GAAAGAATTGGAGAGCCAGATTATGAAGTGGATGAGGACATTTTT
CGAAAGAAAAGACTAACTATAATGGACTTACACCCCGGAGCCGGA
AAGACAAAAAGAATTCTTCCATCAATAGTGAGAGAAGCCTTAAAA
AGGAGGCTGCGAACTTTGATTTTGGCTCCCACGAGAGTGGTGGCG
GCCGAGATGGAAGAGGCCCTACGTGGACTGCCAATCCGTTACCAA
ACCCCAGCTGTAAAATCAGAACACACAGGAAGAGAGATTGTAGAC
CTCATGTGCCATGCAACCTTCACAACAAGACTTTTGTCATCAACC
AGAGTTCCAAACTACAACCTTATAGTAATGGATGAAGCACATTTC
ACCGATCCTTCCAGTGTCGCGGCTAGAGGATACATTTCGACCAGG
GTGGAAATGGGAGAGGCAGCAGCCATCTTCATGACCGCAACCCCT
CCCGGAGCGACAGATCCCTTTCCCCAGAGCAACAGCCCAATAGAA
GACATCGAGAGAGAGATTCCGGAAAGGTCATGGAACACAGGGTTC
GACTGGATAACAGACTACCAAGGGAAAACTGTGTGGTTTGTTCCC
AGCATAAAAGCTGGAAATGACATTGCAAATTGTTTGAGAAAGTCG
GGAAAGAAAGTTATCCAGTTGAGTAGGAAAACCTTTGATACAGAA
TATCCAAAAACGAAACTCACGGACTGGGACTTTGTGGTCACTACA
GACATATCTGAAATGGGGGCTAACTTTAGAGCTGGGAGAGTGATA
GACCCTAGAAGATGCCTCAAGCCAGTTATCCTAACAGATGGGCCA
GAGAGAGTCATCTTAGCAGGTCCTATTCCAGTGACTCCAGCAAGC
GCTGCTCAGAGAAGAGGGCGAATAGGAAGGAACCCAGCACAAGAA
GACGACCAATACGTTTTCTCCGGAGACCCACTAAAAAATGATGAA
GACCATGCCCACTGGACAGAAGCAAAGATGCTGCTTGACAATATC
TACACCCCAGAAGGGATCATTCCAACATTGTTTGGTCCGGAAAGG
GAAAAAACCCAAGCTATTGATGGAGAGTTTCGCCTCAGAGGGGAA
CAAAGGAAGACTTTTGTGGAATTAATGAGGAGAGGAGACCTTCCG
GTGTGGCTGAGTTATAAGGTAGCTTCTGCTGGCATTTCTTACAAA
GATCGGGAATGGTGCTTTACTGGGGAAAGAAATAACCAAATTTTA
GAAGAAAACATGGAGGTTGAAATTTGGACTAGAGAGGGAGAAAAG
AAAAAACTGAGGCCAAAATGGTTAGATGCACGTGTATACGCTGAC
CCCATGGCTTTGAAGGATTTCAAGGAGTTTGCCAGTGGAAGGAAG
AGTATAACTCTCGACATCCTAACAGAGATCGCCAGTTTGCCAACT
TACCTTTCCTCTAGGGCCAAGCTCGCCCTTGACAACATAGTTATG
CTCCACACAACAGAAAGAGGAGGGAGGGCCTATCAACATGCCCTG
AACGAACTTCCGGAGTCACTGGAAACACTCATGCTTGTAGCCTTA
CTAGGAGCTATGACAGCAGGTATCTTCCTGTTTTTCATGCAAGGG
AAAGGAATAGGGAAATTGTCAATGGGTTTGATAACCATTGCGGTG
GCTAGTGGCTTGCTCTGGGTAGCAGAAATTCAACCCCAGTGGATA
GCGGCCTCAATCATACTGGAGTTTTTTCTCATGGTACTGTTGATA
CCAGAACCAGAAAAACAAAGGACCCCACAAGACAATCAATTGATC
TACGTCATATTGACCATTCTCACCATTATTGGTCTAATAGCAGCC
AACGAGATGGGGCTGATAGAAAAAACAAAAACGGATTTTGGGTTT
TACCAGGTAAAAACAGAAACCACCATCCTCGATGTGGACCTGAGA
CCAGCTTCAGCATGGACGCTCTATGCGGTAGCCACCACAATTCTG
ACTCCCATGCTGAGACACACCATAGAAAATACGTCGGCCAACCTA
TCTTTAGCAGCCATCGCCAACCAGGCAGCCGTCCTAATGGGGCTT
GGAAAAGGATGGCCACTCCACAGAATGGACCTCGGTGTGCCGCTG
TTAGCAATGGGATGCTATTCTCAAGTGAACCCAACAACCTTGACA
GCATCCTTAGTCATGCTTTTAGTCCATTATGCAATAATAGGCCCA
GGATTGCAGGCAAAAGCCACAAGAGAGGCCCAGAAAAGGACAGCT
GCTGGAATCATGAAAAATCCCACAGTGGACGGGATAACAGTGATA
GATCTAGAACCAATATCCTATGACCCAAAATTTGAAAAGCAATTA
GGGCAGGTCATGTTACTCGTCTTGTGTGCTGGACAACTACTCTTG
ATGAGAACAACATGGGCTTTCTGTGAAGTTTTGACTTTGGCCACA
GGACCAATCTTGACCTTGTGGGAGGGCAACCCGGGAAGGTTTTGG
AACACGACCATAGCCGTATCTACCGCCAACATTTTCAGGGGAAGT
TATTTGGCAGGAGCTGGACTGGCTTTTTCACTCATAAAGAATGGA
CAAACCCCCAGGAGGGGAACTGGGACCACAGGAGAGACACTGGGA
GAGAAGTGGAAGAGACAGCTAAACTCATTAGACAGGAAAGAGTTT
GAAGAGTATAAAAGAAGTGGAATACTAGAAGTGGACAGGACTGAA
GCCAAGTCCGCCCTGAAAGATGGGTCTAAAATCAAGCATGCAGTA
TCTAGAGGGTCCAGTAAGATCAGATGGATTGTTGAGAGAGGGATG
GTAAAGCCAAAGGGGAAAGTTGTAGATCTTGGCTGTGGGAGAGGA
GGATGGTCTTATTACATGGCGACACTCAAGAACGTGACTGAAGTG
AAAGGGTATACAAAAGGAGGTCCAGGACATGAAGAACCGATTCCC
ATGGCTACTTATGGCTGGAATTTGGTCAAACTCCATTCAGGGGTT
GACGTGTTCTACAAACCCACAGAGCAAGTGGACACCCTGCTCTGT
GATATTGGGGAGTCATCTTCTAATCCAACAATAGAGGAAGGAAGA
ACATTAAGAGTTTTGAAGATGGTGGAGCCATGGCTCTCTTCAAAA
CCTGAATTCTGCATCAAAGTCCTCAACCCCTACATGCCAACAGTC
ATAGAGGAGCTGGAGAAACTGCAGAGAAAACACGGTGGGAACCTT
GTCAGATGCCCGCTGTCCAGGAACTCCACCCATGAGATGTATTGG
GTGTCAGGAGCGTCGGGAAATATTGTGAGCTCTGTGAACACAACA
TCAAAGATGTTGTTGAACAGGTTCACAACAAGGCATAGGAAACCC
ACTTATGAGAAGGACGTAGATCTTGGGGCAGGAACGAGAAGTGTC
TCTACTGAAACAGAAAAACCAGACATGACAATCATTGGGAGAAGG
CTTCAGCGATTGCAAGAGGAGCACAAAGAAACATGGCATTATGAT
CAGGAAAACCCATACAGAACCTGGGCGTATCATGGAAGCTATGAA
GCTCCTTCGACAGGCTCTGCATCCTCCATGGTGAACGGGGTGGTG
AAACTGCTAACAAAACCCTGGGATGTGATTCCAATGGTGACTCAG
TTAGCCATGACAGATACAACCCCTTTTGGGCAACAAAGAGTGTTC
AAAGAGAAGGTGGATACCAGAACGCCGCAACCAAAACCAGGCACA
CGAATGGTTATGACCACGACAGCCAATTGGCTATGGGCCCTCCTT
GGAAAGAAGAAAAATCCCAGACTGTGTACAAGGGAAGAGTTCATC
TCAAAAGTTAGATCAAACGCAGCCATAGGCGCAGTCTTTCAGGAA
GAACAAGGATGGACATCAGCCAGTGAAGCTGTGAATGACAGCCGG
TTTTGGGAACTGGTTGACAAAGAAAGGGCCCTTCACCAGGAAGGG
AAATGTGAATCGTGTGTCTATAACATGATGGGAAAACGTGAGAAA
AAGTTAGGAGAGTTTGGCAGAGCCAAGGGAAGCCGAGCAATCTGG
TACATGTGGCTGGGAGCGCGGTTTCTGGAATTTGAAGCCCTGGGT
TTTTTGAATGAAGATCACTGGTTTGGCAGAGAAAATTCATGGAGT
GGAGTGGAAGGGGAAGGTCTGCACAGATTGGGATACATCCTGGAG
GAGATAGACAAGAAGGATGGAGACCTAATGTATGCTGATGACACA
GCAGGTTGGGACACAAGAATCACTGAGGATGACCTTCAAAATGAG
GAACTGATCACGGAACAGATGGCTCCCCACCACAAGATCCTAGCC
AAAGCCATTTTCAAACTAACCTATCAAAACAAAGTGGTGAAAGTC
CTCAGACCCACACCGAGAGGAGCGGTGATGGATATCATATCCAGG
AAAGACCAAAGAGGTAGTGGACAAGTTGGAACATATGGTTTGAAC
ACATTCACCAACATGGAAGTTCAACTCATCCGCCAAATGGAAGCT
GAAGGAGTCATCACACAAGATGACATGCAGAACCCAAAAGGGTTG
AAAGAAAGAGTTGAGAAATGGCTGAGAGAGTGTGGTGTCGACAGG
TTAAAGAGGATGGCAATCAGTGGAGACGATTGCGTGGTGAAGCCC
CTAGATGAGAGGTTTGGCACTTCCCTCCTCTTCTTGAACGACATG
GGAAAGGTGAGGAAAGACATTCCGCAGTGGGAACCATCTAAGGGA
TGGAAAAACTGGCAAGAGGTTCCTTTTTGCTCCCATCACTTTCAC
AAGATCTTTATGAAGGATGGCCGCTCACTAGTTGTTCCATGTAGA
AACCAGGATGAACTGATAGGGAGAGCCAGAATCTCGCAGGGGGCT
GGATGGAGCTTAAGAGAAACAGCCTGCCTGGGCAAAGCTTACGCC
CAGATGTGGTCGCTTATGTACTTCCACAGAAGGGATCTGCGTTTA
GCCTCCATGGCCATATGCTCAGCAGTTCCAACGGAATGGTTTCCA
ACAAGCAGAACAACATGGTCAATCCATGCTCATCACCAATGGATG
ACCACTGAAGACATGCTCAAAGTGTGGAACAGAGTGTGGATAGAA
GACAACCCCAATATGATTGACAAGACTCCAGTCCATTCGTGGGAA
GATATACCTTACCTAGGGAAAAGAGAGGATTTGTGGTGTGGATCC
CTGATTGGACTTTCTTCTAGAGCCACCTGGGCGAAGAACATTCAC
ACGGCCATAACTCAGGTCAGGAACTTGATCGGAAAAGAGGAATAC
GTGGATTACATGCCAGTAATGAAAAGATACAGTGCTCCTTCAGAG
AGTGAAGGAGTTCTGTAATTACCAACAACAAACACCAAAGGCTAT
TGAAGTCAGGCCACTTGTGCCACGGTTTGAGCAAACCGTGCTGCC
TGTAGCTCCGCCAATAATGGGAGGCGTAATAATCCCTAGGGAGGC
CATGCGCCACGGAAGCTGTACGCGTGGCATATTGGACTAGCGGTT
AGAGGAGACCCCTCCCATCACTGACAAAACGCAGCAAAAGGGGGC
CCGAAGCCAGGAGGAAGCTGTACTCCTGGTGGAAGGACTAGAGGT
TAGAGGAGACCCCCCCAACACAAAAACAGCATATTGACGCTGGGA
AAGACCAGAGATCCTGCTGTCTCTACAACATCAATCCAGGCACAG
AGCGCCGCAAGATGGATTGGTGTTGTTGATCCAACAGGTTCT
DENV-4-Emin
SEQ ID NO: 8
AGTTGTTAGTCTGTGTGGACCGACAAGGACAGTTCCAAATCGGAA
GCTTGCTTAACACAGTTCTAACAGTTTGTTTAAATAGAGAGCAGA
TCTCTGGAAAAATGAACCAACGAAAAAAGGTGGTTAGACCACCTT
TCAATATGCTGAAACGCGAGAGAAACCGCGTATCAACCCCTCAAG
GGTTGGTGAAGAGATTCTCAACCGGACTTTTTTCTGGGAAAGGAC
CCTTACGGATGGTGCTAGCATTCATCACGTTTTTGCGAGTCCTTT
CCATCCCACCAACAGCAGGGATTCTGAAGAGATGGGGACAGTTGA
AGAAAAATAAGGCCATCAAGATACTGATTGGATTCAGGAAGGAGA
TAGGCCGCATGCTGAACATCTTGAACGGGAGAAAAAGGTCAACGA
TAACATTGTTGTGCTTGATTCCCACCGTAATGGCGTTTCACTTGT
CAACAAGAGATGGCGAACCCCTCATGATAGTGGCAAAACATGAAA
GGGGGAGACCTCTCTTGTTTAAGACAACAGAGGGGATCAACAAAT
GCACTCTCATTGCCATGGACTTGGGTGAAATGTGTGAGGACACTG
TCACGTATAAATGCCCCCTACTGGTCAATACCGAACCTGAAGACA
TTGATTGCTGGTGCAACCTCACGTCTACCTGGGTCATGTATGGGA
CATGCACCCAGAGCGGAGAACGGAGACGAGAGAAGCGCTCAGTAG
CTTTGACACCACATTCAGGAATGGGATTGGAAACAAGAGCTGAGA
CATGGATGTCATCGGAAGGGGCTTGGAAGCATGCTCAGAGAGTAG
AGAGCTGGATACTCAGAAACCCAGGATTTGCGCTCTTGGCAGGAT
TTATGGCTTATATGATTGGGCAAACAGGAATCCAGCGAACTGTCT
TCTTTGTCCTAATGATGCTGGTCGCCCCATCCTACGGAATGAGAT
GCGTAGGCGTAGGTAATCGCGATTTCGTTGAGGGAGTGTCAGGCG
GAGCATGGGTCGATCTGGTACTCGAACACGGAGGATGCGTTACGA
CTATGGCACAGGGAAAACCGACATTGGACTTTGAGTTGACTAAGA
CAACCGCTAAAGAGGTCGCACTATTGCGAACATACTGTATCGAAG
CGTCAATTTCGAATATTACAACCGCTACTAGGTGTCCTACACAGG
GCGAACCATACCTTAAAGAGGAACAGGACCAACAGTATATTTGTA
GAAGAGACGTAGTCGATAGGGGGTGGGGAAACGGATGCGGATTGT
TCGGTAAGGGGGGAGTCGTTACATGCGCTAAGTTCTCATGTTCCG
GTAAGATTACCGGTAACTTAGTGCAAATTGAGAATCTCGAATATA
CCGTAGTCGTTACAGTGCATAACGGAGACACACACGCAGTCGGAA
ACGATACATCTAACCATGGCGTAACCGCTACAATTACACCTAGGT
CACCATCCGTTGAGGTTAAGTTGCCCGATTACGGCGAACTGACAC
TCGATTGCGAACCTAGATCCGGAATAGACTTTAACGAAATGATAC
TGATGAAAATGAAAAAAAAGACATGGTTAGTGCATAAGCAATGGT
TTCTCGACTTACCCCTACCTTGGACCGCCGGAGCCGATACTAGCG
AAGTGCACTGGAATTACAAAGAGAGAATGGTTACATTTAAAGTGC
CACACGCTAAGAGACAGGACGTTACAGTGTTAGGGTCACAGGAGG
GAGCTATGCATAGCGCACTAGCCGGAGCAACCGAAGTCGATAGCG
GAGACGGAAATCATATGTTCGCCGGACATCTGAAATGCAAAGTGA
GAATGGAGAAATTGCGGATTAAGGGAATGTCATACACTATGTGTT
CCGGTAAGTTTTCGATTGACAAAGAGATGGCCGAAACGCAACACG
GAACAACAGTCGTTAAGGTTAAGTACGAAGGAGCCGGAGCTCCGT
GTAAAGTGCCAATCGAAATTAGAGACGTTAACAAAGAGAAAGTGG
TCGGAAGAGTGATATCTAGTACACCCCTAGCCGAAAATACGAATT
CCGTTACGAATATCGAACTCGAACCCCCCTTTGGAGACTCATACA
TAGTGATAGGAGTGGGTAATTCCGCACTCACGTTGCACTGGTTCA
GAAAGGGATCATCAATCGGGAAGATGTTCGAATCAACATATAGGG
GAGCGAAAAGAATGGCTATATTGGGCGAAACCGCATGGGACTTCG
GATCAGTCGGAGGACTGTTTACGAGTCTGGGTAAGGCCGTACATC
AGGTATTCGGATCAGTGTATACAACAATGTTTGGCGGAGTGTCAT
GGATGATACGGATACTGATAGGGTTTCTAGTGTTGTGGATCGGAA
CGAACTCACGTAATACGTCTATGGCTATGACATGTATTGCCGTAG
GGGGGATTACACTGTTTCTGGGATTTACAGTGCAAGCAGACATGG
GTTGTGTGGTGTCATGGAGTGGGAGAGAATTGAAGTGTGGAAGCG
GAATTTTTGTGGTTGACAACGTGCACACTTGGACAGAACAGTACA
AATTCCAACCAGAGTCCCCAGCGAGACTAGCGTCTGCAATATTAA
ATGCCCACAAAGATGGGGTCTGTGGAATTAGATCAACCACGAGGC
TGGAAAATGTTATGTGGAAGCAAATAACCAATGAGCTAAACTATG
TTCTCTGGGAAGGAGGACATGATCTCACTGTAGTGGCTGGGGATG
TGAAAGGGGTGTTGACCAAGGGCAAGAGAGCACTCACACCCCCAG
CGAGTGATCTGAAATATTCATGGAAGACATGGGGGAAAGCAAAAA
TCTTCACCCCTGAAGCAAGAAACAGCACATTTTTAATAGACGGAC
CAGACACCTCTGAATGCCCCAATGAACGAAGGGCATGGAATTCTT
TTGAGGTGGAAGACTATGGATTTGGCATGTTCACGACCAACATAT
GGATGAAATTCCGAGAAGGAAGTTCAGAAGTGTGTGACCACAGGT
TAATGTCAGCTGCAATTAAAGACCAGAAAGCTGTGCATGCTGACA
TGGGTTATTGGATAGAGAGCTCAAAAAACCAGACCTGGCAGATAG
AGAGAGCATCTCTTATTGAAGTGAAAACATGTCTGTGGCCCAAGA
CCCATACACTGTGGAGCAATGGAGTGCTGGAAAGCCAGATGCTTA
TTCCAAAATCATATGCAGGCCCTTTTTCACAGCACAATTACCGCC
AGGGCTATGCTACGCAAACCGTGGGTCCATGGCACTTAGGCAAAC
TAGAGATAGACTTTGGAGAATGCCCCGGAACAACAGTCACAATTC
AGGAGAATTGTGACCATAGAGGCCCATCTTTGAGGACCACCACTG
CATCTGGAAAACTAGTCACGCAATGGTGTTGCCGCTCCTGCACGA
TGCCCCCCTTAAGGTTCTTAGGAGAAGATGGGTGCTGGTATGGGA
TGGAGATTAGGCCCTTGAGTGAAAAAGAAGAGAACATGGTCAAAT
CACAGGTGACGGCCGGACAGGGCACATCGGAAACTTTTTCAATGG
GTCTGTTGTGCCTGACCTTGTTTGTGGAAGAATGCTTGAGGAGAA
GAGTCACCAGGAAACACATGATATTAGCTGTGGTAATCACTCTTT
GTGCTATCATCCTGGGGGGCCTCACATGGATGGACTTGCTACGAG
CCCTCATCATGTTGGGGGACACTATGTCTGGTAGAATAGGAGGAC
AGACCCACCTAGCCATCATGGCAGTGTTCAAGATGTCACCGGGAT
ACGTGCTGGGTGTGTTTTTAAGGAAACTCACTTCAAGAGAGACAG
CACTAATGGTAATAGGAATGGCCATGACAACAACACTTTCAATTC
CACATGACCTCATGGAACTCATTGATGGAATATCACTAGGACTAA
TTTTGCTAAAAATAGTAACACAGTTTGACAACACCCAAGTGGGAA
CCTTAGCTCTTTCCTTGACTTTCATAAGATCAACAATGTCATTGG
TCATGGCTTGGAGGACCATTATGGCTGTGTTGTTTGTGGTCACAC
TCATTCCTTTGTGCAGGACAAGCTGTCTTCAAAAACAGTCTCATT
GGGTAGAAATAACAGCACTCATCCTAGGAGCCCAAGCTCTGCCAG
TGTACCTAATGACTCTTATGAAAGGAGCCTCAAGAAGATCTTGGC
CTCTTAACGAAGGCATAATGGCTGTGGGTTTGGTTAGTCTCTTAG
GAAGCGCTCTTTTAAAGAATGATGTCCCTTTAGCTGGCCCAATGG
TGGCAGGAGGCTTACTTCTGGCGGCTTACGTAATGAGTGGCAGCT
CAGCAGATCTGTCACTAGAGAAGGCCGCTAATGTGCAGTGGGATG
AAATGGCAGACATAACAGGCTCAAGTCCAATCATAGAAGTGAAGC
AAGATGAGGATGGCTCTTTCTCCATACGGGACGTCGAGGAAACCA
ATATGATAACCCTTTTGGTGAAACTGGCACTGATAACGGTGTCAG
GTCTCTACCCCTTGGCAATTCCAATCACAATGACCTTATGGTACA
TGTGGCAAGTGAAAACACAAAGATCAGGAGCCCTGTGGGACGTCC
CTTCACCCGCCGCCACTCAAAAAGCCGCACTGTCTGAAGGAGTGT
ACAGGATCATGCAAAGAGGGTTATTCGGGAAAACTCAGGTTGGAG
TAGGGATACACATGGAAGGTGTATTTCACACAATGTGGCATGTTA
CAAGAGGATCGGTGATCTGCCACGAGACTGGGAGATTGGAGCCAT
CTTGGGCTGATGTCAGGAATGACATGATATCATACGGTGGGGGAT
GGAGGCTTGGAGATAAATGGGACAAAGAAGAAGACGTTCAGGTCC
TCGCTATAGAACCAGGGAAAAATCCCAAACATGTCCAAACGAAAC
CTGGCCTTTTCAAGACCCTAACTGGAGAAATTGGAGCAGTAACAT
TAGATTTCAAACCCGGAACGTCTGGTTCTCCCATTATCAACAGGA
AAGGAAAAGTCATCGGACTCTATGGAAATGGAGTGGTCACCAAAT
CAGGTGATTACGTCAGTGCCATAACACAAGCCGAAAGAATTGGAG
AGCCAGATTATGAAGTGGATGAGGACATTTTTCGAAAGAAAAGAC
TAACTATAATGGACTTACACCCCGGAGCCGGAAAGACAAAAAGAA
TTCTTCCATCAATAGTGAGAGAAGCCTTAAAAAGGAGGCTGCGAA
CTTTGATTTTGGCTCCCACGAGAGTGGTGGCGGCCGAGATGGAAG
AGGCCCTACGTGGACTGCCAATCCGTTACCAAACCCCAGCTGTAA
AATCAGAACACACAGGAAGAGAGATTGTAGACCTCATGTGCCATG
CAACCTTCACAACAAGACTTTTGTCATCAACCAGAGTTCCAAACT
ACAACCTTATAGTAATGGATGAAGCACATTTCACCGATCCTTCCA
GTGTCGCGGCTAGAGGATACATTTCGACCAGGGTGGAAATGGGAG
AGGCAGCAGCCATCTTCATGACCGCAACCCCTCCCGGAGCGACAG
ATCCCTTTCCCCAGAGCAACAGCCCAATAGAAGACATCGAGAGAG
AGATTCCGGAAAGGTCATGGAACACAGGGTTCGACTGGATAACAG
ACTACCAAGGGAAAACTGTGTGGTTTGTTCCCAGCATAAAAGCTG
GAAATGACATTGCAAATTGTTTGAGAAAGTCGGGAAAGAAAGTTA
TCCAGTTGAGTAGGAAAACCTTTGATACAGAATATCCAAAAACGA
AACTCACGGACTGGGACTTTGTGGTCACTACAGACATATCTGAAA
TGGGGGCTAACTTTAGAGCTGGGAGAGTGATAGACCCTAGAAGAT
GCCTCAAGCCAGTTATCCTAACAGATGGGCCAGAGAGAGTCATCT
TAGCAGGTCCTATTCCAGTGACTCCAGCAAGCGCTGCTCAGAGAA
GAGGGCGAATAGGAAGGAACCCAGCACAAGAAGACGACCAATACG
TTTTCTCCGGAGACCCACTAAAAAATGATGAAGACCATGCCCACT
GGACAGAAGCAAAGATGCTGCTTGACAATATCTACACCCCAGAAG
GGATCATTCCAACATTGTTTGGTCCGGAAAGGGAAAAAACCCAAG
CTATTGATGGAGAGTTTCGCCTCAGAGGGGAACAAAGGAAGACTT
TTGTGGAATTAATGAGGAGAGGAGACCTTCCGGTGTGGCTGAGTT
ATAAGGTAGCTTCTGCTGGCATTTCTTACAAAGATCGGGAATGGT
GCTTTACTGGGGAAAGAAATAACCAAATTTTAGAAGAAAACATGG
AGGTTGAAATTTGGACTAGAGAGGGAGAAAAGAAAAAACTGAGGC
CAAAATGGTTAGATGCACGTGTATACGCTGACCCCATGGCTTTGA
AGGATTTCAAGGAGTTTGCCAGTGGAAGGAAGAGTATAACTCTCG
ACATCCTAACAGAGATCGCCAGTTTGCCAACTTACCTTTCCTCTA
GGGCCAAGCTCGCCCTTGACAACATAGTTATGCTCCACACAACAG
AAAGAGGAGGGAGGGCCTATCAACATGCCCTGAACGAACTTCCGG
AGTCACTGGAAACACTCATGCTTGTAGCCTTACTAGGAGCTATGA
CAGCAGGTATCTTCCTGTTTTTCATGCAAGGGAAAGGAATAGGGA
AATTGTCAATGGGTTTGATAACCATTGCGGTGGCTAGTGGCTTGC
TCTGGGTAGCAGAAATTCAACCCCAGTGGATAGCGGCCTCAATCA
TACTGGAGTTTTTTCTCATGGTACTGTTGATACCAGAACCAGAAA
AACAAAGGACCCCACAAGACAATCAATTGATCTACGTCATATTGA
CCATTCTCACCATTATTGGTCTAATAGCAGCCAACGAGATGGGGC
TGATAGAAAAAACAAAAACGGATTTTGGGTTTTACCAGGTAAAAA
CAGAAACCACCATCCTCGATGTGGACCTGAGACCAGCTTCAGCAT
GGACGCTCTATGCGGTAGCCACCACAATTCTGACTCCCATGCTGA
GACACACCATAGAAAATACGTCGGCCAACCTATCTTTAGCAGCCA
TCGCCAACCAGGCAGCCGTCCTAATGGGGCTTGGAAAAGGATGGC
CACTCCACAGAATGGACCTCGGTGTGCCGCTGTTAGCAATGGGAT
GCTATTCTCAAGTGAACCCAACAACCTTGACAGCATCCTTAGTCA
TGCTTTTAGTCCATTATGCAATAATAGGCCCAGGATTGCAGGCAA
AAGCCACAAGAGAGGCCCAGAAAAGGACAGCTGCTGGAATCATGA
AAAATCCCACAGTGGACGGGATAACAGTGATAGATCTAGAACCAA
TATCCTATGACCCAAAATTTGAAAAGCAATTAGGGCAGGTCATGT
TACTCGTCTTGTGTGCTGGACAACTACTCTTGATGAGAACAACAT
GGGCTTTCTGTGAAGTTTTGACTTTGGCCACAGGACCAATCTTGA
CCTTGTGGGAGGGCAACCCGGGAAGGTTTTGGAACACGACCATAG
CCGTATCTACCGCCAACATTTTCAGGGGAAGTTATTTGGCAGGAG
CTGGACTGGCTTTTTCACTCATAAAGAATGCACAAACCCCCAGGA
GGGGAACTGGGACCACAGGAGAGACACTGGGAGAGAAGTGGAAGA
GACAGCTAAACTCATTAGACAGGAAAGAGTTTGAAGAGTATAAAA
GAAGTGGAATACTAGAAGTGGACAGGACTGAAGCCAAGTCCGCCC
TGAAAGATGGGTCTAAAATCAAGCATGCAGTATCTAGAGGGTCCA
GTAAGATCAGATGGATTGTTGAGAGAGGGATGGTAAAGCCAAAGG
GGAAAGTTGTAGATCTTGGCTGTGGGAGAGGAGGATGGTCTTATT
ACATGGCGACACTCAAGAACGTGACTGAAGTGAAAGGGTATACAA
AAGGAGGTCCAGGACATGAAGAACCGATTCCCATGGCTACTTATG
GCTGGAATTTGGTCAAACTCCATTCAGGGGTTGACGTGTTCTACA
AACCCACAGAGCAAGTGGACACCCTGCTCTGTGATATTGGGGAGT
CATCTTCTAATCCAACAATAGAGGAAGGAAGAACATTAAGAGTTT
TGAAGATGGTGGAGCCATGGCTCTCTTCAAAACCTGAATTCTGCA
TCAAAGTCCTCAACCCCTACATGCCAACAGTCATAGAGGAGCTGG
AGAAACTGCAGAGAAAACACGGTGGGAACCTTGTCAGATGCCCGC
TGTCCAGGAACTCCACCCATGAGATGTATTGGGTGTCAGGAGCGT
CGGGAAATATTGTGAGCTCTGTGAACACAACATCAAAGATGTTGT
TGAACAGGTTCACAACAAGGCATAGGAAACCCACTTATGAGAAGG
ACGTAGATCTTGGGGCAGGAACGAGAAGTGTCTCTACTGAAACAG
AAAAACCAGACATGACAATCATTGGGAGAAGGCTTCAGCGATTGC
AAGAGGAGCACAAAGAAACATGGCATTATGATCAGGAAAACCCAT
ACAGAACCTGGGCGTATCATGGAAGCTATGAAGCTCCTTCGACAG
GCTCTGCATCCTCCATGGTGAACGGGGTGGTGAAACTGCTAACAA
AACCCTGGGATGTGATTCCAATGGTGACTCAGTTAGCCATGACAG
ATACAACCCCTTTTGGGCAACAAAGAGTGTTCAAAGAGAAGGTGG
ATACCAGAACGCCGCAACCAAAACCAGGCACACGAATGGTTATGA
CCACGACAGCCAATTGGCTATGGGCCCTCCTTGGAAAGAAGAAAA
ATCCCAGACTGTGTACAAGGGAAGAGTTCATCTCAAAAGTTAGAT
CAAACGCAGCCATAGGCGCAGTCTTTCAGGAAGAACAAGGATGGA
CATCAGCCAGTGAAGCTGTGAATGACAGCCGGTTTTGGGAACTGG
TTGACAAAGAAAGGGCCCTTCACCAGGAAGGGAAATGTGAATCGT
GTGTCTATAACATGATGGGAAAACGTGAGAAAAAGTTAGGAGAGT
TTGGCAGAGCCAAGGGAAGCCGAGCAATCTGGTACATGTGGCTGG
GAGCGCGGTTTCTGGAATTTGAAGCCCTGGGTTTTTTGAATGAAG
ATCACTGGTTTGGCAGAGAAAATTCATGGAGTGGAGTGGAAGGGG
AAGGTCTGCACAGATTGGGATACATCCTGGAGGAGATAGACAAGA
AGGATGGAGACCTAATGTATGCTGATGACACAGCAGGTTGGGACA
CAAGAATCACTGAGGATGACCTTCAAAATGAGGAACTGATCACGG
AACAGATGGCTCCCCACCACAAGATCCTAGCCAAAGCCATTTTCA
AACTAACCTATCAAAACAAAGTGGTGAAAGTCCTCAGACCCACAC
CGAGAGGAGCGGTGATGGATATCATATCCAGGAAAGACCAAAGAG
GTAGTGGACAAGTTGGAACATATGGTTTGAACACATTCACCAACA
TGGAAGTTCAACTCATCCGCCAAATGGAAGCTGAAGGAGTCATCA
CACAAGATGACATGCAGAACCCAAAAGGGTTGAAAGAAAGAGTTG
AGAAATGGCTGAGAGAGTGTGGTGTCGACAGGTTAAAGAGGATGG
CAATCAGTGGAGACGATTGCGTGGTGAAGCCCCTAGATGAGAGGT
TTGGCACTTCCCTCCTCTTCTTGAACGACATGGGAAAGGTGAGGA
AAGACATTCCGCAGTGGGAACCATCTAAGGGATGGAAAAACTGGC
AAGAGGTTCCTTTTTGCTCCCATCACTTTCACAAGATCTTTATGA
AGGATGGCCGCTCACTAGTTGTTCCATGTAGAAACCAGGATGAAC
TGATAGGGAGAGCCAGAATCTCGCAGGGGGCTGGATGGAGCTTAA
GAGAAACAGCCTGCCTGGGCAAAGCTTACGCCCAGATGTGGTCGC
TTATGTACTTCCACAGAAGGGATCTGCGTTTAGCCTCCATGGCCA
TATGCTCAGCAGTTCCAACGGAATGGTTTCCAACAAGCAGAACAA
CATGGTCAATCCATGCTCATCACCAATGGATGACCACTGAAGACA
TGCTCAAAGTGTGGAACAGAGTGTGGATAGAAGACAACCCCAATA
TGATTGACAAGACTCCAGTCCATTCGTGGGAAGATATACCTTACC
TAGGGAAAAGAGAGGATTTGTGGTGTGGATCCCTGATTGGACTTT
CTTCTAGAGCCACCTGGGCGAAGAACATTCACACGGCCATAACTC
AGGTCAGGAACTTGATCGGAAAAGAGGAATACGTGGATTACATGC
CAGTAATGAAAAGATACAGTGCTCCTTCAGAGAGTGAAGGAGTTC
TGTAATTACCAACAACAAACACCAAAGGCTATTGAAGTCAGGCCA
CTTGTGCCACGGTTTGAGCAAACCGTGCTGCCTGTAGCTCCGCCA
ATAATGGGAGGCGTAATAATCCCTAGGGAGGCCATGCGCCACGGA
AGCTGTACGCGTGGCATATTGGACTAGCGGTTAGAGGAGACCCCT
CCCATCACTGACAAAACGCAGCAAAAGGGGGCCCGAAGCCAGGAG
GAAGCTGTACTCCTGGTGGAAGGACTAGAGGTTAGAGGAGACCCC
CCCAACACAAAAACAGCATATTGACGCTGGGAAAGACCAGAGATC
CTGCTGTCTCTACAACATCAATCCAGGCACAGAGCGCCGCAAGAT
GGATTGGTGTTGTTGATCCAACAGGTTCT
DENV-1-VN-BID-V1774-2007-E-W/Min
SEQ ID NO: 9
AGTTGTTAGTCTACGTGGACCGACAAGAACAGTTTCGAATCGGAA
GCTTGCTTAACGTAGTTCTTCTTTCAATATGCTGAAACGCGCGAG
AAACCGCGTGTCAACTGTTTCACAGTTGGCGAAGAGATTCTCAAA
AGGATTGCTCTCAGGCCAAGGACCCATGAAATTGGTGATGGCTTT
CATAGCATTCCTAAGATTTCTAGCCATACCCCCAACAGCAGGAAT
TTTGGCTAGATGGGGTTCATTCAAGAAGAGTGGAGCGATCAAAGT
GCTACGGGGTTTCAAGAAAGAAATCTCAAACATGTTGAATATAAT
GAATAGAAGGAAAAGATCTGTGACCATGCTCCTTATGCTGATGCC
TACAGCCTTGGCGTTCCATTTGACTACACGAGGGGGAGAGCCGCA
CATGATAGTCAGCAAGCAGGAAAGAGGAAAGTCACTCTTGTTTAA
GACCTCAGCAGGTGTCAACATGTGCACCCTTATAGCGATGGATTT
GGGAGAGTTATGTGAGGACACAATGACTTACAAATGCCCTCGAAT
CACTGAAACTGAACCAGATGACGTTGATTGTTGGTGTAATGCCAC
AGACACATGGGTGACCTATGGAACATGTTCCCAAACTGGCGAGCA
CCGACGAGACAAACGTTCCGTCGCACTGGCCCCACACGTGGGACT
TGGTTTGGAAACAAGAACCGAAACGTGGATGTCCTCTGAAGGCGC
TTGGAAACAGATACAAAGAGTGGAGACTTGGGCCCTGAGACACCC
AGGATTCACGGTGATAGCCCTTTTTCTAGCACATGCCATAGGAAC
ATCCATCACCCAGAAAGGGATTATTTTCATTTTGTTAATGCTGGT
AACACCATCCATGGCCATGCGATGCGTGGGAATAGGCAGCAGGGA
CTTCGTGGAAGGACTGTCAGGAGCAACTTGGGTAGATGTGGTACT
GGAACATGGAAGTTGCGTCACCACCATGGCAAAAGACAAACCAAC
ATTGGACATTGAACTCTTGAAGACGGAAGTCACAAACCCTGCCGT
CCTGCGCAAACTGTGCATTGAAGCTAAAATATCAAACACCACCAC
CGACTCAAGATGTCCAACACAAGGAGAAGCCACACTAGTGGAAGA
ACAAGACGCGAACTTTGTGTGTCGACGAACGTTTGTGGACAGAGG
CTGGGGCAATGGCTGTGGCCTCTTCGGAAAAGGAAGCCTAATAAC
GTGTGCAAAGTTCAAGTGTGTGACAAAACTGGAAGGAAAGATAGT
TCAATATGAGAACTTGAAATATTCAGTAATAGTCACCGTTCACAC
CGGAGACCAGCATCAAGTGGGAAATGAAAGCACAGAACATGGGAC
AACTGCAACTATAACACCTCAAGCTCCTACGACGGAAATACAGCT
GACCGACTACGGAGCTCTTACATTGGATTGTTCACCTAGAACAGG
ACTAGACTTTAATGAAATGGTGTTGTTGACAATGAAAGAAAAATC
ATGGCTAGTCCACAAACAATGGTTTTTAGACCTACCACTGCCTTG
GACCTCGGGAGCTTCAACATCACAAGAGACTTGGAACAGACAAGA
TTTGCTGGTGACATTTAAGACAGCTCATGCAAAGAAGCAGGAAGT
AGTAGTGTTAGGGTCACAAGAGGGAGCAATGCATACCGCACTAAC
CGGAGCAACCGAAATACAGACTAGCGGAACTACAACGATTTTTGC
CGGTCACCTGAAATGTAGACTGAAGATGGACAAACTGACACTTAA
AGGAATGTCATACGTTATGTGTACGGGATCCTTTAAGCTCGAAAA
AGAGGTTGCCGAAACGCAACACGGAACAGTGCTAGTGCAAATTAA
ATATGAGGGAACCGACGCACCATGTAAGATACCGTTTAGTACGCA
AGACGAAAAGGGCGTTACGCAAAACGGAAGACTGATTACCGCTAA
CCCTATCGTTACAGACAAAGAGAAACCCGTTAACATAGAGGCCGA
ACCACCTTTTGGCGAATCATATATAGTGATAGGCGCAGGCGAAAA
AGCACTAAAGCTGTCATGGTTCAAAAAAGGATCTAGCATAGGGAA
GATGTTCGAAGCAACCGCTAGGGGAGCTAGACGAATGGCAATACT
GGGAGATACCGCATGGGACTTCGGATCGATAGGGGGAGTGTTTAC
TAGCGTAGGTAAGTTGGTGCATCAGATATTCGGAACCGCATACGG
AGTGTTGTTTAGCGGAGTGTCATGGACTATGAAGATAGGGATAGG
AGTGCTATTGACATGGCTCGGACTGAATTCGAGATCGACTAGCCT
ATCTATGACATGCATAGCCGTCGGACTGGTTACACTGTATCTAGG
CGTAATGGTGCAAGCAGATTCAGGATGTGTAATTAATTGGAAAGG
TAGAGAACTCAAGTGTGGAAGTGGCATTTTTGTCACCAATGAAGT
TCACACTTGGACAGAGCAATACAAATTTCAAGCTGACTCCCCTAA
GAGACTATCAGCAGCCATCGGGAAGGCATGGGAGGAGGGTGTGTG
TGGAATTCGATCAGCAACTCGTCTCGAGAACATCATGTGGAAGCA
AATATCAAATGAACTGAATCACATCTTACTTGAAAATGATATGAA
ATTCACAGTGGTTGTAGGAGATGTTGCTGGGATCTTGGCTCAAGG
AAAGAAAATGATTAGGCCACAACCCATGGAATACAAATACTCGTG
GAAAAGCTGGGGAAAGGCTAAAATCATAGGGGCAGATGTACAGAA
CACCACCTTCATCATCGATGGCCCAAACACCCCAGAATGCCCTGA
TGACCAAAGAGCATGGAACATTTGGGAAGTTGAGGACTATGGATT
TGGAATTTTCACGACAAACATATGGCTGAAATTGCGTGATTCCTA
CACCCAAGTGTGTGACCACCGGCTAATGTCAGCTGCCATCAAGGA
CAGCAAGGCAGTTCACGCTGACATGGGGTACTGGATAGAAAGTGA
AAAGAACGAGACCTGGAAGCTGGCAAGAGCCTCATTCATAGAAGT
TAAAACATGTATCTGGCCAAAATCCCACACTCTATGGAGCAATGG
AGTTCTGGAAAGTGAAATGATAATTCCAAAGATCTATGGAGGACC
AATATCTCAGCACAACTACAGACCAGGATATTTTACACAAGCAGC
AGGGCCGTGGCACCTAGGCAAGTTGGAACTGGATTTTGATTTGTG
TGAGGGTACCACAGTTGTTGTGGATGAACATTGTGGAAATCGAGG
ACCATCTCTTAGGACCACAACAGTCACAGGAAAGATAATTCATGA
ATGGTGTTGCAGATCTTGCACGCTACCACCCTTACGTTTCAGAGG
AGAAGATGGGTGCTGGTACGGTATGGAAATCAGACCAGTCAAGGA
AAAGGAAGAAAATCTAGTCAAATCAATGGTCTCTGCAGGGTCAGG
GGAAGTGGACAGCTTTTCACTAGGACTGCTATGCATATCAATAAT
GATCGAGGAGGTGATGAGATCCAGATGGAGTAGAAGAATGCTGAT
GACTGGAACACTGGCTGTGTTCTTCCTTCTCATAATGGGACAATT
GACATGGAACGATCTGATCAGATTATGCATCATGGTTGGAGCCAA
CGCTTCCGACAGGATGGGGATGGGAACGACGTACCTAGCCCTGAT
GGCCACTTTTAAAATGAGACCGATGTTCGCTGTAGGGCTATTATT
TCGCAGACTAACATCCAGAGAAGTTCTTCTTCTAACAATTGGATT
GAGTCTAGTGGCATCTGTGGAGTTACCAAATTCCTTGGAGGAGCT
GGGGGATGGACTTGCAATGGGCATTATGATTTTAAAATTATTGAC
TGACTTTCAATCACATCAGCTGTGGGCTACCTTGCTGTCCTTGAC
ATTTATCAAAACAACGTTTTCCTTGCACTACGCATGGAAGACAAT
GGCTATGGTACTGTCAATTGTATCTCTCTTCCCTTTATGCCTGTC
CACGACCTCCCAAAAAACAACATGGCTTCCGGTGCTATTGGGATC
CCTTGGATGCAAACCACTAACCATGTTTCTTATAGCAGAAAACAA
AATCTGGGGAAGGAGAAGTTGGCCCCTCAATGAAGGAATCATGGC
TGTTGGAATAGTCAGCATCCTACTAAGTTCACTCCTCAAAAATGA
TGTACCGCTAGCTGGGCCACTAATAGCTGGAGGCATGCTAATAGC
ATGTTATGTTATATCTGGAAGCTCAGCCGACCTATCATTAGAGAA
AGCGGCTGAGGTCTCCTGGGAAGAAGAAGCAGAACACTCTGGTGC
CTCACACAACATATTAGTGGAAGTCCAAGATGATGGAACCATGAA
GATAAAGGATGAAGAGAGAGATGACACGCTAACCATTCTCCTTAA
AGCAACTCTGTTAGCAGTTTCAGGGGTGTACCCATTATCAATACC
AGCGACCCTTTTCGTGTGGTACTTTTGGCAGAAAAAGAAACAGAG
ATCTGGAGTGTTATGGGACACACCCAGCCCTCCAGAAGTGGAAAG
AGCAGTTCTTGATGATGGTATCTATAGAATTATGCAGAGAGGACT
GTTGGGCAGGTCCCAAGTAGGGGTAGGAGTTTTCCAAGAAAACGT
GTTCCACACAATGTGGCATGTCACCAGGGGAGCTGTACTCATGTA
TCAAGGGAAGAGATTGGAACCGAGCTGGGCCAGTGTCAAAAAAGA
CCTGATCTCATATGGAGGAGGTTGGAGGCTTCAAGGATCCTGGAA
CACAGGAGAAGAAGTGCAGGTGATTGCTGTTGAACCAGGGAAAAA
CCCCAAAAATGTACAAACAGCGCCGGGCACCTTTAAGACCCCTGA
AGGTGAAGTTGGAGCCATTGCCCTAGACTTTAAACCTGGCACATC
TGGATCTCCCATCGTGAACAGAGAAGGAAAAATAGTAGGTCTTTA
TGGAAATGGAGTAGTGACAACAAGTGGAACCTACGTCAGTGCCAT
AGCTCAAGCCAAAGCATCACAAGAAGGGCCCCTACCAGAGATTGA
AGACGAGGTGTTTAGGAAAAGAAACTTAACAATAATGGACCTACA
TCCAGGATCGGGGAAAACAAGAAGATATCTTCCAGCCATAGTCCG
TGAGGCCATAAAAAGGAAGCTGCGCACACTAATCCTGGCTCCCAC
AAGGGTTGTCGCTTCCGAAATGGCAGAGGCGCTCAAGGGAATGCC
AATAAGGTACCAAACAACAGCAGTGAAGAGTGAACATACAGGAAA
AGAGATAGTTGACCTCATGTGTCACGCCACTTTCACCATGCGCCT
CCTGTCTCCCGTAAGAGTTCCCAATTACAACATGATCATCATGGA
TGAAGCACATTTCACCGATCCATCCAGTATAGCGGCCAGAGGGTA
CATCTCAACCCGAGTGGGCATGGGTGAAGCAGCTGCAATCTTCAT
GACAGCCACTCCCCCAGGATCAGTGGAGGCCTTTCCACAGAGCAA
CGCAGTTATCCAAGATGAGGAAAGAGACATTCCTGAGAGATCATG
GAACTCAGGCTATGAGTGGATCACTGACTTCCCAGGTAAAACAGT
TTGGTTTGTTCCAAGCATTAAATCAGGAAATGACATTGCCAACTG
CTTAAGAAAGAATGGGAAACGGGTGATTCAATTGAGCAGGAAAAC
CTTTGATACAGAGTACCAAAAAACAAAAAACAACGACTGGGACTA
CGTCGTCACAACAGACATCTCCGAAATGGGAGCAAATTTCCGAGC
CGACAGGGTGATAGACCCAAGACGGTGTCTGAAACCGGTAATACT
AAAAGATGGTCCAGAGCGTGTCATTTTAGCAGGACCAATGCCAGT
GACTGTGGCCAGTGCCGCTCAGAGGAGAGGAAGAATTGGAAGGAA
CCACAATAAGGAAGGTGATCAGTACATCTACATGGGACAGCCTTT
AAACAACGATGAAGATCACGCTCACTGGACAGAAGCAAAAATGCT
CCTTGATAATATAAACACACCAGAAGGGATTATCCCAGCCCTCTT
TGAGCCAGAGAGAGAAAAGAGTGCAGCAATAGACGGGGAATACAG
ACTGCGGGGTGAAGCAAGGAAAACGTTTGTGGAGCTCATGAGAAG
AGGAGATCTACCTGTCTGGCTATCCTACAAAGTTGCCTCAGAAGG
CTTCCAGTACTCTGACAGAAGATGGTGCTTTGACGGGGAAAGGAA
CAACCAGGTGTTGGAGGAGAACATGGACGTGGAGATCTGGACAAA
AGAAGGAGAAAGAAAGAAACTACGACCCCGCTGGCTGGATGCCAG
AACATACTCAGACCCACTAGCCCTGCGCGAGTTTAAAGAGTTTGC
AGCAGGGAGAAGAAGCGTCTCAGGTGATTTAATATTAGAAATAGG
GAAGCTTCCACAACACTTGACGCAAAGGGCCCAGAATGCCCTGGA
CAACCTGGTTATGTTGCACAACTCCGAACAAGGAGGCAGAGCCTA
TAGACATGCAATGGAAGAACTGCCAGACACCATAGAAACGTTGAT
GCTCCTAGCTTTGATAGCTGTGTTAACTGGTGGAGTGACACTGTT
CTTCCTATCAGGAAGGGGCCTAGGGAAAACATCTATCGGCCTACT
CTGCGTAATGGCTTCAAGCGTACTGCTATGGATGGCCAGTGTGGA
GCCCCATTGGATAGCGGCCTCCATCATACTGGAGTTCTTCCTGAT
GGTGCTGCTTATTCCAGAGCCAGACAGACAACGCACTCCGCAGGA
CAACCAGCTGGCATATGTGGTGATAGGTTTGTTATTCATGATACT
GACAGTAGCAGCCAATGAGATGGGACTGCTGGAAACCACAAAGAA
AGACTTAGGGATTGGCCATGTGGCTGTTGAAAATCACCACCATGC
CGCAATGCTGGACGTAGACTTACATCCAGCTTCAGCCTGGACCCT
CTATGCAGTGGCCACAACAATTATCACTCCCATGATGAGGCACAC
AATCGAAAACACAACGGCAAACATTTCCCTGACAGCCATTGCAAA
CCAGGCAGCTATATTGATGGGACTTGACAAAGGATGGCCAATATC
GAAGATGGACATAGGAGTTCCACTTCTCGCCTTGGGGTGCTATTC
CCAGGTGAATCCACTGACGCTGACAGCGGCGGTATTGATGCTAGT
GGCTCATTACGCCATAATTGGACCTGGACTGCAAGCAAAAGCTAC
TAGAGAAGCTCAAAAAAGGACAGCGGCCGGAATAATGAAAAATCC
AACCGTTGATGGAATTGTTGCAATAGATTTGGACCCTGTGGTTTA
TGATGCAAAATTTGAAAAACAACTAGGCCAAATAATGTTGTTGAT
ACTATGCACATCACAGATCCTCTTGATGCGGACTACATGGGCCTT
GTGCGAGTCCATCACACTGGCCACTGGACCTCTGACCACGCTTTG
GGAGGGATCTCCAGGAAAATTTTGGAACACCACGATAGCGGTTTC
CATGGCAAACATTTTCAGAGGAAGTTATCTAGCAGGAGCAGGTCT
GGCCTTCTCATTAATGAAATCTCTAGGAGGAGGTAGGAGAGGCAC
GGGAGCCCAAGGGGAAACACTGGGAGAGAAATGGAAAAGACAGCT
GAACCAACTGAGCAAGTCAGAATTTAACACCTATAAAAGGAGTGG
GATTATGGAAGTGGACAGATCCGAAGCCAAAGAGGGATTGAAAAG
AGGAGAAACAACCAAACATGCAGTGTCGAGAGGAACCGCTAAACT
GAGGTGGTTCGTGGAGAGGAACCTTGTGAAACCAGAAGGGAAAGT
CATAGACCTCGGTTGTGGAAGAGGTGGCTGGTCATACTATTGCGC
TGGGCTGAAGAAAGTCACAGAAGTGAAGGGGTATACAAAAGGAGG
ACCTGGACATGAGGAACCAATCCCAATGGCGACCTATGGATGGAA
CCTAGTAAAGCTGCACTCTGGGAAAGACGTATTTTTTATACCACC
TGAGAAATGTGACACCCTTTTGTGTGATATTGGTGAGTCCTCTCC
AAACCCAACTATAGAGGAAGGAAGAACGCTACGCGTCCTAAAGAT
GGTGGAACCATGGCTCAGAGGAAACCAATTTTGCATAAAAATTCT
GAATCCCTACATGCCAAGTGTGGTGGAAACTCTGGAGCAAATGCA
AAGAAAACATGGAGGAATGCTAGTGCGAAATCCACTTTCAAGAAA
TTCTACTCATGAAATGTATTGGGTTTCATGTGGAACAGGAAACAT
TGTGTCAGCAGTAAACATGACATCTAGAATGTTGCTAAATCGATT
CACAATGGCTCACAGGAAACCAACATATGAAAGAGACGTGGACCT
AGGCGCCGGAACAAGACATGTGGCAGTGGAACCAGAGGTAGCCAA
CCTAGATATCATTGGCCAGAGGATAGAGAACATAAAACATGAACA
TAAGTCAACATGGCATTATGATGAGGACAATCCATATAAAACATG
GGCCTATCATGGATCATATGAGGTCAAGCCATCAGGATCAGCCTC
ATCCATGGTCAATGGCGTGGTGAAACTGCTCACCAAACCATGGGA
TGTCATCCCTATGGTCACACAAATAGCCATGACTGACACTACACC
CTTTGGACAACAGAGGGTGTTTAAAGAGAAAGTTGACACACGCAC
ACCAAAAGCAAAACGGGGCACAGCACAAATCATGGAGGTGACAGC
CAAGTGGTTATGGGGTTTTCTTTCTAGAAACAAGAAACCAAGAAT
TTGCACAAGAGAGGAGTTCACAAGAAAAGTTAGGTCAAACGCAGC
CATTGGAGCAGTGTTCGTTGATGAAAATCAATGGAACTCAGCAAA
AGAAGCAGTGGAAGATGAGCGGTTCTGGGACCTTGTGCATAGAGA
GAGGGAGCTTCACAAACAGGGAAAATGTGCTACGTGTGTTTACAA
CATGATGGGGAAGAGAGAGAAAAAGCTAGGAGAGTTCGGAAAGGC
AAAAGGAAGTCGTGCAATATGGTACATGTGGTTGGGAGCACGCTT
TCTAGAGTTCGAAGCTCTTGGTTTCATGAACGAAGATCACTGGTT
CAGCAGAGAGAATTCACTCAGCGGAGTGGAAGGAGAAGGACTCCA
CAAACTTGGATATATACTCAGAGACATATCAAAGATTCCAGGGGG
AAACATGTATGCAGATGACACAGCCGGATGGGATACAAGGATAAC
AGAGGATGACCTTCAGAATGAGGCCAGAATTACTGACATCATGGA
ACCCGAACATGCCCTCCTGGCTAAGTCAATCTTCAAGTTAACCTA
CCAAAATAAGGTGGTAAGGGTACAGAGACCAGCAAAAAATGGAAC
CGTGATGGATGTCATATCCAGACGTGACCAGAGAGGAAGTGGTCA
GGTCGGAACTTATGGCTTAAACACTTTCACCAACATGGAAGCCCA
GCTGATAAGACAAATGGAGTCTGAGGGAATCTTTTCACCCAGCGA
ATTAGAGACCCCAAATTTAGCCGAGAGAGTTCTCGACTGGCTGGA
AAAATATGGCGTCGAAAGGCTGAAAAGAATGGCAATCAGCGGAGA
TGACTGCGTAGTGAAACCAATTGATGATAGGTTTGCAACAGCCTT
GACAGCTCTGAATGATATGGGAAAAGTAAGAAAAGATATACCACA
ATGGGAACCTTCAAAAGGATGGAATGATTGGCAACAAGTGCCTTT
TTGTTCACACCACTTCCACCAGCTGATTATGAAGGATGGGAGGGA
AATAGTGGTGCCATGCCGCAACCAAGATGAACTTGTGGGTAGGGC
TAGAGTATCACAAGGTGCTGGATGGAGCCTGAGAGAAACTGCATG
CCTAGGCAAGTCATATGCACAAATGTGGCAGCTGATGTACTTCCA
CAGGAGAGACCTGAGACTAGCCGCTAATGCTATCTGTTCAGCCGT
TCCAGTTGATTGGATCCCAACCAGCCGTACCACCTGGTCGATCCA
TGCCCATCACCAATGGATGACAACAGAAGACATGCTGTCAGTGTG
GAATAGGGTTTGGATAGAGGAAAACCCATGGATGGAGGACAAAAC
CCACATATCCAGTTGGGGAGATGTTCCATATTTAGGAAAAAGGGA
AGACCAATGGTGTGGATCCCTGATAGGCTTAACAGCAAGGGCCAC
CTGGGCCACCAACATACAAGTGGCCATAAACCAAGTGAGAAGACT
AATTGGGAATGAGAATTATCTAGATTACATGACATCAATGAAGAG
ATTCAAGAACGAGAGTGATCCCGAAGGGGCACTCTGGTGAGTCAA
CACATTTACAAAATAAAGGAAAATAAGAAATCAAACAAGGCAAGA
AGTCAGGCCGGATTAAGCCATAGTACGGTAAGAGCTATGCTGCCT
GTGAGCCCCGTCTAAGGACGTAAAATGAAGTCAGGCCGAAAGCCA
CGGCTTGAGCAAACCGTGCTGCCTGTAGCTCCATCGTGGGGATGT
AAAAACCTGGGAGGCTGCAACCCATGGAAGCTGTACGCATGGGGT
AGCAGACTAGTGGTTAGAGGAGACCCCTCCCGAAACATAACGCAG
CAGCGGGGCCCAACACCAGGGGAAGCTGTACCCTGGTGGTAAGGA
CTAGAGGTTAGAGGAGACCCCCCGCATAACAATAAACAGCATATT
GACGCTGGGAGAGACCAGAGATCCTGCTGTCTCTACAGCATCATT
CCAGGCACAGAACGCCAGAAAATGGAATGGTGCTGTTGAATCAAC
AGGTTCT
DENV-2-NI-BID-V533-2005-E-W/Min
SEQ ID NO: 10
AGTTGTTAGTCTACGTGGACCGACAAAGACAGATTCTTTGAGGGA
GCTAAGCTCAACGTAGTTCTAACAGTTTTTTAATTAGAGAGCAGA
TCTCTGATGAATAACCAACGAAAAAAGGCGAGAAGTACGCCTTTC
AATATGCTGAAACGCGAGAGAAACCGCGTGTCAACTGTGCAACAG
CTGACAAAGAGATTCTCACTTGGAATGCTGCAAGGACGCGGACCA
TTAAAACTGTTCATGGCCCTTGTGGCGTTCCTTCGTTTCCTAACA
ATCCCACCAACAGCAGGGATACTAAAAAGATGGGGAACGATCAAA
AAATCAAAAGCTATCAATGTTTTGAGAGGGTTCAGGAAAGAGATT
GGAAGGATGCTGAACATCTTGAACAAGAGACGCAGGACAGCAGGC
GTGATTGTTATGTTGATTCCAACAGCGATGGCGTTCCATTTAACC
ACACGCAATGGAGAACCACACATGATCGTTGGTAGGCAGGAGAAA
GGGAAAAGTCTTCTGTTCAAAACAGAGGATGGTGTTAACATGTGT
ACTCTCATGGCCATAGACCTTGGTGAATTGTGTGAAGATACAATC
ACGTACAAGTGTCCTCTCCTCAGACAAAATGAACCAGAAGACATA
GATTGTTGGTGCAACTCTACGTCCACATGGGTAACTTATGGGACA
TGTACCACCACAGGAGAACACAGAAGAGAAAAAAGATCAGTGGCG
CTCGTTCCACATGTAGGTATGGGACTGGAGACACGAACTGAAACA
TGGATGTCATCAGAAGGGGCCTGGAAACATGTTCAGAGAATTGAA
ACCTGGATCTTGAGACATCCAGGTTTTACCATAATGGCAGCAATC
CTGGCATACACCATAGGAACGACACATTTCCAAAGGGCCTTGATT
TTCATTTTACTGACAGCTGTCGCTCCTTCAATGACAATGCGCTGC
ATAGGAATATCAAATAGAGACTTCGTAGAAGGGGTTTCAGGAGGA
AGCTGGGTTGACATAGTCTTAGAACATGGAAGTTGTGTGACGACG
ATGGCAAAAAACAAACCAACATTGGATTTTGAACTGATAAAAACA
GAAGCCAAACAACCTGCCACTCTAAGGAAGTACTGTATAGAAGCA
AAGCTGACCAACACAACAACAGAATCGCGTTGCCCAACACAAGGG
GAACCCAGTCTAATGAGGAGCAGGACAAAAGGTTCATCTGCAAAC
ACTCCATGGTAGACAGAGGATGGGGAAATGGATGTGGATTATTTG
GAAAGGGAGGCATTGTGACCTGTGCTATGTTTACATGCAAAAAGA
ACATGGAAGGAAAAGTCGTGCAGCCAGAAAATTTGGAATACACCA
TCGTGATAACACCTCACTCAGGAGAGGAGCACGCTGTAGGTAATG
ACACAGGAAAGCATGGCAAGGAAATCAAAATAACACCACAGAGCT
CCATCACAGAAGCAGAACTGACAGGCTATGGCACTGTCACGATGG
AGTGCTCTCCGAGAACGGGCCTCGACTTCAATGAGATGGTACTGC
TGCAGATGGAAGACAAAGCTTGGCTGGTGCACAGGCAATGGTTCC
TAGACCTGCCGTTACCATGGCTACCCGGAGCGGACACACAAGGAT
CAAATTGGATACAGAAAGAGACATTGGTCACTTTCAAAAATCCCC
ACGCGAAGAAACAGGACGTAGTCGTACTCGGATCACAGGAAGGCG
CAATGCATACCGCATTGACAGGCGCTACAGAGATACAGATGTCTA
GCGGAAATCTGTTGTTTACAGGGCATCTGAAATGTAGACTGAGAA
TGGACAAACTGCAATTGAAGGGAATGTCATACTCTATGTGTACGG
GTAAGTTTAAGATAGTCAAAGAGATAGCCGAAACACAACACGGAA
CAATCGTAATTAGGGTGCAATACGAAGGCGACGGGTCACCATGTA
AGATACCATTCGAAATTACAGACCTCGAAAAAAGACACGTACTCG
GAAGACTGATAACAGTGAATCCGATCGTTACGGAAAAAGACTCAC
CCGTTAATATCGAAGCCGAACCACCATTCGGAGACTCATACATAA
TAATCGGAGTCGAACCCGGACAATTGAAACTGAATTGGTTTAAAA
AAGGGTCATCAATCGGACAAATGTTCGAAACAACTATGAGAGGCG
CTAAGCGTATGGCTATACTCGGAGACACAGCATGGGACTTCGGAT
CCTTAGGGGGAGTGTTTACGTCAATCGGTAAGGCACTACACCAGG
TATTCGGAGCGATATACGGAGCCGCATTTAGCGGAGTGTCGTGGA
CAATGAAGATACTGATCGGAGTGATAATCACATGGATCGGAATGA
ATAGTAGGTCTACAAGTCTATCCGTTAGCTTAGTGTTAGTCGGAG
TCGTTACACTGTATCTAGGCGCTATGGTGCAAGCCGATAGTGGTT
GCGTTGTGAGCTGGAAAAATAAAGAACTGAAATGTGGCAGCGGGA
TCTTCATCACAGATAACGTACACACATGGACAGAACAATATAAGT
TCCAACCAGAATCCCCTTCAAAACTAGCTTCAGCTATCCAAAAAG
CTCATGAAGAGGGCATTTGTGGAATCCGCTCAGTAACAAGATTGG
AGAATCTGATGTGGAAACAAATAACACCAGAATTGAATCATATTC
TATCAGAAAATGAGGTAAAGTTGACCATTATGACAGGAGACATTA
GAGGAATCATGCAGGCAGGAAAACGATCCTTGCGGCCCCAGCCCA
CTGAGCTGAAGTACTCATGGAAAACATGGGGAAAGGCGAAAATGC
TCTCCACAGAGTCTCACAATCAGACCTTTCTTATTGATGGCCCTG
AAACAGCAGAATGCCCCAACACAAACAGAGCCTGGAACTCGCTGG
AAGTTGAAGACTATGGTTTTGGAGTTTTCACCACCAATATATGGC
TGAAATTGAGAGAAAAACAGGATGTATTTTGTGACTCAAAACTCA
TGTCAGCGGCCATTAAAGACAACAGAGCCGTTCATGCTGATATGG
GTTATTGGATAGAAAGTGCACTCAATGACACATGGAAGATGGAGA
AAGCCTCCTTCATTGAAGTTAAAAGCTGCCACTGGCCAAAGTCAC
ACACCCTTTGGAGCAATGGAGTATTAGAAAGTGAGATGATAATCC
CAAAAAATTTTGCCGGGCCAGTGTCACAACACAACTACAGACCAG
GCTACCATACACAAACAGCAGGACCTTGGCATCTAGGTAAGCTTG
AGATGGACTTTGATCTCTGCGAAGGAACTACAGTGGTGGTGACTG
AGGACTGTGGAAATAGAGGACCCTCTTTAAGAACGACCACTGCCT
CTGGAAAACTCATAACAGAATGGTGCTGCCGATCCTGCACACTAC
CACCTCTAAGATACAGAGGTGAGGATGGATGCTGGTACGGGATGG
AAATCAGACCATTGAAAGAGAAAGAAGAGAATTTGGTCAACTCCT
TGGTCACAGCCGGACATGGGCAGATTGACAACTTTTCACTAGGAG
TCTTGGGAATGGCACTGTTCCTGGAAGAAATGCTCAGGACCCGAA
TAGGAACGAAACATGCAATACTGCTAGTTGCAGTATCTTTTGTGA
CATTGATTACTGGGAACATGTCTTTTAGAGACCTGGGAAGAGTGA
TGGTTATGGTGGGCGCTACCATGACGGATGACATAGGTATGGGAG
TGACTTATCTTGCCCTACTAGCAGCTTTTAAGGTTAGACCAACTT
TTGCAGCTGGACTACTCTTAAGAAAACTGACCTCCAAGGAATTGA
TGATGGCCACCATAGGAATCGCACTCCTTTCCCAAAGCACCATAC
CAGAGACCATTCTTGAACTGACTGATGCATTAGCCCTGGGCATGA
TGGTCCTCAAAATAGTGAGAAATATGGAAAAATACCAATTGGCAG
TGACTATCATGGCTATTTCATGTGTCCCAAATGCAGTGATACTGC
AAAACGCATGGAAGGTGAGTTGCACAATATTGGCAGCGGTGTCCG
TTTCACCACTGCTCTTAACATCCTCACAGCAGAAAGCGGATTGGA
TACCACTGGCATTGACGATAAAAGGTCTCAATCCAACAGCCATTT
TTTTAACAACTCTCTCGAGGACCAGCAAGAAAAGGAGCTGGCCGC
TAAATGAAGCTATCATGGCAGTTGGGATGGTGAGCATTTTAGCCA
GTTCTCTCCTAAAGAATGATATTCCTATGACAGGTCCATTAGTGG
CTGGAGGACTCCTCACCGTATGTTACGTGCTCACTGGACGATCGG
CCGATTTGGAACTGGAGAGAGCTGCCGATGTAAAATGGGAAGATC
AGGCAGAAATATCAGGAAGCAGCCCAATCCTGTCAATAACAATAT
CAGAAGATGGCAGCATGTCGATAAAAAATGAAGAGGAAGAACAAA
CACTGACCATACTCATCAGAACGGGATTGTTGGTGATCTCAGGAG
TCTTTCCAGTATCGATACCAATTACGGCAGCAGCATGGTACCTGT
GGGAAGTAAAGAAACAACGGGCTGGAGTACTGTGGGACGTCCCTT
CACCCCCACCAGTGGAAAAAGCCGAACTGGAGGATGGAGCCTACA
GAATCAAGCAAAGAGGGATCCTTGGATATTCTCAGATTGGAGCCG
GAGTTTACAAAGAAGGAACATTCCATACAATGTGGCACGTCACAC
GTGGTGCTGTTCTGATGCATAGAGGGAAGAGGATTGAACCATCAT
GGGCAGATGTCAAGAAAGACCTAATATCATATGGAGGAGGCTGGA
AGCTAGAAGGAGAATGGAAGGAAGGAGAGGAAGTCCAAGTCCTGG
CATTGGAACCTGGAAAAAATCCAAGAGCCGTCCAAACGAAACCTG
GAATATTCAAAACCAACACCGGAACCATAGGCGCCGTATCTCTGG
ACTTTTCCCCTGGAACGTCAGGATCTCCAATCGTCGACAGAAAAG
GAAAAGTTGTGGGTCTTTACGGTAATGGTGTTGTCACAAGGAGTG
GAGCATATGTAAGTGCTATAGCCCAGACCGAAAAAAGCATTGAAG
ACAATCCAGAGATCGAAGATGACATTTTCCGAAAGAAAAGATTGA
CCATCATGGACCTCCATCCAGGAGCAGGAAAGACAAAAAGATACC
TTCCAGCCATAGTTAGAGAAGCCATAAAACGTGGCTTGAGAACAT
TGATCCTGGCTCCCACTAGAGTAGTGGCAGCTGAAATGGAGGAAG
CTCTTAGAGGACTTCCAATAAGATACCAAACTACAGCCATCAAAA
CCGAGCATACCGGGCGGGAGATCGTGGACCTAATGTGTCATGCCA
CATTTACTATGAGGCTGTTATCACCAGTCAGAGTGCCAAATTACA
ACCTGATCATCATGGACGAAGCCCACTTCACAGACCCAGCAAGTA
TAGCAGCTAGAGGATACATTTCAACTCGAGTAGAGATGGGTGAAG
CAGCCGGGATTTTCATGACAGCCACTCCTCCGGGAAGTAGAGACC
CATTTCCTCAGAGCAATGCACCAATTATGGATGAGGAAAGAGAAA
TCCCTGAGCGTTCATGGAATTCAGGACACGAATGGGTCACGGATT
TTAAGGGGAAGACTGTTTGGTTTGTTCCAAGTATAAAAGCAGGAA
ATGATATAGCAGCTTGTCTTAGGAAAAATGGAAAGAAAGTGATAC
AACTCAGTAGGAAGACTTTTGACTCTGAGTATGTTAAGACTAGAG
CCAATGATTGGGACTTTGTGGTCACAACTGACATTTCAGAAATGG
GTGCCAACTTCAAGGCTGAGAGGGTTATAGACCCCAGACGTTGCA
TGAAACCAGTTATACTAACAGATGGCGAGGAGCGGGTGATCTTGG
CTGGACCTATGCCAGTGACCCACTCTAGTGCAGCGCAAAGAAGAG
GGAGAATAGGAAGAAATCCAAAAAATGAAAATGACCAGTACATAT
ACATGGGGGAACCTCTTGAAAATGATGAAGACTGTGCACATTGGA
AAGAAGCTAAAATGCTCCTAGATAACATCAACACACCTGAAGGAA
TCATTCCTAGTATGTTCGAACCAGAGCGTGAAAAAGTGGATGCCA
TTGATGGTGAATACCGTTTGAGAGGAGAAGCAAGGAAAACCTTTG
TGGACCTAATGAGAAGAGGGGACTTACCAGTCTGGTTGGCCTACA
AAGTGGCAGCTGAAGGCATCAACTACGCAGACAGAAAGTGGTGTT
TTGATGGAATTAAGAACAACCAAATACTGGAAGAAAATATGGAAG
TGGAAATCTGGACAAAAGAAGGGGAAAGGAAAAAATTAAAACCCA
GATGGTTGGATGCTAGGATCTATTCTGACCCACTAGCACTAAAAG
AATTCAAGGAATTTGCAGCTGGAAGAAAATCTTTGACCCTGAACC
TAATCACAGAAATGGGTAGGCTTCCAACTTTCATGACTCAGAAAG
CAAGAAACGCACTGGACAACCTGGCTGTGCTGCATACGGCTGAGG
CAGGTGGAAGGGCGTACAATCATGCTCTCAGTGAACTGCCGGAGA
CCCTGGAGACACTGCTCCTACTGACACTTCTGGCAACAGTCACAG
GAGGAATCTTCTTATTCTTAATGAGCGGAAAAGGTATAGGGAAGA
TGACCCTGGGAATGTGTTGCATAATCACGGCTAGTATCCTCCTAT
GGTATGCATTCCAGAACCAGAAAAACAGAGAACACCCCAAGACAA
CCAATTGACCTACGTTGTCATAGCCATCCTCACAGTGGTGGCCGC
AACCATGGCAAACGAGATGGGTTTCCTGGAAAAAACCAAGAAAGA
CCTCGGATTGGGAAGCATTACAACCCAGGAATCTGAGAGCAATAT
CCTGGACATAGATCTACGCCCTGCATCAGCATGGACGCTGTATGC
CGTAGCTACAACATTTGTCACACCAATGTTGAGACATAGCATTGA
AAATTCCTCAGTGAATGTCTCCCTAACAGCCATTGCTAACCAAGC
TACAGTGCTAATGGGTCTTGGGAAAGGATGGCCATTGTCAAAGAT
GGACATTGGAGTTCCCCTCCTTGCCATTGGATGCTATTCACAAGT
CAACCCTATAACTCTCACAGCAGCTCTCCTTTTATTGGTAGCACA
TTATGCCATTATAGGGCCAGGACTTCAAGCAAAAGCAACCAGAGA
AGCCCAGAAAAGAGCAGCAGCAGGCATCATGAAAAACCCAACAGT
CGATGGAATAACAGTGATTGACCTAGAACCAATACCCTATGATCC
AAAATTTGAAAAGCAGTTAGGACAAGTAATGCTCCTAATCCTCTG
CGTGACTCAAGTATTAATGATGAGGACTACATGGGCTTTATGTGA
GGCTCTAACCCTAGCGACCGGGCCCATCTCCACACTGTGGGAAGG
AAATCCAGGGAGGTTTTGGAACACTACCATTGCAGTGTCAATGGC
TAACATCTTTAGGGGGAGCTACTTGGCCGGAGCTGGACTTCTCTT
TTCCATCATGAAAAACACAACAAACACAAGAAGAGGAACTGGCAA
CATAGGAGAGACACTTGGAGAAAAATGGAAAAGTCGATTAAACGC
ACTGGGGAAAAGTGAATTTCAAATCTACAAGAAAAGTGGAATCCA
GGAAGTGGATAGAACCCTAGCAAAAGAAGGTATCAAAAGAGGAGA
AACGGACCACCATGCTGTGTCGCGAGGTTCAGCAAAACTGAGATG
GTTCGTCGAGAGAAATATGGTCACACCGGAAGGGAAGGTGGTGGA
TCTCGGTTGCGGCAGAGGGGGCTGGTCATACTATTGCGGGGGACT
AAAGAATGTAAGAGAAGTCAAAGGCCTAACAAAAGGAGGACCAGG
ACATGAAGAACCCATCCCCATGTCAACATATGGGTGGAATCTAGT
GCGTCTGCAAAGTGGAGTTGACGTTTTCTTCACCCCGCCAGAAAA
GTGTGATACATTGTTGTGTGACATAGGGGAGTCGTCACCAAATCC
CACGATAGAAGCAGGACGAACACTCAGAGTCCTCAACTTAGTGGA
AAATTGGTTGAACAATAACACCCAATTTTGCATAAAGGTCCTCAA
CCCATATATGCCTTCAGTCATAGAAAAAATGGAAGCATTACAAAG
GAAATATGGAGGAGCCTTAGTGAGGAATCCACTCTCACGAAACTC
CACGCATGAAATGTACTGGGTATCTAATGCCACCGGGAACATAGT
GTCATCAGTGAACATGATTTCAAGGATGTTGATTAACAGATTCAC
AATGAAACACAAGAAAGCCACTTACGAGCCAGATGTTGACCTAGG
AAGTGGAACCCGCAACATTGGAATTGAAAGTGAGATACCAAATCT
AGACATAATAGGAAAGAGAATAGAGAAAATAAAACAAGAGCATGA
AACATCATGGCACTATGATCAAGACCACCCATACAAAACGTGGGC
TTACCATGGCAGCTATGAAACAAAACAAACTGGATCAGCATCATC
TATGGTGAACGGAGTGGTCAGATTGCTGACAAAACCTTGGGACGT
CGTCCCTATGGTGACACAGATGGCAATGACAGACACGACTCCTTT
TGGACAACAGCGCGTTTTCAAAGAGAAAGTAGACACGAGAACCCA
AGAACCGAAGGAAGGCACAAAGAAACTGATGAAAATTACGGCAGA
GTGGCTTTGGAAAGAACTAGGAAAGAAAAAGACACCTAGGATGTG
TACCAGAGAAGAATTCACAAGAAAAGTGAGAAGCAATGCAGCCTT
GGGGGCCATATTCACTGATGAGAACAAATGGAAATCGGCACGTGA
GGCTGTTGAAGATGGTAGGTTCTGGGAGCTGGTTGACAGGGAAAG
AAATCTCCATCTTGAAGGAAAGTGTGAAACATGTGTGTACAACAT
GATGGGAAAAAGAGAGAAGAAACTAGGGGAGTTCGGCAAGGCAAA
AGGTAGCAGAGCCATATGGTACATGTGGCTTGGAGCACGCTTCTT
AGAGTTTGAAGCCCTAGGATTCCTGAATGAAGATCACTGGTTCTC
CAGAGGGAACTCCCTGAGTGGAGTGGAAGGAGAAGGGCTGCACAG
GCTAGGCTACATTTTAAGAGACGTGGGCAAGAAGGAAGGGGGGGC
AATGTACGCCGATGATACAGCAGGATGGGACACAAGAATCACACT
AGAAGACTTAAAAAATGAAGAAATGGTAACGAACCACATGAAAGG
AGAACACAAGAAACTAGCCGAGGCCATATTCAAACTAACGTACCA
AAACAAGGTGGTGCGTGTGCAAAGACCAACACCAAGAGGTACAGT
AATGGATATCATATCGAGAAGAGACCAAAGAGGCAGTGGGCAAGT
CGGCACCTATGGCCTTAATACCTTCACCAATATGGAAGCCCAATT
AATTAGACAGATGGAGGGAGAAGGAATCTTCAAAAGCATTCAGCA
CCTGACAGCCACAGAAGAAATCGCTGTACAGAACTGGCTAGCAAG
AGTGGGGCGTGAAAGGCTATCAAGAATGGCAATCAGTGGAGATGA
TTGTGTTGTAAAACCTATAGATGACAGATTTGCAAGTGCTTTAAC
AGCTCTAAATGACATGGGAAAAGTTAGAAAAGATATACAACAATG
GGAACCTTCAAGAGGATGGAACGATTGGACACAAGTGCCTTTCTG
TTCACACCACTTTCATGAGTTAGTCATGAAAGATGGTCGCGTGCT
CGTAGTCCCATGCAGAAACCAAGATGAACTGATTGGTAGAGCCCG
AATTTCCCAGGGAGCTGGGTGGTCTTTGAAAGAGACGGCCTGTTT
GGGAAAGTCTTACGCCCAAATGTGGACTCTGATGTACTTCCACAG
ACGTGACCTCAGACTGGCGGCAAATGCCATCTGCTCGGCAGTCCC
GTCACATTGGGTTCCAACAAGTCGAACAACCTGGTCCATACACGC
TAAGCATGAATGGATGACGACGGAAGACATGCTGGCAGTCTGGAA
CAGGGTGTGGATCCAAGAAAACCCGTGGATGGAAGATAAAACTCC
AGTGGAATCATGGGAAGAAGTCCCATACTTGGGAAAAAGAGAAGA
CCAATGGTGCGGCTCATTGATTGGGCTAACAAGCAGGGCTACCTG
GGCAAAGAACATCCAAACAGCAATAAATCAAGTCAGATCCCTTAT
AGGCAATGAGGAATACACAGACTACATGCCATCCATGAAGAGATT
TAGAAGGGAAGAGGAAGAGGCAGGTGTCCTGTGGTAGAAGGCAAA
ACTAACATGAAACAAGGCTAAAAGTCAGGTCGGATTAAGCCATAG
TACGGAAAAAACTATGCTACCTGTGAGCCCCGTCCAAGGACGTTA
AAAGAAGTCAGGCCATCACAAAATGCCACAGCTTGAGTAAACTGT
GCAGCCTGTAGCTCCACCTGAGGAGGTGTAAAAAACCTGGGAGGC
CACAAACCATGGAAGCTGTACGCATGGCGTAGTGGACTAGCGGTT
AGAGGAGACCCCTCCCTTACAAATCGCAGCAAACAACGGGGGCCC
AAGGTGAGATGAAGCTGTAATCTCACTGGAAGGACTAGAGGTTAG
AGGAGACCCCCCCAAAACAAAAAACAGCATATTGACGCTGGGAAA
GACCAGAGATCCTGCTGTCTCCTCAGCATCATTCCAGGCACAGAA
CGCCAGAAAATGGAATGGTGCTGTTGAATCAACAGGTTCT
DENV-3-VE-BID-V2268-2008-E-W/Min
SEQ ID NO: 11
AGTTGTTAGTCTACGTGGACCGACAAGAACAGTTTCGACTCGGAA
GCTTGCTTAACGTAGTGCTGTCTATCAATATGCTGAAACGCGTGA
GAAACCGTGTGTCAACTGGATCACAGTTGGCGAAGAGATTCTCAA
AAGGACTGCTGAACGGCCAGGGACCAATGAAATTGGTTATGGCGT
TCATAGCTTTCCTCAGATTTCTAGCCATTCCACCAACAGCAGGAG
TCTTGGCTAGATGGGGAACCTTCAAGAAGTCGGGGGCCATTAAGG
TCCTGAAAGGCTTTAAGAAGGAGATCTCAAACATGCTGAGCATAA
TCAACAAACGGAAAAAGACATCGCTCTGTCTCATGATGATATTGC
CAGCAGCACTTGCTTTCCACTTGACTTCACGAGATGGAGAGCCGC
GCATGATTGTGGGGAAGAATGAAAGAGGAAAATCCCTACTTTTTA
AGACAGCCTCTGGAATTAACATGTGCACACTCATAGCCATGGACT
TGGGAGAGATGTGTGATGACACGGTCACTTACAAATGCCCCCATA
TTACCGAAGTGGAACCTGAAGACATTGACTGCTGGTGCAACCTCA
CATCAACATGGGTGACTTATGGAACGTGCAATCAAGCCGGAGAGC
ATAGACGCGATAAGAGATCAGTGGCGTTAGCTCCCCATGTCGGCA
TGGGACTGGATACACGCACCCAAACTTGGATGTCGGCTGAAGGAG
CTTGGAGGCAAGTCGAGAAGGTAGAGACATGGGCCCTTAGGCACC
CAGGGTTCACCATACTAGCCCTATTTCTTGCCCATTACATAGGCA
CTTCCTTGACCCAGAAGGTGGTTATTTTTATACTGCTAATGCTGG
TCACCCCATCCATGACAATGAGATGTGTGGGAGTAGGAAACAGAG
ATTTTGTGGAAGGACTATCAGGAGCTACGTGGGTTGACGTGGTGC
TCGAGCACGGGGGGTGTGTGACCACCATGGCTAAGAACAAGCCCA
CGTTGGATATAGAGCTTCAGAAGACCGAGGCCACCCAACTGGCGA
CCCTAAGGAAGCTATGCATTGAGGGGAAAATCACCAACATAACAA
CTGACTCAAGATGTCCTACCCAAGGGGAAGCGGTTTTGCCTGAGG
AGCAGGACCAAAACTACGTGTGTAAGCATACATACGTAGACAGAG
GCTGGGGGAACGGATGTGGTTTGTTTGGTAAGGGAAGCTTGGTAA
CATGTGCGAAATTTCAATGCCTGGAACCAATAGAGGGAAAAGTGG
TGCAATATGAGAACCTCAAATACACCGTTATCATCACAGTGCACA
CAGGAGACCAACACCAGGTGGGAAATGAAACGCAGGGAGTCACGG
CTGAGATAACACCTCAGGCATCAACCACTGAAGCCATCTTGCCTG
AATATGGAACCCTTGGGCTAGAATGCTCACCACGGACAGGTTTGG
ACTTCAATGAAATGATCTTGCTAACAATGAAGAACAAAGCATGGA
TGGTACATAGACAATGGTTTTTTGACCTACCTCTACCATGGACAT
CAGGAGCTACAACGGAAACACCAACCTGGAACAGGAAGGAGCTTC
TTGTGACATTTAAAAACGCACATGCGAAGAAACAAGAAGTAGTCG
TTTTAGGGTCACAGGAGGGAGCTATGCATACCGCACTAACCGGAG
CGACTGAGATACAGAATAGCGGAGGGACATCAATCTTCGCCGGAC
ACCTTAAGTGTAGATTGAAAATGGACAAACTTGAGCTTAAGGGGA
TGTCATACGCTATGTGTACGAACACATTCGTGCTGAAAAAAGAGG
TAAGCGAAACGCAACACGGAACGATACTGATTAAGGTTGAGTATA
AGGGCGAAGACGTCCCATGTAAGATACCTTTTTCGACAGAGGACG
GACAGGGTAAGGCCCATAACGGAAGACTGATTACCGCTAACCCAG
TGGTGACCAAAAAAGAGGAACCAGTCAATATCGAAGCCGAACCTC
CATTCGGAGAGTCAAACATAGTGATAGGGATAGGCGATAACGCAC
TTAAGATTAACTGGTACAAAAAAGGGTCATCAATCGGTAAGATGT
TTGAGGCAACCGCTAGGGGGGCTAGACGGATGGCCATACTCGGAG
ACACCGCATGGGACTTCGGATCCGTGGGGGGGGTACTTAACTCAC
TCGGTAAGATGGTGCACCAAATTTTCGGATCCGCATATACCGCTC
TATTTAGCGGAGTGTCATGGGTGATGAAAATCGGAATCGGAGTTC
TATTGACATGGATCGGATTGAACTCTAAGAATACATCTATGTCAT
TTTCATGTATCGCAATCGGAATTATTACGCTATATCTCGGAGCCG
TAGTGCAAGCCGACATGGGGTGTGTTATAAACTGGAAAGGCAAAG
AACTCAAGTGTGGAAGTGGAATCTTCGTCACCAACGAGGTCCATA
CCTGGACAGAGCAATACAAATTCCAAGCAGACTCCCCAAAAAGAT
TGGCGACAGCCATTGCAGGCGCTTGGGAGAATGGAGTGTGCGGAA
TTAGGTCAACAACCAGAATGGAGAATCTCCTGTGGAAGCAAATAG
CCAATGAACTGAACTACATATTGTGGGAAAACAATATCAAATTAA
CGGTTGTTGTGGGCGATATAATTGGGGTCTTAGAGCAAGGGAAAA
GAACACTAACACCACAACCCATGGAGCTAAAATACTCATGGAAAA
CGTGGGGAAAGGCAAAAATAGTGACAGCTGAAACACAAAATTCTT
CTTTCATAATAGATGGACCAAACACACCGGAGTGTCCAAGTGCCT
CAAGAGCATGGAATGTGTGGGAGGTGGAAGATTACGGGTTCGGAG
TCTTCACAACCAACATATGGCTGAAACTCCGAGAGGTGTATACCC
AACTATGTGACCATAGGTTAATGTCGGCAGCCGTCAAGGATGAAA
GGGCCGTACATGCCGACATGGGCTATTGGATAGAAAGTCAAAAGA
ATGGAAGTTGGAAGCTAGAAAAAGCATCCCTCATAGAGGTGAAAA
CCTGCACATGGCCAAAATCACATACCCTTTGGAGTAATGGTGTGT
TAGAGAGTGACATGATCATTCCAAAAAGTCTAGCTGGTCCTATCT
CGCAACACAACTACAGGCCCGGGTACCACACCCAGACGGCGGGAC
CTTGGCATTTAGGAAAATTAGAGCTGGACTTCAACTATTGTGAAG
GAACAACAGTTGTCATCACAGAAAACTGTGGGACAAGAGGCCCAT
CATTGAGAACAACAACAGTGTCAGGGAAGTTAATACACGAATGGT
GCTGCCGCTCGTGCACACTTCCTCCCCTGCGATACATGGGAGAAG
ACGGTTGCTGGTATGGCATGGAAATCAGACCCATCAGTGAGAAAG
AAGAAAACATGGTAAAGTCTTTAGTCTCAGCGGGAAGTGGAGAGG
TGGACAACTTCACAATGGGTGTCTTGTGTTTGGCAATCCTCTTTG
AAGAGGTGATGAGAGGAAAATTTGGGAAGAAACACATGATTGCGG
GGGTTTTCTTCACGTTTGTACTCCTTCTCTCAGGGCAAATAACAT
GGAGAGATATGGCGCACACACTAATAATGATTGGGTCCAATGCAT
CTGACAGGATGGGAATGGGCGTCACCTACCTAGCTTTAATTGCAA
CATTTAAAATCCAGCCATTTTTGGCTTTGGGATTTTTCCTAAGAA
AACTGACATCCAGAGAAAATTTATTGTTAGGAGTTGGGCTGGCTA
TGGCAACAACGTTACAACTGCCAGAGGACATTGAACAAATGGCAA
ATGGAATCGCTCTGGGGCTCATGGCTCTCAAACTGATAACACAAT
TTGAAACATACCAACTATGGACAGCATTAATCTCCTTAACGTGTT
CAAATACAATTTTTACGTTGACTGTTGCCTGGAGAACAGCCACCC
TGATTTTGGCTGG
AGTTTCACTTTTACCAGTGTGCCAGTCTTCGAGTATGAGGAAAAC
AGACTGGCTTCCAATGACAGTGGCAGCTATGGGAGTTCCACCTCT
ACCACTTTTTATTTTTAGCTTAAAAGACACACTCAAAAGGAGAAG
CTGGCCACTGAATGAAGGGGTGATGGCTGTTGGGCTTGTGAGCAT
TCTGGCCAGTTCTCTCCTTAGAAATGATGTACCCATGGCTGGACC
ATTAGTGGCCGGGGGCTTGCTGATAGCGTGCTACGTCATAACTGG
CACGTCAGCAGACCTCACCGTAGAAAAAGCAGCAGATGTAACATG
GGAGGAAGAGGCTGAGCAAACAGGAGTGTCCCACAACTTAATGAT
CACAGTTGATGATGATGGAACAATGAGAATAAAAGATGATGAGAC
TGAGAATATCTTAACAGTGCTTTTGAAAACAGCATTACTAATAGT
GTCAGGAATCTTTCCATACTCCATACCCGCAACATTGTTGGTCTG
GCATACTTGGCAAAAGCAAACCCAAAGATCCGGCGTTCTATGGGA
TGTACCCAGCCCTCCAGAGACACAGAAAGCAGAACTGGAAGAAGG
GGTCTATAGGATCAAACAGCAAGGAATTTTTGGGAAAACCCAAGT
AGGGGTTGGAGTACAGAAAGAAGGAGTCTTTCACACCATGTGGCA
CGTTACAAGAGGGGCAGTGTTGACATATAATGGGAAAAGACTGGA
ACCAAATTGGGCTAGCGTGAAAAAAGATCTGATTTCATACGGAGG
AGGATGGAGATTGAGCGCACAATGGCAAAAGGGGGAGGAGGTGCA
GGTTATTGCCGTAGAGCCTGGGAAGAACCCAAAGAACTTTCAAAC
CATGCCAGGCACTTTTCAGACTACAACAGGGGAAATAGGAGCAAT
TGCACTGGATTTCAAGCCCGGAACTTCAGGATCTCCTATCATAAA
CAGAGAGGGAAAGGTAGTGGGACTGTATGGCAATGGAGTGGTTAC
AAAGAATGGTGGCTACGTCAGCGGAATAGCGCAAACGAATGCAGA
ACCAGATGGACCGACACCAGAATTGGAAGAAGAGATGTTCAAAAA
GCGAAATCTAACCATAATGGATCTTCATCCTGGGTCAGGAAAGAC
ACGGAAATACCTTCCAGCTATTGTTAGAGAGGCAATCAAGAGACG
TTTGAGAACTCTAATTCTGGCACCAACAAGGGTGGTTGCAGCTGA
GATGGAAGAAGCATTGAAAGGGCTCCCAATAAGGTACCAAACAAC
AGCAACAAAATCTGAACACACAGGAAGAGAGATTGTTGATCTGAT
GTGCCACGCAACGTTCACAATGCGTCTGCTGTCACCAGTTAGGGT
TCCAAACTATAACTTGATAATAATGGATGAAGCCCATTTCACAGA
CCCAGCCAGTATAGCTGCTAGAGGGTACATATCAACTCGTGTTGG
AATGGGAGAAGCAGCCGCAATATTCATGACAGCAACGCCCCCTGG
AACAGCTGATGCCTTTCCCCAGAGCAACGCTCCAATTCAAGATGA
AGAAAGGGACATACCAGAACGCTCATGGAATTCAGGCAATGAATG
GATAACCGACTTCGCTGGGAAAACGGTGTGGTTTGTCCCCAGCAT
TAAAGCCGGAAATGACATAGCAAATTGCCTGCGGAAAAACGGGAA
AAAGGTCATTCAACTTAGTAGGAAGACTTTTGACACAGAATATCA
GAAAACCAAACTGAATGATTGGGACTTTGTGGTGACGACTGACAT
TTCAGAAATGGGGGCCAATTTCAAAGCAGATAGAGTGATCGACCC
AAGAAGATGTCTCAAACCAGTGATCCTGACAGATGGACCAGAGCG
GGTGATCCTGGCTGGACCAATGCCAGTCACCGCAGCGAGTGCTGC
GCAAAGGAGAGGGAGAGTTGGCAGGAACCCACAAAAAGAAAATGA
CCAGTACATATTCACGGGCCAGCCTCTCAACAATGATGAAGACCA
TGCTCACTGGACAGAAGCAAAAATGCTGCTGGACAACATTAACAC
ACCAGAAGGGATCATACCAGCTCTCTTTGAGCCAGAAAGGGAGAA
GTCAGCCGCCATAGACGGTGAGTATCGCCTGAAAGGTGAGTCCAG
GAAGACTTTCGTGGAACTCATGAGGAGGGGTGACCTTCCAGTCTG
GTTAGCCCATAAAGTAGCATCAGAAGGGATCAAATATACAGATAG
AAAATGGTGCTTTGATGGACAACGTAATAATCAAATTTTAGAAGA
GAACATGGATGTGGAAATCTGGACAAAGGAAGGAGAAAAGAAAAA
ATTGAGACCTAGGTGGCTTGATGCTCGCACCTATTCAGATCCCTT
AGCACTCAAGGAATTCAAGGACTTTGCGGCTGGCAGGAAGTCAAT
AGCCCTTGATCTTGTGACAGAAATAGGAAGAGTGCCTTCACACCT
AGCTCATAGAACGAGAAACGCTCTGGACAATCTGGTGATGCTGCA
TACGTCAGAACATGGCGGTAAGGCCTACAGGCATGCGGTGGAGGA
ACTACCAGAGACAATGGAAACACTCCTACTCTTGGGACTCATGAT
CTTGTTGACAGGTGGAGCAATGCTTTTCTTAATATCAGGTAAAGG
GATTGGAAAGACTTCAATAGGACTCATTTGTGTAATTGCTTCCAG
CGGCATGTTGTGGATGGCCGAAATCCCACTCCAATGGATCGCGTC
GGCCATAGTCCTGGAGTTTTTATGATGGTGTTGCTTATACCAGAA
CCAGAGAAGCAGAGAACCCCCCAAGACAACCAACTCGCATATGTC
GTGATAGGCATACTTACACTGGCAGCAATAATAGCAGCCAATGAA
ATGGGATTGTTGGAAACTACAAAGAGAGATTTAGGAATGTCTAAG
GAGCCAGGTGTTGTTTCTCCAACCAGCTATTTAGATGTGGACTTG
CACCCAGCATCAGCCTGGACATTGTACGCTGTGGCCACTACAGTA
ATAACACCAATGTTAAGACATACCATAGAGAATTCCACAGCAAAT
GTGTCCTTGGCAGCTATAGCCAACCAGGCAGTGGTCCTGATGGGT
TTGGACAAAGGATGGCCAATATCAAAAATGGACTTAGGAGTACCC
CTGCTGGCATTGGGTTGCTATTCACAAGTGAACCCACTGACTCTA
ACAGCGGCAGTACTCTTGCTGATCACACATTATGCCATTATAGGT
CCAGGATTGCAGGCAAAAGCCACCCGTGAAGCTCAGAAAAGGACA
GCTGCTGGAATAATGAAGAATCCAACAGTGGATGGGATAATGACA
ATAGACCTAGATCCTGTAATATATGATTCAAAATTTGAAAAGCAA
CTGGGACAGGTTATGCTCCTGGTTTTGTGTGCAGTTCAACTTTTG
TTAATGAGAACATCATGGGCCTTGTGTGAAGCTTTAACCCTAGCT
ACAGGACCAATAACAACACTCTGGGAAGGATCACCTGGGAAGTTT
TGGAACACCACGATAGCTGTTTCCATGGCGAACATTTTTAGAGGG
AGCTATTTAGCAGGAGCTGGGCTTGCTTTCTCTATTATGAAATCA
GTTGGAACAGGGAAAAGAGGAACAGGCTCACAGGGTGAAACTTTG
GGAGAAAAATGGAAAAAGAAGTTAAATCAATTATCCCGGAAAGAG
TTTGACCTTTACAAGAAATCTGGAATCACTGAGGTGGATAGAACA
GAAGCCAAAGAAGGGTTGAAAAGAGGAGAAATAACACATCATGCC
GTGTCCAGAGGTAGTGCAAAACTTCAATGGTTTGTGGAGAGAAAC
ATGGTCATTCCCGAAGGAAGAGTTATAGACTTGGGCTGTGGAAGA
GGAGGCTGGTCATATTACTGTGCAGGACTGAAAAAAGTCACAGAA
GTGCGAGGATACACAAAAGGCGGTCCAGGACACGAAGAACCAGTA
CCTATGTCCACATATGGATGGAACATAGTTAAGTTAATGAGTGGA
AAGGATGTGTTTTATCTTCCACCTGAAAAGTGTGACACCCTGTTG
TGCGACATTGGAGAATCTTCACCAAGCCCAACAGTGGAAGAAAGC
AGAACTATAAGAGTTTTGAAGATGGTTGAACCATGGCTAAAGAAC
AACCAATTTTGTATTAAAGTATTGAACCCTTACATGCCAACTGTG
ATTGAGCACCTAGAAAGACTACAAAGGAAACATGGAGGAATGCTT
GTGAGAAATCCACTTTCACGAAACTCCACGCACGAAATGTACTGG
ATATCCAATGGCACAGGTAACATTGTCGCTTCAGTCAACATGGTA
TCTAGACTGCTACTGAACAGGTTCACGATGACACACAGAAGACCC
ACCATTGAGAAAGATGTGGATTTAGGAGCAGGAACTCGACATGTT
AATGCGGAACCAGAAACACCCAACATGGATGTCATTGGGGAAAGA
ATAAAAAGGATCAAGGAGGAGCATAATTCAACATGGCACTACGAT
GACGAAAACCCCTACAAAACGTGGGCTTACCATGGATCTTATGAA
GTCAAAGCCACAGGCTCAGCCTCCTCCATGATAAATGGAGTCGTG
AAACTCCTCACTAAACCATGGGATGTGGTGCCCATGGTGACACAG
ATGGCAATGACAGATACAACTCCATTTGGCCAGCAGAGAGTCTTT
AAAGAGAAAGTGGACACCAGGACACCCAGGTCCATGCCAGGAACA
AGAAGGGTTATGGGGATCACAGCGGAGTGGCTCTGGAGAACCCTG
GGAAGGAATAAAAAACCCAGGTTATGCACAAGGGAAGAGTTTACA
AAAAAGGTCAGAACTAACGCAGCCATGGGAGCTGTTTTCACAGAG
GAGAACCAATGGGACAGCGCGAAAGCTGCTGTTGAGGATGAGGAT
TTTTGGAAACTTGTGGACAGAGAACGTGAACTCCACAAACTGGGC
AAGTGTGGAAGCTGTGTTTACAACATGATGGGTAAGAGAGAGAAG
AAACTTGGAGAGTTTGGCAAAGCAAAAGGCAGTAGAGCTATATGG
TACATGTGGTTGGGAGCCAGGTACCTTGAGTTCGAAGCCCTTGGA
TTCTTAAATGAAGACCACTGGTTCTCGCGTGAGAACTCTTACAGT
GGAGTGGAAGGAGAAGGACTGCACAAGCTAGGCTATATATTAAGG
GACATTTCCAAGATACCCGGAGGAGCTATGTATGCTGATGACACA
GCTGGTTGGGACACAAGAATAACAGAAGATGACCTGCACAATGAG
GAAAAGATCACACAGCAAATGGACCCTGAACACAGGCAGTTAGCG
AACGCTATATTTAAGCTCACATACCAAAACAAAGTGGTCAAAGTT
CAACGACCGACTCCAACAGGCACGGTAATGGATATCATATCTAGG
AAAGACCAAAGAGGCAGTGGACAGGTAGGAACTTATGGTCTGAAT
ACATTCACCAACATGGAAGCCCAGTTAATCAGACAAATGGAAGGA
GAAGGTGTGCTGTCAAAGGCAGACCTCGAGAACCCTCATCTGCCA
GAGAAGAAAATTACACAATGGTTGGAAACCAAAGGAGTGGAGAGG
TTAAAAAGAATGGCCATAAGTGGGGATGACTGCGTGGTGAAACCA
ATCGATGACAGGTTCGCTAATGCCCTGCTCGCTCTGAACGACATG
GGAAAGGTTCGGAAAGACATACCTCAATGGCAGCCATCAAAGGGA
TGGCATGATTGGCAACAGGTTCCTTTCTGCTCCCACCACTTTCAT
GAATTGATCATGAAAGATGGAAGAAAGTTGGTGGTTCCCTGCAGA
CCCCAGGACGAACTAATAGGAAGGGCAAGAATCTCTCAAGGAGCG
GGATGGAGCCTTAGAGAAACCGCATGTCTGGGGAAAGCCTACGCT
CAAATGTGGAGTCTCATGTATTTTCACAGAAGAGACCTCAGACTA
GCATCCAACGCCATATGTTCAGCAGTACCAGTCCACTGGGTCCCC
ACAAGTAGAACGACATGGTCTATTCACGCTCACCATCAGTGGATG
ACCACAGAAGACATGCTTACTGTCTGGAACAGAGTGTGGATCGAG
GACAATCCATGGATGGAAGACAAAACTCCAGTCACAACCTGGGAA
AATGTTCCATATCTAGGGAAGAGAGAAGACCAATGGTGCGGATCA
CTTATTGGTCTCACTTCCAGAGCAACCTGGGCCCAGAACATACCC
ACAGCAATTCAACAGGTTAGAAGCCTTATAGGCAATGAAGAGTTT
CTGGACTACATGCCTTCAATGAAGAGATTTAGGAAGGAGGAGGAG
TCGGAGGGAGCCATTTGGTAAAACGTAGGAAGTGAAAAAGAGGTT
AACTGTCAGGCCATATTAAGCCACAGTACGGAAGAAGCTGTGCTG
CCTGTGAGCCCCGTCCAAGGACGTTAAAAGAAGAAGTCAGGCCCC
AAAGCCACGGTTTGAGCAAACCGTGCTGCCTGTAGCTCCGTCGTG
GGGACGTAAAACCTGGGAGGCTGCAAACTGTGGAAGCTGTACGCA
CGGTGTAGCAGACTAGCGGTTAGAGGAGACCCCTCCCATGACACA
ACGCAGCAGCGGGGCCCGAGCACTGAGGGAAGCTGTACCTCCTTG
CAAAGGACTAGAGGTTAGAGGAGACCCCCCGCAAATAAAAACAGC
ATATTGACGCTGGGAGAGACCAGAGATCCTGCTGTCTCCTCAGCA
TCATTCCAGGCACAGAACGCCAGAAAATGGAATGGTGCTGTTGAA
TCAACAGGTTCT
DENV4_Syn_E-W/Min
SEQ ID NO: 12
AGTTGTTAGTCTGTGTGGACCGACAAGGACAGTTCCAAATCGGAA
GCTTGCTTAACACAGTTCTAACAGTTTGTTTAAATAGAGAGCAGA
TCTCTGGAAAAATGAACCAACGAAAAAAGGTGGTTAGACCACCTT
TCAATATGCTGAAACGCGAGAGAAACCGCGTATCAACCCCTCAAG
GGTTGGTGAAGAGATTCTCAACCGGACTTTTTTCTGGGAAAGGAC
CCTTACGGATGGTGCTAGCATTCATCACGTTTTTGCGAGTCCTTT
CCATCCCACCAACAGCAGGGATTCTGAAGAGATGGGGACAGTTGA
AGAAAAATAAGGCCATCAAGATACTGATTGGATTCAGGAAGGAGA
TAGGCCGCATGCTGAACATCTTGAACGGGAGAAAAAGGTCAACGA
TAACATTGTTGTGCTTGATTCCCACCGTAATGGCGTTTCACTTGT
CAACAAGAGATGGCGAACCCCTCATGATAGTGGCAAAACATGAAA
GGGGGAGACCTCTCTTGTTTAAGACAACAGAGGGGATCAACAAAT
GCACTCTCATTGCCATGGACTTGGGTGAAATGTGTGAGGACACTG
TCACGTATAAATGCCCCCTACTGGTCAATACCGAACCTGAAGACA
TTGATTGCTGGTGCAACCTCACGTCTACCTGGGTCATGTATGGGA
CATGCACCCAGAGCGGAGAACGGAGACGAGAGAAGCGCTCAGTAG
CTTTGACACCACATTCAGGAATGGGATTGGAAACAAGAGCTGAGA
CATGGATGTCATCGGAAGGGGCTTGGAAGCATGCTCAGAGAGTAG
AGAGCTGGATACTCAGAAACCCAGGATTTGCGCTCTTGGCAGGAT
TTATGGCTTATATGATTGGGCAAACAGGAATCCAGCGAACTGTCT
TCTTTGTCCTAATGATGCTGGTCGCCCCATCCTACGGAATGCGGT
GCGTAGGAGTAGGAAACAGAGACTTTGTGGAAGGAGTCTCAGGTG
GAGCATGGGTCGACCTGGTGCTAGAACATGGAGGATGCGTCACAA
CCATGGCCCAGGGAAAACCAACCTTGGATTTTGAACTGACTAAGA
CAACAGCTAAGGAAGTGGCTCTGTTAAGAACCTATTGCATTGAAG
CCTCAATATCAAACATAACTACGGCAACAAGATGTCCAACGCAAG
GAGAGCCTTATCTGAAAGAGGAACAGGACCAACAGTACATCTGCC
GGAGAGATGTGGTAGACAGAGGATGGGGCAATGGCTGTGGCTTGT
TTGGAAAAGGAGGAGTTGTGACATGTGCGAAGTTTTCATGTTCGG
GGAAGATAACAGGCAATCTGGTCCAAATTGAGAACCTTGAATACA
CAGTGGTTGTAACAGTCCACAATGGAGACACCCATGCAGTAGGAA
ATGACACATCCAATCATGGAGTTACAGCCACGATAACTCCCAGGT
CACCATCGGTAGAAGTCAAATTGCCGGACTATGGAGAACTAACAC
TCGATTGTGAACCCAGGTCTGGAATTGACTTTAATGAGATGATCC
TAATGAAAATGAAAAAGAAAACATGGCTCGTGCATAAGCAATGGT
TTTTGGATCTGCCTCTTCCATGGACAGCAGGAGCAGACACATCAG
AGGTTCACTGGAATTACAAAGAGAGAATGGTGACGTTCAAGGTTC
CTCATGCCAAGAGACAGGACGTTACAGTGTTAGGGTCACAGGAGG
GAGCTATGCATAGCGCACTAGCCGGAGCAACCGAAGTCGATAGCG
GAGACGGAAATCATATGTTCGCCGGACATCTGAAATGCAAAGTGA
GAATGGAGAAATTGCGGATTAAGGGAATGTCATACACTATGTGTT
CCGGTAAGTTTTCGATTGACAAAGAGATGGCCGAAACGCAACACG
GAACAACAGTCGTTAAGGTTAAGTACGAAGGAGCCGGAGCTCCGT
GTAAAGTGCCAATCGAAATTAGAGACGTTAACAAAGAGAAAGTGG
TCGGAAGAGTGATATCTAGTACACCCCTAGCCGAAAATACGAATT
CCGTTACGAATATCGAACTCGAACCCCCCTTTGGAGACTCATACA
TAGTGATAGGAGTGGGTAATTCCGCACTCACGTTGCACTGGTTCA
GAAAGGGATCATCAATCGGGAAGATGTTCGAATCAACATATAGGG
GAGCGAAAAGAATGGCTATATTGGGCGAAACCGCATGGGACTTCG
GATCAGTCGGAGGACTGTTTACGAGTCTGGGTAAGGCCGTACATC
AGGTATTCGGATCAGTGTATACAACAATGTTTGGCGGAGTGTCAT
GGATGATACGGATACTGATAGGGTTTCTAGTGTTGTGGATCGGAA
CGAACTCACGTAATACGTCTATGGCTATGACATGTATTGCCGTAG
GGGGGATTACACTGTTTCTGGGATTTACAGTGCAAGCAGACATGG
GTTGTGTGGTGTCATGGAGTGGGAGAGAATTGAAGTGTGGAAGCG
GAATTTTTGTGGTTGACAACGTGCACACTTGGACAGAACAGTACA
AATTCCAACCAGAGTCCCCAGCGAGACTAGCGTCTGCAATATTAA
ATGCCCACAAAGATGGGGTCTGTGGAATTAGATCAACCACGAGGC
TGGAAAATGTTATGTGGAAGCAAATAACCAATGAGCTAAACTATG
TTCTCTGGGAAGGAGGACATGATCTCACTGTAGTGGCTGGGGATG
TGAAAGGGGTGTTGACCAAGGGCAAGAGAGCACTCACACCCCCAG
CGAGTGATCTGAAATATTCATGGAAGACATGGGGGAAAGCAAAAA
TCTTCACCCCTGAAGCAAGAAACAGCACATTTTTAATAGACGGAC
CAGACACCTCTGAATGCCCCAATGAACGAAGGGCATGGAATTCTT
TTGAGGTGGAAGACTATGGATTTGGCATGTTCACGACCAACATAT
GGATGAAATTCCGAGAAGGAAGTTCAGAAGTGTGTGACCACAGGT
TAATGTCAGCTGCAATTAAAGACCAGAAAGCTGTGCATGCTGACA
TGGGTTATTGGATAGAGAGCTCAAAAAACCAGACCTGGCAGATAG
AGAGAGCATCTCTTATTGAAGTGAAAACATGTCTGTGGCCCAAGA
CCCATACACTGTGGAGCAATGGAGTGCTGGAAAGCCAGATGCTTA
TTCCAAAATCATATGCAGGCCCTTTTTCACAGCACAATTACCGCC
AGGGCTATGCTACGCAAACCGTGGGTCCATGGCACTTAGGCAAAC
TAGAGATAGACTTTGGAGAATGCCCCGGAACAACAGTCACAATTC
AGGAGAATTGTGACCATAGAGGCCCATCTTTGAGGACCACCACTG
CATCTGGAAAACTAGTCACGCAATGGTGTTGCCGCTCCTGCACGA
TGCCCCCCTTAAGGTTCTTAGGAGAAGATGGGTGCTGGTATGGGA
TGGAGATTAGGCCCTTGAGTGAAAAAGAAGAGAACATGGTCAAAT
CACAGGTGACGGCCGGACAGGGCACATCGGAAACTTTTTCAATGG
GTCTGTTGTGCCTGACCTTGTTTGTGGAAGAATGCTTGAGGAGAA
GAGTCACCAGGAAACACATGATATTAGCTGTGGTAATCACTCTTT
GTGCTATCATCCTGGGGGGCCTCACATGGATGGACTTGCTACGAG
CCCTCATCATGTTGGGGGACACTATGTCTGGTAGAATAGGAGGAC
AGACCCACCTAGCCATCATGGCAGTGTTCAAGATGTCACCGGGAT
ACGTGCTGGGTGTGTTTTTAAGGAAACTCACTTCAAGAGAGACAG
CACTAATGGTAATAGGAATGGCCATGACAACAACACTTTCAATTC
CACATGACCTCATGGAACTCATTGATGGAATATCACTAGGACTAA
TTTTGCTAAAAATAGTAACACAGTTTGACAACACCCAAGTGGGAA
CCTTAGCTCTTTCCTTGACTTTCATAAGATCAACAATGTCATTGG
TCATGGCTTGGAGGACCATTATGGCTGTGTTGTTTGTGGTCACAC
TCATTCCTTTGTGCAGGACAAGCTGTCTTCAAAAACAGTCTCATT
GGGTAGAAATAACAGCACTCATCCTAGGAGCCCAAGCTCTGCCAG
TGTACCTAATGACTCTTATGAAAGGAGCCTCAAGAAGATCTTGGC
CTCTTAACGAAGGCATAATGGCTGTGGGTTTGGTTAGTCTCTTAG
GAAGCGCTCTTTTAAAGAATGATGTCCCTTTAGCTGGCCCAATGG
TGGCAGGAGGCTTACTTCTGGCGGCTTACGTAATGAGTGGCAGCT
CAGCAGATCTGTCACTAGAGAAGGCCGCTAATGTGCAGTGGGATG
AAATGGCAGACATAACAGGCTCAAGTCCAATCATAGAAGTGAAGC
AAGATGAGGATGGCTCTTTCTCCATACGGGACGTCGAGGAAACCA
ATATGATAACCCTTTTGGTGAAACTGGCACTGATAACGGTGTCAG
GTCTCTACCCCTTGGCAATTCCAATCACAATGACCTTATGGTACA
TGTGGCAAGTGAAAACACAAAGATCAGGAGCCCTGTGGGACGTCC
CTTCACCCGCCGCCACTCAAAAAGCCGCACTGTCTGAAGGAGTGT
ACAGGATCATGCAAAGAGGGTTATTCGGGAAAACTCAGGTTGGAG
TAGGGATACACATGGAAGGTGTATTTCACACAATGTGGCATGTTA
CAAGAGGATCGGTGATCTGCCACGAGACTGGGAGATTGGAGCCAT
CTTGGGCTGATGTCAGGAATGACATGATATCATACGGTGGGGGAT
GGAGGCTTGGAGATAAATGGGACAAAGAAGAAGACGTTCAGGTCC
TCGCTATAGAACCAGGGAAAAATCCCAAACATGTCCAAACGAAAC
CTGGCCTTTTCAAGACCCTAACTGGAGAAATTGGAGCAGTAACAT
TAGATTTCAAACCCGGAACGTCTGGTTCTCCCATTATCAACAGGA
AAGGAAAAGTCATCGGACTCTATGGAAATGGAGTGGTCACCAAAT
CAGGTGATTACGTCAGTGCCATAACACAAGCCGAAAGAATTGGAG
AGCCAGATTATGAAGTGGATGAGGACATTTTTCGAAAGAAAAGAC
TAACTATAATGGACTTACACCCCGGAGCCGGAAAGACAAAAAGAA
TTCTTCCATCAATAGTGAGAGAAGCCTTAAAAAGGAGGCTGCGAA
CTTTGATTTTGGCTCCCACGAGAGTGGTGGCGGCCGAGATGGAAG
AGGCCCTACGTGGACTGCCAATCCGTTACCAAACCCCAGCTGTAA
AATCAGAACACACAGGAAGAGAGATTGTAGACCTCATGTGCCATG
CAACCTTCACAACAAGACTTTTGTCATCAACCAGAGTTCCAAACT
ACAACCTTATAGTAATGGATGAAGCACATTTCACCGATCCTTCCA
GTGTCGCGGCTAGAGGATACATTTCGACCAGGGTGGAAATGGGAG
AGGCAGCAGCCATCTTCATGACCGCAACCCCTCCCGGAGCGACAG
ATCCCTTTCCCCAGAGCAACAGCCCAATAGAAGACATCGAGAGAG
AGATTCCGGAAAGGTCATGGAACACAGGGTTCGACTGGATAACAG
ACTACCAAGGGAAAACTGTGTGGTTTGTTCCCAGCATAAAAGCTG
GAAATGACATTGCAAATTGTTTGAGAAAGTCGGGAAAGAAAGTTA
TCCAGTTGAGTAGGAAAACCTTTGATACAGAATATCCAAAAACGA
AACTCACGGACTGGGACTTTGTGGTCACTACAGACATATCTGAAA
TGGGGGCTAACTTTAGAGCTGGGAGAGTGATAGACCCTAGAAGAT
GCCTCAAGCCAGTTATCCTAACAGATGGGCCAGAGAGAGTCATCT
TAGCAGGTCCTATTCCAGTGACTCCAGCAAGCGCTGCTCAGAGAA
GAGGGCGAATAGGAAGGAACCCAGCACAAGAAGACGACCAATACG
TTTTCTCCGGAGACCCACTAAAAAATGATGAAGACCATGCCCACT
GGACAGAAGCAAAGATGCTGCTTGACAATATCTACACCCCAGAAG
GGATCATTCCAACATTGTTTGGTCCGGAAAGGGAAAAAACCCAAG
CTATTGATGGAGAGTTTCGCCTCAGAGGGGAACAAAGGAAGACTT
TTGTGGAATTAATGAGGAGAGGAGACCTTCCGGTGTGGCTGAGTT
ATAAGGTAGCTTCTGCTGGCATTTCTTACAAAGATCGGGAATGGT
GCTTTACTGGGGAAAGAAATAACCAAATTTTAGAAGAAAACATGG
AGGTTGAAATTTGGACTAGAGAGGGAGAAAAGAAAAAACTGAGGC
CAAAATGGTTAGATGCACGTGTATACGCTGACCCCATGGCTTTGA
AGGATTTCAAGGAGTTTGCCAGTGGAAGGAAGAGTATAACTCTCG
ACATCCTAACAGAGATCGCCAGTTTGCCAACTTACCTTTCCTCTA
GGGCCAAGCTCGCCCTTGACAACATAGTTATGCTCCACACAACAG
AAAGAGGAGGGAGGGCCTATCAACATGCCCTGAACGAACTTCCGG
AGTCACTGGAAACACTCATGCTTGTAGCCTTACTAGGAGCTATGA
CAGCAGGTATCTTCCTGTTTTTCATGCAAGGGAAAGGAATAGGGA
AATTGTCAATGGGTTTGATAACCATTGCGGTGGCTAGTGGCTTGC
TCTGGGTAGCAGAAATTCAACCCCAGTGGATAGCGGCCTCAATCA
TACTGGAGTTTTTTCTCATGGTACTGTTGATACCAGAACCAGAAA
AACAAAGGACCCCACAAGACAATCAATTGATCTACGTCATATTGA
CCATTCTCACCATTATTGGTCTAATAGCAGCCAACGAGATGGGGC
TGATAGAAAAAACAAAAACGGATTTTGGGTTTTACCAGGTAAAAA
CAGAAACCACCATCCTCGATGTGGACCTGAGACCAGCTTCAGCAT
GGACGCTCTATGCGGTAGCCACCACAATTCTGACTCCCATGCTGA
GACACACCATAGAAAATACGTCGGCCAACCTATCTTTAGCAGCCA
TCGCCAACCAGGCAGCCGTCCTAATGGGGCTTGGAAAAGGATGGC
CACTCCACAGAATGGACCTCGGTGTGCCGCTGTTAGCAATGGGAT
GCTATTCTCAAGTGAACCCAACAACCTTGACAGCATCCTTAGTCA
TGCTTTTAGTCCATTATGCAATAATAGGCCCAGGATTGCAGGCAA
AAGCCACAAGAGAGGCCCAGAAAAGGACAGCTGCTGGAATCATGA
AAAATCCCACAGTGGACGGGATAACAGTGATAGATCTAGAACCAA
TATCCTATGACCCAAAATTTGAAAAGCAATTAGGGCAGGTCATGT
TACTCGTCTTGTGTGCTGGACAACTACTCTTGATGAGAACAACAT
GGGCTTTCTGTGAAGTTTTGACTTTGGCCACAGGACCAATCTTGA
CCTTGTGGGAGGGCAACCCGGGAAGGTTTTGGAACACGACCATAG
CCGTATCTACCGCCAACATTTTCAGGGGAAGTTATTTGGCAGGAG
CTGGACTGGCTTTTTCACTCATAAAGAATGCACAAACCCCCAGGA
GGGGAACTGGGACCACAGGAGAGACACTGGGAGAGAAGTGGAAGA
GACAGCTAAACTCATTAGACAGGAAAGAGTTTGAAGAGTATAAAA
GAAGTGGAATACTAGAAGTGGACAGGACTGAAGCCAAGTCCGCCC
TGAAAGATGGGTCTAAAATCAAGCATGCAGTATCTAGAGGGTCCA
GTAAGATCAGATGGATTGTTGAGAGAGGGATGGTAAAGCCAAAGG
GGAAAGTTGTAGATCTTGGCTGTGGGAGAGGAGGATGGTCTTATT
ACATGGCGACACTCAAGAACGTGACTGAAGTGAAAGGGTATACAA
AAGGAGGTCCAGGACATGAAGAACCGATTCCCATGGCTACTTATG
GCTGGAATTTGGTCAAACTCCATTCAGGGGTTGACGTGTTCTACA
AACCCACAGAGCAAGTGGACACCCTGCTCTGTGATATTGGGGAGT
CATCTTCTAATCCAACAATAGAGGAAGGAAGAACATTAAGAGTTT
TGAAGATGGTGGAGCCATGGCTCTCTTCAAAACCTGAATTCTGCA
TCAAAGTCCTCAACCCCTACATGCCAACAGTCATAGAGGAGCTGG
AGAAACTGCAGAGAAAACACGGTGGGAACCTTGTCAGATGCCCGC
TGTCCAGGAACTCCACCCATGAGATGTATTGGGTGTCAGGAGCGT
CGGGAAATATTGTGAGCTCTGTGAACACAACATCAAAGATGTTGT
TGAACAGGTTCACAACAAGGCATAGGAAACCCACTTATGAGAAGG
ACGTAGATCTTGGGGCAGGAACGAGAAGTGTCTCTACTGAAACAG
AAAAACCAGACATGACAATCATTGGGAGAAGGCTTCAGCGATTGC
AAGAGGAGCACAAAGAAACATGGCATTATGATCAGGAAAACCCAT
ACAGAACCTGGGCGTATCATGGAAGCTATGAAGCTCCTTCGACAG
GCTCTGCATCCTCCATGGTGAACGGGGTGGTGAAACTGCTAACAA
AACCCTGGGATGTGATTCCAATGGTGACTCAGTTAGCCATGACAG
ATACAACCCCTTTTGGGCAACAAAGAGTGTTCAAAGAGAAGGTGG
ATACCAGAACGCCGCAACCAAAACCAGGCACACGAATGGTTATGA
CCACGACAGCCAATTGGCTATGGGCCCTCCTTGGAAAGAAGAAAA
ATCCCAGACTGTGTACAAGGGAAGAGTTCATCTCAAAAGTTAGAT
CAAACGCAGCCATAGGCGCAGTCTTTCAGGAAGAACAAGGATGGA
CATCAGCCAGTGAAGCTGTGAATGACAGCCGGTTTTGGGAACTGG
TTGACAAAGAAAGGGCCCTTCACCAGGAAGGGAAATGTGAATCGT
GTGTCTATAACATGATGGGAAAACGTGAGAAAAAGTTAGGAGAGT
TTGGCAGAGCCAAGGGAAGCCGAGCAATCTGGTACATGTGGCTGG
GAGCGCGGTTTCTGGAATTTGAAGCCCTGGGTTTTTTGAATGAAG
ATCACTGGTTTGGCAGAGAAAATTCATGGAGTGGAGTGGAAGGGG
AAGGTCTGCACAGATTGGGATACATCCTGGAGGAGATAGACAAGA
AGGATGGAGACCTAATGTATGCTGATGACACAGCAGGTTGGGACA
CAAGAATCACTGAGGATGACCTTCAAAATGAGGAACTGATCACGG
AACAGATGGCTCCCCACCACAAGATCCTAGCCAAAGCCATTTTCA
AACTAACCTATCAAAACAAAGTGGTGAAAGTCCTCAGACCCACAC
CGAGAGGAGCGGTGATGGATATCATATCCAGGAAAGACCAAAGAG
GTAGTGGACAAGTTGGAACATATGGTTTGAACACATTCACCAACA
TGGAAGTTCAACTCATCCGCCAAATGGAAGCTGAAGGAGTCATCA
CACAAGATGACATGCAGAACCCAAAAGGGTTGAAAGAAAGAGTTG
AGAAATGGCTGAGAGAGTGTGGTGTCGACAGGTTAAAGAGGATGG
CAATCAGTGGAGACGATTGCGTGGTGAAGCCCCTAGATGAGAGGT
TTGGCACTTCCCTCCTCTTCTTGAACGACATGGGAAAGGTGAGGA
AAGACATTCCGCAGTGGGAACCATCTAAGGGATGGAAAAACTGGC
AAGAGGTTCCTTTTTGCTCCCATCACTTTCACAAGATCTTTATGA
AGGATGGCCGCTCACTAGTTGTTCCATGTAGAAACCAGGATGAAC
TGATAGGGAGAGCCAGAATCTCGCAGGGGGCTGGATGGAGCTTAA
GAGAAACAGCCTGCCTGGGCAAAGCTTACGCCCAGATGTGGTCGC
TTATGTACTTCCACAGAAGGGATCTGCGTTTAGCCTCCATGGCCA
TATGCTCAGCAGTTCCAACGGAATGGTTTCCAACAAGCAGAACAA
CATGGTCAATCCATGCTCATCACCAATGGATGACCACTGAAGACA
TGCTCAAAGTGTGGAACAGAGTGTGGATAGAAGACAACCCCAATA
TGATTGACAAGACTCCAGTCCATTCGTGGGAAGATATACCTTACC
TAGGGAAAAGAGAGGATTTGTGGTGTGGATCCCTGATTGGACTTT
CTTCTAGAGCCACCTGGGCGAAGAACATTCACACGGCCATAACTC
AGGTCAGGAACTTGATCGGAAAAGAGGAATACGTGGATTACATGC
CAGTAATGAAAAGATACAGTGCTCCTTCAGAGAGTGAAGGAGTTC
TGTAATTACCAACAACAAACACCAAAGGCTATTGAAGTCAGGCCA
CTTGTGCCACGGTTTGAGCAAACCGTGCTGCCTGTAGCTCCGCCA
ATAATGGGAGGCGTAATAATCCCTAGGGAGGCCATGCGCCACGGA
AGCTGTACGCGTGGCATATTGGACTAGCGGTTAGAGGAGACCCCT
CCCATCACTGACAAAACGCAGCAAAAGGGGGCCCGAAGCCAGGAG
GAAGCTGTACTCCTGGTGGAAGGACTAGAGGTTAGAGGAGACCCC
CCCAACACAAAAACAGCATATTGACGCTGGGAAAGACCAGAGATC
CTGCTGTCTCTACAACATCAATCCAGGCACAGAGCGCCGCAAGAT
GGATTGGTGTTGTTGATCCAACAGGTTCT

The present invention provides a composition for inducing an immune response in a subject comprising any of the attenuated viruses described herein and a pharmaceutically acceptable carrier. In various embodiments, the composition is a vaccine composition and the immune response is a protective immune response.

It should be understood that an attenuated virus of the invention, where used to elicit an immune response in a subject (or protective immune response) or to prevent a subject from or reduce the likelihood of becoming afflicted with a virus-associated disease, is administered to the subject in the form of a composition additionally comprising a pharmaceutically acceptable carrier. Pharmaceutically acceptable carriers are well known to those skilled in the art and include, but are not limited to, one or more of 0.01-0.1M and preferably 0.05M phosphate buffer, phosphate-buffered saline (PBS), or 0.9% saline. Such carriers also include aqueous or non-aqueous solutions, suspensions, and emulsions. Aqueous carriers include water, alcoholic/aqueous solutions, emulsions or suspensions, saline and buffered media. Examples of non-aqueous solvents are propylene glycol, polyethylene glycol, vegetable oils such as olive oil, and injectable organic esters such as ethyl oleate. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Solid compositions may comprise nontoxic solid carriers such as, for example, glucose, sucrose, mannitol, sorbitol, lactose, starch, magnesium stearate, cellulose or cellulose derivatives, sodium carbonate and magnesium carbonate. For administration in an aerosol, such as for pulmonary and/or intranasal delivery, an agent or composition is preferably formulated with a nontoxic surfactant, for example, esters or partial esters of C6 to C22 fatty acids or natural glycerides, and a propellant. Additional carriers such as lecithin may be included to facilitate intranasal delivery. Pharmaceutically acceptable carriers can further comprise minor amounts of auxiliary substances such as wetting or emulsifying agents, preservatives and other additives, such as, for example, antimicrobials, antioxidants and chelating agents, which enhance the shelf life and/or effectiveness of the active ingredients. The instant compositions can, as is well known in the art, be formulated so as to provide quick, sustained or delayed release of the active ingredient after administration to a subject.

In various embodiments of the instant composition or vaccine composition, the attenuated virus (i) does not substantially alter the synthesis and processing of viral proteins in an infected cell; (ii) produces similar amounts of virions per infected cell as wt virus; and/or (iii) exhibits substantially lower virion-specific infectivity than wt virus. In further embodiments, the attenuated virus induces a substantially similar immune response in a host animal as the corresponding wt virus.

This invention also provides a modified host cell line specially isolated or engineered to be permissive for an attenuated virus that is inviable in a wild type host cell. IN embodiments wherein the attenuated virus cannot grow in normal (wild type) host cells, it is absolutely dependent on the specific helper cell line for growth. This provides a very high level of safety for the generation of virus for vaccine production. Various embodiments of the instant modified cell line permit the growth of an attenuated virus, wherein the genome of said cell line has been altered to increase the number of genes encoding rare tRNAs.

### Methods of Eliciting an Immune Response

Various embodiments provide for a method of eliciting an immune response in a subject comprising administering to the subject an effective dose of a composition comprising a modified Flavivirus of the present invention. Particular embodiments provide for a method of eliciting a protective immune response in a subject comprising administering to the subject a prophylactically or therapeutically effective dose of a vaccine composition comprising a modified Flavivirus of the present invention. In various embodiments, the immune response is cross-protective against a heterologous Flavivirus virus.

In various embodiments, the method further comprises administering to the subject at least one adjuvant. Non-limiting examples of adjuvants are discussed herein. Particular embodiments provide for a method of eliciting an immune response in a subject, comprising: administering to the subject a prophylactically or therapeutically effective dose of a vaccine composition comprising a modified dengue virus the present invention.

Various embodiments provide for a method of eliciting an immune response in a subject, comprising: administering to the subject an effective dose of a composition comprising a modified dengue virus the present invention.

Various embodiments provide for a method of eliciting a protective immune response in a subject, comprising: administering to the subject a prophylactically or therapeutically effective dose of a vaccine composition comprising a modified dengue virus the present invention.

In various embodiments, the method further comprises administering to the subject at least one adjuvant. Non-limiting examples of adjuvants are described herein.

In various embodiments, the immune response is cross-protective against a heterologous dengue virus.

Various embodiments of the present invention provide for a method of eliciting an immune response in a subject in need thereof, comprising: administering a prime dose of an attenuated Flavivirus produced by a method other than codon-pair deoptimization or codon deoptimization, or increasing of CpG or UpA di-nucleotides, or a modified Flavivirus in which expression of viral proteins is reduced compared to a parent virus, wherein the reduction in expression is the result of recoding the prM, or envelope (E) region; and administering one or more boost dose of the attenuated Flavivirus produced by methods other than codon-pair deoptimization or codon deoptimization, or increasing of CpG or UpA di-nucleotides, or the modified Flavivirus to the subject in need thereof, wherein at least the prime dose or the one or more boost dose is the modified virus. In various embodiments, a first of the one or more boost dose is administered about 2 weeks after the prime dose.

In various embodiments, the Flavivirus is a dengue virus.

In various embodiments, one or both of the E protein-encoding sequence protein-encoding sequence is recoded by reducing the codon pair bias or codon usage bias of the protein-encoding sequence. In various embodiments, reducing the codon-pair bias comprises identifying a codon pair in the parent protein-encoding sequence having a codon-pair score that can be reduced, and reducing the codon-pair bias by substituting the codon pair with a codon pair that has a lower codon-pair score.

In various embodiments, reducing the codon-pair bias comprises rearranging the codons of a parent protein-encoding sequence. In various embodiments, the E protein-encoding sequence is recoded by increasing the number of CpG or UpA di nucleotides compared to a parent virus. In various embodiments, each of the recoded prM/E protein-encoding sequence have a codon pair bias less than −0.05, or less than −0.06, or less than −0.07, or less than −0.08, or less than −0.09, or less than −0.1, or less than −0.11, or less than −0.12, or less than −0.13, or less than −0.14, or less than −0.15, or less than −0.16, or less than −0.17, or less than −0.18, or less than −0.19, or less than −0.2, or less than −0.25, or less than −0.3, or less than −0.35, or less than −0.4, or less than −0.45, or less than −0.5. In various embodiments, one or both of the E protein-encoding sequence is recoded by replacing one or more codons with synonymous codons that are less frequent in the viral host (e.g., human). In some embodiments, the number of codons substituted with synonymous codons is at least 5, or at least 10, or at least 30, or at least 30, or at least 40, or at least 50, or at least 75, or at least 100, or at least 200 or at least 300, or at least 400, or at least 500.

In addition, the present invention provides a method for eliciting a protective immune response in a subject comprising administering to the subject a prophylactically or therapeutically effective dose of any of the vaccine compositions described herein. This invention also provides a method for preventing a subject from becoming afflicted with a virus-associated disease comprising administering to the subject a prophylactically effective dose of any of the instant vaccine compositions. In embodiments of the above methods, the subject has been exposed to a pathogenic virus. “Exposed” to a pathogenic virus means contact with the virus such that infection could result.

The invention further provides a method for delaying the onset, or slowing the rate of progression, of a virus-associated disease in a virus-infected subject comprising administering to the subject a therapeutically effective dose of any of the instant vaccine compositions.

As used herein, “administering” means delivering using any of the various methods and delivery systems known to those skilled in the art. Administering can be performed, for example, intranasally, intraperitoneally, intracerebrally, intravenously, orally, transmucosally, subcutaneously, transdermally, intradermally, intramuscularly, topically, parenterally, via implant, intrathecally, intralymphatically, intralesionally, pericardially, or epidurally. An agent or composition may also be administered in an aerosol, such as for pulmonary and/or intranasal delivery. Administering may be performed, for example, once, a plurality of times, and/or over one or more extended periods.

Eliciting a protective immune response in a subject can be accomplished, for example, by administering a primary dose of a vaccine to a subject, followed after a suitable period of time by one or more subsequent administrations of the vaccine. A suitable period of time between administrations of the vaccine may readily be determined by one skilled in the art, and is usually on the order of several weeks to months. The present invention is not limited, however, to any particular method, route or frequency of administration.

In various embodiments, the present invention provides for a method of eliciting an immune response in a subject in need thereof, comprising: administering a prime dose of an attenuated Flavivirus produced by a method other than codon-pair deoptimization or codon deoptimization, or increasing of CpG or UpA di-nucleotides, or a modified Flavivirus in which expression of viral proteins is reduced compared to a parent virus, wherein the reduction in expression is the result of recoding the prM, or envelope (E) region, or both; and administering one or more boost dose of the attenuated Flavivirus by methods other than codon-pair deoptimization or codon deoptimization, or increasing of CpG or UpA di-nucleotides, or the modified Flavivirus to the subject in need thereof, wherein at least the prime dose or the one or more boost dose is the modified virus.

In various embodiments, the one or more boost dose is administered about 2 weeks after a prime dose. In various embodiments, 2, 3, 4, or 5 boost doses are administered. In various embodiments, the intervals between the boost doses can be 1, 2, 3, 4, 5, 6, 7, 8, 9, or 10 weeks. In additional embodiments, the intervals between the boost doses can be 1, 2, 3, 4, 5, 6, 7, 8, 9, 10, 11 or 12 months. As a non-limiting example, the prime dose can be administered, about two weeks thereafter a first boost dose can be administered, about one month after the first boost dose, a second boost dose can be administered, about 6 months after the second boost dose, a third boost dose can be administered. As another non-limiting example, the prime dose can be administered, about two weeks thereafter a first boost dose can be administered, about six months after the first boost dose, a second boost dose can be administered, about 12 months after the second boost dose, a third boost dose can be administered. In further embodiments, additional boost dosages can be periodically administered; for example, every 5 years, every 10 years, etc.

In various embodiments, the Flavivirus is a dengue virus. In various embodiments, the Flavivirus is selected from the group consisting of dengue fever virus, Zika virus, West Nile virus, yellow fever virus, Japanese encephalitis virus, Spondweni virus, Saint Louis encephalitis virus, and Powassan virus.

In various embodiments, one or both of the E protein-encoding sequence is recoded by lowering the codon pair bias or codon usage bias of the protein-encoding sequence. In various embodiments, the E protein-encoding sequence is recoded by increasing the number of CpG or UpA di nucleotides compared to a parent virus.

In various embodiments, reducing the codon-pair bias comprises identifying a codon pair in the parent protein-encoding sequence having a codon-pair score that can be reduced, and reducing the codon-pair bias by substituting the codon pair with a codon pair that has a lower codon-pair score.

In various embodiments, reducing the codon-pair bias comprises rearranging the codons of a parent protein-encoding sequence.

In various embodiments it includes the increase of the CpG dinucleotide in the modified virus

In various embodiments it includes the increase of the UpA dinucleotide in the modified virus

In various embodiments, the recoded prM/E protein-encoding sequence each have a codon pair bias less than −0.05, or less than −0.06, or less than −0.07, or less than −0.08, or less than −0.09, or less than −0.1, or less than −0.11, or less than −0.12, or less than −0.13, or less than −0.14, or less than −0.15, or less than −0.16, or less than −0.17, or less than −0.18, or less than −0.19, or less than −0.2, or less than −0.25, or less than −0.3, or less than −0.35, or less than −0.4, or less than −0.45, or less than −0.5.

In certain embodiments, the codon pair bias of the recoded prM-protein encoding sequence, E protein-encoding sequence, or both is reduced by at least 0.05, or at least 0.06, or at least 0.07, or at least 0.08, or at least 0.09, or at least 0.1, or at least 0.11, or at least 0.12, or at least 0.13, or at least 0.14, or at least 0.15, or at least 0.16, or at least 0.17, or at least 0.18, or at least 0.19, or at least 0.2, or at least 0.25, or at least 0.3, or at least 0.35, or at least 0.4, or at least 0.45, or at least 0.5, compared to the parent prM/E protein encoding sequence from which it is derived.

In various embodiments, the prM-protein encoding sequence, E protein-encoding sequence, or both are recoded by replacing one or more codons with synonymous codons that are less frequent in the viral host. In some embodiments, the number of codons substituted with synonymous codons is at least 5, or at least 10, or at least 30, or at least 30, or at least 40, or at least 50, or at least 75, or at least 100, or at least 200 or at least 300, or at least 400, or at least 500.

In various embodiments, the prime dose is administered subcutaneously, intramuscularly, intradermally, or intranasally.

In various embodiments, the one or more boost dose is administered intratumorally, intravenously, or intrathecally.

The timing between the prime and boost dosages can vary, for example, depending on the stage of infection or disease (e.g., non-infected, infected, number of days post infection), and the patient's health. In various embodiments, the one or more boost dose is administered about 2 weeks after the prime dose. That is, the prime dose is administered and about two weeks thereafter, a boost dose is administered.

In various embodiments, the dosage amount can vary between the prime and boost dosages. As a non-limiting example, the prime dose can contain fewer copies of the virus compared to the boost dose.

In other embodiments, the type of attenuated virus produced by a method other than codon-pair deoptimization or codon deoptimization, or increasing of CpG or UpA di-nucleotides or modified virus of the present invention can vary between the prime and boost dosages. In one non-limiting example, a modified virus of the present invention can be used in the prime dose and an attenuated virus (produced by a method other than codon-pair deoptimization or codon deoptimization, or increasing of CpG or UpA di-nucleotides) of the same or different family, genus, species, group or order can be used in the boost dose.

In other embodiments, the route of administration can vary between the prime and the boost dose. In a non-limiting example, the prime dose can be administered subcutaneously, and the boost dose can be administered via injection into the tumor; for tumors that are in accessible, or are difficult to access, the boost dose can be administered intravenously.

Certain embodiments of any ofthe instant immunization and therapeutic methods further comprise administering to the subject at least one adjuvant. An “adjuvant” shall mean any agent suitable for enhancing the immunogenicity of an antigen and boosting an immune response in a subject. Numerous adjuvants, including particulate adjuvants, suitable for use with both protein- and nucleic acid-based vaccines, and methods of combining adjuvants with antigens, are well known to those skilled in the art. Suitable adjuvants for nucleic acid based vaccines include, but are not limited to, Quil A, imiquimod, resiquimod, and interleukin-12 delivered in purified protein or nucleic acid form. Adjuvants suitable for use with protein immunization include, but are not limited to, alum, Freund's incomplete adjuvant (FIA), saponin, Quil A, and QS-21.

The invention also provides a kit for immunization of a subject with an attenuated virus of the invention. The kit comprises the attenuated virus, a pharmaceutically acceptable carrier, an applicator, and an instructional material for the use thereof. In further embodiments, the attenuated virus may be one or more dengue virus, one or more Japanese encephalitis virus, one or more West Nile virus, one or more yellow fever virus, one or more Zika virus, etc. More than one virus may be preferred where it is desirable to immunize a host against a number of different isolates of a particular virus. The invention includes other embodiments of kits that are known to those skilled in the art. The instructions can provide any information that is useful for directing the administration of the attenuated viruses.

Throughout this application, various publications, reference texts, textbooks, technical manuals, patents, and patent applications have been referred to. The teachings and disclosures of these publications, patents, patent applications and other documents in their entireties are hereby incorporated by reference into this application to more fully describe the state of the art to which the present invention pertains. However, the citation of a reference herein should not be construed as an acknowledgement that such reference is prior art to the present invention.

It is to be understood and expected that variations in the principles of invention herein disclosed can be made by one skilled in the art and it is intended that such modifications are to be included within the scope of the present invention. The following Examples further illustrate the invention but should not be construed to limit the scope of the invention in any way. Detailed descriptions of conventional methods, such as those employed in the construction of recombinant plasmids, transfection of host cells with viral constructs, polymerase chain reaction (PCR), and immunological techniques can be obtained from numerous publications, including Sambrook et al. (1989) and Coligan et al. (1994). All references mentioned herein are incorporated in their entirety by reference into this application. The contents of WO 2008/121992 and WO 2011/044561 are incorporated by reference.

### EXAMPLES

### Example 1

Rapid design and construction of SAVE-deoptimized, live-attenuated dengue vaccine candidates. To select our target sequences, we started with all prM-E sequences of all DENV virus isolates ofthe last decade and performed an amino acid consensus sequence analysis, available at the NCBI Virus Variation Database. As the consensus sequence usually never exists as an actual virus, it is unwise to use it as the basis of our designs. We prefer natural virus evolution to tell us what works and what does not. Thus, the consensus sequence for each serotype is then searched against the entire Genbank using the tblastn algorithm to identify an actual genome of a replicating virus which is most identical to the consensus sequence; we then used this sequence as our target sequence. In addition, the target sequence must not be unique in the database (possible sequencing artifacts), in which case we select the next highest identity sequence to the consensus, which is represented by two or more independent virus isolates. This search algorithm to determine the most relevant and replicating human isolate result in a sequence that is 1) a genuine human isolate and 2) as homologous to the rest of DENV sequence space as a single sequence can be. Using this approach, we identified the following viruses from which to select out target prM-E antigen sequences: DENV-1/VN/BID-V1774/2007, DENV-2/NI/BID-V533/2005, DENV-3/VE/BID-V2268/2008, DENV-4/US/BID-V2448/1999. We initiated our DENV vaccine program using the E-Min of DENV-2/NI/BID-V533/2005 and generated live-attenuated strains containing the other three E genes selected via this process (FIG. 1). The CPB of wild-type prM/E genes fall within the normal range of human host cell's genes. Following codon pair-‘deoptimization’ via SAVE, the resulting deoptimized prM/E gene segments were now encoded predominately by under-represented human codon-pairs as evident by their extremely negative CPB, and are drastically different from any human gene. The SAVE-deoptimized synthetic prM/E from FIG. 1 were synthesized de novo and using overlapping PCR subcloned individually into the WT DENV genomes—yielding six independent cDNA genomes each containing a synthetically ‘de-optimized’ fragment(s). We constructed infectious cDNA genomes for wt DENV1-4 and then recovered fully infectious, replicating virus for the deoptimized DENV vaccine candidates via transfection of RNA into Vero or BHK cells.

### Example 2

### Leveraging SAVE Platformflexibility to Create Second-Generation Dengue Virus Vaccine Candidates

To fine-tune attenuation and immunogenicity, a second generation of Dengue virus vaccine candidates were constructed using the SAVE platform (FIG. 2). The second generation of dengue vaccine candidates leverages the flexibility of the SAVE platform to reduce the deoptimized region from 2014 bp (E-min) to 997 bp (W-E-Min) or further to 664 bp (W-W-E-Min) while keeping the amino acid sequence 100% identical in a homologous backbone.

### Example 3

### Growth of Heterologous Backbone Dengue Vaccine Candidates in Vero Cells Under Animal-Component-Free Conditions

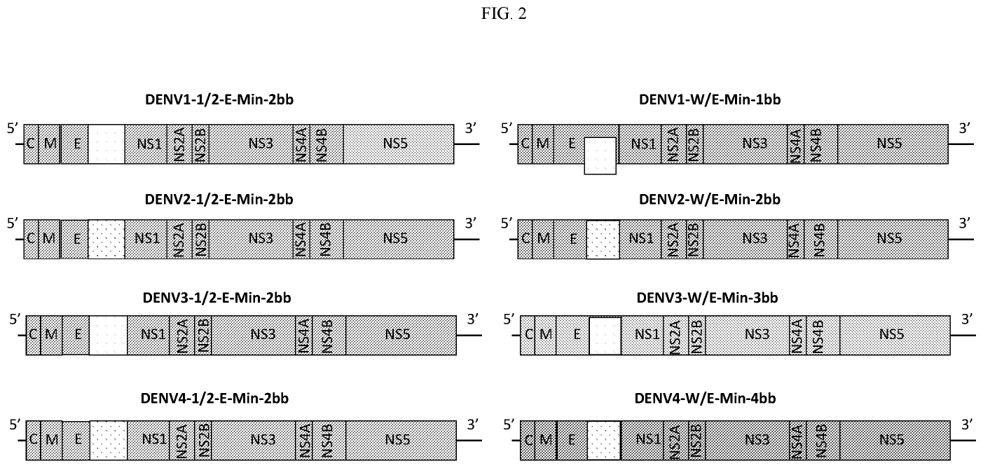

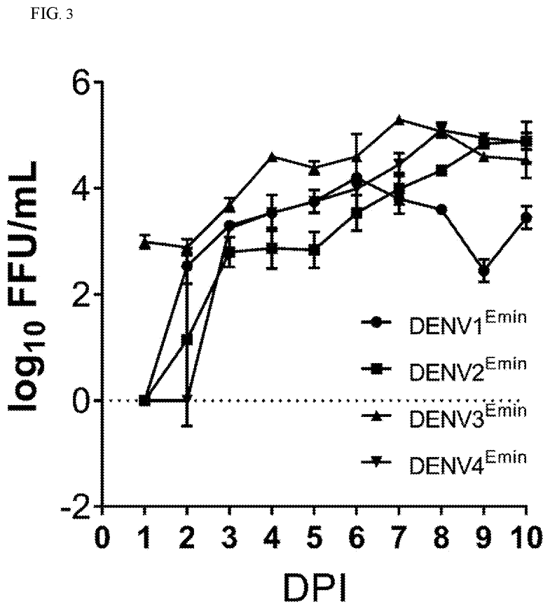

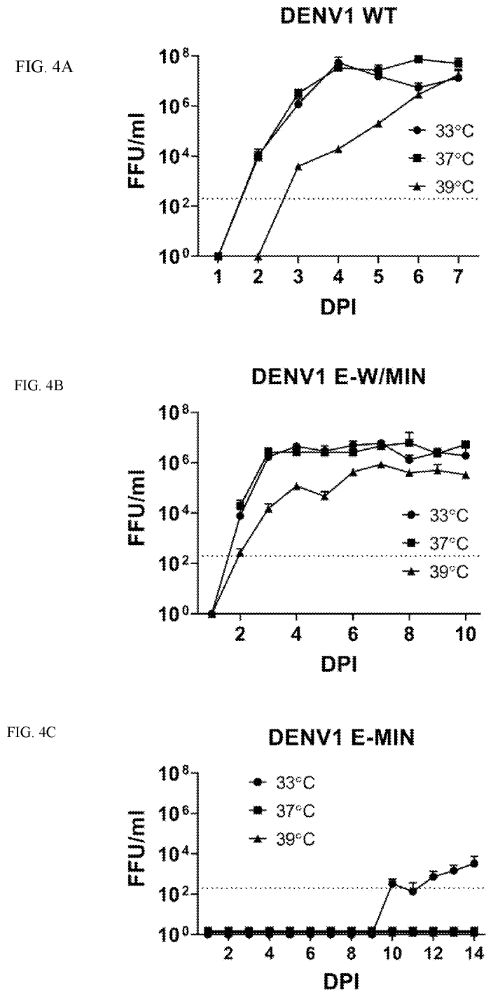

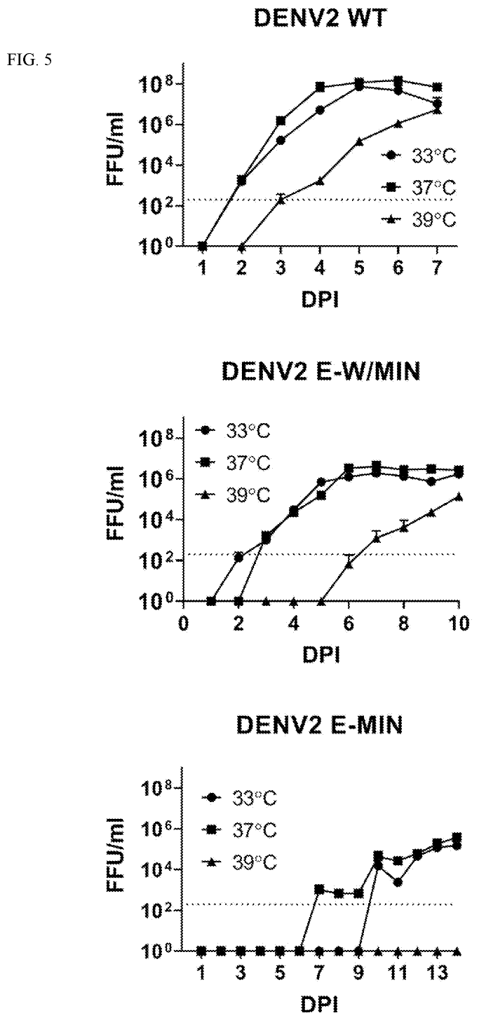

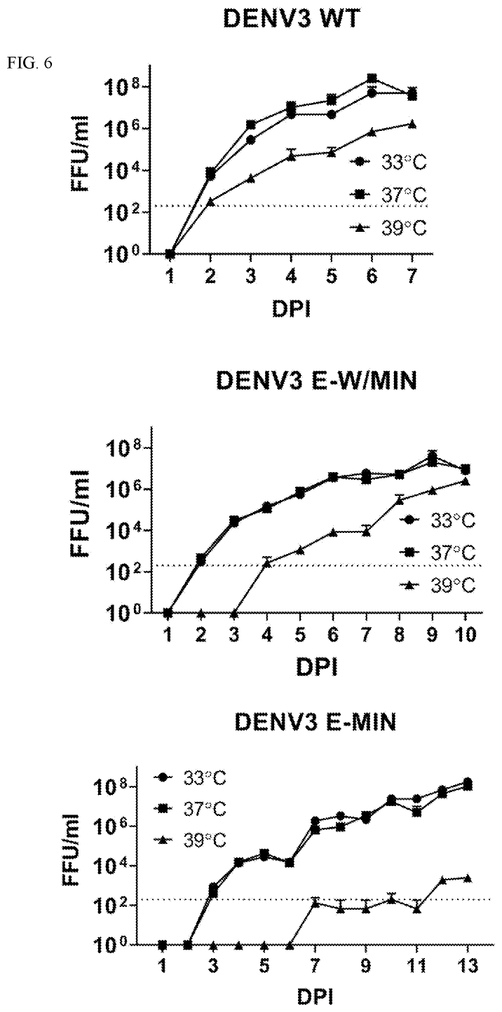

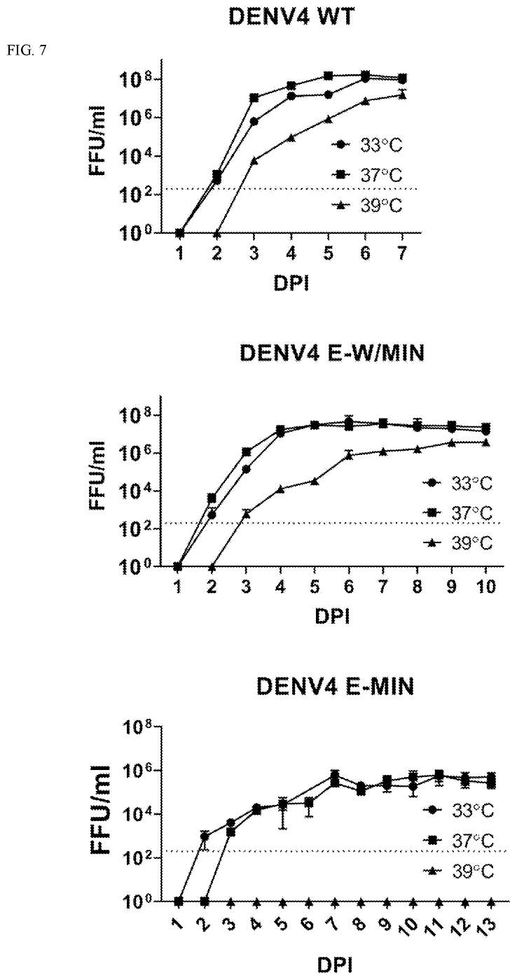

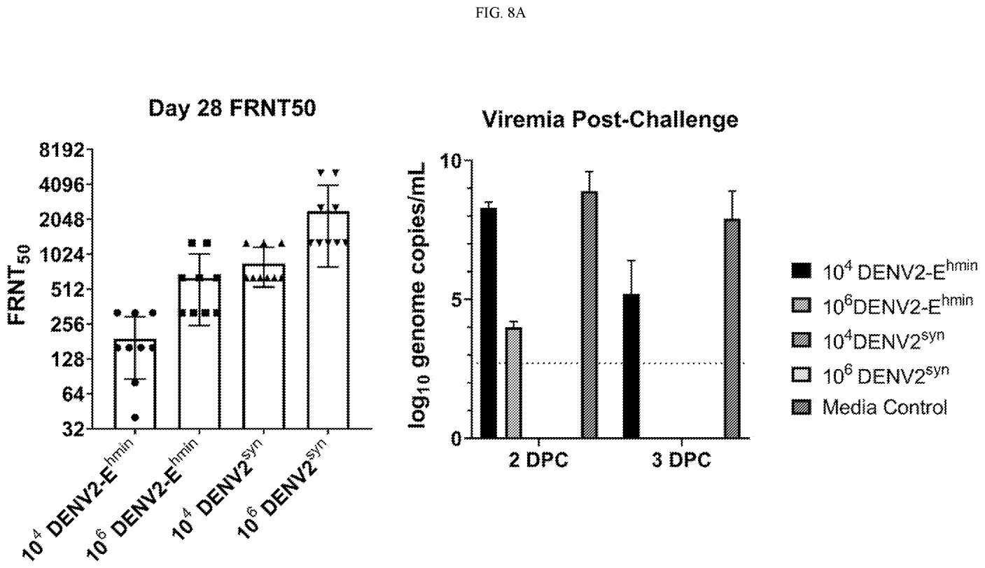

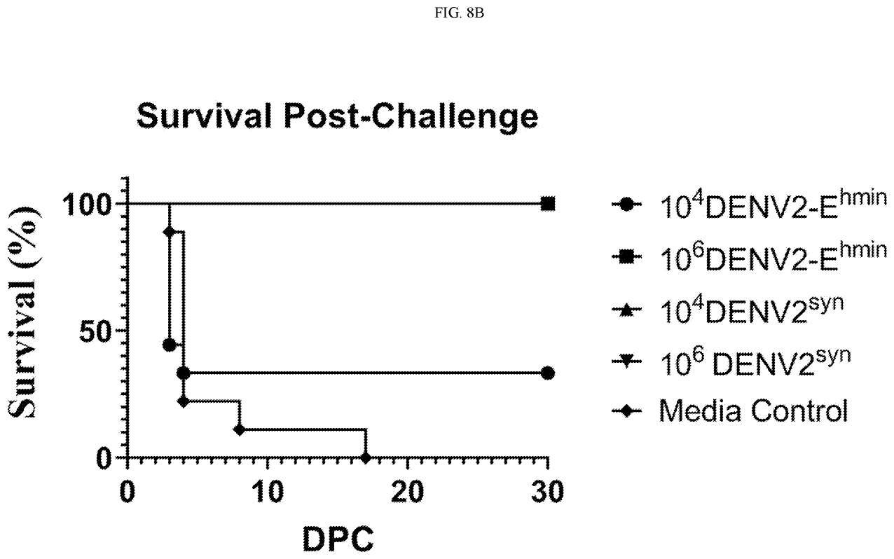

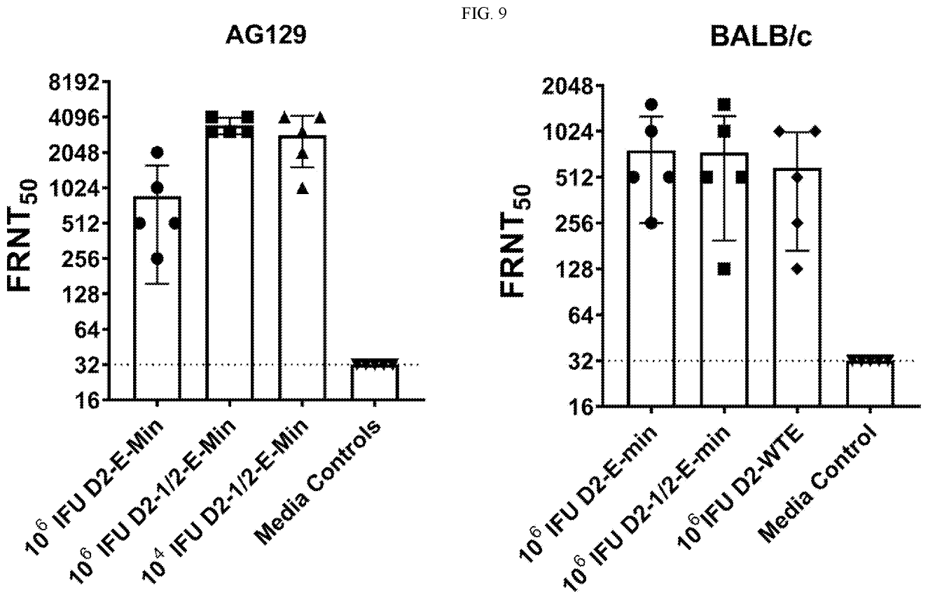

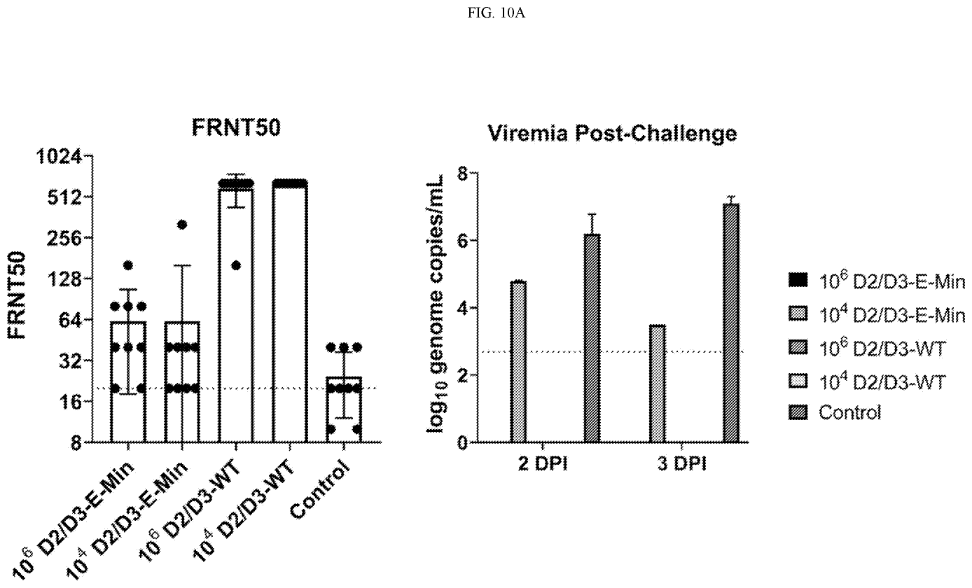

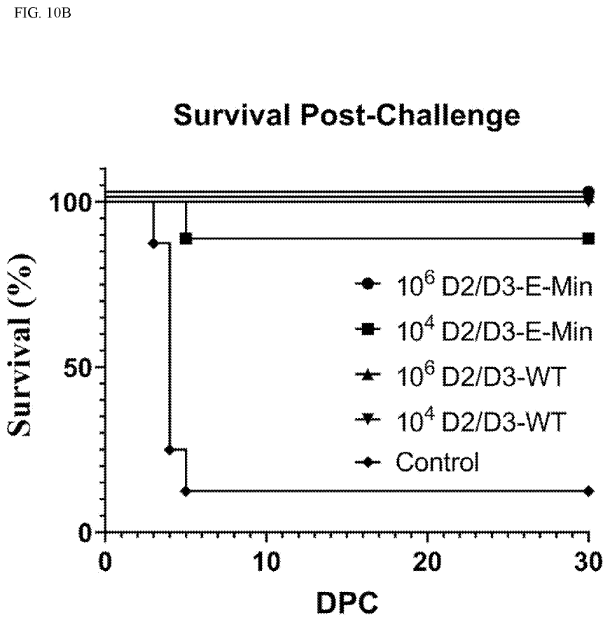

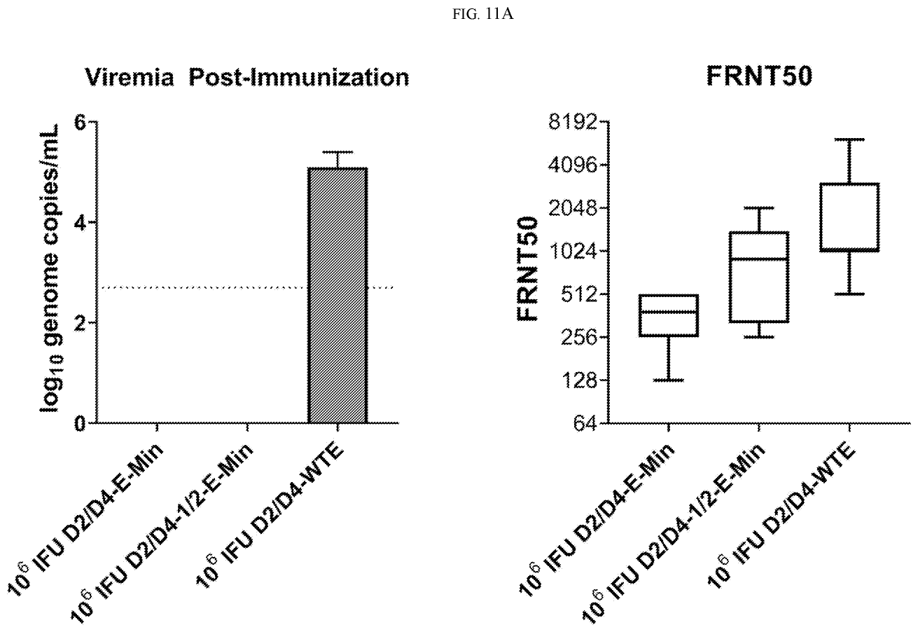

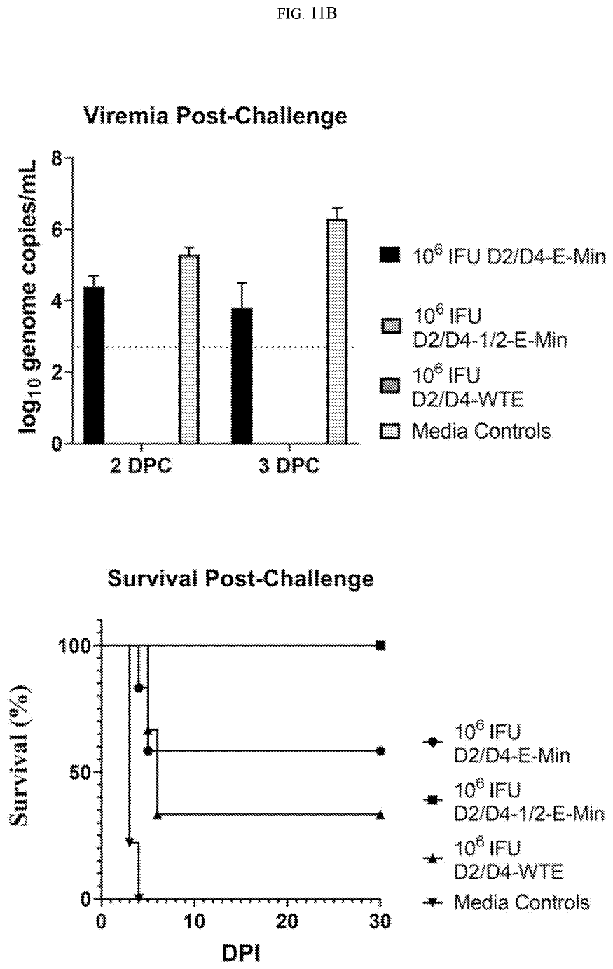

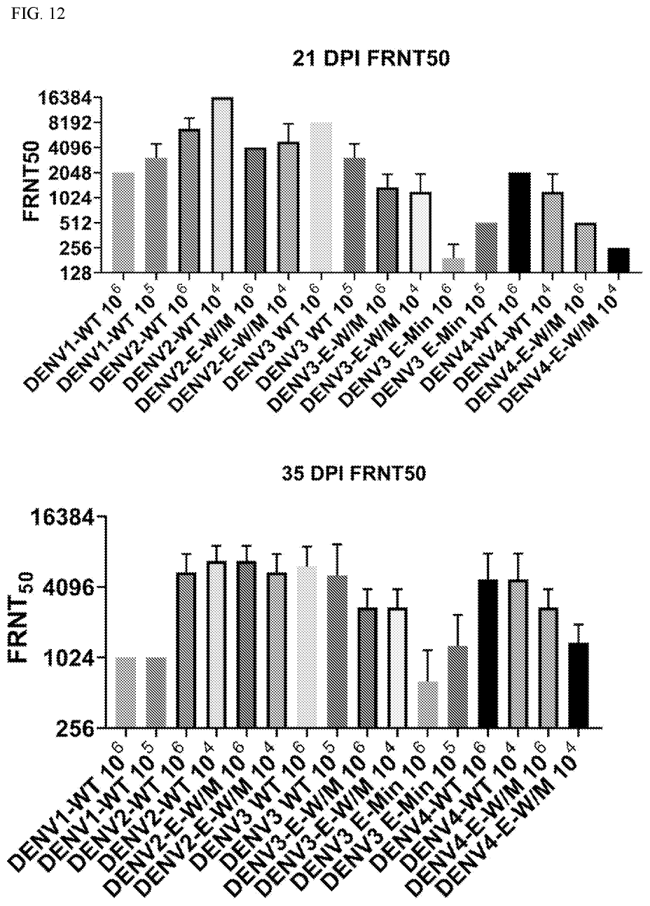

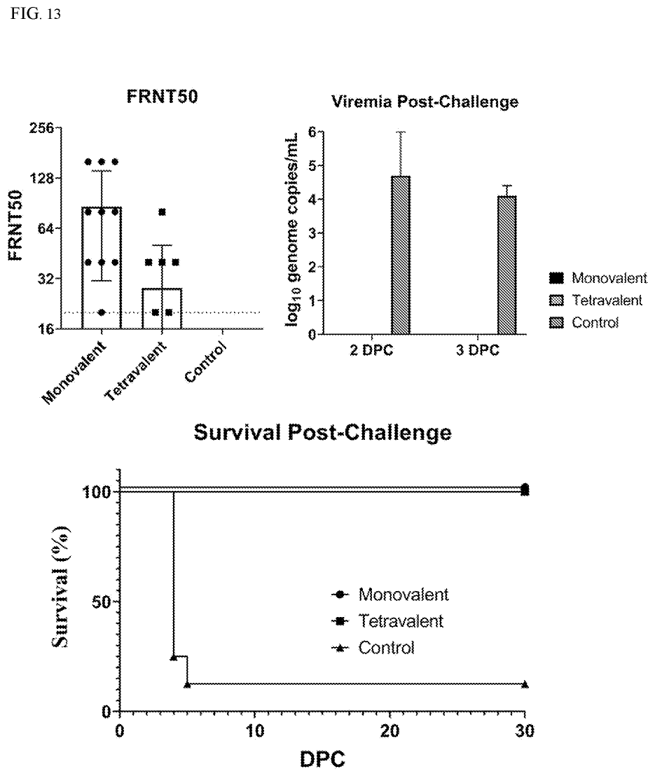

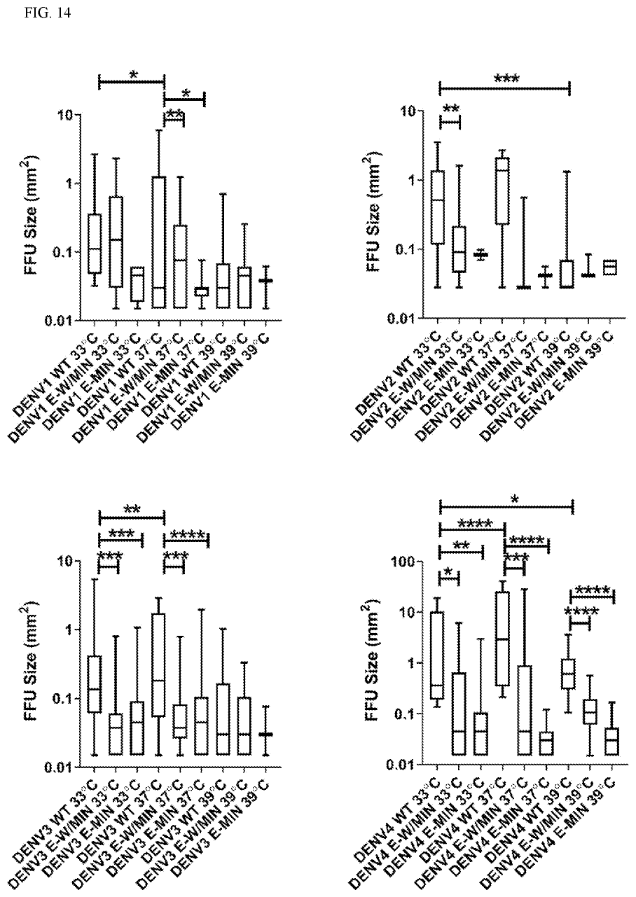

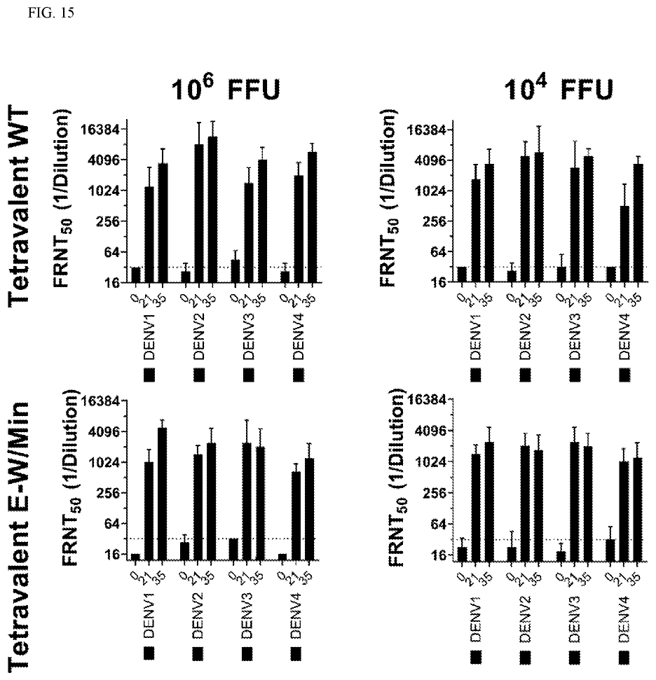

To optimize production conditions for future cGMP manufacture and test for attenuation in vitro, DENV1-4 E-Min were used to infect Vero cells under animal-component free conditions at a MOI of 0.01 and supernatant titrated daily in a multiple-step growth curve over the course of 10 days post-infection. DENV2, DENV3, and DENV4 candidates reached titers of 1-2×10⁵ FFU/ml while DENV1 E-Min titer was reduced by ˜1 log₁₀ compared to the others. (FIG. 3).

### Example 4

### Growth of Homologous Backbone Dengue Virus Type 1 Vaccine Candidates in Vero Cells and Tests for Temperature-Sensitivity as a Mechanism of Attenuation

Growth kinetics conducted at low (33° C.), medium (37° C.), and high (39° C.) temperatures were used to optimize production conditions for the deoptimized candidates, test for attenuation in vitro and examine CPD variants for increase temperature sensitivity as a means of explaining the mechanism of attenuation. Multiple step growth curves were conducted in Vero cells for synthetic DENV1 WT virus (in a full-length DENV1 backbone) and attenuated live vaccine candidates DENV1 E-W/MIN and E-MIN derived from DENV1 WT. Synthetic DENV1 WT grows well in Vero cells at 33° C. and 37° C. Typical of DENY, a transient reduction in virus yield was observed at 39° C., however, titers recovered to 37° C. levels by 7 dpi. DENV1 E-W/MIN, however, reached serviceable titers ˜10-fold lower than DENV1 WT at both 33° C. and 37° C. DENV1 E-MIN was highly attenuated in Vero cells and only reached detectable titers at 33° C. starting on days 10-14 post-infection.

### Example 5

### Growth of Homologous Backbone Dengue Virus Type 2 Vaccine Candidates in Vero Cells and Tests for Temperature-Sensitivity as a Mechanism of Attenuation

Growth kinetics conducted at low (33° C.), medium (37° C.), and high (39° C.) temperatures were used to optimize production conditions for the deoptimized candidates, test for attenuation in vitro and examine CPD variants for increase temperature sensitivity as a means of explaining the mechanism of attenuation. Multiple step growth curves were conducted in Vero cells for synthetic DENV2 WT virus (in a full-length, homologous DENV2 backbone) and attenuated live vaccine candidates DENV2 E-W/MIN and E-MIN derived from DENV2 WT. DENV2 E-W/MIN and E-MIN were both attenuated in vitro with a 1-2 log₁₀ FFU/ml reduction for E-W/MIN and a more pronounced 2-3 log₁₀ FFU/ml reduction for DENV2 E-MIN (FIG. 3). While growth kinetics were delayed, with regular medium replacement every 1-2 days virus titers reached >10⁶ FFU/ml for DENV2 E-W/MIN. Importantly, attenuation was correlated with the extent of deoptimization in the E region. While temperature sensitivity (difference between titers at 37° C. and 39° C.) was similar for DENV2-WT and DENV2 E-W/MIN through day 4 post-infection, there were significant differences on days 5, 6, and 7. Temperature sensitivity was not observed for DENV2 E-MIN because there was no replication at any temperature through 6 DPI. However, starting at day 7-14 DENV2 E-MIN started producing detectable virus (but only at 33° C. and 37° C.).

### Example 6

### Growth of Homologous Backbone Dengue Virus Type 3 Vaccine Candidates in Vero Cells and Tests for Temperature-Sensitivity as a Mechanism of Attenuation

Growth kinetics conducted at low (33° C.), medium (37° C.), and high (39° C.) temperatures were used to optimize production conditions for the deoptimized candidates, test for attenuation in vitro and examine CPD variants for increase temperature sensitivity as a means of explaining the mechanism of attenuation. Multiple step growth curves were conducted in Vero cells for synthetic DENV3 WT virus (in a full-length DENV3 backbone) and attenuated live vaccine candidates DENV3 E-W/MIN and E-MIN derived from DENV3 WT. It was apparent that DENV3 E-W/MIN was more temperature sensitive at 39° C. than the WT with a significantly greater difference observable on days 3, 4, 5, and 7 post-infection. Because DENV3 E-MIN had minimal levels of replication in Vero cells at 39° C., the difference in virus yield from 37° C. to 39° C. were as high as 10⁷ FFU/ml. When grown at permissible temperatures, however, DENV3 E-MIN reached high titers which is promising for cGMP manufacture.

### Example 7

### Growth of Homologous Backbone Dengue Virus Type 4 Vaccine Candidates in Vero Cells and Tests for Temperature-Sensitivity as a Mechanism of Attenuation

Growth kinetics conducted at low (33° C.), medium (37° C.), and high (39° C.) temperatures were used to optimize production conditions for the deoptimized candidates, test for attenuation in vitro and examine CPD variants for increase temperature sensitivity as a means of explaining the mechanism of attenuation. Multiple step growth curves were conducted in Vero cells for synthetic DENV4 WT virus (in a full-length DENV4 backbone) and attenuated live vaccine candidates DENV4 E-W/MIN and E-MIN derived from DENV4 WT. Both DENV4 WT and E-W/MIN grew to high titers in Vero cells, with the peak DENV4 E-W/MIN titers approximately 10-fold lower but still high (˜10⁷ FFU/ml) at 33° C. and 37° C. Both viruses were partially restricted at 39° C., but not to the extent seen with DENV1-3. On day 5 a small but significant (˜5-fold) change in titer from 37° C. to 39° C. compared to WT was observed. DENV4 E-MIN was attenuated in Vero cells with peak titers 100-fold lower than WT. DENV4 E-MIN replication at 33° C. and 37° C. was similar, however, DENV4 E-MIN was not viable at 39° C. with no detectable virus on days 1-14 post-infection.

### Example 8

### Evaluation of the Attenuation, Immunogenicity and Efficacy of DENV2 Vaccine Candidates in AG129 Mice

As described above, the AG129 adult mouse model is gaining acceptance for studies of flavivirus vaccines and therapeutics. We used AG129 mice to test: 1) each synthetically derived wild-type virus DENV at a dose of 10⁶ (positive control); 2) two doses (10⁴ and 10⁶ PFU) of the vaccine candidates. Survival, weight, and clinical sign data were collected daily throughout the course of the experiment. SAVE deoptimized DENV strains were attenuated compared to synthetic wild-type viruses. DENV-2 E-min based on a DENV2 16681 backbone has been shown to be attenuated and immunogenic in neonatal ICR mice. In this immunogenicity/dose escalation study, the vaccine was evaluated for the first time in AG129 mice. Animals were immunized with DENV-2 E-min or DENV-2 16681 at two different concentrations. Resultant neutralizing antibody titers were determined, and the protective efficacy afforded evaluated against a mouse-adapted lethal strain of DENV2, D2S10[3]. None of the animals immunized with either virus at either dose showed clinical signs. Serum was collected from the immunized mice two days after the first immunization and viremia determined by RT-PCR. Viral RNA was detected in 55% (5/9) animals immunized with the higher inoculum of DENV-2 16681 and 11% (1/9) that received the lower inoculum, but was not detected in any of the mice immunized with DENV-2 E-min. The RT-PCR primers used do not target the viral E gene which has been codon deoptimized in this virus and, they successfully amplified DENV-2 E-min viral RNA included in the assay. Consequently, we do not believe that lack of detection of viremia in this study is a result of suboptimal RT-PCR amplification but indicates that the vaccine virus produced very low-level viremia in vivo. Upon completion of the immunization regimen serum was collected from all mice prior to virus challenge. All of the immunized animals had detectable DENV-2 neutralizing antibody titers prior to virus challenge with D2S10. A scatterplot of the data for each vaccine group demonstrating that there was an increase in neutralizing antibody titers with increasing vaccine dose for both DENV-2 E-min and DENV-2 16681, and that titers seen in E-min immunized mice were lower than those in mice immunized with the corresponding dose of DENV-2 16681. Animals in the media control group developed a rapid progressive infection with all animals dying. Mice immunized with the low dose of DENV-2 E-min vaccine were not protected, experiencing high lethality (67%) and a mean day of death (MDD) that was not significantly different to that seen in the media controls. In contrast, animals immunized with the high dose of DENV-2 E-min vaccine and with both doses of DENV-2 16681 experienced significant protection against DENV-2 D2S10 challenge. There was no lethality, or morbidity as indicated by a >10% loss in body weight, in any of these groups during the 30 day observation period. As anticipated high levels of viral RNA were detected in the sera of all media control animals on both of the days sampled. Viral RNA was also detected in all of the animals immunized with the lower dose of E-min on both days. Levels in these animals were comparable to those in controls on day 2 but were significantly lower on day 3. For animals immunized with the high dose of Emin, viral RNA was detected in the serum of 2/4 animals on day 2 with the RNA levels in the viremic animals being significantly lower than controls. On day 3, viral RNA was not detected in the sera of any of the high dose E-min animals. Overall, immunization with the high dose of Emin significantly reduced the number of animals in which viremia was detected (2/8 vs 8/8; p<0.01). For mice immunized with DENV-2 16681, no viral RNA was detected in the serum from any of the animals at either immunization dose on either of the two days sampled.

### Example 9

### Titration of Codon-Pair Deoptimization Leads to an Improved DENV2 Vaccine Candidate in AG129 Mice

To improve the clinical relevance of our DENV2 candidate, we next constructed D2-E-min and D2-1/2-E-min with E sequence derived from DENV-2/NI/BID-V533/2005 in the heterologous DENV2 16681 backbone. We tested D2-1/2-E-min, with the E region consisting of half wt DENV-2/NI/BID-V533/2005 sequence and half CPD, to improve the immunogenicity of our virus as previous studies have shown that the extent of attenuation can be titrated in DENV2 by reduction of CPD sequence. AG129 and BALB/c mice (n=5) were vaccinated with 10⁶ or 10⁴ FFU of D2-E-min, D2-1/2-E-min, or D2-WTE (DENV2 16681 backbone with WT DENV-2/NI/BID-V533/2005 E region). Interestingly, the introduction of the modem DENV-2/NI/BID-V533/2005 E region resulted in increased virulence, with all AG129 mice infected with 10⁶ or 10⁴ FFU requiring euthanasia. No mortality or morbidity was observed in infected BALB/c mice or in any CPD DENV2 group.

Reduction of CPD sequence in the E region was associated with a significant, approximately 4-fold, increase (p=−0.0002, Student's t-test) in the PRNT50 values of serum from D2-1/2-E-min compared to D2-E-Min vaccinated AG129 mice. Additionally, high neutralizing antibody titers were maintained with D2-1/2-E-min vaccination as no significant difference was observed between the 10⁶ (GM=3447) and 104 (GM=2867) FFU vaccinated groups. In immune-competent BALB/c mice, both D2-E-min and D2-1/2-E-min engendered levels of neutralizing antibody comparable to wild-type.

### Example 10

### Evaluation Ofthe Immunogenicity and Protective Efficacy of DENV3 Vaccine Candidates in AG129 Mice

AG129 mice were used to test the immunogenicity and protective efficacy of DENV-3 vaccine candidate DENV D2/D3-E-min, which comprises the DENV-2 strain 16681 backbone with a codon-deoptimized prME cassette from DENV-3/VE/BID-V2268/2008. This study was designed to evaluate the immunogenicity and efficacy of this candidate vaccine against challenge with virulent DENV-3 CO360/94. The first immunization was well tolerated by all of the animals with no major associated weight loss and none of the animals developed clinical signs. Similar results were seen after the second immunization although unexpectedly, one animal in Group 3 (10⁶ FFU DENV D2/D3-WT) developed rapid progressive weight loss and became moribund requiring euthanasia on day 2 after the immunization. A second animal from the same group subsequently died unexpectedly two weeks after the immunization and prior to challenge with virulent DENV-3. After lethal challenge with 1.2×10⁷ PFU DENV-3 CO360/94, animals in the media control group developed a rapid progressive infection that was lethal in 8/9 animals with the mean day of death (MDD) among the animals that died of 4.0±0.8 days. Immunization with the higher inoculum of DENV D2/D3-E-min provided significant protection with no lethality or morbidity (as measured by >10% weight loss) among the animals in the group. Immunization with the lower inoculum of DENV D2/D3-E-min was also protective with only a single animal developing a lethal infection. Animals that received the DENV D2/D3-WT virus prior to DENV-3 challenge were also protected with no lethality in either group, although, 2 animals in the higher inoculum group lost >10% of their original body weight.

### Example 11

### Evaluation Ofthe Immunogenicity and Protective Efficacy of DENV4 Vaccine Candidates in AG129 Mice

AG129 mice (n=12) were used to test the immunogenicity and protective efficacy of heterologous D2/D4-E-min and D2/D4-1/2-E-Min viruses compared to D2/D4-WTE (n=12) and media-immunized mock control mice (n=9) at a dose of 10⁶ FFU delivered subcutaneously. All mice were immunized on days 0 and 21 of the study and sera collected on day 2 to determine viremia and on day 35 for titration of neutralizing antibodies. On day 35, the mice were challenged with interperitoneal injection of 10⁷ PFU DENV4 703/4. Challenged mice were weighed and monitored for morbidity to assess the impact of viral challenge. In addition, 5 animals from each group were anesthetized and blood collected by retro-orbital bleed on day 2 post challenge and the remaining animals on day 3 to determine viremia levels by qRT-PCR. On day 30 post challenge the study was concluded, and serum collected from all surviving animals for determination of post-challenge PRNT50 values. The first immunization with all strains was well tolerated by all groups of animals with no sustained weight loss over the 5 day observation period, however, in the D2/D4-WTE group one mouse had to be euthanized prior to the second immunization and additional animals died after the second immunization. No animals in any ofthe other vaccine groups experienced progressive weight loss. At 2 days post-challenge (DPC), viremia was detected in 4/12 mice inoculated with D2/D4-WTE. For all of the mice immunized with the other two viruses, viremia levels were below the limit of detection of the assay (500 genome equivalents/ml). DENV-4 703/4 challenge produced rapid progressive infection with universal lethality in the media control group. In addition, three animals immunized with D2/D4-E-min experienced lethal infection with the other animals in this group remaining healthy for the duration of the study. Importantly, no lethality or evidence of morbidity as determined by weight loss was seen in any of the D2/D4-1/2-E-min immunized animals after DENV-4 703/4 challenge.

### Example 12

### Immunogenicity of Homologous DENV1-4 Vaccine Candidates in A129 Mice

We have tested DENV1-4 WT as well as vaccine candidates for DENV2-4 for immunogenicity in IFNα receptor knockout mice. All WT viruses were highly immunogenic in these mice, with neutralizing antibody titers >1024. DENV2-E-W/MIN was equally immunogenic to DENV2 WT virus at both a 10⁶ and 10⁴ FFU dose. Immunogenicity of DENV3 viruses waned with decreasing dose and increased deoptimization, however, DENV3 E-W/MIN was still highly immunogenic at a 10⁴ FFU dose (˜1024 FRNT50). We noticed a pronounced improvement in the immunogenicity of DENV4 E-W/MIN and WT as compared to the heterologous DENV4 WT in the DENV2 16681 backbone, which was poorly immunogenic in A129 mice.

### Example 13

### Tetravalent Vaccination in AG129 Mice and Efficacy Vs DENV3 Challenge

AG129 mice, homozygous for a double-knockout of IFNα/β and IFNγ receptors are commonly used to test Flavivirus vaccine candidates due to increased susceptibility to infection. We used AG129 mice to test a tetravalent formulation of our DENV vaccine by vaccinating them on days 0 and 21 with 10⁶ IFU monovalent D2/D3-E-min vaccine (N=16; 10 male and 6 female), tetravalent vaccine containing 10⁶ IFU of each ofthe four monovalent DENV E-min viruses (N=16; 10 male and 6 female), or with media (N=13; 8 male and 5 female). Both ofthe immunizations were well tolerated by all ofthe animals with none of the animals developing clinical signs and no major weight loss (>10%). Serum was collected prior to challenge for measurement of neutralizing antibodies by FRNT50. All of the mice immunized with the monovalent vaccine developed detectable neutralizing antibody titers (range 20-160). In mice immunized with the tetravalent vaccine formulation, titers were generally lower (range <20-80). On day 35, all remaining mice were challenged with 10⁷ IFU DENV3 CO360/94 delivered by the intraperitoneal route. As anticipated, animals in the media control group developed a rapid progressive infection that was lethal in 8/9 (89%) animals with the mean day of death (MDD) among the animals that died of 4.1±0.4 days. Immunization with monovalent DENV D2/D3-E-min or the tetravalent vaccine provided significant protection with no lethality or morbidity (as measured by >10% weight loss) among the animals in either group. Viral RNA was detected in 8/9 control animals with the titer being comparable on days 2 and 3 post challenge.

### Example 14

In vivo LD50 data for tetravalent and each strain of the homologous vaccine shows neuroattenuation. Week-old mice were injected with 10 μl of inoculum containing synthetic wt DENV1-4 viruses as indicated doses. Each mouse was weighed daily for up to 2 weeks and mortality determined by humane early-endpoints (absence of weight gain, paresis, hind-limb paralysis) whenever possible.

| | LD50 | MTD |
|---|---|---|
| Virus | (FFU) | (10 FFU) |
| DENV1 WT | 0.4 | 7 |
| DENV1 E-W/MIN | 2.2 | 10 |
| DENV2 WT | ≤3.4 | 9 |
| DENV2 E-W/MIN | 17.8 | >14 |
| DENV3 WT | 3.7 | 10 |
| DENV3 E-W/MIN | 1,931 | N.D. |
| DENV4 WT | 0.13 | 8 |
| DENV4 E-W/MIN | 2.5 | 11.5 |
| Tetravalent E-W/Min | 7.9 | 10 |

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of”

1. A modified dengue virus, comprising
a recoded prM protein, a recoded envelope (E) protein, or both,
wherein the recoded prM protein has a reduced codon pair bias compared to its parent prM protein encoding sequence, or has at least 5 codons substituted with synonymous codons less frequently used, or has an increased number of CpG or UpA di-nucleotides compared its parent prM protein encoding sequence, and
wherein the recoded E protein has a reduced codon pair bias compared to its parent E protein encoding sequence, or has at least 5 codons substituted with synonymous codons less frequently, or has an increased number of CpG or UpA di-nucleotides compared its parent E protein encoding sequence.

2. The modified dengue virus of claim 1, wherein expression of the prM protein or E protein or both are reduced compared to its parent dengue virus.

3. (canceled)

4. (canceled)

5. (canceled)

6. (canceled)

7. (canceled)

8. (canceled)

9. The modified dengue virus of claim 1, wherein each of the recoded prM or E protein-encoding sequence has a codon pair bias of less than −0.05.

10. The modified dengue virus of claim 1, wherein the codon pair bias of each of the recoded prM or E protein-encoding sequence is reduced by at least 0.05.

11. The modified dengue virus of claim 1, wherein the modified dengue virus is selected from type 1, type 2, type 3, type 4 or a combination thereof.

12. The modified dengue virus of claim 1, where the modified dengue virus is a modified tetravalent dengue virus.

13. A dengue vaccine composition for inducing a protective immune response in a subject, comprising a modified dengue virus of claim 1, and a pharmaceutically acceptable excipient or carrier.

14. A method of eliciting an immune response in a subject, comprising: administering to the subject an effective dose of a composition comprising a modified dengue virus of claim 1.

15. The method of claim 14, wherein the immune response is a protective immune response, and a prophylactically effective or therapeutically effective dose of a vaccine composition of claims is administered.

16. The method of claim 14, further comprising administering to the subject at least one adjuvant.

17. The method of claim 14, wherein the immune response is cross-protective against a heterologous dengue virus.

18. A method of eliciting an immune response in a subject in need thereof, comprising:
administering a prime dose of (i) an attenuated dengue virus produced by a method other than codon-pair deoptimization or codon deoptimization, or increasing of CpG or UpA di-nucleotides, or (ii) a modified dengue virus comprising a recoded prM protein, a recoded envelope (E) protein, or both, wherein the recoded prM protein has a reduced codon pair bias compared to its parent prM protein encoding sequence, or has at least 5 codons substituted with synonymous codons less frequently used, or has an increased number of CpG or UpA di-nucleotides compared its parent prM protein encoding sequence, and wherein the recoded E protein has a reduced codon pair bias compared to its parent E protein encoding sequence, or has at least 5 codons substituted with synonymous codons less frequently, or has an increased number of CpG or UpA di-nucleotides compared its parent E protein encoding sequence; and
administering one or more boost dose of (i) the attenuated dengue virus produced by methods other than codon-pair deoptimization or codon deoptimization, or increasing of CpG or UpA di-nucleotides, or (ii) the modified dengue virus to the subject in need thereof, wherein at least the prime dose or the one or more boost dose is the modified dengue virus.

19. The method of claim 18, wherein a first of the one or more boost dose is administered about 2 weeks after the prime dose.

20. The method of claim 18, wherein expression of the prM protein or E protein or both are reduced compared to its parent dengue virus.

21. (canceled)

22. (canceled)

23. (canceled)

24. (canceled)

25. (canceled)

26. (canceled)

27. The method of claim 18, wherein each of the recoded prM or E protein-encoding sequence has a codon pair bias of less than −0.05.

28. The method of claim 18, wherein the codon pair bias of each of the recoded prM or E protein-encoding sequence is reduced by at least 0.05.

29. The method of claim 18, wherein the modified dengue virus is selected from type 1, type 2, type 3, type 4 or a combination thereof.

30. The method of claim 18, where the modified dengue virus is a modified tetravalent dengue virus.

31. A method of making a modified dengue virus genome comprising:
obtaining a nucleotide sequence encoding the envelope protein of a dengue virus;
recoding the envelope encoding nucleotide sequence to reduce protein expression, and
substituting a nucleic acid having the recoded envelope-encoding nucleotide sequence into a parent dengue virus genome to make a modified dengue virus genome; whereby expression of the recoded envelope-encoding nucleotide sequence is reduced compared to the parent virus.


SEQUENCE LISTING (12 sequences)

SEQ ID NO: 1 - DNA, 10735 bp, Dengue virus
agttgttagtctacgtggaccgacaagaacagtttcgaatcggaagcttgcttaacgtagttctaacagttttttattagagagcagatctctgatgaacaaccaacggaaaaagacggctcgaccgtctttcaatatgctgaaacgcgcgagaaaccgcgtgtcaactgtttcacagttggcgaagagattctcaaaaggattgctctcaggccaaggacccatgaaattggtgatggctttcatagcattcctaagatttctagccatacccccaacagcaggaattttggctagatggggttcattcaagaagagtggagcgatcaaagtgctacggggtttcaagaaagaaatctcaaacatgttgaatataatgaatagaaggaaaagatctgtgaccatgctccttatgctgatgcctacagccttggcgttccatttgactacacgagggggagagccgcacatgatagtcagcaagcaggaaagaggaaagtcactcttgtttaagacctcagcaggtgtcaacatgtgcacccttatagcgatggatttgggagagttatgtgaggacacaatgacttacaaatgccctcgaatcactgaaactgaaccagatgacgttgattgttggtgtaatgccacagacacatgggtgacctatggaacatgttcccaaactggcgagcaccgacgagacaaacgttccgtcgcactggccccacacgtgggacttggtttggaaacaagaaccgaaacgtggatgtcctctgaaggcgcttggaaacagatacaaagagtggagacttgggccctgagacacccaggattcacggtgatagccctttttctagcacatgccataggaacatccatcacccagaaagggattattttcattttgttaatgctggtaacaccatccatggccatgcgatgcgtgggaataggcagcagggacttcgtggaaggactgtcaggagcaacttgggtagatgtggtactggaacatggaagttgcgtcaccaccatggcaaaagacaaaccaacattggacattgaactcttgaagacggaagtcacaaaccctgccgtcctgcgcaaactgtgcattgaagctaaaatatcaaacaccaccaccgactcaagatgtccaacacaaggagaagccacactagtggaagaacaagacgcgaactttgtgtgtcgacgaacgtttgtggacagaggctggggcaatggctgtggcctcttcggaaaaggaagcctaataacgtgtgcaaagttcaagtgtgtgacaaaactggaaggaaagatagttcaatatgagaacttgaaatattcagtaatagtcaccgttcacaccggagaccagcatcaagtgggaaatgaaagcacagaacatgggacaactgcaactataacacctcaagctcctacgacggaaatacagctgaccgactacggagctcttacattggattgttcacctagaacaggactagactttaatgaaatggtgttgttgacaatgaaagaaaaatcatggctagtccacaaacaatggtttttagacctaccactgccttggacctcgggagcttcaacatcacaagagacttggaacagacaagatttgctggtgacatttaagacagctcatgcaaagaagcaggaagtagtcgtactaggatcacaagaaggagcaatgcacactgcgttgaccggagcgacagaaatccaaacgtctggaacgacaacaatttttgcaggacacttgaaatgcagactaaagatggacaaactgactctaaaagggatgtcatatgtgatgtgcacaggctcattcaagctagagaaagaagtggctgagacccagcatggaaccgttctagtgcagattaaatacgaaggaacagatgcaccatgcaagatccctttttcgacccaagatgaaaaaggagtaacccagaatgggagattgataacagctaaccccatagttactgacaaagaaaaaccagtcaacattgaggcagaaccgccctttggtgagagttacatcgtaataggagcaggtgaaaaagctttgaaactaagctggttcaagaaaggaagcagcatagggaaaatgtttgaggcaactgccagaggagcacgaaggatggccatactgggagacaccgcatgggactttggttctataggaggagtgttcacgtctgttggaaaattagtacaccagatttttggaactgcatatggagttttgttcagcggtgtttcctggaccatgaaaataggaataggggttctgctgacatggctgggattaaactcaaggagcacgtccctttcgatgacgtgcattgcagttggcctagtaacactatacctaggagtcatggttcaggcggattcaggatgtgtaattaattggaaaggtagagaactcaagtgtggaagtggcatttttgtcaccaatgaagttcacacttggacagagcaatacaaatttcaagctgactcccctaagagactatcagcagccatcgggaaggcatgggaggagggtgtgtgtggaattcgatcagcaactcgtctcgagaacatcatgtggaagcaaatatcaaatgaactgaatcacatcttacttgaaaatgatatgaaattcacagtggttgtaggagatgttgctgggatcttggctcaaggaaagaaaatgattaggccacaacccatggaatacaaatactcgtggaaaagctggggaaaggctaaaatcataggggcagatgtacagaacaccaccttcatcatcgatggcccaaacaccccagaatgccctgatgaccaaagagcatggaacatttgggaagttgaggactatggatttggaattttcacgacaaacatatggctgaaattgcgtgattcctacacccaagtgtgtgaccaccggctaatgtcagctgccatcaaggacagcaaggcagttcacgctgacatggggtactggatagaaagtgaaaagaacgagacctggaagctggcaagagcctcattcatagaagttaaaacatgtatctggccaaaatcccacactctatggagcaatggagttctggaaagtgaaatgataattccaaagatctatggaggaccaatatctcagcacaactacagaccaggatattttacacaagcagcagggccgtggcacctaggcaagttggaactggattttgatttgtgtgagggtaccacagttgttgtggatgaacattgtggaaatcgaggaccatctcttaggaccacaacagtcacaggaaagataattcatgaatggtgttgcagatcttgcacgctaccacccttacgtttcagaggagaagatgggtgctggtacggtatggaaatcagaccagtcaaggaaaaggaagaaaatctagtcaaatcaatggtctctgcagggtcaggggaagtggacagcttttcactaggactgctatgcatatcaataatgatcgaggaggtgatgagatccagatggagtagaagaatgctgatgactggaacactggctgtgttcttccttctcataatgggacaattgacatggaacgatctgatcagattatgcatcatggttggagccaacgcttccgacaggatggggatgggaacgacgtacctagccctgatggccacttttaaaatgagaccgatgttcgctgtagggctattatttcgcagactaacatccagagaagttcttcttctaacaattggattgagtctagtggcatctgtggagttaccaaattccttggaggagctgggggatggacttgcaatgggcattatgattttaaaattattgactgactttcaatcacatcagctgtgggctaccttgctgtccttgacatttatcaaaacaacgttttccttgcactacgcatggaagacaatggctatggtactgtcaattgtatctctcttccctttatgcctgtccacgacctcccaaaaaacaacatggcttccggtgctattgggatcccttggatgcaaaccactaaccatgtttcttatagcagaaaacaaaatctggggaaggagaagttggcccctcaatgaaggaatcatggctgttggaatagtcagcatcctactaagttcactcctcaaaaatgatgtaccgctagctgggccactaatagctggaggcatgctaatagcatgttatgttatatctggaagctcagccgacctatcattagagaaagcggctgaggtctcctgggaagaagaagcagaacactctggtgcctcacacaacatattagtggaagtccaagatgatggaaccatgaagataaaggatgaagagagagatgacacgctaaccattctccttaaagcaactctgttagcagtttcaggggtgtacccattatcaataccagcgacccttttcgtgtggtacttttggcagaaaaagaaacagagatctggagtgttatgggacacacccagccctccagaagtggaaagagcagttcttgatgatggtatctatagaattatgcagagaggactgttgggcaggtcccaagtaggggtaggagttttccaagaaaacgtgttccacacaatgtggcatgtcaccaggggagctgtactcatgtatcaagggaagagattggaaccgagctgggccagtgtcaaaaaagacctgatctcatatggaggaggttggaggcttcaaggatcctggaacacaggagaagaagtgcaggtgattgctgttgaaccagggaaaaaccccaaaaatgtacaaacagcgccgggcacctttaagacccctgaaggtgaagttggagccattgccctagactttaaacctggcacatctggatctcccatcgtgaacagagaaggaaaaatagtaggtctttatggaaatggagtagtgacaacaagtggaacctacgtcagtgccatagctcaagccaaagcatcacaagaagggcccctaccagagattgaagacgaggtgtttaggaaaagaaacttaacaataatggacctacatccaggatcggggaaaacaagaagatatcttccagccatagtccgtgaggccataaaaaggaagctgcgcacactaatcctggctcccacaagggttgtcgcttccgaaatggcagaggcgctcaagggaatgccaataaggtaccaaacaacagcagtgaagagtgaacatacaggaaaagagatagttgacctcatgtgtcacgccactttcaccatgcgcctcctgtctcccgtaagagttcccaattacaacatgatcatcatggatgaagcacatttcaccgatccatccagtatagcggccagagggtacatctcaacccgagtgggcatgggtgaagcagctgcaatcttcatgacagccactcccccaggatcagtggaggcctttccacagagcaacgcagttatccaagatgaggaaagagacattcctgagagatcatggaactcaggctatgagtggatcactgacttcccaggtaaaacagtttggtttgttccaagcattaaatcaggaaatgacattgccaactgcttaagaaagaatgggaaacgggtgattcaattgagcaggaaaacctttgatacagagtaccaaaaaacaaaaaacaacgactgggactacgtcgtcacaacagacatctccgaaatgggagcaaatttccgagccgacagggtgatagacccaagacggtgtctgaaaccggtaatactaaaagatggtccagagcgtgtcattttagcaggaccaatgccagtgactgtggccagtgccgctcagaggagaggaagaattggaaggaaccacaataaggaaggtgatcagtacatctacatgggacagcctttaaacaacgatgaagatcacgctcactggacagaagcaaaaatgctccttgataatataaacacaccagaagggattatcccagccctctttgagccagagagagaaaagagtgcagcaatagacggggaatacagactgcggggtgaagcaaggaaaacgtttgtggagctcatgagaagaggagatctacctgtctggctatcctacaaagttgcctcagaaggcttccagtactctgacagaagatggtgctttgacggggaaaggaacaaccaggtgttggaggagaacatggacgtggagatctggacaaaagaaggagaaagaaagaaactacgaccccgctggctggatgccagaacatactcagacccactagccctgcgcgagtttaaagagtttgcagcagggagaagaagcgtctcaggtgatttaatattagaaatagggaagcttccacaacacttgacgcaaagggcccagaatgccctggacaacctggttatgttgcacaactccgaacaaggaggcagagcctatagacatgcaatggaagaactgccagacaccatagaaacgttgatgctcctagctttgatagctgtgttaactggtggagtgacactgttcttcctatcaggaaggggcctagggaaaacatctatcggcctactctgcgtaatggcttcaagcgtactgctatggatggccagtgtggagccccattggatagcggcctccatcatactggagttcttcctgatggtgctgcttattccagagccagacagacaacgcactccgcaggacaaccagctggcatatgtggtgataggtttgttattcatgatactgacagtagcagccaatgagatgggactgctggaaaccacaaagaaagacttagggattggccatgtggctgttgaaaatcaccaccatgccgcaatgctggacgtagacttacatccagcttcagcctggaccctctatgcagtggccacaacaattatcactcccatgatgaggcacacaatcgaaaacacaacggcaaacatttccctgacagccattgcaaaccaggcagctatattgatgggacttgacaaaggatggccaatatcgaagatggacataggagttccacttctcgccttggggtgctattcccaggtgaatccactgacgctgacagcggcggtattgatgctagtggctcattacgccataattggacctggactgcaagcaaaagctactagagaagctcaaaaaaggacagcggccggaataatgaaaaatccaaccgttgatggaattgttgcaatagatttggaccctgtggtttatgatgcaaaatttgaaaaacaactaggccaaataatgttgttgatactatgcacatcacagatcctcttgatgcggactacatgggccttgtgcgagtccatcacactggccactggacctctgaccacgctttgggagggatctccaggaaaattttggaacaccacgatagcggtttccatggcaaacattttcagaggaagttatctagcaggagcaggtctggccttctcattaatgaaatctctaggaggaggtaggagaggcacgggagcccaaggggaaacactgggagagaaatggaaaagacagctgaaccaactgagcaagtcagaatttaacacctataaaaggagtgggattatggaagtggacagatccgaagccaaagagggattgaaaagaggagaaacaaccaaacatgcagtgtcgagaggaaccgctaaactgaggtggttcgtggagaggaaccttgtgaaaccagaagggaaagtcatagacctcggttgtggaagaggtggctggtcatactattgcgctgggctgaagaaagtcacagaagtgaaggggtatacaaaaggaggacctggacatgaggaaccaatcccaatggcgacctatggatggaacctagtaaagctgcactctgggaaagacgtattttttataccacctgagaaatgtgacacccttttgtgtgatattggtgagtcctctccaaacccaactatagaggaaggaagaacgctacgcgtcctaaagatggtggaaccatggctcagaggaaaccaattttgcataaaaattctgaatccctacatgccaagtgtggtggaaactctggagcaaatgcaaagaaaacatggaggaatgctagtgcgaaatccactttcaagaaattctactcatgaaatgtattgggtttcatgtggaacaggaaacattgtgtcagcagtaaacatgacatctagaatgttgctaaatcgattcacaatggctcacaggaaaccaacatatgaaagagacgtggacctaggcgccggaacaagacatgtggcagtggaaccagaggtagccaacctagatatcattggccagaggatagagaacataaaacatgaacataagtcaacatggcattatgatgaggacaatccatataaaacatgggcctatcatggatcatatgaggtcaagccatcaggatcagcctcatccatggtcaatggcgtggtgaaactgctcaccaaaccatgggatgtcatccctatggtcacacaaatagccatgactgacactacaccctttggacaacagagggtgtttaaagagaaagttgacacacgcacaccaaaagcaaaacggggcacagcacaaatcatggaggtgacagccaagtggttatggggttttctttctagaaacaagaaaccaagaatttgcacaagagaggagttcacaagaaaagttaggtcaaacgcagccattggagcagtgttcgttgatgaaaatcaatggaactcagcaaaagaagcagtggaagatgagcggttctgggaccttgtgcatagagagagggagcttcacaaacagggaaaatgtgctacgtgtgtttacaacatgatggggaagagagagaaaaagctaggagagttcggaaaggcaaaaggaagtcgtgcaatatggtacatgtggttgggagcacgctttctagagttcgaagctcttggtttcatgaacgaagatcactggttcagcagagagaattcactcagcggagtggaaggagaaggactccacaaacttggatatatactcagagacatatcaaagattccagggggaaacatgtatgcagatgacacagccggatgggatacaaggataacagaggatgaccttcagaatgaggccagaattactgacatcatggaacccgaacatgccctcctggctaagtcaatcttcaagttaacctaccaaaataaggtggtaagggtacagagaccagcaaaaaatggaaccgtgatggatgtcatatccagacgtgaccagagaggaagtggtcaggtcggaacttatggcttaaacactttcaccaacatggaagcccagctgataagacaaatggagtctgagggaatcttttcacccagcgaattagagaccccaaatttagccgagagagttctcgactggctggaaaaatatggcgtcgaaaggctgaaaagaatggcaatcagcggagatgactgcgtagtgaaaccaattgatgataggtttgcaacagccttgacagctctgaatgatatgggaaaagtaagaaaagatataccacaatgggaaccttcaaaaggatggaatgattggcaacaagtgcctttttgttcacaccacttccaccagctgattatgaaggatgggagggaaatagtggtgccatgccgcaaccaagatgaacttgtgggtagggctagagtatcacaaggtgctggatggagcctgagagaaactgcatgcctaggcaagtcatatgcacaaatgtggcagctgatgtacttccacaggagagacctgagactagccgctaatgctatctgttcagccgttccagttgattggatcccaaccagccgtaccacctggtcgatccatgcccatcaccaatggatgacaacagaagacatgctgtcagtgtggaatagggtttggatagaggaaaacccatggatggaggacaaaacccacatatccagttggggagatgttccatatttaggaaaaagggaagaccaatggtgtggatccctgataggcttaacagcaagggccacctgggccaccaacatacaagtggccataaaccaagtgagaagactaattgggaatgagaattatctagattacatgacatcaatgaagagattcaagaacgagagtgatcccgaaggggcactctggtgagtcaacacatttacaaaataaaggaaaataagaaatcaaacaaggcaagaagtcaggccggattaagccatagtacggtaagagctatgctgcctgtgagccccgtctaaggacgtaaaatgaagtcaggccgaaagccacggcttgagcaaaccgtgctgcctgtagctccatcgtggggatgtaaaaacctgggaggctgcaacccatggaagctgtacgcatggggtagcagactagtggttagaggagacccctcccgaaacataacgcagcagcggggcccaacaccaggggaagctgtaccctggtggtaaggactagaggttagaggagaccccccgcataacaataaacagcatattgacgctgggagagaccagagatcctgctgtctctacagcatcattccaggcacagaacgccagaaaatggaatggtgctgttgaatcaacaggttct

SEQ ID NO: 2 - DNA, 10735 bp, Dengue virus
agttgttagtctacgtggaccgacaagaacagtttcgaatcggaagcttgcttaacgtagttctaacagttttttattagagagcagatctctgatgaacaaccaacggaaaaagacggctcgaccgtctttcaatatgctgaaacgcgcgagaaaccgcgtgtcaactgtttcacagttggcgaagagattctcaaaaggattgctctcaggccaaggacccatgaaattggtgatggctttcatagcattcctaagatttctagccatacccccaacagcaggaattttggctagatggggttcattcaagaagagtggagcgatcaaagtgctacggggtttcaagaaagaaatctcaaacatgttgaatataatgaatagaaggaaaagatctgtgaccatgctccttatgctgatgcctacagccttggcgttccatttgactacacgagggggagagccgcacatgatagtcagcaagcaggaaagaggaaagtcactcttgtttaagacctcagcaggtgtcaacatgtgcacccttatagcgatggatttgggagagttatgtgaggacacaatgacttacaaatgccctcgaatcactgaaactgaaccagatgacgttgattgttggtgtaatgccacagacacatgggtgacctatggaacatgttcccaaactggcgagcaccgacgagacaaacgttccgtcgcactggccccacacgtgggacttggtttggaaacaagaaccgaaacgtggatgtcctctgaaggcgcttggaaacagatacaaagagtggagacttgggccctgagacacccaggattcacggtgatagccctttttctagcacatgccataggaacatccatcacccagaaagggattattttcattttgttaatgctggtaacaccatccatggccatgcgatgcgtagggatagggtcacgcgatttcgtcgaaggactatccggagcgacatgggtcgacgtcgtactcgaacacggatcatgcgttacgactatggctaaagacaaacctacactagacatagagttactgaaaaccgaagttacgaatcccgccgtactgcgaaaattgtgtatcgaagcgaaaattagcaatacgactacagactctaggtgtcctacacaaggcgaagcgacattggtcgaagaacaggacgctaacttcgtatgtagacgaacattcgtcgatagggggtggggaaacggatgcggattgttcggtaaaggatcactgattacatgcgcaaagttcaaatgcgttacgaaactggaaggtaaaatagtgcaatacgaaaacctaaagtatagcgtaatcgttacagtgcataccggagaccaacatcaggtcggaaacgaatcaaccgaacacggaacaaccgcaacaattacaccacaggcacctacaaccgaaattcaactaaccgattacggagctcttacactagattgctcacctagaaccggattggactttaacgaaatggtactgttgactatgaaggagaaatcatggttagtgcataaacaatggtttctagacctaccactaccatggactagcggagcgagtacgtcacaggaaacatggaatagacaggacctattggtgacatttaagaccgcacacgctaagaagcaggaagtcgtagtgttagggtcacaagagggagcaatgcataccgcactaaccggagcaaccgaaatacagactagcggaactacaacgatttttgccggtcacctgaaatgtagactgaagatggacaaactgacacttaaaggaatgtcatacgttatgtgtacgggatcctttaagctcgaaaaagaggttgccgaaacgcaacacggaacagtgctagtgcaaattaaatatgagggaaccgacgcaccatgtaagataccgtttagtacgcaagacgaaaagggcgttacgcaaaacggaagactgattaccgctaaccctatcgttacagacaaagagaaacccgttaacatagaggccgaaccaccttttggcgaatcatatatagtgataggcgcaggcgaaaaagcactaaagctgtcatggttcaaaaaaggatctagcatagggaagatgttcgaagcaaccgctaggggagctagacgaatggcaatactgggagataccgcatgggacttcggatcgatagggggagtgtttactagcgtaggtaagttggtgcatcagatattcggaaccgcatacggagtgttgtttagcggagtgtcatggactatgaagatagggataggagtgctattgacatggctcggactgaattcgagatcgactagcctatctatgacatgcatagccgtcggactggttacactgtatctaggcgtaatggtgcaagcagattcaggatgtgtaattaattggaaaggtagagaactcaagtgtggaagtggcatttttgtcaccaatgaagttcacacttggacagagcaatacaaatttcaagctgactcccctaagagactatcagcagccatcgggaaggcatgggaggagggtgtgtgtggaattcgatcagcaactcgtctcgagaacatcatgtggaagcaaatatcaaatgaactgaatcacatcttacttgaaaatgatatgaaattcacagtggttgtaggagatgttgctgggatcttggctcaaggaaagaaaatgattaggccacaacccatggaatacaaatactcgtggaaaagctggggaaaggctaaaatcataggggcagatgtacagaacaccaccttcatcatcgatggcccaaacaccccagaatgccctgatgaccaaagagcatggaacatttgggaagttgaggactatggatttggaattttcacgacaaacatatggctgaaattgcgtgattcctacacccaagtgtgtgaccaccggctaatgtcagctgccatcaaggacagcaaggcagttcacgctgacatggggtactggatagaaagtgaaaagaacgagacctggaagctggcaagagcctcattcatagaagttaaaacatgtatctggccaaaatcccacactctatggagcaatggagttctggaaagtgaaatgataattccaaagatctatggaggaccaatatctcagcacaactacagaccaggatattttacacaagcagcagggccgtggcacctaggcaagttggaactggattttgatttgtgtgagggtaccacagttgttgtggatgaacattgtggaaatcgaggaccatctcttaggaccacaacagtcacaggaaagataattcatgaatggtgttgcagatcttgcacgctaccacccttacgtttcagaggagaagatgggtgctggtacggtatggaaatcagaccagtcaaggaaaaggaagaaaatctagtcaaatcaatggtctctgcagggtcaggggaagtggacagcttttcactaggactgctatgcatatcaataatgatcgaggaggtgatgagatccagatggagtagaagaatgctgatgactggaacactggctgtgttcttccttctcataatgggacaattgacatggaacgatctgatcagattatgcatcatggttggagccaacgcttccgacaggatggggatgggaacgacgtacctagccctgatggccacttttaaaatgagaccgatgttcgctgtagggctattatttcgcagactaacatccagagaagttcttcttctaacaattggattgagtctagtggcatctgtggagttaccaaattccttggaggagctgggggatggacttgcaatgggcattatgattttaaaattattgactgactttcaatcacatcagctgtgggctaccttgctgtccttgacatttatcaaaacaacgttttccttgcactacgcatggaagacaatggctatggtactgtcaattgtatctctcttccctttatgcctgtccacgacctcccaaaaaacaacatggcttccggtgctattgggatcccttggatgcaaaccactaaccatgtttcttatagcagaaaacaaaatctggggaaggagaagttggcccctcaatgaaggaatcatggctgttggaatagtcagcatcctactaagttcactcctcaaaaatgatgtaccgctagctgggccactaatagctggaggcatgctaatagcatgttatgttatatctggaagctcagccgacctatcattagagaaagcggctgaggtctcctgggaagaagaagcagaacactctggtgcctcacacaacatattagtggaagtccaagatgatggaaccatgaagataaaggatgaagagagagatgacacgctaaccattctccttaaagcaactctgttagcagtttcaggggtgtacccattatcaataccagcgacccttttcgtgtggtacttttggcagaaaaagaaacagagatctggagtgttatgggacacacccagccctccagaagtggaaagagcagttcttgatgatggtatctatagaattatgcagagaggactgttgggcaggtcccaagtaggggtaggagttttccaagaaaacgtgttccacacaatgtggcatgtcaccaggggagctgtactcatgtatcaagggaagagattggaaccgagctgggccagtgtcaaaaaagacctgatctcatatggaggaggttggaggcttcaaggatcctggaacacaggagaagaagtgcaggtgattgctgttgaaccagggaaaaaccccaaaaatgtacaaacagcgccgggcacctttaagacccctgaaggtgaagttggagccattgccctagactttaaacctggcacatctggatctcccatcgtgaacagagaaggaaaaatagtaggtctttatggaaatggagtagtgacaacaagtggaacctacgtcagtgccatagctcaagccaaagcatcacaagaagggcccctaccagagattgaagacgaggtgtttaggaaaagaaacttaacaataatggacctacatccaggatcggggaaaacaagaagatatcttccagccatagtccgtgaggccataaaaaggaagctgcgcacactaatcctggctcccacaagggttgtcgcttccgaaatggcagaggcgctcaagggaatgccaataaggtaccaaacaacagcagtgaagagtgaacatacaggaaaagagatagttgacctcatgtgtcacgccactttcaccatgcgcctcctgtctcccgtaagagttcccaattacaacatgatcatcatggatgaagcacatttcaccgatccatccagtatagcggccagagggtacatctcaacccgagtgggcatgggtgaagcagctgcaatcttcatgacagccactcccccaggatcagtggaggcctttccacagagcaacgcagttatccaagatgaggaaagagacattcctgagagatcatggaactcaggctatgagtggatcactgacttcccaggtaaaacagtttggtttgttccaagcattaaatcaggaaatgacattgccaactgcttaagaaagaatgggaaacgggtgattcaattgagcaggaaaacctttgatacagagtaccaaaaaacaaaaaacaacgactgggactacgtcgtcacaacagacatctccgaaatgggagcaaatttccgagccgacagggtgatagacccaagacggtgtctgaaaccggtaatactaaaagatggtccagagcgtgtcattttagcaggaccaatgccagtgactgtggccagtgccgctcagaggagaggaagaattggaaggaaccacaataaggaaggtgatcagtacatctacatgggacagcctttaaacaacgatgaagatcacgctcactggacagaagcaaaaatgctccttgataatataaacacaccagaagggattatcccagccctctttgagccagagagagaaaagagtgcagcaatagacggggaatacagactgcggggtgaagcaaggaaaacgtttgtggagctcatgagaagaggagatctacctgtctggctatcctacaaagttgcctcagaaggcttccagtactctgacagaagatggtgctttgacggggaaaggaacaaccaggtgttggaggagaacatggacgtggagatctggacaaaagaaggagaaagaaagaaactacgaccccgctggctggatgccagaacatactcagacccactagccctgcgcgagtttaaagagtttgcagcagggagaagaagcgtctcaggtgatttaatattagaaatagggaagcttccacaacacttgacgcaaagggcccagaatgccctggacaacctggttatgttgcacaactccgaacaaggaggcagagcctatagacatgcaatggaagaactgccagacaccatagaaacgttgatgctcctagctttgatagctgtgttaactggtggagtgacactgttcttcctatcaggaaggggcctagggaaaacatctatcggcctactctgcgtaatggcttcaagcgtactgctatggatggccagtgtggagccccattggatagcggcctccatcatactggagttcttcctgatggtgctgcttattccagagccagacagacaacgcactccgcaggacaaccagctggcatatgtggtgataggtttgttattcatgatactgacagtagcagccaatgagatgggactgctggaaaccacaaagaaagacttagggattggccatgtggctgttgaaaatcaccaccatgccgcaatgctggacgtagacttacatccagcttcagcctggaccctctatgcagtggccacaacaattatcactcccatgatgaggcacacaatcgaaaacacaacggcaaacatttccctgacagccattgcaaaccaggcagctatattgatgggacttgacaaaggatggccaatatcgaagatggacataggagttccacttctcgccttggggtgctattcccaggtgaatccactgacgctgacagcggcggtattgatgctagtggctcattacgccataattggacctggactgcaagcaaaagctactagagaagctcaaaaaaggacagcggccggaataatgaaaaatccaaccgttgatggaattgttgcaatagatttggaccctgtggtttatgatgcaaaatttgaaaaacaactaggccaaataatgttgttgatactatgcacatcacagatcctcttgatgcggactacatgggccttgtgcgagtccatcacactggccactggacctctgaccacgctttgggagggatctccaggaaaattttggaacaccacgatagcggtttccatggcaaacattttcagaggaagttatctagcaggagcaggtctggccttctcattaatgaaatctctaggaggaggtaggagaggcacgggagcccaaggggaaacactgggagagaaatggaaaagacagctgaaccaactgagcaagtcagaatttaacacctataaaaggagtgggattatggaagtggacagatccgaagccaaagagggattgaaaagaggagaaacaaccaaacatgcagtgtcgagaggaaccgctaaactgaggtggttcgtggagaggaaccttgtgaaaccagaagggaaagtcatagacctcggttgtggaagaggtggctggtcatactattgcgctgggctgaagaaagtcacagaagtgaaggggtatacaaaaggaggacctggacatgaggaaccaatcccaatggcgacctatggatggaacctagtaaagctgcactctgggaaagacgtattttttataccacctgagaaatgtgacacccttttgtgtgatattggtgagtcctctccaaacccaactatagaggaaggaagaacgctacgcgtcctaaagatggtggaaccatggctcagaggaaaccaattttgcataaaaattctgaatccctacatgccaagtgtggtggaaactctggagcaaatgcaaagaaaacatggaggaatgctagtgcgaaatccactttcaagaaattctactcatgaaatgtattgggtttcatgtggaacaggaaacattgtgtcagcagtaaacatgacatctagaatgttgctaaatcgattcacaatggctcacaggaaaccaacatatgaaagagacgtggacctaggcgccggaacaagacatgtggcagtggaaccagaggtagccaacctagatatcattggccagaggatagagaacataaaacatgaacataagtcaacatggcattatgatgaggacaatccatataaaacatgggcctatcatggatcatatgaggtcaagccatcaggatcagcctcatccatggtcaatggcgtggtgaaactgctcaccaaaccatgggatgtcatccctatggtcacacaaatagccatgactgacactacaccctttggacaacagagggtgtttaaagagaaagttgacacacgcacaccaaaagcaaaacggggcacagcacaaatcatggaggtgacagccaagtggttatggggttttctttctagaaacaagaaaccaagaatttgcacaagagaggagttcacaagaaaagttaggtcaaacgcagccattggagcagtgttcgttgatgaaaatcaatggaactcagcaaaagaagcagtggaagatgagcggttctgggaccttgtgcatagagagagggagcttcacaaacagggaaaatgtgctacgtgtgtttacaacatgatggggaagagagagaaaaagctaggagagttcggaaaggcaaaaggaagtcgtgcaatatggtacatgtggttgggagcacgctttctagagttcgaagctcttggtttcatgaacgaagatcactggttcagcagagagaattcactcagcggagtggaaggagaaggactccacaaacttggatatatactcagagacatatcaaagattccagggggaaacatgtatgcagatgacacagccggatgggatacaaggataacagaggatgaccttcagaatgaggccagaattactgacatcatggaacccgaacatgccctcctggctaagtcaatcttcaagttaacctaccaaaataaggtggtaagggtacagagaccagcaaaaaatggaaccgtgatggatgtcatatccagacgtgaccagagaggaagtggtcaggtcggaacttatggcttaaacactttcaccaacatggaagcccagctgataagacaaatggagtctgagggaatcttttcacccagcgaattagagaccccaaatttagccgagagagttctcgactggctggaaaaatatggcgtcgaaaggctgaaaagaatggcaatcagcggagatgactgcgtagtgaaaccaattgatgataggtttgcaacagccttgacagctctgaatgatatgggaaaagtaagaaaagatataccacaatgggaaccttcaaaaggatggaatgattggcaacaagtgcctttttgttcacaccacttccaccagctgattatgaaggatgggagggaaatagtggtgccatgccgcaaccaagatgaacttgtgggtagggctagagtatcacaaggtgctggatggagcctgagagaaactgcatgcctaggcaagtcatatgcacaaatgtggcagctgatgtacttccacaggagagacctgagactagccgctaatgctatctgttcagccgttccagttgattggatcccaaccagccgtaccacctggtcgatccatgcccatcaccaatggatgacaacagaagacatgctgtcagtgtggaatagggtttggatagaggaaaacccatggatggaggacaaaacccacatatccagttggggagatgttccatatttaggaaaaagggaagaccaatggtgtggatccctgataggcttaacagcaagggccacctgggccaccaacatacaagtggccataaaccaagtgagaagactaattgggaatgagaattatctagattacatgacatcaatgaagagattcaagaacgagagtgatcccgaaggggcactctggtgagtcaacacatttacaaaataaaggaaaataagaaatcaaacaaggcaagaagtcaggccggattaagccatagtacggtaagagctatgctgcctgtgagccccgtctaaggacgtaaaatgaagtcaggccgaaagccacggcttgagcaaaccgtgctgcctgtagctccatcgtggggatgtaaaaacctgggaggctgcaacccatggaagctgtacgcatggggtagcagactagtggttagaggagacccctcccgaaacataacgcagcagcggggcccaacaccaggggaagctgtaccctggtggtaaggactagaggttagaggagaccccccgcataacaataaacagcatattgacgctgggagagaccagagatcctgctgtctctacagcatcattccaggcacagaacgccagaaaatggaatggtgctgttgaatcaacaggttct

SEQ ID NO: 3 - DNA, 10725 bp, Dengue virus
agttgttagtctacgtggaccgacaaagacagattctttgagggagctaagctcaacgtagttctaacagttttttaattagagagcagatctctgatgaataaccaacgaaaaaaggcgagaagtacgcctttcaatatgctgaaacgcgagagaaaccgcgtgtcaactgtgcaacagctgacaaagagattctcacttggaatgctgcaaggacgcggaccattaaaactgttcatggcccttgtggcgttccttcgtttcctaacaatcccaccaacagcagggatactaaaaagatggggaacgatcaaaaaatcaaaagctatcaatgttttgagagggttcaggaaagagattggaaggatgctgaacatcttgaacaagagacgcaggacagcaggcgtgattgttatgttgattccaacagcgatggcgttccatttaaccacacgcaatggagaaccacacatgatcgttggtaggcaggagaaagggaaaagtcttctgttcaaaacagaggatggtgttaacatgtgtactctcatggccatagaccttggtgaattgtgtgaagatacaatcacgtacaagtgtcctctcctcagacaaaatgaaccagaagacatagattgttggtgcaactctacgtccacatgggtaacttatgggacatgtaccaccacaggagaacacagaagagaaaaaagatcagtggcgctcgttccacatgtaggtatgggactggagacacgaactgaaacatggatgtcatcagaaggggcctggaaacatgttcagagaattgaaacctggatcttgagacatccaggttttaccataatggcagcaatcctggcatacaccataggaacgacacatttccaaagggccttgattttcattttactgacagctgtcgctccttcaatgacaatgcgctgcataggaatatcaaatagagacttcgtagaaggggtttcaggaggaagctgggttgacatagtcttagaacatggaagttgtgtgacgacgatggcaaaaaacaaaccaacattggattttgaactgataaaaacagaagccaaacaacctgccactctaaggaagtactgtatagaagcaaagctgaccaacacaacaacagaatcgcgttgcccaacacaaggggaacccagtctaaatgaggagcaggacaaaaggttcatctgcaaacactccatggtagacagaggatggggaaatggatgtggattatttggaaagggaggcattgtgacctgtgctatgtttacatgcaaaaagaacatggaaggaaaagtcgtgcagccagaaaatttggaatacaccatcgtgataacacctcactcaggagaggagcacgctgtaggtaatgacacaggaaagcatggcaaggaaatcaaaataacaccacagagctccatcacagaagcagaactgacaggctatggcactgtcacgatggagtgctctccgagaacgggcctcgacttcaatgagatggtactgctgcagatggaagacaaagcttggctggtgcacaggcaatggttcctagacctgccgttaccatggctacccggagcggacacacaaggatcaaattggatacagaaagagacattggtcactttcaaaaatccccacgcgaagaaacaggatgtcgttgtcttagggtctcaagaaggggccatgcacacggcactcacaggggccacagaaatccagatgtcatcaggaaacttactgttcacaggacatctcaagtgcaggctgagaatggacaaactacagctcaaaggaatgtcatactctatgtgtacaggaaagtttaaaattgtgaaggaaatagcagaaacacaacatggaacaatagttatcagagtacaatatgaaggggacggttctccatgcaagatcccttttgagataacagatttggaaaaaagacacgtcttaggtcgcttgattacagttaacccaatcgtaacagaaaaagatagcccagtcaacatagaagcagaacctccattcggagacagctacatcattataggagtagagccgggacaattgaaactcaattggtttaagaagggaagttccatcggccaaatgtttgagacaacaatgagaggagcaaagagaatggccattttaggtgacacagcctgggactttggatccctgggaggagtgtttacatctataggaaaggctctccaccaagttttcggagcaatctatggggctgcttttagtggggtctcatggactatgaaaatcctcataggagtcatcatcacatggataggaatgaattcacgtagcacctcactgtctgtgtcgctagtattggtgggcgttgtgacactgtacctgggagctatggtgcaagctgatagtggttgcgttgtgagctggaaaaataaagaactgaaatgtggcagcgggatcttcatcacagataacgtacacacatggacagaacaatataagttccaaccagaatccccttcaaaactagcttcagctatccaaaaagctcatgaagagggcatttgtggaatccgctcagtaacaagattggagaatctgatgtggaaacaaataacaccagaattgaatcatattctatcagaaaatgaggtaaagttgaccattatgacaggagacattagaggaatcatgcaggcaggaaaacgatccttgcggccccagcccactgagctgaagtactcatggaaaacatggggaaaggcgaaaatgctctccacagagtctcacaatcagacctttcttattgatggccctgaaacagcagaatgccccaacacaaacagagcctggaactcgctggaagttgaagactatggttttggagttttcaccaccaatatatggctgaaattgagagaaaaacaggatgtattttgtgactcaaaactcatgtcagcggccattaaagacaacagagccgttcatgctgatatgggttattggatagaaagtgcactcaatgacacatggaagatggagaaagcctccttcattgaagttaaaagctgccactggccaaagtcacacaccctttggagcaatggagtattagaaagtgagatgataatcccaaaaaattttgccgggccagtgtcacaacacaactacagaccaggctaccatacacaaacagcaggaccttggcatctaggtaagcttgagatggactttgatctctgcgaaggaactacagtggtggtgactgaggactgtggaaatagaggaccctctttaagaacgaccactgcctctggaaaactcataacagaatggtgctgccgatcctgcacactaccacctctaagatacagaggtgaggatggatgctggtacgggatggaaatcagaccattgaaagagaaagaagagaatttggtcaactccttggtcacagccggacatgggcagattgacaacttttcactaggagtcttgggaatggcactgttcctggaagaaatgctcaggacccgaataggaacgaaacatgcaatactgctagttgcagtatcttttgtgacattgattactgggaacatgtcttttagagacctgggaagagtgatggttatggtgggcgctaccatgacggatgacataggtatgggagtgacttatcttgccctactagcagcttttaaggttagaccaacttttgcagctggactactcttaagaaaactgacctccaaggaattgatgatggccaccataggaatcgcactcctttcccaaagcaccataccagagaccattcttgaactgactgatgcattagccctgggcatgatggtcctcaaaatagtgagaaatatggaaaaataccaattggcagtgactatcatggctatttcatgtgtcccaaatgcagtgatactgcaaaacgcatggaaggtgagttgcacaatattggcagcggtgtccgtttcaccactgctcttaacatcctcacagcagaaagcggattggataccactggcattgacgataaaaggtctcaatccaacagccatttttttaacaactctctcgaggaccagcaagaaaaggagctggccgctaaatgaagctatcatggcagttgggatggtgagcattttagccagttctctcctaaagaatgatattcctatgacaggtccattagtggctggaggactcctcaccgtatgttacgtgctcactggacgatcggccgatttggaactggagagagctgccgatgtaaaatgggaagatcaggcagaaatatcaggaagcagcccaatcctgtcaataacaatatcagaagatggcagcatgtcgataaaaaatgaagaggaagaacaaacactgaccatactcatcagaacgggattgttggtgatctcaggagtctttccagtatcgataccaattacggcagcagcatggtacctgtgggaagtaaagaaacaacgggctggagtactgtgggacgtcccttcacccccaccagtggaaaaagccgaactggaggatggagcctacagaatcaagcaaagagggatccttggatattctcagattggagccggagtttacaaagaaggaacattccatacaatgtggcacgtcacacgtggtgctgttctgatgcatagagggaagaggattgaaccatcatgggcagatgtcaagaaagacctaatatcatatggaggaggctggaagctagaaggagaatggaaggaaggagaggaagtccaagtcctggcattggaacctggaaaaaatccaagagccgtccaaacgaaacctggaatattcaaaaccaacaccggaaccataggcgccgtatctctggacttttcccctggaacgtcaggatctccaatcgtcgacagaaaaggaaaagttgtgggtctttacggtaatggtgttgtcacaaggagtggagcatatgtaagtgctatagcccagaccgaaaaaagcattgaagacaatccagagatcgaagatgacattttccgaaagaaaagattgaccatcatggacctccatccaggagcaggaaagacaaaaagataccttccagccatagttagagaagccataaaacgtggcttgagaacattgatcctggctcccactagagtagtggcagctgaaatggaggaagctcttagaggacttccaataagataccaaactacagccatcaaaaccgagcataccgggcgggagatcgtggacctaatgtgtcatgccacatttactatgaggctgttatcaccagtcagagtgccaaattacaacctgatcatcatggacgaagcccacttcacagacccagcaagtatagcagctagaggatacatttcaactcgagtagagatgggtgaagcagccgggattttcatgacagccactcctccgggaagtagagacccatttcctcagagcaatgcaccaattatggatgaggaaagagaaatccctgagcgttcatggaattcaggacacgaatgggtcacggattttaaggggaagactgtttggtttgttccaagtataaaagcaggaaatgatatagcagcttgtcttaggaaaaatggaaagaaagtgatacaactcagtaggaagacttttgactctgagtatgttaagactagagccaatgattgggactttgtggtcacaactgacatttcagaaatgggtgccaacttcaaggctgagagggttatagaccccagacgttgcatgaaaccagttatactaacagatggcgaggagcgggtgatcttggctggacctatgccagtgacccactctagtgcagcgcaaagaagagggagaataggaagaaatccaaaaaatgaaaatgaccagtacatatacatgggggaacctcttgaaaatgatgaagactgtgcacattggaaagaagctaaaatgctcctagataacatcaacacacctgaaggaatcattcctagtatgttcgaaccagagcgtgaaaaagtggatgccattgatggtgaataccgtttgagaggagaagcaaggaaaacctttgtggacctaatgagaagaggggacttaccagtctggttggcctacaaagtggcagctgaaggcatcaactacgcagacagaaagtggtgttttgatggaattaagaacaaccaaatactggaagaaaatatggaagtggaaatctggacaaaagaaggggaaaggaaaaaattaaaacccagatggttggatgctaggatctattctgacccactagcactaaaagaattcaaggaatttgcagctggaagaaaatctttgaccctgaacctaatcacagaaatgggtaggcttccaactttcatgactcagaaagcaagaaacgcactggacaacctggctgtgctgcatacggctgaggcaggtggaagggcgtacaatcatgctctcagtgaactgccggagaccctggagacactgctcctactgacacttctggcaacagtcacaggaggaatcttcttattcttaatgagcggaaaaggtatagggaagatgaccctgggaatgtgttgcataatcacggctagtatcctcctatggtatgcacagatacaaccacactggatagcagcttcaataatactggagttttttctcatagttttgctcattccagaaccagaaaaacagagaacaccccaagacaaccaattgacctacgttgtcatagccatcctcacagtggtggccgcaaccatggcaaacgagatgggtttcctggaaaaaaccaagaaagacctcggattgggaagcattacaacccaggaatctgagagcaatatcctggacatagatctacgccctgcatcagcatggacgctgtatgccgtagctacaacatttgtcacaccaatgttgagacatagcattgaaaattcctcagtgaatgtctccctaacagccattgctaaccaagctacagtgctaatgggtcttgggaaaggatggccattgtcaaagatggacattggagttcccctccttgccattggatgctattcacaagtcaaccctataactctcacagcagctctccttttattggtagcacattatgccattatagggccaggacttcaagcaaaagcaaccagagaagcccagaaaagagcagcagcaggcatcatgaaaaacccaacagtcgatggaataacagtgattgacctagaaccaataccctatgatccaaaatttgaaaagcagttaggacaagtaatgctcctaatcctctgcgtgactcaagtattaatgatgaggactacatgggctttatgtgaggctctaaccctagcgaccgggcccatctccacactgtgggaaggaaatccagggaggttttggaacactaccattgcagtgtcaatggctaacatctttagggggagctacttggccggagctggacttctcttttccatcatgaaaaacacaacaaacacaagaagaggaactggcaacataggagagacacttggagaaaaatggaaaagtcgattaaacgcactggggaaaagtgaatttcaaatctacaagaaaagtggaatccaggaagtggatagaaccctagcaaaagaaggtatcaaaagaggagaaacggaccaccatgctgtgtcgcgaggttcagcaaaactgagatggttcgtcgagagaaatatggtcacaccggaagggaaggtggtggatctcggttgcggcagagggggctggtcatactattgcgggggactaaagaatgtaagagaagtcaaaggcctaacaaaaggaggaccaggacatgaagaacccatccccatgtcaacatatgggtggaatctagtgcgtctgcaaagtggagttgacgttttcttcaccccgccagaaaagtgtgatacattgttgtgtgacataggggagtcgtcaccaaatcccacgatagaagcaggacgaacactcagagtcctcaacttagtggaaaattggttgaacaataacacccaattttgcataaaggtcctcaacccatatatgccttcagtcatagaaaaaatggaagcattacaaaggaaatatggaggagccttagtgaggaatccactctcacgaaactccacgcatgaaatgtactgggtatctaatgccaccgggaacatagtgtcatcagtgaacatgatttcaaggatgttgattaacagattcacaatgaaacacaagaaagccacttacgagccagatgttgacctaggaagtggaacccgcaacattggaattgaaagtgagataccaaatctagacataataggaaagagaatagagaaaataaaacaagagcatgaaacatcatggcactatgatcaagaccacccatacaaaacgtgggcttaccatggcagctatgaaacaaaacaaactggatcagcatcatctatggtgaacggagtggtcagattgctgacaaaaccttgggacgtcgtccctatggtgacacagatggcaatgacagacacgactccttttggacaacagcgcgttttcaaagagaaagtagacacgagaacccaagaaccgaaggaaggcacaaagaaactgatgaaaattacggcagagtggctttggaaagaactaggaaagaaaaagacacctaggatgtgtaccagagaagaattcacaagaaaagtgagaagcaatgcagccttgggggccatattcactgatgagaacaaatggaaatcggcacgtgaggctgttgaagatggtaggttctgggagctggttgacagggaaagaaatctccatcttgaaggaaagtgtgaaacatgtgtgtacaacatgatgggaaaaagagagaagaaactaggggagttcggcaaggcaaaaggtagcagagccatatggtacatgtggcttggagcacgcttcttagagtttgaagccctaggattcctgaatgaagatcactggttctccagagggaactccctgagtggagtggaaggagaagggctgcacaggctaggctacattttaagagacgtgggcaagaaggaagggggggcaatgtacgccgatgatacagcaggatgggacacaagaatcacactagaagacttaaaaaatgaagaaatggtaacgaaccacatgaaaggagaacacaagaaactagccgaggccatattcaaactaacgtaccaaaacaaggtggtgcgtgtgcaaagaccaacaccaagaggtacagtaatggatatcatatcgagaagagaccaaagaggcagtgggcaagtcggcacctatggccttaataccttcaccaatatggaagcccaattaattagacagatggagggagaaggaatcttcaaaagcattcagcacctgacagccacagaagaaatcgctgtacagaactggctagcaagagtggggcgtgaaaggctatcaagaatggcaatcagtggagatgattgtgttgtaaaacctatagatgacagatttgcaagtgctttaacagctctaaatgacatgggaaaagttagaaaagatatacaacaatgggaaccttcaagaggatggaacgattggacacaagtgcctttctgttcacaccactttcatgagttagtcatgaaagatggtcgcgtgctcgtagtcccatgcagaaaccaagatgaactgattggtagagcccgaatttcccagggagctgggtggtctttgaaagagacggcctgtttgggaaagtcttacgcccaaatgtggactctgatgtacttccacagacgtgacctcagactggcggcaaatgccatctgctcggcagtcccgtcacattgggttccaacaagtcgaacaacctggtccatacacgctaagcatgaatggatgacgacggaagacatgctggcagtctggaacagggtgtggatccaagaaaacccgtggatggaagataaaactccagtggaatcatgggaagaagtcccatacttgggaaaaagagaagaccaatggtgcggctcattgattgggctaacaagcagggctacctgggcaaagaacatccaaacagcaataaatcaagtcagatcccttataggcaatgaggaatacacagactacatgccatccatgaagagatttagaagggaagaggaagaggcaggtgtcctgtggtagaaggcaaaactaacatgaaacaaggctaaaagtcaggtcggattaagccatagtacggaaaaaactatgctacctgtgagccccgtccaaggacgttaaaagaagtcaggccatcacaaaatgccacagcttgagtaaactgtgcagcctgtagctccacctgaggaggtgtaaaaaacctgggaggccacaaaccatggaagctgtacgcatggcgtagtggactagcggttagaggagacccctcccttacaaatcgcagcaaacaacgggggcccaaggtgagatgaagctgtaatctcactggaaggactagaggttagaggagacccccccaaaacaaaaaacagcatattgacgctgggaaagaccagagatcctgctgtctcctcagcatcattccaggcacagaacgccagaaaatggaatggtgctgttgaatcaacaggttct

SEQ ID NO: 4 - DNA, 10725 bp, Dengue virus
agttgttagtctacgtggaccgacaaagacagattctttgagggagctaagctcaacgtagttctaacagttttttaattagagagcagatctctgatgaataaccaacgaaaaaaggcgagaagtacgcctttcaatatgctgaaacgcgagagaaaccgcgtgtcaactgtgcaacagctgacaaagagattctcacttggaatgctgcaaggacgcggaccattaaaactgttcatggcccttgtggcgttccttcgtttcctaacaatcccaccaacagcagggatactaaaaagatggggaacgatcaaaaaatcaaaagctatcaatgttttgagagggttcaggaaagagattggaaggatgctgaacatcttgaacaagagacgcaggacagcaggcgtgattgttatgttgattccaacagcgatggcgttccatttaaccacacgcaatggagaaccacacatgatcgttggtaggcaggagaaagggaaaagtcttctgttcaaaacagaggatggtgttaacatgtgtactctcatggccatagaccttggtgaattgtgtgaagatacaatcacgtacaagtgtcctctcctcagacaaaatgaaccagaagacatagattgttggtgcaactctacgtccacatgggtaacttatgggacatgtaccaccacaggagaacacagaagagaaaaaagatcagtggcgctcgttccacatgtaggtatgggactggagacacgaactgaaacatggatgtcatcagaaggggcctggaaacatgttcagagaattgaaacctggatcttgagacatccaggttttaccataatggcagcaatcctggcatacaccataggaacgacacatttccaaagggccttgattttcattttactgacagctgtcgctccttcaatgacaatgagatgcatagggatatcgaatcgcgatttcgtcgaaggcgtatccggagggtcatgggtcgacatcgtactcgaacacggatcatgcgttacgactatggctaagaataagcctacactagacttcgaactgattaagacagaggctaagcaaccggcaacattgcgtaagtactgtatcgaagcgaaactgactaacactacaaccgaatctagatgccctacacagggcgaacctagtctgaacgaagagcaagacaaaaggtttatatgcaaacactctatggtcgatcgcggatggggaaacggatgcggattgttcggtaaggggggaatcgttacatgcgctatgtttacatgtaaaaaaaatatggagggaaaggtcgtgcaaccagagaatctcgaatatacaatcgtaatcacaccgcatagcggagaggaacacgcagtcggaaacgataccggaaaacacggaaaagagattaagattacaccacagagctccataaccgaagccgaactgacagggtacggaacagtgacaatggaatgctcacctagaacaggcctagactttaacgaaatggtgttactgcaaatggaagacaaagcatggttagtgcataggcaatggtttttagacctaccactaccatggttgcccggagccgatacacagggatcgaactggatacagaaagagacactcgttacgtttaaaaacccacacgctaaaaaacaggacgtagtcgtactcggatcacaggaaggcgcaatgcataccgcattgacaggcgctacagagatacagatgtctagcggaaatctgttgtttacagggcatctgaaatgtagactgagaatggacaaactgcaattgaagggaatgtcatactctatgtgtacgggtaagtttaagatagtcaaagagatagccgaaacacaacacggaacaatcgtaattagggtgcaatacgaaggcgacgggtcaccatgtaagataccattcgaaattacagacctcgaaaaaagacacgtactcggaagactgataacagtgaatccgatcgttacggaaaaagactcacccgttaatatcgaagccgaaccaccattcggagactcatacataataatcggagtcgaacccggacaattgaaactgaattggtttaaaaaagggtcatcaatcggacaaatgttcgaaacaactatgagaggcgctaagcgtatggctatactcggagacacagcatgggacttcggatccttagggggagtgtttacgtcaatcggtaaggcactacaccaggtattcggagcgatatacggagccgcatttagcggagtgtcgtggacaatgaagatactgatcggagtgataatcacatggatcggaatgaatagtaggtctacaagtctatccgttagcttagtgttagtcggagtcgttacactgtatctaggcgctatggtgcaagccgatagtggttgcgttgtgagctggaaaaataaagaactgaaatgtggcagcgggatcttcatcacagataacgtacacacatggacagaacaatataagttccaaccagaatccccttcaaaactagcttcagctatccaaaaagctcatgaagagggcatttgtggaatccgctcagtaacaagattggagaatctgatgtggaaacaaataacaccagaattgaatcatattctatcagaaaatgaggtaaagttgaccattatgacaggagacattagaggaatcatgcaggcaggaaaacgatccttgcggccccagcccactgagctgaagtactcatggaaaacatggggaaaggcgaaaatgctctccacagagtctcacaatcagacctttcttattgatggccctgaaacagcagaatgccccaacacaaacagagcctggaactcgctggaagttgaagactatggttttggagttttcaccaccaatatatggctgaaattgagagaaaaacaggatgtattttgtgactcaaaactcatgtcagcggccattaaagacaacagagccgttcatgctgatatgggttattggatagaaagtgcactcaatgacacatggaagatggagaaagcctccttcattgaagttaaaagctgccactggccaaagtcacacaccctttggagcaatggagtattagaaagtgagatgataatcccaaaaaattttgccgggccagtgtcacaacacaactacagaccaggctaccatacacaaacagcaggaccttggcatctaggtaagcttgagatggactttgatctctgcgaaggaactacagtggtggtgactgaggactgtggaaatagaggaccctctttaagaacgaccactgcctctggaaaactcataacagaatggtgctgccgatcctgcacactaccacctctaagatacagaggtgaggatggatgctggtacgggatggaaatcagaccattgaaagagaaagaagagaatttggtcaactccttggtcacagccggacatgggcagattgacaacttttcactaggagtcttgggaatggcactgttcctggaagaaatgctcaggacccgaataggaacgaaacatgcaatactgctagttgcagtatcttttgtgacattgattactgggaacatgtcttttagagacctgggaagagtgatggttatggtgggcgctaccatgacggatgacataggtatgggagtgacttatcttgccctactagcagcttttaaggttagaccaacttttgcagctggactactcttaagaaaactgacctccaaggaattgatgatggccaccataggaatcgcactcctttcccaaagcaccataccagagaccattcttgaactgactgatgcattagccctgggcatgatggtcctcaaaatagtgagaaatatggaaaaataccaattggcagtgactatcatggctatttcatgtgtcccaaatgcagtgatactgcaaaacgcatggaaggtgagttgcacaatattggcagcggtgtccgtttcaccactgctcttaacatcctcacagcagaaagcggattggataccactggcattgacgataaaaggtctcaatccaacagccatttttttaacaactctctcgaggaccagcaagaaaaggagctggccgctaaatgaagctatcatggcagttgggatggtgagcattttagccagttctctcctaaagaatgatattcctatgacaggtccattagtggctggaggactcctcaccgtatgttacgtgctcactggacgatcggccgatttggaactggagagagctgccgatgtaaaatgggaagatcaggcagaaatatcaggaagcagcccaatcctgtcaataacaatatcagaagatggcagcatgtcgataaaaaatgaagaggaagaacaaacactgaccatactcatcagaacgggattgttggtgatctcaggagtctttccagtatcgataccaattacggcagcagcatggtacctgtgggaagtaaagaaacaacgggctggagtactgtgggacgtcccttcacccccaccagtggaaaaagccgaactggaggatggagcctacagaatcaagcaaagagggatccttggatattctcagattggagccggagtttacaaagaaggaacattccatacaatgtggcacgtcacacgtggtgctgttctgatgcatagagggaagaggattgaaccatcatgggcagatgtcaagaaagacctaatatcatatggaggaggctggaagctagaaggagaatggaaggaaggagaggaagtccaagtcctggcattggaacctggaaaaaatccaagagccgtccaaacgaaacctggaatattcaaaaccaacaccggaaccataggcgccgtatctctggacttttcccctggaacgtcaggatctccaatcgtcgacagaaaaggaaaagttgtgggtctttacggtaatggtgttgtcacaaggagtggagcatatgtaagtgctatagcccagaccgaaaaaagcattgaagacaatccagagatcgaagatgacattttccgaaagaaaagattgaccatcatggacctccatccaggagcaggaaagacaaaaagataccttccagccatagttagagaagccataaaacgtggcttgagaacattgatcctggctcccactagagtagtggcagctgaaatggaggaagctcttagaggacttccaataagataccaaactacagccatcaaaaccgagcataccgggcgggagatcgtggacctaatgtgtcatgccacatttactatgaggctgttatcaccagtcagagtgccaaattacaacctgatcatcatggacgaagcccacttcacagacccagcaagtatagcagctagaggatacatttcaactcgagtagagatgggtgaagcagccgggattttcatgacagccactcctccgggaagtagagacccatttcctcagagcaatgcaccaattatggatgaggaaagagaaatccctgagcgttcatggaattcaggacacgaatgggtcacggattttaaggggaagactgtttggtttgttccaagtataaaagcaggaaatgatatagcagcttgtcttaggaaaaatggaaagaaagtgatacaactcagtaggaagacttttgactctgagtatgttaagactagagccaatgattgggactttgtggtcacaactgacatttcagaaatgggtgccaacttcaaggctgagagggttatagaccccagacgttgcatgaaaccagttatactaacagatggcgaggagcgggtgatcttggctggacctatgccagtgacccactctagtgcagcgcaaagaagagggagaataggaagaaatccaaaaaatgaaaatgaccagtacatatacatgggggaacctcttgaaaatgatgaagactgtgcacattggaaagaagctaaaatgctcctagataacatcaacacacctgaaggaatcattcctagtatgttcgaaccagagcgtgaaaaagtggatgccattgatggtgaataccgtttgagaggagaagcaaggaaaacctttgtggacctaatgagaagaggggacttaccagtctggttggcctacaaagtggcagctgaaggcatcaactacgcagacagaaagtggtgttttgatggaattaagaacaaccaaatactggaagaaaatatggaagtggaaatctggacaaaagaaggggaaaggaaaaaattaaaacccagatggttggatgctaggatctattctgacccactagcactaaaagaattcaaggaatttgcagctggaagaaaatctttgaccctgaacctaatcacagaaatgggtaggcttccaactttcatgactcagaaagcaagaaacgcactggacaacctggctgtgctgcatacggctgaggcaggtggaagggcgtacaatcatgctctcagtgaactgccggagaccctggagacactgctcctactgacacttctggcaacagtcacaggaggaatcttcttattcttaatgagcggaaaaggtatagggaagatgaccctgggaatgtgttgcataatcacggctagtatcctcctatggtatgcacagatacaaccacactggatagcagcttcaataatactggagttttttctcatagttttgctcattccagaaccagaaaaacagagaacaccccaagacaaccaattgacctacgttgtcatagccatcctcacagtggtggccgcaaccatggcaaacgagatgggtttcctggaaaaaaccaagaaagacctcggattgggaagcattacaacccaggaatctgagagcaatatcctggacatagatctacgccctgcatcagcatggacgctgtatgccgtagctacaacatttgtcacaccaatgttgagacatagcattgaaaattcctcagtgaatgtctccctaacagccattgctaaccaagctacagtgctaatgggtcttgggaaaggatggccattgtcaaagatggacattggagttcccctccttgccattggatgctattcacaagtcaaccctataactctcacagcagctctccttttattggtagcacattatgccattatagggccaggacttcaagcaaaagcaaccagagaagcccagaaaagagcagcagcaggcatcatgaaaaacccaacagtcgatggaataacagtgattgacctagaaccaataccctatgatccaaaatttgaaaagcagttaggacaagtaatgctcctaatcctctgcgtgactcaagtattaatgatgaggactacatgggctttatgtgaggctctaaccctagcgaccgggcccatctccacactgtgggaaggaaatccagggaggttttggaacactaccattgcagtgtcaatggctaacatctttagggggagctacttggccggagctggacttctcttttccatcatgaaaaacacaacaaacacaagaagaggaactggcaacataggagagacacttggagaaaaatggaaaagtcgattaaacgcactggggaaaagtgaatttcaaatctacaagaaaagtggaatccaggaagtggatagaaccctagcaaaagaaggtatcaaaagaggagaaacggaccaccatgctgtgtcgcgaggttcagcaaaactgagatggttcgtcgagagaaatatggtcacaccggaagggaaggtggtggatctcggttgcggcagagggggctggtcatactattgcgggggactaaagaatgtaagagaagtcaaaggcctaacaaaaggaggaccaggacatgaagaacccatccccatgtcaacatatgggtggaatctagtgcgtctgcaaagtggagttgacgttttcttcaccccgccagaaaagtgtgatacattgttgtgtgacataggggagtcgtcaccaaatcccacgatagaagcaggacgaacactcagagtcctcaacttagtggaaaattggttgaacaataacacccaattttgcataaaggtcctcaacccatatatgccttcagtcatagaaaaaatggaagcattacaaaggaaatatggaggagccttagtgaggaatccactctcacgaaactccacgcatgaaatgtactgggtatctaatgccaccgggaacatagtgtcatcagtgaacatgatttcaaggatgttgattaacagattcacaatgaaacacaagaaagccacttacgagccagatgttgacctaggaagtggaacccgcaacattggaattgaaagtgagataccaaatctagacataataggaaagagaatagagaaaataaaacaagagcatgaaacatcatggcactatgatcaagaccacccatacaaaacgtgggcttaccatggcagctatgaaacaaaacaaactggatcagcatcatctatggtgaacggagtggtcagattgctgacaaaaccttgggacgtcgtccctatggtgacacagatggcaatgacagacacgactccttttggacaacagcgcgttttcaaagagaaagtagacacgagaacccaagaaccgaaggaaggcacaaagaaactgatgaaaattacggcagagtggctttggaaagaactaggaaagaaaaagacacctaggatgtgtaccagagaagaattcacaagaaaagtgagaagcaatgcagccttgggggccatattcactgatgagaacaaatggaaatcggcacgtgaggctgttgaagatggtaggttctgggagctggttgacagggaaagaaatctccatcttgaaggaaagtgtgaaacatgtgtgtacaacatgatgggaaaaagagagaagaaactaggggagttcggcaaggcaaaaggtagcagagccatatggtacatgtggcttggagcacgcttcttagagtttgaagccctaggattcctgaatgaagatcactggttctccagagggaactccctgagtggagtggaaggagaagggctgcacaggctaggctacattttaagagacgtgggcaagaaggaagggggggcaatgtacgccgatgatacagcaggatgggacacaagaatcacactagaagacttaaaaaatgaagaaatggtaacgaaccacatgaaaggagaacacaagaaactagccgaggccatattcaaactaacgtaccaaaacaaggtggtgcgtgtgcaaagaccaacaccaagaggtacagtaatggatatcatatcgagaagagaccaaagaggcagtgggcaagtcggcacctatggccttaataccttcaccaatatggaagcccaattaattagacagatggagggagaaggaatcttcaaaagcattcagcacctgacagccacagaagaaatcgctgtacagaactggctagcaagagtggggcgtgaaaggctatcaagaatggcaatcagtggagatgattgtgttgtaaaacctatagatgacagatttgcaagtgctttaacagctctaaatgacatgggaaaagttagaaaagatatacaacaatgggaaccttcaagaggatggaacgattggacacaagtgcctttctgttcacaccactttcatgagttagtcatgaaagatggtcgcgtgctcgtagtcccatgcagaaaccaagatgaactgattggtagagcccgaatttcccagggagctgggtggtctttgaaagagacggcctgtttgggaaagtcttacgcccaaatgtggactctgatgtacttccacagacgtgacctcagactggcggcaaatgccatctgctcggcagtcccgtcacattgggttccaacaagtcgaacaacctggtccatacacgctaagcatgaatggatgacgacggaagacatgctggcagtctggaacagggtgtggatccaagaaaacccgtggatggaagataaaactccagtggaatcatgggaagaagtcccatacttgggaaaaagagaagaccaatggtgcggctcattgattgggctaacaagcagggctacctgggcaaagaacatccaaacagcaataaatcaagtcagatcccttataggcaatgaggaatacacagactacatgccatccatgaagagatttagaagggaagaggaagaggcaggtgtcctgtggtagaaggcaaaactaacatgaaacaaggctaaaagtcaggtcggattaagccatagtacggaaaaaactatgctacctgtgagccccgtccaaggacgttaaaagaagtcaggccatcacaaaatgccacagcttgagtaaactgtgcagcctgtagctccacctgaggaggtgtaaaaaacctgggaggccacaaaccatggaagctgtacgcatggcgtagtggactagcggttagaggagacccctcccttacaaatcgcagcaaacaacgggggcccaaggtgagatgaagctgtaatctcactggaaggactagaggttagaggagacccccccaaaacaaaaaacagcatattgacgctgggaaagaccagagatcctgctgtctcctcagcatcattccaggcacagaacgccagaaaatggaatggtgctgttgaatcaacaggttct

SEQ ID NO: 5 - DNA, 10708 bp, Dengue virus
agttgttagtctacgtggaccgacaagaacagtttcgactcggaagcttgcttaacgtagtgctaacagttttttattagagagcagatctctgatgaacaaccaacggaagaagacgggaaaaccgtctatcaatatgctgaaacgcgtgagaaaccgtgtgtcaactggatcacagttggcgaagagattctcaaaaggactgctgaacggccagggaccaatgaaattggttatggcgttcatagctttcctcagatttctagccattccaccaacagcaggagtcttggctagatggggaaccttcaagaagtcgggggccattaaggtcctgaaaggctttaagaaggagatctcaaacatgctgagcataatcaacaaacggaaaaagacatcgctctgtctcatgatgatattgccagcagcacttgctttccacttgacttcacgagatggagagccgcgcatgattgtggggaagaatgaaagaggaaaatccctactttttaagacagcctctggaattaacatgtgcacactcatagccatggacttgggagagatgtgtgatgacacggtcacttacaaatgcccccatattaccgaagtggaacctgaagacattgactgctggtgcaacctcacatcaacatgggtgacttatggaacgtgcaatcaagccggagagcatagacgcgataagagatcagtggcgttagctccccatgtcggcatgggactggatacacgcacccaaacttggatgtcggctgaaggagcttggaggcaagtcgagaaggtagagacatgggcccttaggcacccagggttcaccatactagccctatttcttgcccattacataggcacttccttgacccagaaggtggttatttttatactgctaatgctggtcaccccatccatgacaatgagatgtgtgggagtaggaaacagagattttgtggaaggactatcaggagctacgtgggttgacgtggtgctcgagcacggggggtgtgtgaccaccatggctaagaacaagcccacgttggatatagagcttcagaagaccgaggccacccaactggcgaccctaaggaagctatgcattgaggggaaaatcaccaacataacaactgactcaagatgtcctacccaaggggaagcggttttgcctgaggagcaggaccaaaactacgtgtgtaagcatacatacgtagacagaggctgggggaacggatgtggtttgtttggtaagggaagcttggtaacatgtgcgaaatttcaatgcctggaaccaatagagggaaaagtggtgcaatatgagaacctcaaatacaccgttatcatcacagtgcacacaggagaccaacaccaggtgggaaatgaaacgcagggagtcacggctgagataacacctcaggcatcaaccactgaagccatcttgcctgaatatggaacccttgggctagaatgctcaccacggacaggtttggacttcaatgaaatgatcttgctaacaatgaagaacaaagcatggatggtacatagacaatggttttttgacctacctctaccatggacatcaggagctacaacggaaacaccaacctggaacaggaaggagcttcttgtgacatttaaaaacgcacatgcgaagaaacaagaagtagttgttcttgggtcgcaagagggagcaatgcataccgcattgacaggagccacagaaatccaaaactcaggaggcacaagcatttttgcggggcacttaaaatgtagacttaagatggacaaattagaactcaaggggatgagctatgcaatgtgcacgaatacctttgtgttgaagaaagaagtctcagaaacgcagcatgggacaatacttatcaaggtcgagtacaaaggggaagatgtaccttgcaagattcctttctccacagaggatggacaagggaaagctcacaatggcagactgattacagccaacccagtggtgactaagaaggaggagcctgtcaatattgaggctgaacctccttttggggaaagcaatatagtaattggaattggagacaacgccttgaaaatcaactggtacaagaagggaagctctattgggaagatgttcgaggccactgctagaggtgcaaggcgcatggccatcttgggagacacagcttgggactttggatcagtgggtggtgttctgaactcattaggcaaaatggtgcaccaaatattcggaagtgcttacacagccctattcagtggagtctcttgggtaatgaaaattggaataggagttctcttgacttggatagggttgaattcaaaaaacacatccatgtcattttcatgcattgcgataggaatcatcacactctatctgggagctgtggtgcaagctgacatggggtgtgttataaactggaaaggcaaagaactcaagtgtggaagtggaatcttcgtcaccaacgaggtccatacctggacagagcaatacaaattccaagcagactccccaaaaagattggcgacagccattgcaggcgcttgggagaatggagtgtgcggaattaggtcaacaaccagaatggagaatctcctgtggaagcaaatagccaatgaactgaactacatattgtgggaaaacaatatcaaattaacggttgttgtgggcgatataattggggtcttagagcaagggaaaagaacactaacaccacaacccatggagctaaaatactcatggaaaacgtggggaaaggcaaaaatagtgacagctgaaacacaaaattcttctttcataatagatggaccaaacacaccggagtgtccaagtgcctcaagagcatggaatgtgtgggaggtggaagattacgggttcggagtcttcacaaccaacatatggctgaaactccgagaggtgtatacccaactatgtgaccataggttaatgtcggcagccgtcaaggatgaaagggccgtacatgccgacatgggctattggatagaaagtcaaaagaatggaagttggaagctagaaaaagcatccctcatagaggtgaaaacctgcacatggccaaaatcacataccctttggagtaatggtgtgttagagagtgacatgatcattccaaaaagtctagctggtcctatctcgcaacacaactacaggcccgggtaccacacccagacggcgggaccttggcatttaggaaaattagagctggacttcaactattgtgaaggaacaacagttgtcatcacagaaaactgtgggacaagaggcccatcattgagaacaacaacagtgtcagggaagttaatacacgaatggtgctgccgctcgtgcacacttcctcccctgcgatacatgggagaagacggttgctggtatggcatggaaatcagacccatcagtgagaaagaagaaaacatggtaaagtctttagtctcagcgggaagtggagaggtggacaacttcacaatgggtgtcttgtgtttggcaatcctctttgaagaggtgatgagaggaaaatttgggaagaaacacatgattgcgggggttttcttcacgtttgtactccttctctcagggcaaataacatggagagatatggcgcacacactaataatgattgggtccaatgcatctgacaggatgggaatgggcgtcacctacctagctttaattgcaacatttaaaatccagccatttttggctttgggatttttcctaagaaaactgacatccagagaaaatttattgttaggagttgggctggctatggcaacaacgttacaactgccagaggacattgaacaaatggcaaatggaatcgctctggggctcatggctctcaaactgataacacaatttgaaacataccaactatggacagcattaatctccttaacgtgttcaaatacaatttttacgttgactgttgcctggagaacagccaccctgattttggctggagtttcacttttaccagtgtgccagtcttcgagtatgaggaaaacagactggcttccaatgacagtggcagctatgggagttccacctctaccactttttatttttagcttaaaagacacactcaaaaggagaagctggccactgaatgaaggggtgatggctgttgggcttgtgagcattctggccagttctctccttagaaatgatgtacccatggctggaccattagtggccgggggcttgctgatagcgtgctacgtcataactggcacgtcagcagacctcaccgtagaaaaagcagcagatgtaacatgggaggaagaggctgagcaaacaggagtgtcccacaacttaatgatcacagttgatgatgatggaacaatgagaataaaagatgatgagactgagaatatcttaacagtgcttttgaaaacagcattactaatagtgtcaggaatctttccatactccatacccgcaacattgttggtctggcatacttggcaaaagcaaacccaaagatccggcgttctatgggatgtacccagccctccagagacacagaaagcagaactggaagaaggggtctataggatcaaacagcaaggaatttttgggaaaacccaagtaggggttggagtacagaaagaaggagtctttcacaccatgtggcacgttacaagaggggcagtgttgacatataatgggaaaagactggaaccaaattgggctagcgtgaaaaaagatctgatttcatacggaggaggatggagattgagcgcacaatggcaaaagggggaggaggtgcaggttattgccgtagagcctgggaagaacccaaagaactttcaaaccatgccaggcacttttcagactacaacaggggaaataggagcaattgcactggatttcaagcccggaacttcaggatctcctatcataaacagagagggaaaggtagtgggactgtatggcaatggagtggttacaaagaatggtggctacgtcagcggaatagcgcaaacgaatgcagaaccagatggaccgacaccagaattggaagaagagatgttcaaaaagcgaaatctaaccataatggatcttcatcctgggtcaggaaagacacggaaataccttccagctattgttagagaggcaatcaagagacgtttgagaactctaattctggcaccaacaagggtggttgcagctgagatggaagaagcattgaaagggctcccaataaggtaccaaacaacagcaacaaaatctgaacacacaggaagagagattgttgatctgatgtgccacgcaacgttcacaatgcgtctgctgtcaccagttagggttccaaactataacttgataataatggatgaagcccatttcacagacccagccagtatagctgctagagggtacatatcaactcgtgttggaatgggagaagcagccgcaatattcatgacagcaacgccccctggaacagctgatgcctttccccagagcaacgctccaattcaagatgaagaaagggacataccagaacgctcatggaattcaggcaatgaatggataaccgacttcgctgggaaaacggtgtggtttgtccccagcattaaagccggaaatgacatagcaaattgcctgcggaaaaacgggaaaaaggtcattcaacttagtaggaagacttttgacacagaatatcagaaaaccaaactgaatgattgggactttgtggtgacgactgacatttcagaaatgggggccaatttcaaagcagatagagtgatcgacccaagaagatgtctcaaaccagtgatcctgacagatggaccagagcgggtgatcctggctggaccaatgccagtcaccgcagcgagtgctgcgcaaaggagagggagagttggcaggaacccacaaaaagaaaatgaccagtacatattcacgggccagcctctcaacaatgatgaagaccatgctcactggacagaagcaaaaatgctgctggacaacattaacacaccagaagggatcataccagctctctttgagccagaaagggagaagtcagccgccatagacggtgagtatcgcctgaaaggtgagtccaggaagactttcgtggaactcatgaggaggggtgaccttccagtctggttagcccataaagtagcatcagaagggatcaaatatacagatagaaaatggtgctttgatggacaacgtaataatcaaattttagaagagaacatggatgtggaaatctggacaaaggaaggagaaaagaaaaaattgagacctaggtggcttgatgctcgcacctattcagatcccttagcactcaaggaattcaaggactttgcggctggcaggaagtcaatagcccttgatcttgtgacagaaataggaagagtgccttcacacctagctcatagaacgagaaacgctctggacaatctggtgatgctgcatacgtcagaacatggcggtaaggcctacaggcatgcggtggaggaactaccagagacaatggaaacactcctactcttgggactcatgatcttgttgacaggtggagcaatgcttttcttaatatcaggtaaagggattggaaagacttcaataggactcatttgtgtaattgcttccagcggcatgttgtggatggccgaaatcccactccaatggatcgcgtcggccatagtcctggagttttttatgatggtgttgcttataccagaaccagagaagcagagaaccccccaagacaaccaactcgcatatgtcgtgataggcatacttacactggcagcaataatagcagccaatgaaatgggattgttggaaactacaaagagagatttaggaatgtctaaggagccaggtgttgtttctccaaccagctatttagatgtggacttgcacccagcatcagcctggacattgtacgctgtggccactacagtaataacaccaatgttaagacataccatagagaattccacagcaaatgtgtccttggcagctatagccaaccaggcagtggtcctgatgggtttggacaaaggatggccaatatcaaaaatggacttaggagtacccctgctggcattgggttgctattcacaagtgaacccactgactctaacagcggcagtactcttgctgatcacacattatgccattataggtccaggattgcaggcaaaagccacccgtgaagctcagaaaaggacagctgctggaataatgaagaatccaacagtggatgggataatgacaatagacctagatcctgtaatatatgattcaaaatttgaaaagcaactgggacaggttatgctcctggttttgtgtgcagttcaacttttgttaatgagaacatcatgggccttgtgtgaagctttaaccctagctacaggaccaataacaacactctgggaaggatcacctgggaagttttggaacaccacgatagctgtttccatggcgaacatttttagagggagctatttagcaggagctgggcttgctttctctattatgaaatcagttggaacagggaaaagaggaacaggctcacagggtgaaactttgggagaaaaatggaaaaagaagttaaatcaattatcccggaaagagtttgacctttacaagaaatctggaatcactgaggtggatagaacagaagccaaagaagggttgaaaagaggagaaataacacatcatgccgtgtccagaggtagtgcaaaacttcaatggtttgtggagagaaacatggtcattcccgaaggaagagttatagacttgggctgtggaagaggaggctggtcatattactgtgcaggactgaaaaaagtcacagaagtgcgaggatacacaaaaggcggtccaggacacgaagaaccagtacctatgtccacatatggatggaacatagttaagttaatgagtggaaaggatgtgttttatcttccacctgaaaagtgtgacaccctgttgtgcgacattggagaatcttcaccaagcccaacagtggaagaaagcagaactataagagttttgaagatggttgaaccatggctaaagaacaaccaattttgtattaaagtattgaacccttacatgccaactgtgattgagcacctagaaagactacaaaggaaacatggaggaatgcttgtgagaaatccactttcacgaaactccacgcacgaaatgtactggatatccaatggcacaggtaacattgtcgcttcagtcaacatggtatctagactgctactgaacaggttcacgatgacacacagaagacccaccattgagaaagatgtggatttaggagcaggaactcgacatgttaatgcggaaccagaaacacccaacatggatgtcattggggaaagaataaaaaggatcaaggaggagcataattcaacatggcactacgatgacgaaaacccctacaaaacgtgggcttaccatggatcttatgaagtcaaagccacaggctcagcctcctccatgataaatggagtcgtgaaactcctcactaaaccatgggatgtggtgcccatggtgacacagatggcaatgacagatacaactccatttggccagcagagagtctttaaagagaaagtggacaccaggacacccaggtccatgccaggaacaagaagggttatggggatcacagcggagtggctctggagaaccctgggaaggaataaaaaacccaggttatgcacaagggaagagtttacaaaaaaggtcagaactaacgcagccatgggagctgttttcacagaggagaaccaatgggacagcgcgaaagctgctgttgaggatgaggatttttggaaacttgtggacagagaacgtgaactccacaaactgggcaagtgtggaagctgtgtttacaacatgatgggtaagagagagaagaaacttggagagtttggcaaagcaaaaggcagtagagctatatggtacatgtggttgggagccaggtaccttgagttcgaagcccttggattcttaaatgaagaccactggttctcgcgtgagaactcttacagtggagtggaaggagaaggactgcacaagctaggctatatattaagggacatttccaagatacccggaggagctatgtatgctgatgacacagctggttgggacacaagaataacagaagatgacctgcacaatgaggaaaagatcacacagcaaatggaccctgaacacaggcagttagcgaacgctatatttaagctcacataccaaaacaaagtggtcaaagttcaacgaccgactccaacaggcacggtaatggatatcatatctaggaaagaccaaagaggcagtggacaggtaggaacttatggtctgaatacattcaccaacatggaagcccagttaatcagacaaatggaaggagaaggtgtgctgtcaaaggcagacctcgagaaccctcatctgccagagaagaaaattacacaatggttggaaaccaaaggagtggagaggttaaaaagaatggccataagtggggatgactgcgtggtgaaaccaatcgatgacaggttcgctaatgccctgctcgctctgaacgacatgggaaaggttcggaaagacatacctcaatggcagccatcaaagggatggcatgattggcaacaggttcctttctgctcccaccactttcatgaattgatcatgaaagatggaagaaagttggtggttccctgcagaccccaggacgaactaataggaagggcaagaatctctcaaggagcgggatggagccttagagaaaccgcatgtctggggaaagcctacgctcaaatgtggagtctcatgtattttcacagaagagacctcagactagcatccaacgccatatgttcagcagtaccagtccactgggtccccacaagtagaacgacatggtctattcacgctcaccatcagtggatgaccacagaagacatgcttactgtctggaacagagtgtggatcgaggacaatccatggatggaagacaaaactccagtcacaacctgggaaaatgttccatatctagggaagagagaagaccaatggtgcggatcacttattggtctcacttccagagcaacctgggcccagaacatacccacagcaattcaacaggttagaagccttataggcaatgaagagtttctggactacatgccttcaatgaagagatttaggaaggaggaggagtcggagggagccatttggtaaaacgtaggaagtgaaaaagaggttaactgtcaggccatattaagccacagtacggaagaagctgtgctgcctgtgagccccgtccaaggacgttaaaagaagaagtcaggccccaaagccacggtttgagcaaaccgtgctgcctgtagctccgtcgtggggacgtaaaacctgggaggctgcaaactgtggaagctgtacgcacggtgtagcagactagcggttagaggagacccctcccatgacacaacgcagcagcggggcccgagcactgagggaagctgtacctccttgcaaaggactagaggttagaggagaccccccgcaaataaaaacagcatattgacgctgggagagaccagagatcctgctgtctcctcagcatcattccaggcacagaacgccagaaaatggaatggtgctgttgaatcaacaggttct

SEQ ID NO: 6 - DNA, 10708 bp, Dengue virus
agttgttagtctacgtggaccgacaagaacagtttcgactcggaagcttgcttaacgtagtgctaacagttttttattagagagcagatctctgatgaacaaccaacggaagaagacgggaaaaccgtctatcaatatgctgaaacgcgtgagaaaccgtgtgtcaactggatcacagttggcgaagagattctcaaaaggactgctgaacggccagggaccaatgaaattggttatggcgttcatagctttcctcagatttctagccattccaccaacagcaggagtcttggctagatggggaaccttcaagaagtcgggggccattaaggtcctgaaaggctttaagaaggagatctcaaacatgctgagcataatcaacaaacggaaaaagacatcgctctgtctcatgatgatattgccagcagcacttgctttccacttgacttcacgagatggagagccgcgcatgattgtggggaagaatgaaagaggaaaatccctactttttaagacagcctctggaattaacatgtgcacactcatagccatggacttgggagagatgtgtgatgacacggtcacttacaaatgcccccatattaccgaagtggaacctgaagacattgactgctggtgcaacctcacatcaacatgggtgacttatggaacgtgcaatcaagccggagagcatagacgcgataagagatcagtggcgttagctccccatgtcggcatgggactggatacacgcacccaaacttggatgtcggctgaaggagcttggaggcaagtcgagaaggtagagacatgggcccttaggcacccagggttcaccatactagccctatttcttgcccattacataggcacttccttgacccagaaggtggttatttttatactgctaatgctggtcaccccatccatgacaatgagatgcgtgggcgtagggaatagagactttgtagagggattgagcggagcgacatgggtcgacgtcgtactcgaacacggggggtgcgtgacgactatggctaagaataagcctacactcgatatcgaactgcaaaaaaccgaagcgacacaattggcgacattgcgcaaactatgtatcgaaggtaagattacgaacattacaaccgatagtagatgccctacacaaggcgaagccgttctacctgaggagcaagaccaaaactacgtatgtaagcatacatacgttgatagggggtgggggaacggatgcggattgttcggtaaggggtcattggtcacatgcgctaagttccaatgcttagagcctatcgagggtaaggtggtacaatacgaaaaccttaagtataccgtgataattacagttcataccggagaccaacaccaagtgggaaacgagacacagggagtgacagccgaaattacgcctcaggctagtacaaccgaagcgatactacccgaatacggaacattggggttggagtgttcacctagaaccggattggactttaacgaaatgatactgttgactatgaaaaacaaggcatggatggtgcataggcaatggttttttgacctaccattgccttggactagcggagcgacaaccgaaacacctacatggaatagaaaagagctcctagtgacatttaaaaacgctcacgctaagaagcaggaagtggtcgttttagggtcacaggagggagctatgcataccgcactaaccggagcgactgagatacagaatagcggagggacatcaatcttcgccggacaccttaagtgtagattgaaaatggacaaacttgagcttaaggggatgtcatacgctatgtgtacgaacacattcgtgctgaaaaaagaggtaagcgaaacgcaacacggaacgatactgattaaggttgagtataagggcgaagacgtcccatgtaagatacctttttcgacagaggacggacagggtaaggcccataacggaagactgattaccgctaacccagtggtgaccaaaaaagaggaaccagtcaatatcgaagccgaacctccattcggagagtcaaacatagtgatagggataggcgataacgcacttaagattaactggtacaaaaaagggtcatcaatcggtaagatgtttgaggcaaccgctaggggggctagacggatggccatactcggagacaccgcatgggacttcggatccgtggggggggtacttaactcactcggtaagatggtgcaccaaattttcggatccgcatataccgctctatttagcggagtgtcatgggtgatgaaaatcggaatcggagttctattgacatggatcggattgaactctaagaatacatctatgtcattttcatgtatcgcaatcggaattattacgctatatctcggagccgtagtgcaagccgacatggggtgtgttataaactggaaaggcaaagaactcaagtgtggaagtggaatcttcgtcaccaacgaggtccatacctggacagagcaatacaaattccaagcagactccccaaaaagattggcgacagccattgcaggcgcttgggagaatggagtgtgcggaattaggtcaacaaccagaatggagaatctcctgtggaagcaaatagccaatgaactgaactacatattgtgggaaaacaatatcaaattaacggttgttgtgggcgatataattggggtcttagagcaagggaaaagaacactaacaccacaacccatggagctaaaatactcatggaaaacgtggggaaaggcaaaaatagtgacagctgaaacacaaaattcttctttcataatagatggaccaaacacaccggagtgtccaagtgcctcaagagcatggaatgtgtgggaggtggaagattacgggttcggagtcttcacaaccaacatatggctgaaactccgagaggtgtatacccaactatgtgaccataggttaatgtcggcagccgtcaaggatgaaagggccgtacatgccgacatgggctattggatagaaagtcaaaagaatggaagttggaagctagaaaaagcatccctcatagaggtgaaaacctgcacatggccaaaatcacataccctttggagtaatggtgtgttagagagtgacatgatcattccaaaaagtctagctggtcctatctcgcaacacaactacaggcccgggtaccacacccagacggcgggaccttggcatttaggaaaattagagctggacttcaactattgtgaaggaacaacagttgtcatcacagaaaactgtgggacaagaggcccatcattgagaacaacaacagtgtcagggaagttaatacacgaatggtgctgccgctcgtgcacacttcctcccctgcgatacatgggagaagacggttgctggtatggcatggaaatcagacccatcagtgagaaagaagaaaacatggtaaagtctttagtctcagcgggaagtggagaggtggacaacttcacaatgggtgtcttgtgtttggcaatcctctttgaagaggtgatgagaggaaaatttgggaagaaacacatgattgcgggggttttcttcacgtttgtactccttctctcagggcaaataacatggagagatatggcgcacacactaataatgattgggtccaatgcatctgacaggatgggaatgggcgtcacctacctagctttaattgcaacatttaaaatccagccatttttggctttgggatttttcctaagaaaactgacatccagagaaaatttattgttaggagttgggctggctatggcaacaacgttacaactgccagaggacattgaacaaatggcaaatggaatcgctctggggctcatggctctcaaactgataacacaatttgaaacataccaactatggacagcattaatctccttaacgtgttcaaatacaatttttacgttgactgttgcctggagaacagccaccctgattttggctggagtttcacttttaccagtgtgccagtcttcgagtatgaggaaaacagactggcttccaatgacagtggcagctatgggagttccacctctaccactttttatttttagcttaaaagacacactcaaaaggagaagctggccactgaatgaaggggtgatggctgttgggcttgtgagcattctggccagttctctccttagaaatgatgtacccatggctggaccattagtggccgggggcttgctgatagcgtgctacgtcataactggcacgtcagcagacctcaccgtagaaaaagcagcagatgtaacatgggaggaagaggctgagcaaacaggagtgtcccacaacttaatgatcacagttgatgatgatggaacaatgagaataaaagatgatgagactgagaatatcttaacagtgcttttgaaaacagcattactaatagtgtcaggaatctttccatactccatacccgcaacattgttggtctggcatacttggcaaaagcaaacccaaagatccggcgttctatgggatgtacccagccctccagagacacagaaagcagaactggaagaaggggtctataggatcaaacagcaaggaatttttgggaaaacccaagtaggggttggagtacagaaagaaggagtctttcacaccatgtggcacgttacaagaggggcagtgttgacatataatgggaaaagactggaaccaaattgggctagcgtgaaaaaagatctgatttcatacggaggaggatggagattgagcgcacaatggcaaaagggggaggaggtgcaggttattgccgtagagcctgggaagaacccaaagaactttcaaaccatgccaggcacttttcagactacaacaggggaaataggagcaattgcactggatttcaagcccggaacttcaggatctcctatcataaacagagagggaaaggtagtgggactgtatggcaatggagtggttacaaagaatggtggctacgtcagcggaatagcgcaaacgaatgcagaaccagatggaccgacaccagaattggaagaagagatgttcaaaaagcgaaatctaaccataatggatcttcatcctgggtcaggaaagacacggaaataccttccagctattgttagagaggcaatcaagagacgtttgagaactctaattctggcaccaacaagggtggttgcagctgagatggaagaagcattgaaagggctcccaataaggtaccaaacaacagcaacaaaatctgaacacacaggaagagagattgttgatctgatgtgccacgcaacgttcacaatgcgtctgctgtcaccagttagggttccaaactataacttgataataatggatgaagcccatttcacagacccagccagtatagctgctagagggtacatatcaactcgtgttggaatgggagaagcagccgcaatattcatgacagcaacgccccctggaacagctgatgcctttccccagagcaacgctccaattcaagatgaagaaagggacataccagaacgctcatggaattcaggcaatgaatggataaccgacttcgctgggaaaacggtgtggtttgtccccagcattaaagccggaaatgacatagcaaattgcctgcggaaaaacgggaaaaaggtcattcaacttagtaggaagacttttgacacagaatatcagaaaaccaaactgaatgattgggactttgtggtgacgactgacatttcagaaatgggggccaatttcaaagcagatagagtgatcgacccaagaagatgtctcaaaccagtgatcctgacagatggaccagagcgggtgatcctggctggaccaatgccagtcaccgcagcgagtgctgcgcaaaggagagggagagttggcaggaacccacaaaaagaaaatgaccagtacatattcacgggccagcctctcaacaatgatgaagaccatgctcactggacagaagcaaaaatgctgctggacaacattaacacaccagaagggatcataccagctctctttgagccagaaagggagaagtcagccgccatagacggtgagtatcgcctgaaaggtgagtccaggaagactttcgtggaactcatgaggaggggtgaccttccagtctggttagcccataaagtagcatcagaagggatcaaatatacagatagaaaatggtgctttgatggacaacgtaataatcaaattttagaagagaacatggatgtggaaatctggacaaaggaaggagaaaagaaaaaattgagacctaggtggcttgatgctcgcacctattcagatcccttagcactcaaggaattcaaggactttgcggctggcaggaagtcaatagcccttgatcttgtgacagaaataggaagagtgccttcacacctagctcatagaacgagaaacgctctggacaatctggtgatgctgcatacgtcagaacatggcggtaaggcctacaggcatgcggtggaggaactaccagagacaatggaaacactcctactcttgggactcatgatcttgttgacaggtggagcaatgcttttcttaatatcaggtaaagggattggaaagacttcaataggactcatttgtgtaattgcttccagcggcatgttgtggatggccgaaatcccactccaatggatcgcgtcggccatagtcctggagttttttatgatggtgttgcttataccagaaccagagaagcagagaaccccccaagacaaccaactcgcatatgtcgtgataggcatacttacactggcagcaataatagcagccaatgaaatgggattgttggaaactacaaagagagatttaggaatgtctaaggagccaggtgttgtttctccaaccagctatttagatgtggacttgcacccagcatcagcctggacattgtacgctgtggccactacagtaataacaccaatgttaagacataccatagagaattccacagcaaatgtgtccttggcagctatagccaaccaggcagtggtcctgatgggtttggacaaaggatggccaatatcaaaaatggacttaggagtacccctgctggcattgggttgctattcacaagtgaacccactgactctaacagcggcagtactcttgctgatcacacattatgccattataggtccaggattgcaggcaaaagccacccgtgaagctcagaaaaggacagctgctggaataatgaagaatccaacagtggatgggataatgacaatagacctagatcctgtaatatatgattcaaaatttgaaaagcaactgggacaggttatgctcctggttttgtgtgcagttcaacttttgttaatgagaacatcatgggccttgtgtgaagctttaaccctagctacaggaccaataacaacactctgggaaggatcacctgggaagttttggaacaccacgatagctgtttccatggcgaacatttttagagggagctatttagcaggagctgggcttgctttctctattatgaaatcagttggaacagggaaaagaggaacaggctcacagggtgaaactttgggagaaaaatggaaaaagaagttaaatcaattatcccggaaagagtttgacctttacaagaaatctggaatcactgaggtggatagaacagaagccaaagaagggttgaaaagaggagaaataacacatcatgccgtgtccagaggtagtgcaaaacttcaatggtttgtggagagaaacatggtcattcccgaaggaagagttatagacttgggctgtggaagaggaggctggtcatattactgtgcaggactgaaaaaagtcacagaagtgcgaggatacacaaaaggcggtccaggacacgaagaaccagtacctatgtccacatatggatggaacatagttaagttaatgagtggaaaggatgtgttttatcttccacctgaaaagtgtgacaccctgttgtgcgacattggagaatcttcaccaagcccaacagtggaagaaagcagaactataagagttttgaagatggttgaaccatggctaaagaacaaccaattttgtattaaagtattgaacccttacatgccaactgtgattgagcacctagaaagactacaaaggaaacatggaggaatgcttgtgagaaatccactttcacgaaactccacgcacgaaatgtactggatatccaatggcacaggtaacattgtcgcttcagtcaacatggtatctagactgctactgaacaggttcacgatgacacacagaagacccaccattgagaaagatgtggatttaggagcaggaactcgacatgttaatgcggaaccagaaacacccaacatggatgtcattggggaaagaataaaaaggatcaaggaggagcataattcaacatggcactacgatgacgaaaacccctacaaaacgtgggcttaccatggatcttatgaagtcaaagccacaggctcagcctcctccatgataaatggagtcgtgaaactcctcactaaaccatgggatgtggtgcccatggtgacacagatggcaatgacagatacaactccatttggccagcagagagtctttaaagagaaagtggacaccaggacacccaggtccatgccaggaacaagaagggttatggggatcacagcggagtggctctggagaaccctgggaaggaataaaaaacccaggttatgcacaagggaagagtttacaaaaaaggtcagaactaacgcagccatgggagctgttttcacagaggagaaccaatgggacagcgcgaaagctgctgttgaggatgaggatttttggaaacttgtggacagagaacgtgaactccacaaactgggcaagtgtggaagctgtgtttacaacatgatgggtaagagagagaagaaacttggagagtttggcaaagcaaaaggcagtagagctatatggtacatgtggttgggagccaggtaccttgagttcgaagcccttggattcttaaatgaagaccactggttctcgcgtgagaactcttacagtggagtggaaggagaaggactgcacaagctaggctatatattaagggacatttccaagatacccggaggagctatgtatgctgatgacacagctggttgggacacaagaataacagaagatgacctgcacaatgaggaaaagatcacacagcaaatggaccctgaacacaggcagttagcgaacgctatatttaagctcacataccaaaacaaagtggtcaaagttcaacgaccgactccaacaggcacggtaatggatatcatatctaggaaagaccaaagaggcagtggacaggtaggaacttatggtctgaatacattcaccaacatggaagcccagttaatcagacaaatggaaggagaaggtgtgctgtcaaaggcagacctcgagaaccctcatctgccagagaagaaaattacacaatggttggaaaccaaaggagtggagaggttaaaaagaatggccataagtggggatgactgcgtggtgaaaccaatcgatgacaggttcgctaatgccctgctcgctctgaacgacatgggaaaggttcggaaagacatacctcaatggcagccatcaaagggatggcatgattggcaacaggttcctttctgctcccaccactttcatgaattgatcatgaaagatggaagaaagttggtggttccctgcagaccccaggacgaactaataggaagggcaagaatctctcaaggagcgggatggagccttagagaaaccgcatgtctggggaaagcctacgctcaaatgtggagtctcatgtattttcacagaagagacctcagactagcatccaacgccatatgttcagcagtaccagtccactgggtccccacaagtagaacgacatggtctattcacgctcaccatcagtggatgaccacagaagacatgcttactgtctggaacagagtgtggatcgaggacaatccatggatggaagacaaaactccagtcacaacctgggaaaatgttccatatctagggaagagagaagaccaatggtgcggatcacttattggtctcacttccagagcaacctgggcccagaacatacccacagcaattcaacaggttagaagccttataggcaatgaagagtttctggactacatgccttcaatgaagagatttaggaaggaggaggagtcggagggagccatttggtaaaacgtaggaagtgaaaaagaggttaactgtcaggccatattaagccacagtacggaagaagctgtgctgcctgtgagccccgtccaaggacgttaaaagaagaagtcaggccccaaagccacggtttgagcaaaccgtgctgcctgtagctccgtcgtggggacgtaaaacctgggaggctgcaaactgtggaagctgtacgcacggtgtagcagactagcggttagaggagacccctcccatgacacaacgcagcagcggggcccgagcactgagggaagctgtacctccttgcaaaggactagaggttagaggagaccccccgcaaataaaaacagcatattgacgctgggagagaccagagatcctgctgtctcctcagcatcattccaggcacagaacgccagaaaatggaatggtgctgttgaatcaacaggttct

SEQ ID NO: 7 - DNA, 10649 bp, Dengue virus
agttgttagtctgtgtggaccgacaaggacagttccaaatcggaagcttgcttaacacagttctaacagtttgtttaaatagagagcagatctctggaaaaatgaaccaacgaaaaaaggtggttagaccacctttcaatatgctgaaacgcgagagaaaccgcgtatcaacccctcaagggttggtgaagagattctcaaccggacttttttctgggaaaggacccttacggatggtgctagcattcatcacgtttttgcgagtcctttccatcccaccaacagcagggattctgaagagatggggacagttgaagaaaaataaggccatcaagatactgattggattcaggaaggagataggccgcatgctgaacatcttgaacgggagaaaaaggtcaacgataacattgttgtgcttgattcccaccgtaatggcgtttcacttgtcaacaagagatggcgaacccctcatgatagtggcaaaacatgaaagggggagacctctcttgtttaagacaacagaggggatcaacaaatgcactctcattgccatggacttgggtgaaatgtgtgaggacactgtcacgtataaatgccccctactggtcaataccgaacctgaagacattgattgctggtgcaacctcacgtctacctgggtcatgtatgggacatgcacccagagcggagaacggagacgagagaagcgctcagtagctttgacaccacattcaggaatgggattggaaacaagagctgagacatggatgtcatcggaaggggcttggaagcatgctcagagagtagagagctggatactcagaaacccaggatttgcgctcttggcaggatttatggcttatatgattgggcaaacaggaatccagcgaactgtcttctttgtcctaatgatgctggtcgccccatcctacggaatgcggtgcgtaggagtaggaaacagagactttgtggaaggagtctcaggtggagcatgggtcgacctggtgctagaacatggaggatgcgtcacaaccatggcccagggaaaaccaaccttggattttgaactgactaagacaacagctaaggaagtggctctgttaagaacctattgcattgaagcctcaatatcaaacataactacggcaacaagatgtccaacgcaaggagagccttatctgaaagaggaacaggaccaacagtacatctgccggagagatgtggtagacagaggatggggcaatggctgtggcttgtttggaaaaggaggagttgtgacatgtgcgaagttttcatgttcggggaagataacaggcaatctggtccaaattgagaaccttgaatacacagtggttgtaacagtccacaatggagacacccatgcagtaggaaatgacacatccaatcatggagttacagccacgataactcccaggtcaccatcggtagaagtcaaattgccggactatggagaactaacactcgattgtgaacccaggtctggaattgactttaatgagatgatcctaatgaaaatgaaaaagaaaacatggctcgtgcataagcaatggtttttggatctgcctcttccatggacagcaggagcagacacatcagaggttcactggaattacaaagagagaatggtgacgttcaaggttcctcatgccaagagacaggatgtgacagtgctgggatctcaggaaggagccatgcattctgccctcgctggagccacagaagtggactccggtgatggaaatcacatgtttgcaggacatctcaagtgcaaagtccgtatggagaaattgagaatcaagggaatgtcatacacgatgtgttcaggaaagttttcaattgacaaagagatggcagaaacacagcatgggacaacagtggtgaaagtcaagtatgaaggtgctggagctccgtgtaaagtccccatagagataagagatgtaaacaaggaaaaagtggttgggcgcgttatctcatccacccctttggctgagaataccaacagtgtaaccaacatagaattagaacccccctttggggacagctacattgtgataggtgttggaaacagcgcattaacactccattggttcaggaaagggagttccattggcaagatgtttgagtccacatacagaggtgcaaaacgaatggccattctaggtgaaacagcttgggattttggttccgttggtggactgttcacatcattgggaaaggctgtacaccaggtttttggaagcgtgtatacaaccatgtttggaggagtctcatggatgattagaatcctaattgggttcttagtgttgtggattggcacgaattcaaggaacacttcaatggccatgacgtgcatagctgttggaggaattactctgtttttgggcttcacagttcaagcggacatgggttgtgtggtgtcatggagtgggagagaattgaagtgtggaagcggaatttttgtggttgacaacgtgcacacttggacagaacagtacaaattccaaccagagtccccagcgagactagcgtctgcaatattaaatgcccacaaagatggggtctgtggaattagatcaaccacgaggctggaaaatgttatgtggaagcaaataaccaatgagctaaactatgttctctgggaaggaggacatgatctcactgtagtggctggggatgtgaaaggggtgttgaccaagggcaagagagcactcacacccccagcgagtgatctgaaatattcatggaagacatgggggaaagcaaaaatcttcacccctgaagcaagaaacagcacatttttaatagacggaccagacacctctgaatgccccaatgaacgaagggcatggaattcttttgaggtggaagactatggatttggcatgttcacgaccaacatatggatgaaattccgagaaggaagttcagaagtgtgtgaccacaggttaatgtcagctgcaattaaagaccagaaagctgtgcatgctgacatgggttattggatagagagctcaaaaaaccagacctggcagatagagagagcatctcttattgaagtgaaaacatgtctgtggcccaagacccatacactgtggagcaatggagtgctggaaagccagatgcttattccaaaatcatatgcaggccctttttcacagcacaattaccgccagggctatgctacgcaaaccgtgggtccatggcacttaggcaaactagagatagactttggagaatgccccggaacaacagtcacaattcaggagaattgtgaccatagaggcccatctttgaggaccaccactgcatctggaaaactagtcacgcaatggtgttgccgctcctgcacgatgccccccttaaggttcttaggagaagatgggtgctggtatgggatggagattaggcccttgagtgaaaaagaagagaacatggtcaaatcacaggtgacggccggacagggcacatcggaaactttttcaatgggtctgttgtgcctgaccttgtttgtggaagaatgcttgaggagaagagtcaccaggaaacacatgatattagctgtggtaatcactctttgtgctatcatcctggggggcctcacatggatggacttgctacgagccctcatcatgttgggggacactatgtctggtagaataggaggacagacccacctagccatcatggcagtgttcaagatgtcaccgggatacgtgctgggtgtgtttttaaggaaactcacttcaagagagacagcactaatggtaataggaatggccatgacaacaacactttcaattccacatgacctcatggaactcattgatggaatatcactaggactaattttgctaaaaatagtaacacagtttgacaacacccaagtgggaaccttagctctttccttgactttcataagatcaacaatgtcattggtcatggcttggaggaccattatggctgtgttgtttgtggtcacactcattcctttgtgcaggacaagctgtcttcaaaaacagtctcattgggtagaaataacagcactcatcctaggagcccaagctctgccagtgtacctaatgactcttatgaaaggagcctcaagaagatcttggcctcttaacgaaggcataatggctgtgggtttggttagtctcttaggaagcgctcttttaaagaatgatgtccctttagctggcccaatggtggcaggaggcttacttctggcggcttacgtaatgagtggcagctcagcagatctgtcactagagaaggccgctaatgtgcagtgggatgaaatggcagacataacaggctcaagtccaatcatagaagtgaagcaagatgaggatggctctttctccatacgggacgtcgaggaaaccaatatgataacccttttggtgaaactggcactgataacggtgtcaggtctctaccccttggcaattccaatcacaatgaccttatggtacatgtggcaagtgaaaacacaaagatcaggagccctgtgggacgtcccttcacccgccgccactcaaaaagccgcactgtctgaaggagtgtacaggatcatgcaaagagggttattcgggaaaactcaggttggagtagggatacacatggaaggtgtatttcacacaatgtggcatgttacaagaggatcggtgatctgccacgagactgggagattggagccatcttgggctgatgtcaggaatgacatgatatcatacggtgggggatggaggcttggagataaatgggacaaagaagaagacgttcaggtcctcgctatagaaccagggaaaaatcccaaacatgtccaaacgaaacctggccttttcaagaccctaactggagaaattggagcagtaacattagatttcaaacccggaacgtctggttctcccattatcaacaggaaaggaaaagtcatcggactctatggaaatggagtggtcaccaaatcaggtgattacgtcagtgccataacacaagccgaaagaattggagagccagattatgaagtggatgaggacatttttcgaaagaaaagactaactataatggacttacaccccggagccggaaagacaaaaagaattcttccatcaatagtgagagaagccttaaaaaggaggctgcgaactttgattttggctcccacgagagtggtggcggccgagatggaagaggccctacgtggactgccaatccgttaccaaaccccagctgtaaaatcagaacacacaggaagagagattgtagacctcatgtgccatgcaaccttcacaacaagacttttgtcatcaaccagagttccaaactacaaccttatagtaatggatgaagcacatttcaccgatccttccagtgtcgcggctagaggatacatttcgaccagggtggaaatgggagaggcagcagccatcttcatgaccgcaacccctcccggagcgacagatccctttccccagagcaacagcccaatagaagacatcgagagagagattccggaaaggtcatggaacacagggttcgactggataacagactaccaagggaaaactgtgtggtttgttcccagcataaaagctggaaatgacattgcaaattgtttgagaaagtcgggaaagaaagttatccagttgagtaggaaaacctttgatacagaatatccaaaaacgaaactcacggactgggactttgtggtcactacagacatatctgaaatgggggctaactttagagctgggagagtgatagaccctagaagatgcctcaagccagttatcctaacagatgggccagagagagtcatcttagcaggtcctattccagtgactccagcaagcgctgctcagagaagagggcgaataggaaggaacccagcacaagaagacgaccaatacgttttctccggagacccactaaaaaatgatgaagaccatgcccactggacagaagcaaagatgctgcttgacaatatctacaccccagaagggatcattccaacattgtttggtccggaaagggaaaaaacccaagctattgatggagagtttcgcctcagaggggaacaaaggaagacttttgtggaattaatgaggagaggagaccttccggtgtggctgagttataaggtagcttctgctggcatttcttacaaagatcgggaatggtgctttactggggaaagaaataaccaaattttagaagaaaacatggaggttgaaatttggactagagagggagaaaagaaaaaactgaggccaaaatggttagatgcacgtgtatacgctgaccccatggctttgaaggatttcaaggagtttgccagtggaaggaagagtataactctcgacatcctaacagagatcgccagtttgccaacttacctttcctctagggccaagctcgcccttgacaacatagttatgctccacacaacagaaagaggagggagggcctatcaacatgccctgaacgaacttccggagtcactggaaacactcatgcttgtagccttactaggagctatgacagcaggtatcttcctgtttttcatgcaagggaaaggaatagggaaattgtcaatgggtttgataaccattgcggtggctagtggcttgctctgggtagcagaaattcaaccccagtggatagcggcctcaatcatactggagttttttctcatggtactgttgataccagaaccagaaaaacaaaggaccccacaagacaatcaattgatctacgtcatattgaccattctcaccattattggtctaatagcagccaacgagatggggctgatagaaaaaacaaaaacggattttgggttttaccaggtaaaaacagaaaccaccatcctcgatgtggacctgagaccagcttcagcatggacgctctatgcggtagccaccacaattctgactcccatgctgagacacaccatagaaaatacgtcggccaacctatctttagcagccatcgccaaccaggcagccgtcctaatggggcttggaaaaggatggccactccacagaatggacctcggtgtgccgctgttagcaatgggatgctattctcaagtgaacccaacaaccttgacagcatccttagtcatgcttttagtccattatgcaataataggcccaggattgcaggcaaaagccacaagagaggcccagaaaaggacagctgctggaatcatgaaaaatcccacagtggacgggataacagtgatagatctagaaccaatatcctatgacccaaaatttgaaaagcaattagggcaggtcatgttactcgtcttgtgtgctggacaactactcttgatgagaacaacatgggctttctgtgaagttttgactttggccacaggaccaatcttgaccttgtgggagggcaacccgggaaggttttggaacacgaccatagccgtatctaccgccaacattttcaggggaagttatttggcaggagctggactggctttttcactcataaagaatgcacaaacccccaggaggggaactgggaccacaggagagacactgggagagaagtggaagagacagctaaactcattagacaggaaagagtttgaagagtataaaagaagtggaatactagaagtggacaggactgaagccaagtccgccctgaaagatgggtctaaaatcaagcatgcagtatctagagggtccagtaagatcagatggattgttgagagagggatggtaaagccaaaggggaaagttgtagatcttggctgtgggagaggaggatggtcttattacatggcgacactcaagaacgtgactgaagtgaaagggtatacaaaaggaggtccaggacatgaagaaccgattcccatggctacttatggctggaatttggtcaaactccattcaggggttgacgtgttctacaaacccacagagcaagtggacaccctgctctgtgatattggggagtcatcttctaatccaacaatagaggaaggaagaacattaagagttttgaagatggtggagccatggctctcttcaaaacctgaattctgcatcaaagtcctcaacccctacatgccaacagtcatagaggagctggagaaactgcagagaaaacacggtgggaaccttgtcagatgcccgctgtccaggaactccacccatgagatgtattgggtgtcaggagcgtcgggaaatattgtgagctctgtgaacacaacatcaaagatgttgttgaacaggttcacaacaaggcataggaaacccacttatgagaaggacgtagatcttggggcaggaacgagaagtgtctctactgaaacagaaaaaccagacatgacaatcattgggagaaggcttcagcgattgcaagaggagcacaaagaaacatggcattatgatcaggaaaacccatacagaacctgggcgtatcatggaagctatgaagctccttcgacaggctctgcatcctccatggtgaacggggtggtgaaactgctaacaaaaccctgggatgtgattccaatggtgactcagttagccatgacagatacaaccccttttgggcaacaaagagtgttcaaagagaaggtggataccagaacgccgcaaccaaaaccaggcacacgaatggttatgaccacgacagccaattggctatgggccctccttggaaagaagaaaaatcccagactgtgtacaagggaagagttcatctcaaaagttagatcaaacgcagccataggcgcagtctttcaggaagaacaaggatggacatcagccagtgaagctgtgaatgacagccggttttgggaactggttgacaaagaaagggcccttcaccaggaagggaaatgtgaatcgtgtgtctataacatgatgggaaaacgtgagaaaaagttaggagagtttggcagagccaagggaagccgagcaatctggtacatgtggctgggagcgcggtttctggaatttgaagccctgggttttttgaatgaagatcactggtttggcagagaaaattcatggagtggagtggaaggggaaggtctgcacagattgggatacatcctggaggagatagacaagaaggatggagacctaatgtatgctgatgacacagcaggttgggacacaagaatcactgaggatgaccttcaaaatgaggaactgatcacggaacagatggctccccaccacaagatcctagccaaagccattttcaaactaacctatcaaaacaaagtggtgaaagtcctcagacccacaccgagaggagcggtgatggatatcatatccaggaaagaccaaagaggtagtggacaagttggaacatatggtttgaacacattcaccaacatggaagttcaactcatccgccaaatggaagctgaaggagtcatcacacaagatgacatgcagaacccaaaagggttgaaagaaagagttgagaaatggctgagagagtgtggtgtcgacaggttaaagaggatggcaatcagtggagacgattgcgtggtgaagcccctagatgagaggtttggcacttccctcctcttcttgaacgacatgggaaaggtgaggaaagacattccgcagtgggaaccatctaagggatggaaaaactggcaagaggttcctttttgctcccatcactttcacaagatctttatgaaggatggccgctcactagttgttccatgtagaaaccaggatgaactgatagggagagccagaatctcgcagggggctggatggagcttaagagaaacagcctgcctgggcaaagcttacgcccagatgtggtcgcttatgtacttccacagaagggatctgcgtttagcctccatggccatatgctcagcagttccaacggaatggtttccaacaagcagaacaacatggtcaatccatgctcatcaccaatggatgaccactgaagacatgctcaaagtgtggaacagagtgtggatagaagacaaccccaatatgattgacaagactccagtccattcgtgggaagatataccttacctagggaaaagagaggatttgtggtgtggatccctgattggactttcttctagagccacctgggcgaagaacattcacacggccataactcaggtcaggaacttgatcggaaaagaggaatacgtggattacatgccagtaatgaaaagatacagtgctccttcagagagtgaaggagttctgtaattaccaacaacaaacaccaaaggctattgaagtcaggccacttgtgccacggtttgagcaaaccgtgctgcctgtagctccgccaataatgggaggcgtaataatccctagggaggccatgcgccacggaagctgtacgcgtggcatattggactagcggttagaggagacccctcccatcactgacaaaacgcagcaaaagggggcccgaagccaggaggaagctgtactcctggtggaaggactagaggttagaggagacccccccaacacaaaaacagcatattgacgctgggaaagaccagagatcctgctgtctctacaacatcaatccaggcacagagcgccgcaagatggattggtgttgttgatccaacaggttct

SEQ ID NO: 8 - DNA, 10649 bp, Dengue virus
agttgttagtctgtgtggaccgacaaggacagttccaaatcggaagcttgcttaacacagttctaacagtttgtttaaatagagagcagatctctggaaaaatgaaccaacgaaaaaaggtggttagaccacctttcaatatgctgaaacgcgagagaaaccgcgtatcaacccctcaagggttggtgaagagattctcaaccggacttttttctgggaaaggacccttacggatggtgctagcattcatcacgtttttgcgagtcctttccatcccaccaacagcagggattctgaagagatggggacagttgaagaaaaataaggccatcaagatactgattggattcaggaaggagataggccgcatgctgaacatcttgaacgggagaaaaaggtcaacgataacattgttgtgcttgattcccaccgtaatggcgtttcacttgtcaacaagagatggcgaacccctcatgatagtggcaaaacatgaaagggggagacctctcttgtttaagacaacagaggggatcaacaaatgcactctcattgccatggacttgggtgaaatgtgtgaggacactgtcacgtataaatgccccctactggtcaataccgaacctgaagacattgattgctggtgcaacctcacgtctacctgggtcatgtatgggacatgcacccagagcggagaacggagacgagagaagcgctcagtagctttgacaccacattcaggaatgggattggaaacaagagctgagacatggatgtcatcggaaggggcttggaagcatgctcagagagtagagagctggatactcagaaacccaggatttgcgctcttggcaggatttatggcttatatgattgggcaaacaggaatccagcgaactgtcttctttgtcctaatgatgctggtcgccccatcctacggaatgagatgcgtaggcgtaggtaatcgcgatttcgttgagggagtgtcaggcggagcatgggtcgatctggtactcgaacacggaggatgcgttacgactatggcacagggaaaaccgacattggactttgagttgactaagacaaccgctaaagaggtcgcactattgcgaacatactgtatcgaagcgtcaatttcgaatattacaaccgctactaggtgtcctacacagggcgaaccataccttaaagaggaacaggaccaacagtatatttgtagaagagacgtagtcgatagggggtggggaaacggatgcggattgttcggtaaggggggagtcgttacatgcgctaagttctcatgttccggtaagattaccggtaacttagtgcaaattgagaatctcgaatataccgtagtcgttacagtgcataacggagacacacacgcagtcggaaacgatacatctaaccatggcgtaaccgctacaattacacctaggtcaccatccgttgaggttaagttgcccgattacggcgaactgacactcgattgcgaacctagatccggaatagactttaacgaaatgatactgatgaaaatgaaaaaaaagacatggttagtgcataagcaatggtttctcgacttacccctaccttggaccgccggagccgatactagcgaagtgcactggaattacaaagagagaatggttacatttaaagtgccacacgctaagagacaggacgttacagtgttagggtcacaggagggagctatgcatagcgcactagccggagcaaccgaagtcgatagcggagacggaaatcatatgttcgccggacatctgaaatgcaaagtgagaatggagaaattgcggattaagggaatgtcatacactatgtgttccggtaagttttcgattgacaaagagatggccgaaacgcaacacggaacaacagtcgttaaggttaagtacgaaggagccggagctccgtgtaaagtgccaatcgaaattagagacgttaacaaagagaaagtggtcggaagagtgatatctagtacacccctagccgaaaatacgaattccgttacgaatatcgaactcgaacccccctttggagactcatacatagtgataggagtgggtaattccgcactcacgttgcactggttcagaaagggatcatcaatcgggaagatgttcgaatcaacatataggggagcgaaaagaatggctatattgggcgaaaccgcatgggacttcggatcagtcggaggactgtttacgagtctgggtaaggccgtacatcaggtattcggatcagtgtatacaacaatgtttggcggagtgtcatggatgatacggatactgatagggtttctagtgttgtggatcggaacgaactcacgtaatacgtctatggctatgacatgtattgccgtaggggggattacactgtttctgggatttacagtgcaagcagacatgggttgtgtggtgtcatggagtgggagagaattgaagtgtggaagcggaatttttgtggttgacaacgtgcacacttggacagaacagtacaaattccaaccagagtccccagcgagactagcgtctgcaatattaaatgcccacaaagatggggtctgtggaattagatcaaccacgaggctggaaaatgttatgtggaagcaaataaccaatgagctaaactatgttctctgggaaggaggacatgatctcactgtagtggctggggatgtgaaaggggtgttgaccaagggcaagagagcactcacacccccagcgagtgatctgaaatattcatggaagacatgggggaaagcaaaaatcttcacccctgaagcaagaaacagcacatttttaatagacggaccagacacctctgaatgccccaatgaacgaagggcatggaattcttttgaggtggaagactatggatttggcatgttcacgaccaacatatggatgaaattccgagaaggaagttcagaagtgtgtgaccacaggttaatgtcagctgcaattaaagaccagaaagctgtgcatgctgacatgggttattggatagagagctcaaaaaaccagacctggcagatagagagagcatctcttattgaagtgaaaacatgtctgtggcccaagacccatacactgtggagcaatggagtgctggaaagccagatgcttattccaaaatcatatgcaggccctttttcacagcacaattaccgccagggctatgctacgcaaaccgtgggtccatggcacttaggcaaactagagatagactttggagaatgccccggaacaacagtcacaattcaggagaattgtgaccatagaggcccatctttgaggaccaccactgcatctggaaaactagtcacgcaatggtgttgccgctcctgcacgatgccccccttaaggttcttaggagaagatgggtgctggtatgggatggagattaggcccttgagtgaaaaagaagagaacatggtcaaatcacaggtgacggccggacagggcacatcggaaactttttcaatgggtctgttgtgcctgaccttgtttgtggaagaatgcttgaggagaagagtcaccaggaaacacatgatattagctgtggtaatcactctttgtgctatcatcctggggggcctcacatggatggacttgctacgagccctcatcatgttgggggacactatgtctggtagaataggaggacagacccacctagccatcatggcagtgttcaagatgtcaccgggatacgtgctgggtgtgtttttaaggaaactcacttcaagagagacagcactaatggtaataggaatggccatgacaacaacactttcaattccacatgacctcatggaactcattgatggaatatcactaggactaattttgctaaaaatagtaacacagtttgacaacacccaagtgggaaccttagctctttccttgactttcataagatcaacaatgtcattggtcatggcttggaggaccattatggctgtgttgtttgtggtcacactcattcctttgtgcaggacaagctgtcttcaaaaacagtctcattgggtagaaataacagcactcatcctaggagcccaagctctgccagtgtacctaatgactcttatgaaaggagcctcaagaagatcttggcctcttaacgaaggcataatggctgtgggtttggttagtctcttaggaagcgctcttttaaagaatgatgtccctttagctggcccaatggtggcaggaggcttacttctggcggcttacgtaatgagtggcagctcagcagatctgtcactagagaaggccgctaatgtgcagtgggatgaaatggcagacataacaggctcaagtccaatcatagaagtgaagcaagatgaggatggctctttctccatacgggacgtcgaggaaaccaatatgataacccttttggtgaaactggcactgataacggtgtcaggtctctaccccttggcaattccaatcacaatgaccttatggtacatgtggcaagtgaaaacacaaagatcaggagccctgtgggacgtcccttcacccgccgccactcaaaaagccgcactgtctgaaggagtgtacaggatcatgcaaagagggttattcgggaaaactcaggttggagtagggatacacatggaaggtgtatttcacacaatgtggcatgttacaagaggatcggtgatctgccacgagactgggagattggagccatcttgggctgatgtcaggaatgacatgatatcatacggtgggggatggaggcttggagataaatgggacaaagaagaagacgttcaggtcctcgctatagaaccagggaaaaatcccaaacatgtccaaacgaaacctggccttttcaagaccctaactggagaaattggagcagtaacattagatttcaaacccggaacgtctggttctcccattatcaacaggaaaggaaaagtcatcggactctatggaaatggagtggtcaccaaatcaggtgattacgtcagtgccataacacaagccgaaagaattggagagccagattatgaagtggatgaggacatttttcgaaagaaaagactaactataatggacttacaccccggagccggaaagacaaaaagaattcttccatcaatagtgagagaagccttaaaaaggaggctgcgaactttgattttggctcccacgagagtggtggcggccgagatggaagaggccctacgtggactgccaatccgttaccaaaccccagctgtaaaatcagaacacacaggaagagagattgtagacctcatgtgccatgcaaccttcacaacaagacttttgtcatcaaccagagttccaaactacaaccttatagtaatggatgaagcacatttcaccgatccttccagtgtcgcggctagaggatacatttcgaccagggtggaaatgggagaggcagcagccatcttcatgaccgcaacccctcccggagcgacagatccctttccccagagcaacagcccaatagaagacatcgagagagagattccggaaaggtcatggaacacagggttcgactggataacagactaccaagggaaaactgtgtggtttgttcccagcataaaagctggaaatgacattgcaaattgtttgagaaagtcgggaaagaaagttatccagttgagtaggaaaacctttgatacagaatatccaaaaacgaaactcacggactgggactttgtggtcactacagacatatctgaaatgggggctaactttagagctgggagagtgatagaccctagaagatgcctcaagccagttatcctaacagatgggccagagagagtcatcttagcaggtcctattccagtgactccagcaagcgctgctcagagaagagggcgaataggaaggaacccagcacaagaagacgaccaatacgttttctccggagacccactaaaaaatgatgaagaccatgcccactggacagaagcaaagatgctgcttgacaatatctacaccccagaagggatcattccaacattgtttggtccggaaagggaaaaaacccaagctattgatggagagtttcgcctcagaggggaacaaaggaagacttttgtggaattaatgaggagaggagaccttccggtgtggctgagttataaggtagcttctgctggcatttcttacaaagatcgggaatggtgctttactggggaaagaaataaccaaattttagaagaaaacatggaggttgaaatttggactagagagggagaaaagaaaaaactgaggccaaaatggttagatgcacgtgtatacgctgaccccatggctttgaaggatttcaaggagtttgccagtggaaggaagagtataactctcgacatcctaacagagatcgccagtttgccaacttacctttcctctagggccaagctcgcccttgacaacatagttatgctccacacaacagaaagaggagggagggcctatcaacatgccctgaacgaacttccggagtcactggaaacactcatgcttgtagccttactaggagctatgacagcaggtatcttcctgtttttcatgcaagggaaaggaatagggaaattgtcaatgggtttgataaccattgcggtggctagtggcttgctctgggtagcagaaattcaaccccagtggatagcggcctcaatcatactggagttttttctcatggtactgttgataccagaaccagaaaaacaaaggaccccacaagacaatcaattgatctacgtcatattgaccattctcaccattattggtctaatagcagccaacgagatggggctgatagaaaaaacaaaaacggattttgggttttaccaggtaaaaacagaaaccaccatcctcgatgtggacctgagaccagcttcagcatggacgctctatgcggtagccaccacaattctgactcccatgctgagacacaccatagaaaatacgtcggccaacctatctttagcagccatcgccaaccaggcagccgtcctaatggggcttggaaaaggatggccactccacagaatggacctcggtgtgccgctgttagcaatgggatgctattctcaagtgaacccaacaaccttgacagcatccttagtcatgcttttagtccattatgcaataataggcccaggattgcaggcaaaagccacaagagaggcccagaaaaggacagctgctggaatcatgaaaaatcccacagtggacgggataacagtgatagatctagaaccaatatcctatgacccaaaatttgaaaagcaattagggcaggtcatgttactcgtcttgtgtgctggacaactactcttgatgagaacaacatgggctttctgtgaagttttgactttggccacaggaccaatcttgaccttgtgggagggcaacccgggaaggttttggaacacgaccatagccgtatctaccgccaacattttcaggggaagttatttggcaggagctggactggctttttcactcataaagaatgcacaaacccccaggaggggaactgggaccacaggagagacactgggagagaagtggaagagacagctaaactcattagacaggaaagagtttgaagagtataaaagaagtggaatactagaagtggacaggactgaagccaagtccgccctgaaagatgggtctaaaatcaagcatgcagtatctagagggtccagtaagatcagatggattgttgagagagggatggtaaagccaaaggggaaagttgtagatcttggctgtgggagaggaggatggtcttattacatggcgacactcaagaacgtgactgaagtgaaagggtatacaaaaggaggtccaggacatgaagaaccgattcccatggctacttatggctggaatttggtcaaactccattcaggggttgacgtgttctacaaacccacagagcaagtggacaccctgctctgtgatattggggagtcatcttctaatccaacaatagaggaaggaagaacattaagagttttgaagatggtggagccatggctctcttcaaaacctgaattctgcatcaaagtcctcaacccctacatgccaacagtcatagaggagctggagaaactgcagagaaaacacggtgggaaccttgtcagatgcccgctgtccaggaactccacccatgagatgtattgggtgtcaggagcgtcgggaaatattgtgagctctgtgaacacaacatcaaagatgttgttgaacaggttcacaacaaggcataggaaacccacttatgagaaggacgtagatcttggggcaggaacgagaagtgtctctactgaaacagaaaaaccagacatgacaatcattgggagaaggcttcagcgattgcaagaggagcacaaagaaacatggcattatgatcaggaaaacccatacagaacctgggcgtatcatggaagctatgaagctccttcgacaggctctgcatcctccatggtgaacggggtggtgaaactgctaacaaaaccctgggatgtgattccaatggtgactcagttagccatgacagatacaaccccttttgggcaacaaagagtgttcaaagagaaggtggataccagaacgccgcaaccaaaaccaggcacacgaatggttatgaccacgacagccaattggctatgggccctccttggaaagaagaaaaatcccagactgtgtacaagggaagagttcatctcaaaagttagatcaaacgcagccataggcgcagtctttcaggaagaacaaggatggacatcagccagtgaagctgtgaatgacagccggttttgggaactggttgacaaagaaagggcccttcaccaggaagggaaatgtgaatcgtgtgtctataacatgatgggaaaacgtgagaaaaagttaggagagtttggcagagccaagggaagccgagcaatctggtacatgtggctgggagcgcggtttctggaatttgaagccctgggttttttgaatgaagatcactggtttggcagagaaaattcatggagtggagtggaaggggaaggtctgcacagattgggatacatcctggaggagatagacaagaaggatggagacctaatgtatgctgatgacacagcaggttgggacacaagaatcactgaggatgaccttcaaaatgaggaactgatcacggaacagatggctccccaccacaagatcctagccaaagccattttcaaactaacctatcaaaacaaagtggtgaaagtcctcagacccacaccgagaggagcggtgatggatatcatatccaggaaagaccaaagaggtagtggacaagttggaacatatggtttgaacacattcaccaacatggaagttcaactcatccgccaaatggaagctgaaggagtcatcacacaagatgacatgcagaacccaaaagggttgaaagaaagagttgagaaatggctgagagagtgtggtgtcgacaggttaaagaggatggcaatcagtggagacgattgcgtggtgaagcccctagatgagaggtttggcacttccctcctcttcttgaacgacatgggaaaggtgaggaaagacattccgcagtgggaaccatctaagggatggaaaaactggcaagaggttcctttttgctcccatcactttcacaagatctttatgaaggatggccgctcactagttgttccatgtagaaaccaggatgaactgatagggagagccagaatctcgcagggggctggatggagcttaagagaaacagcctgcctgggcaaagcttacgcccagatgtggtcgcttatgtacttccacagaagggatctgcgtttagcctccatggccatatgctcagcagttccaacggaatggtttccaacaagcagaacaacatggtcaatccatgctcatcaccaatggatgaccactgaagacatgctcaaagtgtggaacagagtgtggatagaagacaaccccaatatgattgacaagactccagtccattcgtgggaagatataccttacctagggaaaagagaggatttgtggtgtggatccctgattggactttcttctagagccacctgggcgaagaacattcacacggccataactcaggtcaggaacttgatcggaaaagaggaatacgtggattacatgccagtaatgaaaagatacagtgctccttcagagagtgaaggagttctgtaattaccaacaacaaacaccaaaggctattgaagtcaggccacttgtgccacggtttgagcaaaccgtgctgcctgtagctccgccaataatgggaggcgtaataatccctagggaggccatgcgccacggaagctgtacgcgtggcatattggactagcggttagaggagacccctcccatcactgacaaaacgcagcaaaagggggcccgaagccaggaggaagctgtactcctggtggaaggactagaggttagaggagacccccccaacacaaaaacagcatattgacgctgggaaagaccagagatcctgctgtctctacaacatcaatccaggcacagagcgccgcaagatggattggtgttgttgatccaacaggttct

SEQ ID NO: 9 - DNA, 10735 bp, Dengue virus
agttgttagtctacgtggaccgacaagaacagtttcgaatcggaagcttgcttaacgtagttctaacagttttttattagagagcagatctctgatgaacaaccaacggaaaaagacggctcgaccgtctttcaatatgctgaaacgcgcgagaaaccgcgtgtcaactgtttcacagttggcgaagagattctcaaaaggattgctctcaggccaaggacccatgaaattggtgatggctttcatagcattcctaagatttctagccatacccccaacagcaggaattttggctagatggggttcattcaagaagagtggagcgatcaaagtgctacggggtttcaagaaagaaatctcaaacatgttgaatataatgaatagaaggaaaagatctgtgaccatgctccttatgctgatgcctacagccttggcgttccatttgactacacgagggggagagccgcacatgatagtcagcaagcaggaaagaggaaagtcactcttgtttaagacctcagcaggtgtcaacatgtgcacccttatagcgatggatttgggagagttatgtgaggacacaatgacttacaaatgccctcgaatcactgaaactgaaccagatgacgttgattgttggtgtaatgccacagacacatgggtgacctatggaacatgttcccaaactggcgagcaccgacgagacaaacgttccgtcgcactggccccacacgtgggacttggtttggaaacaagaaccgaaacgtggatgtcctctgaaggcgcttggaaacagatacaaagagtggagacttgggccctgagacacccaggattcacggtgatagccctttttctagcacatgccataggaacatccatcacccagaaagggattattttcattttgttaatgctggtaacaccatccatggccatgcgatgcgtgggaataggcagcagggacttcgtggaaggactgtcaggagcaacttgggtagatgtggtactggaacatggaagttgcgtcaccaccatggcaaaagacaaaccaacattggacattgaactcttgaagacggaagtcacaaaccctgccgtcctgcgcaaactgtgcattgaagctaaaatatcaaacaccaccaccgactcaagatgtccaacacaaggagaagccacactagtggaagaacaagacgcgaactttgtgtgtcgacgaacgtttgtggacagaggctggggcaatggctgtggcctcttcggaaaaggaagcctaataacgtgtgcaaagttcaagtgtgtgacaaaactggaaggaaagatagttcaatatgagaacttgaaatattcagtaatagtcaccgttcacaccggagaccagcatcaagtgggaaatgaaagcacagaacatgggacaactgcaactataacacctcaagctcctacgacggaaatacagctgaccgactacggagctcttacattggattgttcacctagaacaggactagactttaatgaaatggtgttgttgacaatgaaagaaaaatcatggctagtccacaaacaatggtttttagacctaccactgccttggacctcgggagcttcaacatcacaagagacttggaacagacaagatttgctggtgacatttaagacagctcatgcaaagaagcaggaagtagtagtgttagggtcacaagagggagcaatgcataccgcactaaccggagcaaccgaaatacagactagcggaactacaacgatttttgccggtcacctgaaatgtagactgaagatggacaaactgacacttaaaggaatgtcatacgttatgtgtacgggatcctttaagctcgaaaaagaggttgccgaaacgcaacacggaacagtgctagtgcaaattaaatatgagggaaccgacgcaccatgtaagataccgtttagtacgcaagacgaaaagggcgttacgcaaaacggaagactgattaccgctaaccctatcgttacagacaaagagaaacccgttaacatagaggccgaaccaccttttggcgaatcatatatagtgataggcgcaggcgaaaaagcactaaagctgtcatggttcaaaaaaggatctagcatagggaagatgttcgaagcaaccgctaggggagctagacgaatggcaatactgggagataccgcatgggacttcggatcgatagggggagtgtttactagcgtaggtaagttggtgcatcagatattcggaaccgcatacggagtgttgtttagcggagtgtcatggactatgaagatagggataggagtgctattgacatggctcggactgaattcgagatcgactagcctatctatgacatgcatagccgtcggactggttacactgtatctaggcgtaatggtgcaagcagattcaggatgtgtaattaattggaaaggtagagaactcaagtgtggaagtggcatttttgtcaccaatgaagttcacacttggacagagcaatacaaatttcaagctgactcccctaagagactatcagcagccatcgggaaggcatgggaggagggtgtgtgtggaattcgatcagcaactcgtctcgagaacatcatgtggaagcaaatatcaaatgaactgaatcacatcttacttgaaaatgatatgaaattcacagtggttgtaggagatgttgctgggatcttggctcaaggaaagaaaatgattaggccacaacccatggaatacaaatactcgtggaaaagctggggaaaggctaaaatcataggggcagatgtacagaacaccaccttcatcatcgatggcccaaacaccccagaatgccctgatgaccaaagagcatggaacatttgggaagttgaggactatggatttggaattttcacgacaaacatatggctgaaattgcgtgattcctacacccaagtgtgtgaccaccggctaatgtcagctgccatcaaggacagcaaggcagttcacgctgacatggggtactggatagaaagtgaaaagaacgagacctggaagctggcaagagcctcattcatagaagttaaaacatgtatctggccaaaatcccacactctatggagcaatggagttctggaaagtgaaatgataattccaaagatctatggaggaccaatatctcagcacaactacagaccaggatattttacacaagcagcagggccgtggcacctaggcaagttggaactggattttgatttgtgtgagggtaccacagttgttgtggatgaacattgtggaaatcgaggaccatctcttaggaccacaacagtcacaggaaagataattcatgaatggtgttgcagatcttgcacgctaccacccttacgtttcagaggagaagatgggtgctggtacggtatggaaatcagaccagtcaaggaaaaggaagaaaatctagtcaaatcaatggtctctgcagggtcaggggaagtggacagcttttcactaggactgctatgcatatcaataatgatcgaggaggtgatgagatccagatggagtagaagaatgctgatgactggaacactggctgtgttcttccttctcataatgggacaattgacatggaacgatctgatcagattatgcatcatggttggagccaacgcttccgacaggatggggatgggaacgacgtacctagccctgatggccacttttaaaatgagaccgatgttcgctgtagggctattatttcgcagactaacatccagagaagttcttcttctaacaattggattgagtctagtggcatctgtggagttaccaaattccttggaggagctgggggatggacttgcaatgggcattatgattttaaaattattgactgactttcaatcacatcagctgtgggctaccttgctgtccttgacatttatcaaaacaacgttttccttgcactacgcatggaagacaatggctatggtactgtcaattgtatctctcttccctttatgcctgtccacgacctcccaaaaaacaacatggcttccggtgctattgggatcccttggatgcaaaccactaaccatgtttcttatagcagaaaacaaaatctggggaaggagaagttggcccctcaatgaaggaatcatggctgttggaatagtcagcatcctactaagttcactcctcaaaaatgatgtaccgctagctgggccactaatagctggaggcatgctaatagcatgttatgttatatctggaagctcagccgacctatcattagagaaagcggctgaggtctcctgggaagaagaagcagaacactctggtgcctcacacaacatattagtggaagtccaagatgatggaaccatgaagataaaggatgaagagagagatgacacgctaaccattctccttaaagcaactctgttagcagtttcaggggtgtacccattatcaataccagcgacccttttcgtgtggtacttttggcagaaaaagaaacagagatctggagtgttatgggacacacccagccctccagaagtggaaagagcagttcttgatgatggtatctatagaattatgcagagaggactgttgggcaggtcccaagtaggggtaggagttttccaagaaaacgtgttccacacaatgtggcatgtcaccaggggagctgtactcatgtatcaagggaagagattggaaccgagctgggccagtgtcaaaaaagacctgatctcatatggaggaggttggaggcttcaaggatcctggaacacaggagaagaagtgcaggtgattgctgttgaaccagggaaaaaccccaaaaatgtacaaacagcgccgggcacctttaagacccctgaaggtgaagttggagccattgccctagactttaaacctggcacatctggatctcccatcgtgaacagagaaggaaaaatagtaggtctttatggaaatggagtagtgacaacaagtggaacctacgtcagtgccatagctcaagccaaagcatcacaagaagggcccctaccagagattgaagacgaggtgtttaggaaaagaaacttaacaataatggacctacatccaggatcggggaaaacaagaagatatcttccagccatagtccgtgaggccataaaaaggaagctgcgcacactaatcctggctcccacaagggttgtcgcttccgaaatggcagaggcgctcaagggaatgccaataaggtaccaaacaacagcagtgaagagtgaacatacaggaaaagagatagttgacctcatgtgtcacgccactttcaccatgcgcctcctgtctcccgtaagagttcccaattacaacatgatcatcatggatgaagcacatttcaccgatccatccagtatagcggccagagggtacatctcaacccgagtgggcatgggtgaagcagctgcaatcttcatgacagccactcccccaggatcagtggaggcctttccacagagcaacgcagttatccaagatgaggaaagagacattcctgagagatcatggaactcaggctatgagtggatcactgacttcccaggtaaaacagtttggtttgttccaagcattaaatcaggaaatgacattgccaactgcttaagaaagaatgggaaacgggtgattcaattgagcaggaaaacctttgatacagagtaccaaaaaacaaaaaacaacgactgggactacgtcgtcacaacagacatctccgaaatgggagcaaatttccgagccgacagggtgatagacccaagacggtgtctgaaaccggtaatactaaaagatggtccagagcgtgtcattttagcaggaccaatgccagtgactgtggccagtgccgctcagaggagaggaagaattggaaggaaccacaataaggaaggtgatcagtacatctacatgggacagcctttaaacaacgatgaagatcacgctcactggacagaagcaaaaatgctccttgataatataaacacaccagaagggattatcccagccctctttgagccagagagagaaaagagtgcagcaatagacggggaatacagactgcggggtgaagcaaggaaaacgtttgtggagctcatgagaagaggagatctacctgtctggctatcctacaaagttgcctcagaaggcttccagtactctgacagaagatggtgctttgacggggaaaggaacaaccaggtgttggaggagaacatggacgtggagatctggacaaaagaaggagaaagaaagaaactacgaccccgctggctggatgccagaacatactcagacccactagccctgcgcgagtttaaagagtttgcagcagggagaagaagcgtctcaggtgatttaatattagaaatagggaagcttccacaacacttgacgcaaagggcccagaatgccctggacaacctggttatgttgcacaactccgaacaaggaggcagagcctatagacatgcaatggaagaactgccagacaccatagaaacgttgatgctcctagctttgatagctgtgttaactggtggagtgacactgttcttcctatcaggaaggggcctagggaaaacatctatcggcctactctgcgtaatggcttcaagcgtactgctatggatggccagtgtggagccccattggatagcggcctccatcatactggagttcttcctgatggtgctgcttattccagagccagacagacaacgcactccgcaggacaaccagctggcatatgtggtgataggtttgttattcatgatactgacagtagcagccaatgagatgggactgctggaaaccacaaagaaagacttagggattggccatgtggctgttgaaaatcaccaccatgccgcaatgctggacgtagacttacatccagcttcagcctggaccctctatgcagtggccacaacaattatcactcccatgatgaggcacacaatcgaaaacacaacggcaaacatttccctgacagccattgcaaaccaggcagctatattgatgggacttgacaaaggatggccaatatcgaagatggacataggagttccacttctcgccttggggtgctattcccaggtgaatccactgacgctgacagcggcggtattgatgctagtggctcattacgccataattggacctggactgcaagcaaaagctactagagaagctcaaaaaaggacagcggccggaataatgaaaaatccaaccgttgatggaattgttgcaatagatttggaccctgtggtttatgatgcaaaatttgaaaaacaactaggccaaataatgttgttgatactatgcacatcacagatcctcttgatgcggactacatgggccttgtgcgagtccatcacactggccactggacctctgaccacgctttgggagggatctccaggaaaattttggaacaccacgatagcggtttccatggcaaacattttcagaggaagttatctagcaggagcaggtctggccttctcattaatgaaatctctaggaggaggtaggagaggcacgggagcccaaggggaaacactgggagagaaatggaaaagacagctgaaccaactgagcaagtcagaatttaacacctataaaaggagtgggattatggaagtggacagatccgaagccaaagagggattgaaaagaggagaaacaaccaaacatgcagtgtcgagaggaaccgctaaactgaggtggttcgtggagaggaaccttgtgaaaccagaagggaaagtcatagacctcggttgtggaagaggtggctggtcatactattgcgctgggctgaagaaagtcacagaagtgaaggggtatacaaaaggaggacctggacatgaggaaccaatcccaatggcgacctatggatggaacctagtaaagctgcactctgggaaagacgtattttttataccacctgagaaatgtgacacccttttgtgtgatattggtgagtcctctccaaacccaactatagaggaaggaagaacgctacgcgtcctaaagatggtggaaccatggctcagaggaaaccaattttgcataaaaattctgaatccctacatgccaagtgtggtggaaactctggagcaaatgcaaagaaaacatggaggaatgctagtgcgaaatccactttcaagaaattctactcatgaaatgtattgggtttcatgtggaacaggaaacattgtgtcagcagtaaacatgacatctagaatgttgctaaatcgattcacaatggctcacaggaaaccaacatatgaaagagacgtggacctaggcgccggaacaagacatgtggcagtggaaccagaggtagccaacctagatatcattggccagaggatagagaacataaaacatgaacataagtcaacatggcattatgatgaggacaatccatataaaacatgggcctatcatggatcatatgaggtcaagccatcaggatcagcctcatccatggtcaatggcgtggtgaaactgctcaccaaaccatgggatgtcatccctatggtcacacaaatagccatgactgacactacaccctttggacaacagagggtgtttaaagagaaagttgacacacgcacaccaaaagcaaaacggggcacagcacaaatcatggaggtgacagccaagtggttatggggttttctttctagaaacaagaaaccaagaatttgcacaagagaggagttcacaagaaaagttaggtcaaacgcagccattggagcagtgttcgttgatgaaaatcaatggaactcagcaaaagaagcagtggaagatgagcggttctgggaccttgtgcatagagagagggagcttcacaaacagggaaaatgtgctacgtgtgtttacaacatgatggggaagagagagaaaaagctaggagagttcggaaaggcaaaaggaagtcgtgcaatatggtacatgtggttgggagcacgctttctagagttcgaagctcttggtttcatgaacgaagatcactggttcagcagagagaattcactcagcggagtggaaggagaaggactccacaaacttggatatatactcagagacatatcaaagattccagggggaaacatgtatgcagatgacacagccggatgggatacaaggataacagaggatgaccttcagaatgaggccagaattactgacatcatggaacccgaacatgccctcctggctaagtcaatcttcaagttaacctaccaaaataaggtggtaagggtacagagaccagcaaaaaatggaaccgtgatggatgtcatatccagacgtgaccagagaggaagtggtcaggtcggaacttatggcttaaacactttcaccaacatggaagcccagctgataagacaaatggagtctgagggaatcttttcacccagcgaattagagaccccaaatttagccgagagagttctcgactggctggaaaaatatggcgtcgaaaggctgaaaagaatggcaatcagcggagatgactgcgtagtgaaaccaattgatgataggtttgcaacagccttgacagctctgaatgatatgggaaaagtaagaaaagatataccacaatgggaaccttcaaaaggatggaatgattggcaacaagtgcctttttgttcacaccacttccaccagctgattatgaaggatgggagggaaatagtggtgccatgccgcaaccaagatgaacttgtgggtagggctagagtatcacaaggtgctggatggagcctgagagaaactgcatgcctaggcaagtcatatgcacaaatgtggcagctgatgtacttccacaggagagacctgagactagccgctaatgctatctgttcagccgttccagttgattggatcccaaccagccgtaccacctggtcgatccatgcccatcaccaatggatgacaacagaagacatgctgtcagtgtggaatagggtttggatagaggaaaacccatggatggaggacaaaacccacatatccagttggggagatgttccatatttaggaaaaagggaagaccaatggtgtggatccctgataggcttaacagcaagggccacctgggccaccaacatacaagtggccataaaccaagtgagaagactaattgggaatgagaattatctagattacatgacatcaatgaagagattcaagaacgagagtgatcccgaaggggcactctggtgagtcaacacatttacaaaataaaggaaaataagaaatcaaacaaggcaagaagtcaggccggattaagccatagtacggtaagagctatgctgcctgtgagccccgtctaaggacgtaaaatgaagtcaggccgaaagccacggcttgagcaaaccgtgctgcctgtagctccatcgtggggatgtaaaaacctgggaggctgcaacccatggaagctgtacgcatggggtagcagactagtggttagaggagacccctcccgaaacataacgcagcagcggggcccaacaccaggggaagctgtaccctggtggtaaggactagaggttagaggagaccccccgcataacaataaacagcatattgacgctgggagagaccagagatcctgctgtctctacagcatcattccaggcacagaacgccagaaaatggaatggtgctgttgaatcaacaggttct

SEQ ID NO: 10 - DNA, 10724 bp, Dengue virus
agttgttagtctacgtggaccgacaaagacagattctttgagggagctaagctcaacgtagttctaacagttttttaattagagagcagatctctgatgaataaccaacgaaaaaaggcgagaagtacgcctttcaatatgctgaaacgcgagagaaaccgcgtgtcaactgtgcaacagctgacaaagagattctcacttggaatgctgcaaggacgcggaccattaaaactgttcatggcccttgtggcgttccttcgtttcctaacaatcccaccaacagcagggatactaaaaagatggggaacgatcaaaaaatcaaaagctatcaatgttttgagagggttcaggaaagagattggaaggatgctgaacatcttgaacaagagacgcaggacagcaggcgtgattgttatgttgattccaacagcgatggcgttccatttaaccacacgcaatggagaaccacacatgatcgttggtaggcaggagaaagggaaaagtcttctgttcaaaacagaggatggtgttaacatgtgtactctcatggccatagaccttggtgaattgtgtgaagatacaatcacgtacaagtgtcctctcctcagacaaaatgaaccagaagacatagattgttggtgcaactctacgtccacatgggtaacttatgggacatgtaccaccacaggagaacacagaagagaaaaaagatcagtggcgctcgttccacatgtaggtatgggactggagacacgaactgaaacatggatgtcatcagaaggggcctggaaacatgttcagagaattgaaacctggatcttgagacatccaggttttaccataatggcagcaatcctggcatacaccataggaacgacacatttccaaagggccttgattttcattttactgacagctgtcgctccttcaatgacaatgcgctgcataggaatatcaaatagagacttcgtagaaggggtttcaggaggaagctgggttgacatagtcttagaacatggaagttgtgtgacgacgatggcaaaaaacaaaccaacattggattttgaactgataaaaacagaagccaaacaacctgccactctaaggaagtactgtatagaagcaaagctgaccaacacaacaacagaatcgcgttgcccaacacaaggggaacccagtctaatgaggagcaggacaaaaggttcatctgcaaacactccatggtagacagaggatggggaaatggatgtggattatttggaaagggaggcattgtgacctgtgctatgtttacatgcaaaaagaacatggaaggaaaagtcgtgcagccagaaaatttggaatacaccatcgtgataacacctcactcaggagaggagcacgctgtaggtaatgacacaggaaagcatggcaaggaaatcaaaataacaccacagagctccatcacagaagcagaactgacaggctatggcactgtcacgatggagtgctctccgagaacgggcctcgacttcaatgagatggtactgctgcagatggaagacaaagcttggctggtgcacaggcaatggttcctagacctgccgttaccatggctacccggagcggacacacaaggatcaaattggatacagaaagagacattggtcactttcaaaaatccccacgcgaagaaacaggacgtagtcgtactcggatcacaggaaggcgcaatgcataccgcattgacaggcgctacagagatacagatgtctagcggaaatctgttgtttacagggcatctgaaatgtagactgagaatggacaaactgcaattgaagggaatgtcatactctatgtgtacgggtaagtttaagatagtcaaagagatagccgaaacacaacacggaacaatcgtaattagggtgcaatacgaaggcgacgggtcaccatgtaagataccattcgaaattacagacctcgaaaaaagacacgtactcggaagactgataacagtgaatccgatcgttacggaaaaagactcacccgttaatatcgaagccgaaccaccattcggagactcatacataataatcggagtcgaacccggacaattgaaactgaattggtttaaaaaagggtcatcaatcggacaaatgttcgaaacaactatgagaggcgctaagcgtatggctatactcggagacacagcatgggacttcggatccttagggggagtgtttacgtcaatcggtaaggcactacaccaggtattcggagcgatatacggagccgcatttagcggagtgtcgtggacaatgaagatactgatcggagtgataatcacatggatcggaatgaatagtaggtctacaagtctatccgttagcttagtgttagtcggagtcgttacactgtatctaggcgctatggtgcaagccgatagtggttgcgttgtgagctggaaaaataaagaactgaaatgtggcagcgggatcttcatcacagataacgtacacacatggacagaacaatataagttccaaccagaatccccttcaaaactagcttcagctatccaaaaagctcatgaagagggcatttgtggaatccgctcagtaacaagattggagaatctgatgtggaaacaaataacaccagaattgaatcatattctatcagaaaatgaggtaaagttgaccattatgacaggagacattagaggaatcatgcaggcaggaaaacgatccttgcggccccagcccactgagctgaagtactcatggaaaacatggggaaaggcgaaaatgctctccacagagtctcacaatcagacctttcttattgatggccctgaaacagcagaatgccccaacacaaacagagcctggaactcgctggaagttgaagactatggttttggagttttcaccaccaatatatggctgaaattgagagaaaaacaggatgtattttgtgactcaaaactcatgtcagcggccattaaagacaacagagccgttcatgctgatatgggttattggatagaaagtgcactcaatgacacatggaagatggagaaagcctccttcattgaagttaaaagctgccactggccaaagtcacacaccctttggagcaatggagtattagaaagtgagatgataatcccaaaaaattttgccgggccagtgtcacaacacaactacagaccaggctaccatacacaaacagcaggaccttggcatctaggtaagcttgagatggactttgatctctgcgaaggaactacagtggtggtgactgaggactgtggaaatagaggaccctctttaagaacgaccactgcctctggaaaactcataacagaatggtgctgccgatcctgcacactaccacctctaagatacagaggtgaggatggatgctggtacgggatggaaatcagaccattgaaagagaaagaagagaatttggtcaactccttggtcacagccggacatgggcagattgacaacttttcactaggagtcttgggaatggcactgttcctggaagaaatgctcaggacccgaataggaacgaaacatgcaatactgctagttgcagtatcttttgtgacattgattactgggaacatgtcttttagagacctgggaagagtgatggttatggtgggcgctaccatgacggatgacataggtatgggagtgacttatcttgccctactagcagcttttaaggttagaccaacttttgcagctggactactcttaagaaaactgacctccaaggaattgatgatggccaccataggaatcgcactcctttcccaaagcaccataccagagaccattcttgaactgactgatgcattagccctgggcatgatggtcctcaaaatagtgagaaatatggaaaaataccaattggcagtgactatcatggctatttcatgtgtcccaaatgcagtgatactgcaaaacgcatggaaggtgagttgcacaatattggcagcggtgtccgtttcaccactgctcttaacatcctcacagcagaaagcggattggataccactggcattgacgataaaaggtctcaatccaacagccatttttttaacaactctctcgaggaccagcaagaaaaggagctggccgctaaatgaagctatcatggcagttgggatggtgagcattttagccagttctctcctaaagaatgatattcctatgacaggtccattagtggctggaggactcctcaccgtatgttacgtgctcactggacgatcggccgatttggaactggagagagctgccgatgtaaaatgggaagatcaggcagaaatatcaggaagcagcccaatcctgtcaataacaatatcagaagatggcagcatgtcgataaaaaatgaagaggaagaacaaacactgaccatactcatcagaacgggattgttggtgatctcaggagtctttccagtatcgataccaattacggcagcagcatggtacctgtgggaagtaaagaaacaacgggctggagtactgtgggacgtcccttcacccccaccagtggaaaaagccgaactggaggatggagcctacagaatcaagcaaagagggatccttggatattctcagattggagccggagtttacaaagaaggaacattccatacaatgtggcacgtcacacgtggtgctgttctgatgcatagagggaagaggattgaaccatcatgggcagatgtcaagaaagacctaatatcatatggaggaggctggaagctagaaggagaatggaaggaaggagaggaagtccaagtcctggcattggaacctggaaaaaatccaagagccgtccaaacgaaacctggaatattcaaaaccaacaccggaaccataggcgccgtatctctggacttttcccctggaacgtcaggatctccaatcgtcgacagaaaaggaaaagttgtgggtctttacggtaatggtgttgtcacaaggagtggagcatatgtaagtgctatagcccagaccgaaaaaagcattgaagacaatccagagatcgaagatgacattttccgaaagaaaagattgaccatcatggacctccatccaggagcaggaaagacaaaaagataccttccagccatagttagagaagccataaaacgtggcttgagaacattgatcctggctcccactagagtagtggcagctgaaatggaggaagctcttagaggacttccaataagataccaaactacagccatcaaaaccgagcataccgggcgggagatcgtggacctaatgtgtcatgccacatttactatgaggctgttatcaccagtcagagtgccaaattacaacctgatcatcatggacgaagcccacttcacagacccagcaagtatagcagctagaggatacatttcaactcgagtagagatgggtgaagcagccgggattttcatgacagccactcctccgggaagtagagacccatttcctcagagcaatgcaccaattatggatgaggaaagagaaatccctgagcgttcatggaattcaggacacgaatgggtcacggattttaaggggaagactgtttggtttgttccaagtataaaagcaggaaatgatatagcagcttgtcttaggaaaaatggaaagaaagtgatacaactcagtaggaagacttttgactctgagtatgttaagactagagccaatgattgggactttgtggtcacaactgacatttcagaaatgggtgccaacttcaaggctgagagggttatagaccccagacgttgcatgaaaccagttatactaacagatggcgaggagcgggtgatcttggctggacctatgccagtgacccactctagtgcagcgcaaagaagagggagaataggaagaaatccaaaaaatgaaaatgaccagtacatatacatgggggaacctcttgaaaatgatgaagactgtgcacattggaaagaagctaaaatgctcctagataacatcaacacacctgaaggaatcattcctagtatgttcgaaccagagcgtgaaaaagtggatgccattgatggtgaataccgtttgagaggagaagcaaggaaaacctttgtggacctaatgagaagaggggacttaccagtctggttggcctacaaagtggcagctgaaggcatcaactacgcagacagaaagtggtgttttgatggaattaagaacaaccaaatactggaagaaaatatggaagtggaaatctggacaaaagaaggggaaaggaaaaaattaaaacccagatggttggatgctaggatctattctgacccactagcactaaaagaattcaaggaatttgcagctggaagaaaatctttgaccctgaacctaatcacagaaatgggtaggcttccaactttcatgactcagaaagcaagaaacgcactggacaacctggctgtgctgcatacggctgaggcaggtggaagggcgtacaatcatgctctcagtgaactgccggagaccctggagacactgctcctactgacacttctggcaacagtcacaggaggaatcttcttattcttaatgagcggaaaaggtatagggaagatgaccctgggaatgtgttgcataatcacggctagtatcctcctatggtatgcacagatacaaccacactggatagcagcttcaataatactggagttttttctcatagttttgctcattccagaaccagaaaaacagagaacaccccaagacaaccaattgacctacgttgtcatagccatcctcacagtggtggccgcaaccatggcaaacgagatgggtttcctggaaaaaaccaagaaagacctcggattgggaagcattacaacccaggaatctgagagcaatatcctggacatagatctacgccctgcatcagcatggacgctgtatgccgtagctacaacatttgtcacaccaatgttgagacatagcattgaaaattcctcagtgaatgtctccctaacagccattgctaaccaagctacagtgctaatgggtcttgggaaaggatggccattgtcaaagatggacattggagttcccctccttgccattggatgctattcacaagtcaaccctataactctcacagcagctctccttttattggtagcacattatgccattatagggccaggacttcaagcaaaagcaaccagagaagcccagaaaagagcagcagcaggcatcatgaaaaacccaacagtcgatggaataacagtgattgacctagaaccaataccctatgatccaaaatttgaaaagcagttaggacaagtaatgctcctaatcctctgcgtgactcaagtattaatgatgaggactacatgggctttatgtgaggctctaaccctagcgaccgggcccatctccacactgtgggaaggaaatccagggaggttttggaacactaccattgcagtgtcaatggctaacatctttagggggagctacttggccggagctggacttctcttttccatcatgaaaaacacaacaaacacaagaagaggaactggcaacataggagagacacttggagaaaaatggaaaagtcgattaaacgcactggggaaaagtgaatttcaaatctacaagaaaagtggaatccaggaagtggatagaaccctagcaaaagaaggtatcaaaagaggagaaacggaccaccatgctgtgtcgcgaggttcagcaaaactgagatggttcgtcgagagaaatatggtcacaccggaagggaaggtggtggatctcggttgcggcagagggggctggtcatactattgcgggggactaaagaatgtaagagaagtcaaaggcctaacaaaaggaggaccaggacatgaagaacccatccccatgtcaacatatgggtggaatctagtgcgtctgcaaagtggagttgacgttttcttcaccccgccagaaaagtgtgatacattgttgtgtgacataggggagtcgtcaccaaatcccacgatagaagcaggacgaacactcagagtcctcaacttagtggaaaattggttgaacaataacacccaattttgcataaaggtcctcaacccatatatgccttcagtcatagaaaaaatggaagcattacaaaggaaatatggaggagccttagtgaggaatccactctcacgaaactccacgcatgaaatgtactgggtatctaatgccaccgggaacatagtgtcatcagtgaacatgatttcaaggatgttgattaacagattcacaatgaaacacaagaaagccacttacgagccagatgttgacctaggaagtggaacccgcaacattggaattgaaagtgagataccaaatctagacataataggaaagagaatagagaaaataaaacaagagcatgaaacatcatggcactatgatcaagaccacccatacaaaacgtgggcttaccatggcagctatgaaacaaaacaaactggatcagcatcatctatggtgaacggagtggtcagattgctgacaaaaccttgggacgtcgtccctatggtgacacagatggcaatgacagacacgactccttttggacaacagcgcgttttcaaagagaaagtagacacgagaacccaagaaccgaaggaaggcacaaagaaactgatgaaaattacggcagagtggctttggaaagaactaggaaagaaaaagacacctaggatgtgtaccagagaagaattcacaagaaaagtgagaagcaatgcagccttgggggccatattcactgatgagaacaaatggaaatcggcacgtgaggctgttgaagatggtaggttctgggagctggttgacagggaaagaaatctccatcttgaaggaaagtgtgaaacatgtgtgtacaacatgatgggaaaaagagagaagaaactaggggagttcggcaaggcaaaaggtagcagagccatatggtacatgtggcttggagcacgcttcttagagtttgaagccctaggattcctgaatgaagatcactggttctccagagggaactccctgagtggagtggaaggagaagggctgcacaggctaggctacattttaagagacgtgggcaagaaggaagggggggcaatgtacgccgatgatacagcaggatgggacacaagaatcacactagaagacttaaaaaatgaagaaatggtaacgaaccacatgaaaggagaacacaagaaactagccgaggccatattcaaactaacgtaccaaaacaaggtggtgcgtgtgcaaagaccaacaccaagaggtacagtaatggatatcatatcgagaagagaccaaagaggcagtgggcaagtcggcacctatggccttaataccttcaccaatatggaagcccaattaattagacagatggagggagaaggaatcttcaaaagcattcagcacctgacagccacagaagaaatcgctgtacagaactggctagcaagagtggggcgtgaaaggctatcaagaatggcaatcagtggagatgattgtgttgtaaaacctatagatgacagatttgcaagtgctttaacagctctaaatgacatgggaaaagttagaaaagatatacaacaatgggaaccttcaagaggatggaacgattggacacaagtgcctttctgttcacaccactttcatgagttagtcatgaaagatggtcgcgtgctcgtagtcccatgcagaaaccaagatgaactgattggtagagcccgaatttcccagggagctgggtggtctttgaaagagacggcctgtttgggaaagtcttacgcccaaatgtggactctgatgtacttccacagacgtgacctcagactggcggcaaatgccatctgctcggcagtcccgtcacattgggttccaacaagtcgaacaacctggtccatacacgctaagcatgaatggatgacgacggaagacatgctggcagtctggaacagggtgtggatccaagaaaacccgtggatggaagataaaactccagtggaatcatgggaagaagtcccatacttgggaaaaagagaagaccaatggtgcggctcattgattgggctaacaagcagggctacctgggcaaagaacatccaaacagcaataaatcaagtcagatcccttataggcaatgaggaatacacagactacatgccatccatgaagagatttagaagggaagaggaagaggcaggtgtcctgtggtagaaggcaaaactaacatgaaacaaggctaaaagtcaggtcggattaagccatagtacggaaaaaactatgctacctgtgagccccgtccaaggacgttaaaagaagtcaggccatcacaaaatgccacagcttgagtaaactgtgcagcctgtagctccacctgaggaggtgtaaaaaacctgggaggccacaaaccatggaagctgtacgcatggcgtagtggactagcggttagaggagacccctcccttacaaatcgcagcaaacaacgggggcccaaggtgagatgaagctgtaatctcactggaaggactagaggttagaggagacccccccaaaacaaaaaacagcatattgacgctgggaaagaccagagatcctgctgtctcctcagcatcattccaggcacagaacgccagaaaatggaatggtgctgttgaatcaacaggttct

SEQ ID NO: 11 - DNA, 10708 bp, Dengue virus
agttgttagtctacgtggaccgacaagaacagtttcgactcggaagcttgcttaacgtagtgctaacagttttttattagagagcagatctctgatgaacaaccaacggaagaagacgggaaaaccgtctatcaatatgctgaaacgcgtgagaaaccgtgtgtcaactggatcacagttggcgaagagattctcaaaaggactgctgaacggccagggaccaatgaaattggttatggcgttcatagctttcctcagatttctagccattccaccaacagcaggagtcttggctagatggggaaccttcaagaagtcgggggccattaaggtcctgaaaggctttaagaaggagatctcaaacatgctgagcataatcaacaaacggaaaaagacatcgctctgtctcatgatgatattgccagcagcacttgctttccacttgacttcacgagatggagagccgcgcatgattgtggggaagaatgaaagaggaaaatccctactttttaagacagcctctggaattaacatgtgcacactcatagccatggacttgggagagatgtgtgatgacacggtcacttacaaatgcccccatattaccgaagtggaacctgaagacattgactgctggtgcaacctcacatcaacatgggtgacttatggaacgtgcaatcaagccggagagcatagacgcgataagagatcagtggcgttagctccccatgtcggcatgggactggatacacgcacccaaacttggatgtcggctgaaggagcttggaggcaagtcgagaaggtagagacatgggcccttaggcacccagggttcaccatactagccctatttcttgcccattacataggcacttccttgacccagaaggtggttatttttatactgctaatgctggtcaccccatccatgacaatgagatgtgtgggagtaggaaacagagattttgtggaaggactatcaggagctacgtgggttgacgtggtgctcgagcacggggggtgtgtgaccaccatggctaagaacaagcccacgttggatatagagcttcagaagaccgaggccacccaactggcgaccctaaggaagctatgcattgaggggaaaatcaccaacataacaactgactcaagatgtcctacccaaggggaagcggttttgcctgaggagcaggaccaaaactacgtgtgtaagcatacatacgtagacagaggctgggggaacggatgtggtttgtttggtaagggaagcttggtaacatgtgcgaaatttcaatgcctggaaccaatagagggaaaagtggtgcaatatgagaacctcaaatacaccgttatcatcacagtgcacacaggagaccaacaccaggtgggaaatgaaacgcagggagtcacggctgagataacacctcaggcatcaaccactgaagccatcttgcctgaatatggaacccttgggctagaatgctcaccacggacaggtttggacttcaatgaaatgatcttgctaacaatgaagaacaaagcatggatggtacatagacaatggttttttgacctacctctaccatggacatcaggagctacaacggaaacaccaacctggaacaggaaggagcttcttgtgacatttaaaaacgcacatgcgaagaaacaagaagtagtcgttttagggtcacaggagggagctatgcataccgcactaaccggagcgactgagatacagaatagcggagggacatcaatcttcgccggacaccttaagtgtagattgaaaatggacaaacttgagcttaaggggatgtcatacgctatgtgtacgaacacattcgtgctgaaaaaagaggtaagcgaaacgcaacacggaacgatactgattaaggttgagtataagggcgaagacgtcccatgtaagatacctttttcgacagaggacggacagggtaaggcccataacggaagactgattaccgctaacccagtggtgaccaaaaaagaggaaccagtcaatatcgaagccgaacctccattcggagagtcaaacatagtgatagggataggcgataacgcacttaagattaactggtacaaaaaagggtcatcaatcggtaagatgtttgaggcaaccgctaggggggctagacggatggccatactcggagacaccgcatgggacttcggatccgtggggggggtacttaactcactcggtaagatggtgcaccaaattttcggatccgcatataccgctctatttagcggagtgtcatgggtgatgaaaatcggaatcggagttctattgacatggatcggattgaactctaagaatacatctatgtcattttcatgtatcgcaatcggaattattacgctatatctcggagccgtagtgcaagccgacatggggtgtgttataaactggaaaggcaaagaactcaagtgtggaagtggaatcttcgtcaccaacgaggtccatacctggacagagcaatacaaattccaagcagactccccaaaaagattggcgacagccattgcaggcgcttgggagaatggagtgtgcggaattaggtcaacaaccagaatggagaatctcctgtggaagcaaatagccaatgaactgaactacatattgtgggaaaacaatatcaaattaacggttgttgtgggcgatataattggggtcttagagcaagggaaaagaacactaacaccacaacccatggagctaaaatactcatggaaaacgtggggaaaggcaaaaatagtgacagctgaaacacaaaattcttctttcataatagatggaccaaacacaccggagtgtccaagtgcctcaagagcatggaatgtgtgggaggtggaagattacgggttcggagtcttcacaaccaacatatggctgaaactccgagaggtgtatacccaactatgtgaccataggttaatgtcggcagccgtcaaggatgaaagggccgtacatgccgacatgggctattggatagaaagtcaaaagaatggaagttggaagctagaaaaagcatccctcatagaggtgaaaacctgcacatggccaaaatcacataccctttggagtaatggtgtgttagagagtgacatgatcattccaaaaagtctagctggtcctatctcgcaacacaactacaggcccgggtaccacacccagacggcgggaccttggcatttaggaaaattagagctggacttcaactattgtgaaggaacaacagttgtcatcacagaaaactgtgggacaagaggcccatcattgagaacaacaacagtgtcagggaagttaatacacgaatggtgctgccgctcgtgcacacttcctcccctgcgatacatgggagaagacggttgctggtatggcatggaaatcagacccatcagtgagaaagaagaaaacatggtaaagtctttagtctcagcgggaagtggagaggtggacaacttcacaatgggtgtcttgtgtttggcaatcctctttgaagaggtgatgagaggaaaatttgggaagaaacacatgattgcgggggttttcttcacgtttgtactccttctctcagggcaaataacatggagagatatggcgcacacactaataatgattgggtccaatgcatctgacaggatgggaatgggcgtcacctacctagctttaattgcaacatttaaaatccagccatttttggctttgggatttttcctaagaaaactgacatccagagaaaatttattgttaggagttgggctggctatggcaacaacgttacaactgccagaggacattgaacaaatggcaaatggaatcgctctggggctcatggctctcaaactgataacacaatttgaaacataccaactatggacagcattaatctccttaacgtgttcaaatacaatttttacgttgactgttgcctggagaacagccaccctgattttggctggagtttcacttttaccagtgtgccagtcttcgagtatgaggaaaacagactggcttccaatgacagtggcagctatgggagttccacctctaccactttttatttttagcttaaaagacacactcaaaaggagaagctggccactgaatgaaggggtgatggctgttgggcttgtgagcattctggccagttctctccttagaaatgatgtacccatggctggaccattagtggccgggggcttgctgatagcgtgctacgtcataactggcacgtcagcagacctcaccgtagaaaaagcagcagatgtaacatgggaggaagaggctgagcaaacaggagtgtcccacaacttaatgatcacagttgatgatgatggaacaatgagaataaaagatgatgagactgagaatatcttaacagtgcttttgaaaacagcattactaatagtgtcaggaatctttccatactccatacccgcaacattgttggtctggcatacttggcaaaagcaaacccaaagatccggcgttctatgggatgtacccagccctccagagacacagaaagcagaactggaagaaggggtctataggatcaaacagcaaggaatttttgggaaaacccaagtaggggttggagtacagaaagaaggagtctttcacaccatgtggcacgttacaagaggggcagtgttgacatataatgggaaaagactggaaccaaattgggctagcgtgaaaaaagatctgatttcatacggaggaggatggagattgagcgcacaatggcaaaagggggaggaggtgcaggttattgccgtagagcctgggaagaacccaaagaactttcaaaccatgccaggcacttttcagactacaacaggggaaataggagcaattgcactggatttcaagcccggaacttcaggatctcctatcataaacagagagggaaaggtagtgggactgtatggcaatggagtggttacaaagaatggtggctacgtcagcggaatagcgcaaacgaatgcagaaccagatggaccgacaccagaattggaagaagagatgttcaaaaagcgaaatctaaccataatggatcttcatcctgggtcaggaaagacacggaaataccttccagctattgttagagaggcaatcaagagacgtttgagaactctaattctggcaccaacaagggtggttgcagctgagatggaagaagcattgaaagggctcccaataaggtaccaaacaacagcaacaaaatctgaacacacaggaagagagattgttgatctgatgtgccacgcaacgttcacaatgcgtctgctgtcaccagttagggttccaaactataacttgataataatggatgaagcccatttcacagacccagccagtatagctgctagagggtacatatcaactcgtgttggaatgggagaagcagccgcaatattcatgacagcaacgccccctggaacagctgatgcctttccccagagcaacgctccaattcaagatgaagaaagggacataccagaacgctcatggaattcaggcaatgaatggataaccgacttcgctgggaaaacggtgtggtttgtccccagcattaaagccggaaatgacatagcaaattgcctgcggaaaaacgggaaaaaggtcattcaacttagtaggaagacttttgacacagaatatcagaaaaccaaactgaatgattgggactttgtggtgacgactgacatttcagaaatgggggccaatttcaaagcagatagagtgatcgacccaagaagatgtctcaaaccagtgatcctgacagatggaccagagcgggtgatcctggctggaccaatgccagtcaccgcagcgagtgctgcgcaaaggagagggagagttggcaggaacccacaaaaagaaaatgaccagtacatattcacgggccagcctctcaacaatgatgaagaccatgctcactggacagaagcaaaaatgctgctggacaacattaacacaccagaagggatcataccagctctctttgagccagaaagggagaagtcagccgccatagacggtgagtatcgcctgaaaggtgagtccaggaagactttcgtggaactcatgaggaggggtgaccttccagtctggttagcccataaagtagcatcagaagggatcaaatatacagatagaaaatggtgctttgatggacaacgtaataatcaaattttagaagagaacatggatgtggaaatctggacaaaggaaggagaaaagaaaaaattgagacctaggtggcttgatgctcgcacctattcagatcccttagcactcaaggaattcaaggactttgcggctggcaggaagtcaatagcccttgatcttgtgacagaaataggaagagtgccttcacacctagctcatagaacgagaaacgctctggacaatctggtgatgctgcatacgtcagaacatggcggtaaggcctacaggcatgcggtggaggaactaccagagacaatggaaacactcctactcttgggactcatgatcttgttgacaggtggagcaatgcttttcttaatatcaggtaaagggattggaaagacttcaataggactcatttgtgtaattgcttccagcggcatgttgtggatggccgaaatcccactccaatggatcgcgtcggccatagtcctggagttttttatgatggtgttgcttataccagaaccagagaagcagagaaccccccaagacaaccaactcgcatatgtcgtgataggcatacttacactggcagcaataatagcagccaatgaaatgggattgttggaaactacaaagagagatttaggaatgtctaaggagccaggtgttgtttctccaaccagctatttagatgtggacttgcacccagcatcagcctggacattgtacgctgtggccactacagtaataacaccaatgttaagacataccatagagaattccacagcaaatgtgtccttggcagctatagccaaccaggcagtggtcctgatgggtttggacaaaggatggccaatatcaaaaatggacttaggagtacccctgctggcattgggttgctattcacaagtgaacccactgactctaacagcggcagtactcttgctgatcacacattatgccattataggtccaggattgcaggcaaaagccacccgtgaagctcagaaaaggacagctgctggaataatgaagaatccaacagtggatgggataatgacaatagacctagatcctgtaatatatgattcaaaatttgaaaagcaactgggacaggttatgctcctggttttgtgtgcagttcaacttttgttaatgagaacatcatgggccttgtgtgaagctttaaccctagctacaggaccaataacaacactctgggaaggatcacctgggaagttttggaacaccacgatagctgtttccatggcgaacatttttagagggagctatttagcaggagctgggcttgctttctctattatgaaatcagttggaacagggaaaagaggaacaggctcacagggtgaaactttgggagaaaaatggaaaaagaagttaaatcaattatcccggaaagagtttgacctttacaagaaatctggaatcactgaggtggatagaacagaagccaaagaagggttgaaaagaggagaaataacacatcatgccgtgtccagaggtagtgcaaaacttcaatggtttgtggagagaaacatggtcattcccgaaggaagagttatagacttgggctgtggaagaggaggctggtcatattactgtgcaggactgaaaaaagtcacagaagtgcgaggatacacaaaaggcggtccaggacacgaagaaccagtacctatgtccacatatggatggaacatagttaagttaatgagtggaaaggatgtgttttatcttccacctgaaaagtgtgacaccctgttgtgcgacattggagaatcttcaccaagcccaacagtggaagaaagcagaactataagagttttgaagatggttgaaccatggctaaagaacaaccaattttgtattaaagtattgaacccttacatgccaactgtgattgagcacctagaaagactacaaaggaaacatggaggaatgcttgtgagaaatccactttcacgaaactccacgcacgaaatgtactggatatccaatggcacaggtaacattgtcgcttcagtcaacatggtatctagactgctactgaacaggttcacgatgacacacagaagacccaccattgagaaagatgtggatttaggagcaggaactcgacatgttaatgcggaaccagaaacacccaacatggatgtcattggggaaagaataaaaaggatcaaggaggagcataattcaacatggcactacgatgacgaaaacccctacaaaacgtgggcttaccatggatcttatgaagtcaaagccacaggctcagcctcctccatgataaatggagtcgtgaaactcctcactaaaccatgggatgtggtgcccatggtgacacagatggcaatgacagatacaactccatttggccagcagagagtctttaaagagaaagtggacaccaggacacccaggtccatgccaggaacaagaagggttatggggatcacagcggagtggctctggagaaccctgggaaggaataaaaaacccaggttatgcacaagggaagagtttacaaaaaaggtcagaactaacgcagccatgggagctgttttcacagaggagaaccaatgggacagcgcgaaagctgctgttgaggatgaggatttttggaaacttgtggacagagaacgtgaactccacaaactgggcaagtgtggaagctgtgtttacaacatgatgggtaagagagagaagaaacttggagagtttggcaaagcaaaaggcagtagagctatatggtacatgtggttgggagccaggtaccttgagttcgaagcccttggattcttaaatgaagaccactggttctcgcgtgagaactcttacagtggagtggaaggagaaggactgcacaagctaggctatatattaagggacatttccaagatacccggaggagctatgtatgctgatgacacagctggttgggacacaagaataacagaagatgacctgcacaatgaggaaaagatcacacagcaaatggaccctgaacacaggcagttagcgaacgctatatttaagctcacataccaaaacaaagtggtcaaagttcaacgaccgactccaacaggcacggtaatggatatcatatctaggaaagaccaaagaggcagtggacaggtaggaacttatggtctgaatacattcaccaacatggaagcccagttaatcagacaaatggaaggagaaggtgtgctgtcaaaggcagacctcgagaaccctcatctgccagagaagaaaattacacaatggttggaaaccaaaggagtggagaggttaaaaagaatggccataagtggggatgactgcgtggtgaaaccaatcgatgacaggttcgctaatgccctgctcgctctgaacgacatgggaaaggttcggaaagacatacctcaatggcagccatcaaagggatggcatgattggcaacaggttcctttctgctcccaccactttcatgaattgatcatgaaagatggaagaaagttggtggttccctgcagaccccaggacgaactaataggaagggcaagaatctctcaaggagcgggatggagccttagagaaaccgcatgtctggggaaagcctacgctcaaatgtggagtctcatgtattttcacagaagagacctcagactagcatccaacgccatatgttcagcagtaccagtccactgggtccccacaagtagaacgacatggtctattcacgctcaccatcagtggatgaccacagaagacatgcttactgtctggaacagagtgtggatcgaggacaatccatggatggaagacaaaactccagtcacaacctgggaaaatgttccatatctagggaagagagaagaccaatggtgcggatcacttattggtctcacttccagagcaacctgggcccagaacatacccacagcaattcaacaggttagaagccttataggcaatgaagagtttctggactacatgccttcaatgaagagatttaggaaggaggaggagtcggagggagccatttggtaaaacgtaggaagtgaaaaagaggttaactgtcaggccatattaagccacagtacggaagaagctgtgctgcctgtgagccccgtccaaggacgttaaaagaagaagtcaggccccaaagccacggtttgagcaaaccgtgctgcctgtagctccgtcgtggggacgtaaaacctgggaggctgcaaactgtggaagctgtacgcacggtgtagcagactagcggttagaggagacccctcccatgacacaacgcagcagcggggcccgagcactgagggaagctgtacctccttgcaaaggactagaggttagaggagaccccccgcaaataaaaacagcatattgacgctgggagagaccagagatcctgctgtctcctcagcatcattccaggcacagaacgccagaaaatggaatggtgctgttgaatcaacaggttct

SEQ ID NO: 12 - DNA, 10649 bp, Dengue virus
agttgttagtctgtgtggaccgacaaggacagttccaaatcggaagcttgcttaacacagttctaacagtttgtttaaatagagagcagatctctggaaaaatgaaccaacgaaaaaaggtggttagaccacctttcaatatgctgaaacgcgagagaaaccgcgtatcaacccctcaagggttggtgaagagattctcaaccggacttttttctgggaaaggacccttacggatggtgctagcattcatcacgtttttgcgagtcctttccatcccaccaacagcagggattctgaagagatggggacagttgaagaaaaataaggccatcaagatactgattggattcaggaaggagataggccgcatgctgaacatcttgaacgggagaaaaaggtcaacgataacattgttgtgcttgattcccaccgtaatggcgtttcacttgtcaacaagagatggcgaacccctcatgatagtggcaaaacatgaaagggggagacctctcttgtttaagacaacagaggggatcaacaaatgcactctcattgccatggacttgggtgaaatgtgtgaggacactgtcacgtataaatgccccctactggtcaataccgaacctgaagacattgattgctggtgcaacctcacgtctacctgggtcatgtatgggacatgcacccagagcggagaacggagacgagagaagcgctcagtagctttgacaccacattcaggaatgggattggaaacaagagctgagacatggatgtcatcggaaggggcttggaagcatgctcagagagtagagagctggatactcagaaacccaggatttgcgctcttggcaggatttatggcttatatgattgggcaaacaggaatccagcgaactgtcttctttgtcctaatgatgctggtcgccccatcctacggaatgcggtgcgtaggagtaggaaacagagactttgtggaaggagtctcaggtggagcatgggtcgacctggtgctagaacatggaggatgcgtcacaaccatggcccagggaaaaccaaccttggattttgaactgactaagacaacagctaaggaagtggctctgttaagaacctattgcattgaagcctcaatatcaaacataactacggcaacaagatgtccaacgcaaggagagccttatctgaaagaggaacaggaccaacagtacatctgccggagagatgtggtagacagaggatggggcaatggctgtggcttgtttggaaaaggaggagttgtgacatgtgcgaagttttcatgttcggggaagataacaggcaatctggtccaaattgagaaccttgaatacacagtggttgtaacagtccacaatggagacacccatgcagtaggaaatgacacatccaatcatggagttacagccacgataactcccaggtcaccatcggtagaagtcaaattgccggactatggagaactaacactcgattgtgaacccaggtctggaattgactttaatgagatgatcctaatgaaaatgaaaaagaaaacatggctcgtgcataagcaatggtttttggatctgcctcttccatggacagcaggagcagacacatcagaggttcactggaattacaaagagagaatggtgacgttcaaggttcctcatgccaagagacaggacgttacagtgttagggtcacaggagggagctatgcatagcgcactagccggagcaaccgaagtcgatagcggagacggaaatcatatgttcgccggacatctgaaatgcaaagtgagaatggagaaattgcggattaagggaatgtcatacactatgtgttccggtaagttttcgattgacaaagagatggccgaaacgcaacacggaacaacagtcgttaaggttaagtacgaaggagccggagctccgtgtaaagtgccaatcgaaattagagacgttaacaaagagaaagtggtcggaagagtgatatctagtacacccctagccgaaaatacgaattccgttacgaatatcgaactcgaacccccctttggagactcatacatagtgataggagtgggtaattccgcactcacgttgcactggttcagaaagggatcatcaatcgggaagatgttcgaatcaacatataggggagcgaaaagaatggctatattgggcgaaaccgcatgggacttcggatcagtcggaggactgtttacgagtctgggtaaggccgtacatcaggtattcggatcagtgtatacaacaatgtttggcggagtgtcatggatgatacggatactgatagggtttctagtgttgtggatcggaacgaactcacgtaatacgtctatggctatgacatgtattgccgtaggggggattacactgtttctgggatttacagtgcaagcagacatgggttgtgtggtgtcatggagtgggagagaattgaagtgtggaagcggaatttttgtggttgacaacgtgcacacttggacagaacagtacaaattccaaccagagtccccagcgagactagcgtctgcaatattaaatgcccacaaagatggggtctgtggaattagatcaaccacgaggctggaaaatgttatgtggaagcaaataaccaatgagctaaactatgttctctgggaaggaggacatgatctcactgtagtggctggggatgtgaaaggggtgttgaccaagggcaagagagcactcacacccccagcgagtgatctgaaatattcatggaagacatgggggaaagcaaaaatcttcacccctgaagcaagaaacagcacatttttaatagacggaccagacacctctgaatgccccaatgaacgaagggcatggaattcttttgaggtggaagactatggatttggcatgttcacgaccaacatatggatgaaattccgagaaggaagttcagaagtgtgtgaccacaggttaatgtcagctgcaattaaagaccagaaagctgtgcatgctgacatgggttattggatagagagctcaaaaaaccagacctggcagatagagagagcatctcttattgaagtgaaaacatgtctgtggcccaagacccatacactgtggagcaatggagtgctggaaagccagatgcttattccaaaatcatatgcaggccctttttcacagcacaattaccgccagggctatgctacgcaaaccgtgggtccatggcacttaggcaaactagagatagactttggagaatgccccggaacaacagtcacaattcaggagaattgtgaccatagaggcccatctttgaggaccaccactgcatctggaaaactagtcacgcaatggtgttgccgctcctgcacgatgccccccttaaggttcttaggagaagatgggtgctggtatgggatggagattaggcccttgagtgaaaaagaagagaacatggtcaaatcacaggtgacggccggacagggcacatcggaaactttttcaatgggtctgttgtgcctgaccttgtttgtggaagaatgcttgaggagaagagtcaccaggaaacacatgatattagctgtggtaatcactctttgtgctatcatcctggggggcctcacatggatggacttgctacgagccctcatcatgttgggggacactatgtctggtagaataggaggacagacccacctagccatcatggcagtgttcaagatgtcaccgggatacgtgctgggtgtgtttttaaggaaactcacttcaagagagacagcactaatggtaataggaatggccatgacaacaacactttcaattccacatgacctcatggaactcattgatggaatatcactaggactaattttgctaaaaatagtaacacagtttgacaacacccaagtgggaaccttagctctttccttgactttcataagatcaacaatgtcattggtcatggcttggaggaccattatggctgtgttgtttgtggtcacactcattcctttgtgcaggacaagctgtcttcaaaaacagtctcattgggtagaaataacagcactcatcctaggagcccaagctctgccagtgtacctaatgactcttatgaaaggagcctcaagaagatcttggcctcttaacgaaggcataatggctgtgggtttggttagtctcttaggaagcgctcttttaaagaatgatgtccctttagctggcccaatggtggcaggaggcttacttctggcggcttacgtaatgagtggcagctcagcagatctgtcactagagaaggccgctaatgtgcagtgggatgaaatggcagacataacaggctcaagtccaatcatagaagtgaagcaagatgaggatggctctttctccatacgggacgtcgaggaaaccaatatgataacccttttggtgaaactggcactgataacggtgtcaggtctctaccccttggcaattccaatcacaatgaccttatggtacatgtggcaagtgaaaacacaaagatcaggagccctgtgggacgtcccttcacccgccgccactcaaaaagccgcactgtctgaaggagtgtacaggatcatgcaaagagggttattcgggaaaactcaggttggagtagggatacacatggaaggtgtatttcacacaatgtggcatgttacaagaggatcggtgatctgccacgagactgggagattggagccatcttgggctgatgtcaggaatgacatgatatcatacggtgggggatggaggcttggagataaatgggacaaagaagaagacgttcaggtcctcgctatagaaccagggaaaaatcccaaacatgtccaaacgaaacctggccttttcaagaccctaactggagaaattggagcagtaacattagatttcaaacccggaacgtctggttctcccattatcaacaggaaaggaaaagtcatcggactctatggaaatggagtggtcaccaaatcaggtgattacgtcagtgccataacacaagccgaaagaattggagagccagattatgaagtggatgaggacatttttcgaaagaaaagactaactataatggacttacaccccggagccggaaagacaaaaagaattcttccatcaatagtgagagaagccttaaaaaggaggctgcgaactttgattttggctcccacgagagtggtggcggccgagatggaagaggccctacgtggactgccaatccgttaccaaaccccagctgtaaaatcagaacacacaggaagagagattgtagacctcatgtgccatgcaaccttcacaacaagacttttgtcatcaaccagagttccaaactacaaccttatagtaatggatgaagcacatttcaccgatccttccagtgtcgcggctagaggatacatttcgaccagggtggaaatgggagaggcagcagccatcttcatgaccgcaacccctcccggagcgacagatccctttccccagagcaacagcccaatagaagacatcgagagagagattccggaaaggtcatggaacacagggttcgactggataacagactaccaagggaaaactgtgtggtttgttcccagcataaaagctggaaatgacattgcaaattgtttgagaaagtcgggaaagaaagttatccagttgagtaggaaaacctttgatacagaatatccaaaaacgaaactcacggactgggactttgtggtcactacagacatatctgaaatgggggctaactttagagctgggagagtgatagaccctagaagatgcctcaagccagttatcctaacagatgggccagagagagtcatcttagcaggtcctattccagtgactccagcaagcgctgctcagagaagagggcgaataggaaggaacccagcacaagaagacgaccaatacgttttctccggagacccactaaaaaatgatgaagaccatgcccactggacagaagcaaagatgctgcttgacaatatctacaccccagaagggatcattccaacattgtttggtccggaaagggaaaaaacccaagctattgatggagagtttcgcctcagaggggaacaaaggaagacttttgtggaattaatgaggagaggagaccttccggtgtggctgagttataaggtagcttctgctggcatttcttacaaagatcgggaatggtgctttactggggaaagaaataaccaaattttagaagaaaacatggaggttgaaatttggactagagagggagaaaagaaaaaactgaggccaaaatggttagatgcacgtgtatacgctgaccccatggctttgaaggatttcaaggagtttgccagtggaaggaagagtataactctcgacatcctaacagagatcgccagtttgccaacttacctttcctctagggccaagctcgcccttgacaacatagttatgctccacacaacagaaagaggagggagggcctatcaacatgccctgaacgaacttccggagtcactggaaacactcatgcttgtagccttactaggagctatgacagcaggtatcttcctgtttttcatgcaagggaaaggaatagggaaattgtcaatgggtttgataaccattgcggtggctagtggcttgctctgggtagcagaaattcaaccccagtggatagcggcctcaatcatactggagttttttctcatggtactgttgataccagaaccagaaaaacaaaggaccccacaagacaatcaattgatctacgtcatattgaccattctcaccattattggtctaatagcagccaacgagatggggctgatagaaaaaacaaaaacggattttgggttttaccaggtaaaaacagaaaccaccatcctcgatgtggacctgagaccagcttcagcatggacgctctatgcggtagccaccacaattctgactcccatgctgagacacaccatagaaaatacgtcggccaacctatctttagcagccatcgccaaccaggcagccgtcctaatggggcttggaaaaggatggccactccacagaatggacctcggtgtgccgctgttagcaatgggatgctattctcaagtgaacccaacaaccttgacagcatccttagtcatgcttttagtccattatgcaataataggcccaggattgcaggcaaaagccacaagagaggcccagaaaaggacagctgctggaatcatgaaaaatcccacagtggacgggataacagtgatagatctagaaccaatatcctatgacccaaaatttgaaaagcaattagggcaggtcatgttactcgtcttgtgtgctggacaactactcttgatgagaacaacatgggctttctgtgaagttttgactttggccacaggaccaatcttgaccttgtgggagggcaacccgggaaggttttggaacacgaccatagccgtatctaccgccaacattttcaggggaagttatttggcaggagctggactggctttttcactcataaagaatgcacaaacccccaggaggggaactgggaccacaggagagacactgggagagaagtggaagagacagctaaactcattagacaggaaagagtttgaagagtataaaagaagtggaatactagaagtggacaggactgaagccaagtccgccctgaaagatgggtctaaaatcaagcatgcagtatctagagggtccagtaagatcagatggattgttgagagagggatggtaaagccaaaggggaaagttgtagatcttggctgtgggagaggaggatggtcttattacatggcgacactcaagaacgtgactgaagtgaaagggtatacaaaaggaggtccaggacatgaagaaccgattcccatggctacttatggctggaatttggtcaaactccattcaggggttgacgtgttctacaaacccacagagcaagtggacaccctgctctgtgatattggggagtcatcttctaatccaacaatagaggaaggaagaacattaagagttttgaagatggtggagccatggctctcttcaaaacctgaattctgcatcaaagtcctcaacccctacatgccaacagtcatagaggagctggagaaactgcagagaaaacacggtgggaaccttgtcagatgcccgctgtccaggaactccacccatgagatgtattgggtgtcaggagcgtcgggaaatattgtgagctctgtgaacacaacatcaaagatgttgttgaacaggttcacaacaaggcataggaaacccacttatgagaaggacgtagatcttggggcaggaacgagaagtgtctctactgaaacagaaaaaccagacatgacaatcattgggagaaggcttcagcgattgcaagaggagcacaaagaaacatggcattatgatcaggaaaacccatacagaacctgggcgtatcatggaagctatgaagctccttcgacaggctctgcatcctccatggtgaacggggtggtgaaactgctaacaaaaccctgggatgtgattccaatggtgactcagttagccatgacagatacaaccccttttgggcaacaaagagtgttcaaagagaaggtggataccagaacgccgcaaccaaaaccaggcacacgaatggttatgaccacgacagccaattggctatgggccctccttggaaagaagaaaaatcccagactgtgtacaagggaagagttcatctcaaaagttagatcaaacgcagccataggcgcagtctttcaggaagaacaaggatggacatcagccagtgaagctgtgaatgacagccggttttgggaactggttgacaaagaaagggcccttcaccaggaagggaaatgtgaatcgtgtgtctataacatgatgggaaaacgtgagaaaaagttaggagagtttggcagagccaagggaagccgagcaatctggtacatgtggctgggagcgcggtttctggaatttgaagccctgggttttttgaatgaagatcactggtttggcagagaaaattcatggagtggagtggaaggggaaggtctgcacagattgggatacatcctggaggagatagacaagaaggatggagacctaatgtatgctgatgacacagcaggttgggacacaagaatcactgaggatgaccttcaaaatgaggaactgatcacggaacagatggctccccaccacaagatcctagccaaagccattttcaaactaacctatcaaaacaaagtggtgaaagtcctcagacccacaccgagaggagcggtgatggatatcatatccaggaaagaccaaagaggtagtggacaagttggaacatatggtttgaacacattcaccaacatggaagttcaactcatccgccaaatggaagctgaaggagtcatcacacaagatgacatgcagaacccaaaagggttgaaagaaagagttgagaaatggctgagagagtgtggtgtcgacaggttaaagaggatggcaatcagtggagacgattgcgtggtgaagcccctagatgagaggtttggcacttccctcctcttcttgaacgacatgggaaaggtgaggaaagacattccgcagtgggaaccatctaagggatggaaaaactggcaagaggttcctttttgctcccatcactttcacaagatctttatgaaggatggccgctcactagttgttccatgtagaaaccaggatgaactgatagggagagccagaatctcgcagggggctggatggagcttaagagaaacagcctgcctgggcaaagcttacgcccagatgtggtcgcttatgtacttccacagaagggatctgcgtttagcctccatggccatatgctcagcagttccaacggaatggtttccaacaagcagaacaacatggtcaatccatgctcatcaccaatggatgaccactgaagacatgctcaaagtgtggaacagagtgtggatagaagacaaccccaatatgattgacaagactccagtccattcgtgggaagatataccttacctagggaaaagagaggatttgtggtgtggatccctgattggactttcttctagagccacctgggcgaagaacattcacacggccataactcaggtcaggaacttgatcggaaaagaggaatacgtggattacatgccagtaatgaaaagatacagtgctccttcagagagtgaaggagttctgtaattaccaacaacaaacaccaaaggctattgaagtcaggccacttgtgccacggtttgagcaaaccgtgctgcctgtagctccgccaataatgggaggcgtaataatccctagggaggccatgcgccacggaagctgtacgcgtggcatattggactagcggttagaggagacccctcccatcactgacaaaacgcagcaaaagggggcccgaagccaggaggaagctgtactcctggtggaaggactagaggttagaggagacccccccaacacaaaaacagcatattgacgctgggaaagaccagagatcctgctgtctctacaacatcaatccaggcacagagcgccgcaagatggattggtgttgttgatccaacaggttct